SEQUENCE LISTING (163 sequences)

SEQ ID NO: 1 - protein, 569 aa, synthetic construct
  REGION 1..569: note = huIL-18RBeta-mFc
FNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRIEGRMDVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 2 - protein, 367 aa, synthetic construct
  REGION 1..367: note = huIL-18RBeta-HisAvi
FNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGSGGLNDIFEAQKIEWHETVRFQSHHHHHH

SEQ ID NO: 3 - protein, 367 aa, synthetic construct
  REGION 1..367: note = cyIL-18RBeta-HisAvi
FNISGCSTKKLLWAYSTRSEEEFILFCDLPEPQKSHFCHRSQLSPTQVPDHLPFTDSKDLPDVQWYRQPSNGDPLEDIRKSYPHIIQDKCTLRFLTPGVNNTGSYICRPKMIKSPYDVACCVKMILEVKPQSNASCENSASHKQDLLLGSTGSISCPSLSCQSDAQSPEVTWYKNGKLLSMTRSNRIVVDEVYIYHQGTYVCDYTQSDTASSWTVRAVVQVRTIVGDTKLKPDILDPVESTLEVELGKPLTISCKARFGFERVFNPVMKWYIKDADLEWEVSVPEAKSIKSTLKDEIIERNIILEKVTQRDLRRTFVCFVQNSIGNTTQSVQLKEKRGSGGLNDIFEAQKIEWHETVRFQSHHHHHH

SEQ ID NO: 4 - protein, 368 aa, synthetic construct
  REGION 1..368: note = muIL-18RBeta-HisAvi
FNHSACATKKLLWTYSARGAENFVLFCDLQELQEQKFSHASQLSPTQSPAHKPCSGSQKDLSDVQWYMQPRSGSPLEEISRNSPHMQSEGMLHILAPQTNSIWSYICRPRIRSPQDMACCIKTVLEVKPQRNVSCGNTAQDEQVLLLGSTGSIHCPSLSCQSDVQSPEMTWYKDGRLLPEHKKNPIEMADIYVFNQGLYVCDYTQSDNVSSWTVRAVVKVRTIGKDINVKPEILDPITDTLDVELGKPLTLPCRVQFGFQRLSKPVIKWYVKESTQEWEMSVFEEKRIQSTFKNEVIERTIFLREVTQRDLSRKFVCFAQNSIGNTTRTIRLRKKEEDGSGGLNDIFEAQKIEWHETVRFQSHHHHHH

SEQ ID NO: 5 - protein, 453 aa, synthetic construct
  REGION 1..453: note = heavy chain (aa) (P20)
QVQLQQSGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 214 aa, synthetic construct
  REGION 1..214: note = light chain (aa) (P20)
AIQLTQSPSSLSASVGDRVTITCRASQGISSALAWYQQKPGKAPKFLIYDASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 124 aa, synthetic construct
  REGION 1..124: note = VH region (aa) (P20)
QVQLQQSGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSS

SEQ ID NO: 8 - protein, 5 aa, synthetic construct
  REGION 1..5: note = CDR-H1 (Kabat) (P20, P20-V1, P20-V2, P20-V3)
NYYWS

SEQ ID NO: 9 - protein, 16 aa, synthetic construct
  REGION 1..16: note = CDR-H2 (Kabat) (P20, P20-V1, P20-V2, P20-V3)
YIFYSGTTNYNPSLKS

SEQ ID NO: 10 - protein, 16 aa, synthetic construct
  REGION 1..16: note = CDR-H3 (Kabat, Chothia, AbM) (P20, P20-V1, P20-V2, P20-V3)
DVGIAARNSYYYVMDV

SEQ ID NO: 11 - protein, 7 aa, synthetic construct
  REGION 1..7: note = CDR-H1 (Chothia) (P20, P20-V1, P20-V2, P20-V3)
GGSINNY

SEQ ID NO: 12 - protein, 5 aa, synthetic construct
  REGION 1..5: note = CDR-H2 (Chothia) (P20, P20-V1, P20-V2, P20-V3)
FYSGT

SEQ ID NO: 13 - protein, 10 aa, synthetic construct
  REGION 1..10: note = CDR-H1 (AbM) (P20, P20-V1, P20-V2, P20-V3)
GGSINNYYWS

SEQ ID NO: 14 - protein, 9 aa, synthetic construct
  REGION 1..9: note = CDR-H2 (AbM) (P20, P20-V1, P20-V2, P20-V3)
YIFYSGTTN

SEQ ID NO: 15 - protein, 6 aa, synthetic construct
  REGION 1..6: note = CDR-H1 (Contact) (P20, P20-V1, P20-V2, P20-V3)
NNYYWS

SEQ ID NO: 16 - protein, 12 aa, synthetic construct
  REGION 1..12: note = CDR-H2 (Contact) (P20, P20-V1, P20-V2, P20-V3)
WIGYIFYSGTTN

SEQ ID NO: 17 - protein, 17 aa, synthetic construct
  REGION 1..17: note = CDR-H3 (Contact) (P20, P20-V1, P20-V2, P20-V3)
ARDVGIAARNSYYYVMD

SEQ ID NO: 18 - protein, 8 aa, synthetic construct
  REGION 1..8: note = CDR-H1 (IMGT) (P20, P20-V1, P20-V2, P20-V3)
GGSINNYY

SEQ ID NO: 19 - protein, 7 aa, synthetic construct
  REGION 1..7: note = CDR-H2 (IMGT) (P20, P20-V1, P20-V2, P20-V3)
IFYSGTT

SEQ ID NO: 20 - protein, 18 aa, synthetic construct
  REGION 1..18: note = CDR-H3 (IMGT) (P20, P20-V1, P20-V2, P20-V3)
ARDVGIAARNSYYYVMDV

SEQ ID NO: 21 - protein, 107 aa, synthetic construct
  REGION 1..107: note = VL region (aa) (P20)
AIQLTQSPSSLSASVGDRVTITCRASQGISSALAWYQQKPGKAPKFLIYDASSLESGVPSRFSGSGSGTDFTLTISSLQPEDFATYYCQQFNSYPITFGQGTRLEIK

SEQ ID NO: 22 - protein, 11 aa, synthetic construct
  REGION 1..11: note = CDR-L1 (Kabat, Chothia, AbM) (P20, P20-V1, P20-V2, P20-V3, P21, P21-V1)
RASQGISSALA

SEQ ID NO: 23 - protein, 7 aa, synthetic construct
  REGION 1..7: note = CDR-L2 (Kabat, Chothia, AbM) (P20)
DASSLES

SEQ ID NO: 24 - protein, 9 aa, synthetic construct
  REGION 1..9: note = CDR-L3 (Kabat, Chothia, AbM, IMGT) (P20, P20-V1, P20-V2, P20-V3)
QQFNSYPIT

SEQ ID NO: 25 - protein, 7 aa, synthetic construct
  REGION 1..7: note = CDR-L1 (Contact) (P20, P20-V1, P20-V2, P20-V3, P21, P21-V1)
SSALAWY

SEQ ID NO: 26 - protein, 10 aa, synthetic construct
  REGION 1..10: note = CDR-L2 (Contact) (P20)
FLIYDASSLE

SEQ ID NO: 27 - protein, 8 aa, synthetic construct
  REGION 1..8: note = CDR-L3 (Contact) (P20, P20-V1, P20-V2, P20-V3)
QQFNSYPI

SEQ ID NO: 28 - protein, 6 aa, synthetic construct
  REGION 1..6: note = CDR-L1 (IMGT) (P20, P20-V1, P20-V2, P20-V3, P21, P21-V1)
QGISSA

SEQ ID NO: 29 - skipped

SEQ ID NO: 30 - protein, 329 aa, synthetic construct
  REGION 1..329: note = heavy chain constant domain (aa)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 107 aa, synthetic construct [sequence omitted]

SEQ ID NO: 32 - protein, 453 aa, synthetic construct
  REGION 1..453: note = heavy chain (aa) (P20-V1, P20-V2, P20-V3)
QVQLQESGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 214 aa, synthetic construct
  REGION 1..214: note = light chain (aa) (P20-V1)
AIQLTQSPSSLSASVGDRVTITCRASQGISSALAWYQQKPGKAPKFLIYEASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 124 aa, synthetic construct
  REGION 1..124: note = VH region (aa) (P20-V1, P20-V2, P20-V3)
QVQLQESGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSS

SEQ ID NO: 35 - protein, 107 aa, synthetic construct
  REGION 1..107: note = VL region (aa) (P20-V1)
AIQLTQSPSSLSASVGDRVTITCRASQGISSALAWYQQKPGKAPKFLIYEASSLESGVPSRFSGSGSGTDFTLTISSLQPEDFATYYCQQFNSYPITFGQGTRLEIK

SEQ ID NO: 36 - protein, 7 aa, synthetic construct
  REGION 1..7: note = CDR-L2 (Kabat, Chothia, AbM) (P20-V1)
EASSLES

SEQ ID NO: 37 - protein, 10 aa, synthetic construct
  REGION 1..10: note = CDR-L2 (Contact) (P20-V1)
FLIYEASSLE

SEQ ID NO: 38 - skipped

SEQ ID NO: 39 - protein, 214 aa, synthetic construct
  REGION 1..214: note = light chain (aa) (P20-V2)
AIQLTQSPSSLSASVGDRVTITCRASQGISSALAWYQQKPGKAPKFLIYEGSSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 107 aa, synthetic construct
  REGION 1..107: note = VL region (P20-V2)
AIQLTQSPSSLSASVGDRVTITCRASQGISSALAWYQQKPGKAPKFLIYEGSSLESGVPSRFSGSGSGTDFTLTISSLQPEDFATYYCQQFNSYPITFGQGTRLEIK

SEQ ID NO: 41 - protein, 7 aa, synthetic construct
  REGION 1..7: note = CDR-L2 (Kabat, Chothia) (P20-V2)
EGSSLES

SEQ ID NO: 42 - protein, 10 aa, synthetic construct
  REGION 1..10: note = CDR-L2 (Contact) (P20-V2)
FLIYEGSSLE

SEQ ID NO: 43 - skipped

SEQ ID NO: 44 - protein, 214 aa, synthetic construct
  REGION 1..214: note = light chain (aa) (P20-V3)
AIQLTQSPSSLSASVGDRVTITCRASQGISSALAWYQQKPGKAPKFLIYGASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 107 aa, synthetic construct
  REGION 1..107: note = VL region (aa) (P20-V3)
AIQLTQSPSSLSASVGDRVTITCRASQGISSALAWYQQKPGKAPKFLIYGASSLESGVPSRFSGSGSGTDFTLTISSLQPEDFATYYCQQFNSYPITFGQGTRLEIK

SEQ ID NO: 46 - protein, 7 aa, synthetic construct
  REGION 1..7: note = CDR-L2 (Kabat, Chothia) (P20-V3)
GASSLES

SEQ ID NO: 47 - protein, 10 aa, synthetic construct
  REGION 1..10: note = CDR-L2 (Contact) (P20-V3)
FLIYGASSLE

SEQ ID NO: 48 - skipped

SEQ ID NO: 49 - protein, 452 aa, synthetic construct
  REGION 1..452: note = heavy chain (aa) (P8)
QVQLVQSGAEVKKPGASVRVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
  REGION 1..214: note = light chain (aa) (P8)
DIRMTQSPSSLSASVGDRVTITCRASQSISSYLNWYQQKPGKAPKLLIYDASSLQSGVPSRFSGSGSGTDFTLTMSSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 123 aa, synthetic construct
  REGION 1..123: note = VH region (P8)
QVQLVQSGAEVKKPGASVRVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSS

SEQ ID NO: 52 - protein, 5 aa, synthetic construct
  REGION 1..5: note = CDR-H1 (Kabat) (P8, P8-V1)
SYYMH

SEQ ID NO: 53 - protein, 17 aa, synthetic construct
  REGION 1..17: note = CDR-H2 (Kabat) (P8, P8-V1)
IINPRGGYTTYAQKFQG

SEQ ID NO: 54 - protein, 14 aa, synthetic construct
  REGION 1..14: note = CDR-H3 (Kabat, Chothia, AbM) (P8, P8-V1)
EGRVSPTDYYGMDV

SEQ ID NO: 55 - protein, 7 aa, synthetic construct
  REGION 1..7: note = CDR-H1 (Chothia) (P8, P8-V1)
GYTFTSY

SEQ ID NO: 56 - protein, 6 aa, synthetic construct
  REGION 1..6: note = CDR-H2 (Chothia) (P8, P8-V1)
NPRGGY

SEQ ID NO: 57 - protein, 10 aa, synthetic construct
  REGION 1..10: note = CDR-H1 (AbM) (P8, P8-V1)
GYTFTSYYMH

SEQ ID NO: 58 - protein, 10 aa, synthetic construct
  REGION 1..10: note = CDR-H2 (AbM) (P8, P8-V1)
IINPRGGYTT

SEQ ID NO: 59 - protein, 6 aa, synthetic construct
  REGION 1..6: note = CDR-H1 (Contact) (P8, P8-V1)
TSYYMH

SEQ ID NO: 60 - protein, 13 aa, synthetic construct
  REGION 1..13: note = CDR-H2 (Contact) (P8, P8-V1)
WMGIINPRGGYTT

SEQ ID NO: 61 - protein, 15 aa, synthetic construct
  REGION 1..15: note = CDR-H3 (Contact) (P8, P8-V1)
AKEGRVSPTDYYGMD

SEQ ID NO: 62 - protein, 8 aa, synthetic construct
  REGION 1..8: note = CDR-H1 (IMGT) (P8, P8-V1)
GYTFTSYY

SEQ ID NO: 63 - protein, 8 aa, synthetic construct
  REGION 1..8: note = CDR-H2 (IMGT) (P8, P8-V1)
INPRGGYT

SEQ ID NO: 64 - protein, 16 aa, synthetic construct
  REGION 1..16: note = CDR-H3 (IMGT) (P8, P8-V1)
AKEGRVSPTDYYGMDV

SEQ ID NO: 65 - protein, 107 aa, synthetic construct
  REGION 1..107: note = VL region (P8)
DIRMTQSPSSLSASVGDRVTITCRASQSISSYLNWYQQKPGKAPKLLIYDASSLQSGVPSRFSGSGSGTDFTLTMSSLQPEDFATYYCQQSYSTPLTFGGGTKVEIK

SEQ ID NO: 66 - protein, 11 aa, synthetic construct
  REGION 1..11: note = CDR-L1 (Kabat, Chothia, AbM) (P8, P8-V1)
RASQSISSYLN

SEQ ID NO: 67 - protein, 7 aa, synthetic construct
  REGION 1..7: note = CDR-L2 (Kabat, Chothia,AbM) (P8, P8-V1)
DASSLQS

SEQ ID NO: 68 - protein, 9 aa, synthetic construct
  REGION 1..9: note = CDR-L3 (Kabat, Chothia,AbM, IMGT) (P8, P8-V1)
QQSYSTPLT

SEQ ID NO: 69 - protein, 7 aa, synthetic construct
  REGION 1..7: note = CDR-L1 (Contact) (P8, P8-V1)
SSYLNWY

SEQ ID NO: 70 - protein, 10 aa, synthetic construct
  REGION 1..10: note = CDR-L2 (Contact) (P8, P8-V1)
LLIYDASSLQ

SEQ ID NO: 71 - protein, 8 aa, synthetic construct
  REGION 1..8: note = CDR-L3 (Contact) (P8, P8-V1)
QQSYSTPL

SEQ ID NO: 72 - protein, 6 aa, synthetic construct
  REGION 1..6: note = CDR-L1 (IMGT) (P8, P8-V1)
QSISSY

SEQ ID NO: 73 - protein, 452 aa, synthetic construct
  REGION 1..452: note = heavy chain (aa) (P8-V1)
QVQLVQSGAEVKKPGASVKVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 214 aa, synthetic construct
  REGION 1..214: note = light chain (aa) (P8-V1)
DIQMTQSPSSLSASVGDRVTITCRASQSISSYLNWYQQKPGKAPKLLIYDASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 123 aa, synthetic construct
  REGION 1..123: note = VH region (aa) (P8-V1)
QVQLVQSGAEVKKPGASVKVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSS

SEQ ID NO: 76 - protein, 107 aa, synthetic construct
  REGION 1..107: note = VL region (aa) (P8-V1)
DIQMTQSPSSLSASVGDRVTITCRASQSISSYLNWYQQKPGKAPKLLIYDASSLQSGVPSRFSGSGSGTDFTLTISSLQPEDFATYYCQQSYSTPLTFGGGTKVEIK

SEQ ID NO: 77 - protein, 452 aa, synthetic construct
  REGION 1..452: note = heavy chain (P21)
QVQLQQSGPGLVKPSETLSLTCTVSGGSISNYYWTWIRQPPGKGLEWIGYISDSGSTNYNPSLKSRVTISVDTSKNQFSLKLSSVTAADTAVYYCARETIVVRGFYFYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 214 aa, synthetic construct
  REGION 1..214: note = light chain (P21)
AIQLTQSPSSLSASVGDRVTITCRASQGISSALAWYQQRPGKAPKLLIYDASNWESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 123 aa, synthetic construct
  REGION 1..123: note = VH region (P21)
QVQLQQSGPGLVKPSETLSLTCTVSGGSISNYYWTWIRQPPGKGLEWIGYISDSGSTNYNPSLKSRVTISVDTSKNQFSLKLSSVTAADTAVYYCARETIVVRGFYFYVMDVWGQGTTVTVSS

SEQ ID NO: 80 - protein, 5 aa, synthetic construct
  REGION 1..5: note = CDR-H1 (Kabat) (P21, P21-V1)
NYYWT

SEQ ID NO: 81 - protein, 16 aa, synthetic construct
  REGION 1..16: note = CDR-H2 (Kabat) (P21, P21-V1)
YISDSGSTNYNPSLKS

SEQ ID NO: 82 - protein, 15 aa, synthetic construct
  REGION 1..15: note = CDR-H3 (Kabat, Chothia, AbM) (P21, P21-V1)
ETIVVRGFYFYVMDV

SEQ ID NO: 83 - protein, 7 aa, synthetic construct
  REGION 1..7: note = CDR-H1 (Chothia) (P21, P21-V1)
GGSISNY

SEQ ID NO: 84 - protein, 5 aa, synthetic construct
  REGION 1..5: note = CDR-H2 (Chothia) (P21, P21-V1)
SDSGS

SEQ ID NO: 85 - protein, 10 aa, synthetic construct
  REGION 1..10: note = CDR-H1 (AbM) (P21, P21-V1)
GGSISNYYWT

SEQ ID NO: 86 - protein, 9 aa, synthetic construct
  REGION 1..9: note = CDR-H2 (AbM) (P21, P21-V1)
YISDSGSTN

SEQ ID NO: 87 - protein, 6 aa, synthetic construct
  REGION 1..6: note = CDR-H1 (Contact) (P21, P21-V1)
SNYYWT

SEQ ID NO: 88 - protein, 12 aa, synthetic construct
  REGION 1..12: note = CDR-H2 (Contact) (P21, P21-V1)
WIGYISDSGSTN

SEQ ID NO: 89 - protein, 16 aa, synthetic construct
  REGION 1..16: note = CDR-H3 (Contact) (P21, P21-V1)
ARETIVVRGFYFYVMD

SEQ ID NO: 90 - protein, 8 aa, synthetic construct
  REGION 1..8: note = CDR-H1 (IMGT) (P21, P21-V1)
GGSISNYY

SEQ ID NO: 91 - protein, 7 aa, synthetic construct
  REGION 1..7: note = CDR-H2 (IMGT) (P21, P21-V1)
ISDSGST

SEQ ID NO: 92 - protein, 17 aa, synthetic construct
  REGION 1..17: note = CDR-H3 (IMGT) (P21, P21-V1)
ARETIVVRGFYFYVMDV

SEQ ID NO: 93 - protein, 107 aa, synthetic construct
  REGION 1..107: note = VL region (P21)
AIQLTQSPSSLSASVGDRVTITCRASQGISSALAWYQQRPGKAPKLLIYDASNWESGVPSRFSGSGSGTDFTLTISSLQPEDFATYYCQQFNSYPWTFGQGTKVEIK

SEQ ID NO: 94 - protein, 7 aa, synthetic construct
  REGION 1..7: note = CDR-L2 (Kabat, Chothia, AbM) (P21)
DASNWES

SEQ ID NO: 95 - protein, 9 aa, synthetic construct
  REGION 1..9: note = CDR-L3 (Kabat, Chothia, AbM, IMGT) (P21, P21-V1)
QQFNSYPWT

SEQ ID NO: 96 - protein, 10 aa, synthetic construct
  REGION 1..10: note = CDR-L2 (Contact) (P21)
LLIYDASNWE

SEQ ID NO: 97 - protein, 8 aa, synthetic construct
  REGION 1..8: note = CDR-L3 (Contact) (P21, P21-V1)
QQFNSYPW

SEQ ID NO: 98 - protein, 452 aa, synthetic construct
  REGION 1..452: note = heavy chain (aa) (P21-V1)
QVQLQESGPGLVKPSETLSLTCTVSGGSISNYYWTWIRQPPGKGLEWIGYISDSGSTNYNPSLKSRVTISVDTSKNQFSLKLSSVTAADTAVYYCARETIVVRGFYFYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 214 aa, synthetic construct
  REGION 1..214: note = light chain (aa) (P21-V1)
AIQLTQSPSSLSASVGDRVTITCRASQGISSALAWYQQKPGKAPKLLIYEASNWESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 123 aa, synthetic construct
  REGION 1..123: note = VH region (aa) (P21-V1)
QVQLQESGPGLVKPSETLSLTCTVSGGSISNYYWTWIRQPPGKGLEWIGYISDSGSTNYNPSLKSRVTISVDTSKNQFSLKLSSVTAADTAVYYCARETIVVRGFYFYVMDVWGQGTTVTVSS

SEQ ID NO: 101 - protein, 107 aa, synthetic construct
  REGION 1..107: note = VL region (aa) (P21-V1)
AIQLTQSPSSLSASVGDRVTITCRASQGISSALAWYQQKPGKAPKLLIYEASNWESGVPSRFSGSGSGTDFTLTISSLQPEDFATYYCQQFNSYPWTFGQGTKVEIK

SEQ ID NO: 102 - protein, 7 aa, synthetic construct
  REGION 1..7: note = CDR-L2 (Kabat, Chothia, AbM) (P21-V1)
EASNWES

SEQ ID NO: 103 - protein, 10 aa, synthetic construct
  REGION 1..10: note = CDR-L2 (Contact) (P21-V1)
LLIYEASNWE

SEQ ID NO: 104 - protein, 330 aa, synthetic construct
  REGION 1..330: note = hIgG1 HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 990 bp, synthetic construct
  misc_feature 1..990: note = hIgG1 HC constant region DNA
  source 1..990: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaag

SEQ ID NO: 106 - protein, 329 aa, synthetic construct
  REGION 1..329: note = hIgG1 HC constant region without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - DNA, 987 bp, synthetic construct
  misc_feature 1..987: note = hIgG1 HC constant region DNA without C-terminal lysine
  source 1..987: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 108 - protein, 330 aa, synthetic construct
  REGION 1..330: note = hIgG1.3f HC constant region; IgG 1.3 comprises mutations L234A, L235E, and G237A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 990 bp, synthetic construct
  misc_feature 1..990: note = hIgG1.3f HC constant region DNA sequence
  source 1..990: mol_type = other DNA
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaag

SEQ ID NO: 110 - protein, 329 aa, synthetic construct; identical to SEQ ID NO: 30
  REGION 1..329: note = hIgG1.3f HC constant region without the C-terminal lysine; IgG 1.3 mutations L234A, L235E, and G237A

SEQ ID NO: 111 - DNA, 987 bp, synthetic construct
  misc_feature 1..987: note = hIgG1.3f HC constant region without the C-terminal lysine; DNA sequence
  source 1..987: mol_type = other DNA
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 112 - protein, 330 aa, synthetic construct
  REGION 1..330: note = hIgG1fa L235E, P238K (LEPK)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 990 bp, synthetic construct
  misc_feature 1..990: note = hIgG1fa LEPK HC constant region DNA sequence
  source 1..990: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcgaggggggaaagtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 114 - protein, 329 aa, synthetic construct
  REGION 1..329: note = hIgG1fa LEPK HC constant region without the C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - DNA, 987 bp, synthetic construct
  misc_feature 1..987: note = hIgG1fa LEPK HC constant region without the C-terminal lysine DNA sequence
  source 1..987: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcgaggggggaaagtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 116 - protein, 326 aa, synthetic construct
  REGION 1..326: note = hIgG2.3 HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - DNA, 978 bp, synthetic construct
  misc_feature 1..978: note = hIgG2.3 HC constant region DNA
  source 1..978: mol_type = other DNA
gcgtcgaccaagggcccctctgtgtttcctctggccccttgctcccggtccacctctgagtctaccgctgctctgggctgcctggtcaaggactacttccccgagcctgtgaccgtgtcctggaactctggcgctctgacctccggcgtgcacacctttccagccgtgctgcagtcctccggcctgtactctctgtcctccgtcgtgaccgtgccctcctccaacttcggcacccagacctacacctgtaacgtggaccacaagccctccaacaccaaggtggacaagaccgtggaacggaagtcctgcgtggaatgccctccttgccctgcacctcctgtggctggcaaatccgtgttcctgttccccccaaagcccaaggacaccctgatgatctcccggacccccgaagtgacctgcgtggtggtggatgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcaccaggactggctgaacggcaaagagtacaagtgcaaggtgtccaacaagggcctgcctgcccccatcgaaaagaccatctccaagacaaagggccagccccgcgagcctcaggtgtacacactgcctcccagccgggaagagatgaccaagaaccaggtgtccctgacctgtctggtcaagggcttctacccctccgatatcgccgtggaatgggagtccaacggccagcccgagaacaactacaagaccaccccccccatgctggactccgacggctcattcttcctgtactccaagctgacagtggacaagtcccggtggcagcagggcaacgtgttctcctgctctgtgatgcacgaggccctgcacaaccactacacccagaagtccctgtccctgagccccggcaaa

SEQ ID NO: 118 - protein, 325 aa, synthetic construct
  REGION 1..325: note = hIgG2.3 HC constant region, without C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 119 - DNA, 975 bp, synthetic construct
  misc_feature 1..975: note = hIgG2.3 HC constant region, without C-terminal lysine DNA
  source 1..975: mol_type = other DNA
gcgtcgaccaagggcccctctgtgtttcctctggccccttgctcccggtccacctctgagtctaccgctgctctgggctgcctggtcaaggactacttccccgagcctgtgaccgtgtcctggaactctggcgctctgacctccggcgtgcacacctttccagccgtgctgcagtcctccggcctgtactctctgtcctccgtcgtgaccgtgccctcctccaacttcggcacccagacctacacctgtaacgtggaccacaagccctccaacaccaaggtggacaagaccgtggaacggaagtcctgcgtggaatgccctccttgccctgcacctcctgtggctggcaaatccgtgttcctgttccccccaaagcccaaggacaccctgatgatctcccggacccccgaagtgacctgcgtggtggtggatgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcaccaggactggctgaacggcaaagagtacaagtgcaaggtgtccaacaagggcctgcctgcccccatcgaaaagaccatctccaagacaaagggccagccccgcgagcctcaggtgtacacactgcctcccagccgggaagagatgaccaagaaccaggtgtccctgacctgtctggtcaagggcttctacccctccgatatcgccgtggaatgggagtccaacggccagcccgagaacaactacaagaccaccccccccatgctggactccgacggctcattcttcctgtactccaagctgacagtggacaagtcccggtggcagcagggcaacgtgttctcctgctctgtgatgcacgaggccctgcacaaccactacacccagaagtccctgtccctgagccccggc

SEQ ID NO: 120 - protein, 326 aa, synthetic construct
  REGION 1..326: note = hIgG2.3 P238K HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - DNA, 978 bp, synthetic construct; identical to SEQ ID NO: 117
  misc_feature 1..978: note = hIgG2.3 P238K HC constant region DNA sequence
  source 1..978: mol_type = other DNA

SEQ ID NO: 122 - protein, 325 aa, synthetic construct
  REGION 1..325: note = hIgG2.3 P238K HC constant region without the C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 123 - DNA, 975 bp, synthetic construct; identical to SEQ ID NO: 119
  misc_feature 1..975: note = hIgG2.3 P238K HC constant region without the C-terminal lysine DNA sequence
  source 1..975: mol_type = other DNA

SEQ ID NO: 124 - protein, 107 aa, synthetic construct [sequence omitted]

SEQ ID NO: 125 - DNA, 321 bp, synthetic construct
  misc_feature 1..321: note = Exemplary LC constant region DNA sequence
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 126 - protein, 599 aa, synthetic construct
  REGION 1..599: note = huIL-18RBeta
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 127 - DNA, 372 bp, synthetic construct
  misc_feature 1..372: note = VH region (nt) (P20-V1)
  source 1..372: mol_type = other DNA
caagtgcaactacaagaaagtggtccaggtctggtgaaaccatccgagacactcagtctgacctgcaccgtgtccggcggctccatcaacaactattactggtcctggatcagacagcctcctggcaagggactggaatggatcggctacattttctactctggcaccaccaactacaacccctccctgaagagcagagtgacaatctctgtggatacctccaagaaccagttctccctgaaactgtccagcgtgacagccgctgatacagccgtctactactgcgccagagatgtgggcatcgccgccaggaactcctactactacgtgatggacgtgtggggccagggcaccaccgtcaccgtgagctct

SEQ ID NO: 128 - DNA, 321 bp, synthetic construct
  misc_feature 1..321: note = VL region (nt) (P20-V1)
  source 1..321: mol_type = other DNA
gcaatccaactaactcaaagtccaagtagtctgtccgcttccgtgggcgacagagtgactatcacctgcagagcctctcagggcatctcttctgctctggcctggtaccagcagaagcctggcaaggctcctaagttcctgatctacgaagccagctctctggaatctggcgtgccttctcggttctccggctccggatctggcaccgacttcaccctgaccatctcctctctgcaacctgaggactttgccacctactactgccagcagttcaactcctaccccatcaccttcggccagggcaccagactggaaatcaag

SEQ ID NO: 129 - DNA, 1359 bp, synthetic construct
  misc_feature 1..1359: note = Heavy chain (nt) (P20-V1)
  source 1..1359: mol_type = other DNA
caagtgcaactacaagaaagtggtccaggtctggtgaaaccatccgagacactcagtctgacctgcaccgtgtccggcggctccatcaacaactattactggtcctggatcagacagcctcctggcaagggactggaatggatcggctacattttctactctggcaccaccaactacaacccctccctgaagagcagagtgacaatctctgtggatacctccaagaaccagttctccctgaaactgtccagcgtgacagccgctgatacagccgtctactactgcgccagagatgtgggcatcgccgccaggaactcctactactacgtgatggacgtgtggggccagggcaccaccgtcaccgtgagctctgcttctaccaagggccctagcgtatttcctctggcccctagctccaagagcacctccggcggaaccgctgctctgggctgtctggtgaaggactacttccctgagcccgtgaccgtgtcttggaactccggtgccctcacctctggcgtgcataccttccctgctgtgctgcagtccagcggactgtactccctgtcttccgtggtgaccgtgccttcctcctctctgggcactcaaacatacatctgcaatgtgaaccacaagcccagcaacaccaaagtggacaagcgggtggaacccaagtcctgcgacaagacccacacctgtcctccttgcccagctcccgaagccgagggcgctccttctgtctttctgttccctcctaaacctaaggacaccctgatgatctctagaacccctgaagtgacctgcgtcgtcgtggacgtgtcccacgaggaccctgaggtgaagttcaactggtacgttgacggcgtggaagtgcataacgccaagacaaagcctagagaagagcagtacaactctacctaccgggtggtgtctgtgctgaccgtgctgcaccaggactggctgaacggcaaagagtacaagtgcaaggtgtccaacaaggctctgcccgcacccatcgagaagaccatctctaaggccaaaggccagccaagagagcctcaggtgtacaccctgcctccatccagagaagagatgaccaagaaccaagtgtctctgacctgtctggtgaagggcttctatccttctgacatcgccgtggagtgggaatctaatggccagcctgagaacaactataagaccacacctcctgtgctggactccgacggctctttcttcctgtacagcaagctcaccgtggataagtcccggtggcagcagggcaacgtgttctcttgctccgtgatgcacgaggccctgcacaatcactacacccagaagtccctgtctctgtctcctgga

SEQ ID NO: 130 - DNA, 642 bp, synthetic construct
  misc_feature 1..642: note = Light chain (nt) (P20-V1)
  source 1..642: mol_type = other DNA
gcaatccaactaactcaaagtccaagtagtctgtccgcttccgtgggcgacagagtgactatcacctgcagagcctctcagggcatctcttctgctctggcctggtaccagcagaagcctggcaaggctcctaagttcctgatctacgaagccagctctctggaatctggcgtgccttctcggttctccggctccggatctggcaccgacttcaccctgaccatctcctctctgcaacctgaggactttgccacctactactgccagcagttcaactcctaccccatcaccttcggccagggcaccagactggaaatcaagcggaccgtggccgctccatctgtgttcatctttcctccttccgatgaacagctgaagtctggaacagcttccgtggtgtgcctgctgaacaacttctatcctagagaggctaaagtgcagtggaaggtggacaacgccctgcagtccggcaatagccaagagtccgtcaccgagcaggattccaaggactccacctactccctgtccagcacactcacactgagcaaagccgactacgagaagcacaaggtgtacgcctgcgaggtgacccaccagggcctgtctagccccgtcaccaagtccttcaaccggggcgagtgt

SEQ ID NO: 131 - DNA, 369 bp, synthetic construct
  misc_feature 1..369: note = VH region (nt) (P8-V1)
  source 1..369: mol_type = other DNA
caagtgcaactagtgcaaagtggtgcagaagtcaagaagcctggcgcttccgtgaaggtgtcctgcaaggcctctggctataccttcacctcctactacatgcactgggtgcggcaggctcctggacaaggcctggagtggatgggcatcatcaaccccagaggcggctacaccacctacgcccagaagttccagggcagagtgaccatgaccagagataccagcacctctacagtgtacatggaactgtcctctctgcgctccgaggacaccgccgtgtactactgtgccaaagagggacgggtgtctcctaccgactactacggcatggacgtgtggggccagggcaccacagttacagtgtccagc

SEQ ID NO: 132 - DNA, 321 bp, synthetic construct
  misc_feature 1..321: note = VL region (nt) (P8-V1)
  source 1..321: mol_type = other DNA
gatatccaaatgactcaaagtccaagtagtctctctgcttccgtgggcgacagagtgaccatcacctgtagagcctctcagtccatcagcagctatctgaactggtaccagcagaagcctggcaaagctccaaagctgctgatctacgacgcctccagcctgcagtctggcgtcccctctcggttctccggatctggctccggcacagattttaccctgaccatctcctctctgcagcctgaggacttcgccacctactactgccagcaatcctactccacacctctgaccttcggcggaggcaccaaggtggaaatcaag

SEQ ID NO: 133 - DNA, 1356 bp, synthetic construct
  misc_feature 1..1356: note = Heavy chain (nt) (P8-V1)
  source 1..1356: mol_type = other DNA
caagtgcaactagtgcaaagtggtgcagaagtcaagaagcctggcgcttccgtgaaggtgtcctgcaaggcctctggctataccttcacctcctactacatgcactgggtgcggcaggctcctggacaaggcctggagtggatgggcatcatcaaccccagaggcggctacaccacctacgcccagaagttccagggcagagtgaccatgaccagagataccagcacctctacagtgtacatggaactgtcctctctgcgctccgaggacaccgccgtgtactactgtgccaaagagggacgggtgtctcctaccgactactacggcatggacgtgtggggccagggcaccacagttacagtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 134 - DNA, 642 bp, synthetic construct
  misc_feature 1..642: note = Light chain (nt) (P8-V1)
  source 1..642: mol_type = other DNA
gatatccaaatgactcaaagtccaagtagtctctctgcttccgtgggcgacagagtgaccatcacctgtagagcctctcagtccatcagcagctatctgaactggtaccagcagaagcctggcaaagctccaaagctgctgatctacgacgcctccagcctgcagtctggcgtcccctctcggttctccggatctggctccggcacagattttaccctgaccatctcctctctgcagcctgaggacttcgccacctactactgccagcaatcctactccacacctctgaccttcggcggaggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 135 - DNA, 369 bp, synthetic construct; identical to SEQ ID NO: 131
  misc_feature 1..369: note = VH region (nt) (P20)
  source 1..369: mol_type = other DNA

SEQ ID NO: 136 - DNA, 321 bp, synthetic construct
  misc_feature 1..321: note = VL region (nt) (P20)
  source 1..321: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacgacgcgagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaag

SEQ ID NO: 137 - DNA, 1359 bp, synthetic construct
  misc_feature 1..1359: note = Heavy chain (nt) (P20)
  source 1..1359: mol_type = other DNA
caagtccaacttcagcagagcggaccgggactggtcaagccctccgagactctgtccctcacttgcaccgtgtccggaggttctatcaacaactactactggtcctggattcggcagcctcctgggaagggcctcgagtggatcggctacatcttctactccggcactaccaattacaacccgtcgctgaagtcccgcgtgaccatttccgtggacacttcgaagaaccagttcagcctgaagctctccagcgtgaccgctgccgataccgccgtctactactgcgctcgcgatgtcggaatcgcggcacggaactcatattactacgtgatggacgtctggggacagggaaccaccgtgaccgtcagcagcgcaagcaccaagggtccgagcgtgttccctctggccccatcctcgaagtcaacttccggtggaactgccgccctgggatgcttggtcaaggactacttcccggaacccgtgaccgtgtcatggaattcgggggcccttacctcgggcgtgcataccttccccgcggtgttgcagtccagcggcctctattccctctcatccgtcgtgactgtgccgagttcctcgctgggcacccagacctacatctgcaacgtgaaccataagccctccaacaccaaggtcgacaaacgggtggagcctaagtcctgcgacaagacgcacacttgcccaccttgtcccgctcctgaagccgagggagcgccttcagtgttcctgtttcccccgaaacccaaggataccctgatgatctcccggacccccgaagtgacctgtgtggtggtggatgtcagccacgaagatccggaagtcaagttcaattggtacgtggacggcgtggaagtgcacaacgctaagactaagccgcgcgaggaacagtacaactcgacttaccgcgtcgtgtccgtgctgactgtgctgcatcaggactggctgaacggaaaagagtacaagtgcaaagtgtccaacaaggcactgcctgcgccaattgaaaagaccatcagcaaggccaagggacagcccagggaacctcaagtgtacacgctcccgccgagccgggaagaaatgaccaagaaccaagtgtcactgacatgcctcgtgaagggattctacccctcggacattgccgtggagtgggagtccaacgggcagccggagaacaactataagaccaccccaccggtattggactcggacggctccttcttcctgtactcgaagctgactgtggacaagtccagatggcaacaggggaacgttttcagctgctccgtgatgcacgaagcgctgcacaaccactacacccagaagtccctgtctctgtcccccggc

SEQ ID NO: 138 - DNA, 642 bp, synthetic construct
  misc_feature 1..642: note = Light chain (nt) (P20)
  source 1..642: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacgacgcgagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaagcggactgtggcggcacctagcgtgttcatcttccctccatcggatgaacagctgaagtccggaactgccagcgtcgtgtgcctgctgaacaatttctacccccgcgaggctaaggtccagtggaaggtcgacaacgcgctgcagtccggaaactcccaggaatccgtgaccgaacaggactcgaaggactccacttactcgctgagctccaccctgacgctttcgaaggcagactacgagaagcataaagtgtacgcctgcgaagtgacccaccagggcctgagctcccctgtgaccaagtccttcaaccgcggggagtgc

SEQ ID NO: 139 - DNA, 372 bp, synthetic construct
  misc_feature 1..372: note = VH region (nt) (P20-V2)
  source 1..372: mol_type = other DNA
caggtgcagctgcaagaaagcggaccgggactggtcaagccctccgagactctgtccctcacttgcaccgtgtccggaggttctatcaacaactactactggtcctggattcggcagcctcctgggaagggcctcgagtggatcggctacatcttctactccggcactaccaattacaacccgtcgctgaagtcccgcgtgaccatttccgtggacacttcgaagaaccagttcagcctgaagctctccagcgtgaccgctgccgataccgccgtctactactgcgctcgcgatgtcggaatcgcggcacggaactcatattactacgtgatggacgtctggggacagggaaccaccgtgaccgtcagcagc

SEQ ID NO: 140 - DNA, 321 bp, synthetic construct
  misc_feature 1..321: note = VL region (nt) (P20-V2)
  source 1..321: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacgaaggcagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaag

SEQ ID NO: 141 - DNA, 1359 bp, synthetic construct
  misc_feature 1..1359: note = Heavy chain (nt) (P20-V2)
  source 1..1359: mol_type = other DNA
caggtgcagctgcaagaaagcggaccgggactggtcaagccctccgagactctgtccctcacttgcaccgtgtccggaggttctatcaacaactactactggtcctggattcggcagcctcctgggaagggcctcgagtggatcggctacatcttctactccggcactaccaattacaacccgtcgctgaagtcccgcgtgaccatttccgtggacacttcgaagaaccagttcagcctgaagctctccagcgtgaccgctgccgataccgccgtctactactgcgctcgcgatgtcggaatcgcggcacggaactcatattactacgtgatggacgtctggggacagggaaccaccgtgaccgtcagcagcgcaagcaccaagggtccgagcgtgttccctctggccccatcctcgaagtcaacttccggtggaactgccgccctgggatgcttggtcaaggactacttcccggaacccgtgaccgtgtcatggaattcgggggcccttacctcgggcgtgcataccttccccgcggtgttgcagtccagcggcctctattccctctcatccgtcgtgactgtgccgagttcctcgctgggcacccagacctacatctgcaacgtgaaccataagccctccaacaccaaggtcgacaaacgggtggagcctaagtcctgcgacaagacgcacacttgcccaccttgtcccgctcctgaagccgagggagcgccttcagtgttcctgtttcccccgaaacccaaggataccctgatgatctcccggacccccgaagtgacctgtgtggtggtggatgtcagccacgaagatccggaagtcaagttcaattggtacgtggacggcgtggaagtgcacaacgctaagactaagccgcgcgaggaacagtacaactcgacttaccgcgtcgtgtccgtgctgactgtgctgcatcaggactggctgaacggaaaagagtacaagtgcaaagtgtccaacaaggcactgcctgcgccaattgaaaagaccatcagcaaggccaagggacagcccagggaacctcaagtgtacacgctcccgccgagccgggaagaaatgaccaagaaccaagtgtcactgacatgcctcgtgaagggattctacccctcggacattgccgtggagtgggagtccaacgggcagccggagaacaactataagaccaccccaccggtattggactcggacggctccttcttcctgtactcgaagctgactgtggacaagtccagatggcaacaggggaacgttttcagctgctccgtgatgcacgaagcgctgcacaaccactacacccagaagtccctgtctctgtcccccggc

SEQ ID NO: 142 - DNA, 642 bp, synthetic construct
  misc_feature 1..642: note = Light chain (nt) (P20-V2)
  source 1..642: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacgaaggcagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 143 - DNA, 372 bp, synthetic construct; identical to SEQ ID NO: 139
  misc_feature 1..372: note = VH region (nt) (P20-V3)
  source 1..372: mol_type = other DNA

SEQ ID NO: 144 - DNA, 321 bp, synthetic construct
  misc_feature 1..321: note = VL region (nt) (P20-V3)
  source 1..321: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacggcgcgagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaag

SEQ ID NO: 145 - DNA, 1359 bp, synthetic construct; identical to SEQ ID NO: 141
  misc_feature 1..1359: note = Heavy chain (nt) (P20-V3)
  source 1..1359: mol_type = other DNA

SEQ ID NO: 146 - DNA, 642 bp, synthetic construct
  misc_feature 1..642: note = Light chain (nt) (P20-V3)
  source 1..642: mol_type = other DNA
gctattcaacttacccagtccccgtcctccctgagcgcctcagtgggggatcgggtcaccatcacttgtcgggcctcacaaggcatctcctcggccctcgcttggtatcagcagaaacccggaaaggcccccaagttcctgatctacggcgcgagcagcctggagtcgggagtgccgtcaaggttcagcggatccggctctggaaccgacttcaccctgactatttcctcactgcaaccggaagatttcgccacctactactgccagcaattcaactcctacccgattacctttggccagggtacacgcctcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 147 - DNA, 369 bp, synthetic construct
  misc_feature 1..369: note = VH region (nt) (P8)
  source 1..369: mol_type = other DNA
caagtacaactcgtgcagtccggagcggaagtgaaaaagcctggagcgtcagtgcgcgtcagttgcaaagctagcggctacactttcacctcctactacatgcactgggtccgccaagcaccgggacagggcttggagtggatggggatcattaacccacggggcggatacaccacttatgctcagaagttccagggccgcgtgactatgactagggacaccagcacttccaccgtgtacatggaactgtcgtcgctgcggtcggaagatactgccgtctactactgcgccaaggaaggcagagtgtcccccaccgactactacggaatggacgtctggggacagggcaccaccgtcaccgtgtcctcg

SEQ ID NO: 148 - DNA, 321 bp, synthetic construct
  misc_feature 1..321: note = VL region (nt) (P8)
  source 1..321: mol_type = other DNA
gatattagaatgacccaatccccctcctccctgtcggcgtcagtgggggatcgcgtgaccatcacttgccgggcgtcacaaagcatttcctcctatctcaactggtaccagcagaagcccggaaaggcccctaagctcctgatctacgacgcctcaagcttgcaatctggagtgccgagccgcttctccggatccggctctggcactgacttcaccctgaccatgagcagcctgcagcccgaggacttcgccacttactactgtcagcagtcgtactcaacgccactcactttcggcggcggaaccaaggtcgagatcaag

SEQ ID NO: 149 - DNA, 1356 bp, synthetic construct
  misc_feature 1..1356: note = Heavy chain (nt) (P8)
  source 1..1356: mol_type = other DNA
caagtacaactcgtgcagtccggagcggaagtgaaaaagcctggagcgtcagtgcgcgtcagttgcaaagctagcggctacactttcacctcctactacatgcactgggtccgccaagcaccgggacagggcttggagtggatggggatcattaacccacggggcggatacaccacttatgctcagaagttccagggccgcgtgactatgactagggacaccagcacttccaccgtgtacatggaactgtcgtcgctgcggtcggaagatactgccgtctactactgcgccaaggaaggcagagtgtcccccaccgactactacggaatggacgtctggggacagggcaccaccgtcaccgtgtcctcggcgtccaccaagggccccagcgtgtttcctctggccccgtcatccaagtccacctcgggcggaactgctgccctgggatgcctggtcaaggactacttcccggaacccgtgactgtgtcctggaactcgggagcgctcaccagcggtgttcacaccttccccgcggtgcttcagagctctgggctctatagcctgagcagcgtcgtgaccgtgccgagctccagcctgggcacccaaacctacatctgcaacgtgaaccataagccctcgaacaccaaggtcgataagcgcgtggagcccaagtcctgtgacaagacccacacgtgtcctccttgccctgcaccggaagccgagggtgctccatccgtgttcctctttccaccgaagcccaaagatactctcatgatctcccggacccccgaagtcacttgcgtggtggtggatgtgtcccacgaggatcccgaagtcaagttcaattggtacgtggacggtgtcgaggtgcacaatgccaagactaagcccagggaagaacagtacaacagcacctaccgggtggtgtccgtgctgacggtgctgcatcaggactggctgaacgggaaggagtacaagtgcaaagtgtccaacaaggccctgcctgcacctatcgagaaaacaatctccaaggccaagggacagccgcgggaacctcaggtctacaccctgccaccatcccgggaagagatgactaagaaccaagtgtccctgacttgtctggtcaagggattctacccgtccgacatcgccgtggagtgggagtccaatgggcagccggagaacaactacaagaccacacctcctgtgctcgactccgacggctcgttcttcctgtactcgaagctcactgtggacaagtcacgatggcagcagggaaacgtgttctcctgctcggtcatgcacgaagccctgcacaaccactatacccagaagtcactgtcactgtcgccggga

SEQ ID NO: 150 - DNA, 642 bp, synthetic construct
  misc_feature 1..642: note = Light chain (nt) (P8)
  source 1..642: mol_type = other DNA
gatattagaatgacccaatccccctcctccctgtcggcgtcagtgggggatcgcgtgaccatcacttgccgggcgtcacaaagcatttcctcctatctcaactggtaccagcagaagcccggaaaggcccctaagctcctgatctacgacgcctcaagcttgcaatctggagtgccgagccgcttctccggatccggctctggcactgacttcaccctgaccatgagcagcctgcagcccgaggacttcgccacttactactgtcagcagtcgtactcaacgccactcactttcggcggcggaaccaaggtcgagatcaagcggaccgtggcagctccgtccgtgtttatcttccctccgtcggacgagcaattgaagtccggtaccgcgtccgtcgtgtgcctgctgaacaacttctacccgagggaagccaaagtgcagtggaaagtcgacaatgcactgcagtccgggaacagccaggaatccgtgaccgaacaggactcgaaggatagcacctactccctgtcctccactctgaccctgtcgaaggccgactacgagaagcataaggtctacgcttgcgaagtgacacaccagggactgagctcccctgtgaccaagtcgttcaaccggggcgaatgt

SEQ ID NO: 151 - DNA, 369 bp, synthetic construct
  misc_feature 1..369: note = VH region (nt) (P21)
  source 1..369: mol_type = other DNA
caagtgcaactgcagcagtcaggccctggcctcgtaaagccttcggagactctgtccctgacgtgtaccgtcagcgggggatccatctccaactactactggacctggatcaggcagccgcctggaaagggtctggagtggatcggatacatcagcgactccgggtccaccaattacaacccctcgctgaagtcccgggtcaccatttccgtggacacctcaaagaaccagttcagcctgaagctgagctccgttaccgcggctgacacagccgtgtactactgcgcccgggaaaccatcgtcgtgcggggattctacttctacgtgatggacgtctggggccagggaaccactgtgactgtgtcctcc

SEQ ID NO: 152 - DNA, 321 bp, synthetic construct
  misc_feature 1..321: note = VL region (nt) (P21)
  source 1..321: mol_type = other DNA
gccattcaactcacacagtccccgtcctctctgagcgcgtccgtgggtgatcgcgtgaccattacttgccgcgcctcacaaggcatcagctcggcgctcgcttggtaccagcagcggcctggaaaggcccctaagctgctgatctatgacgcctccaactgggagtcaggggtgccttcgaggttctccggaagcggatccggaactgacttcacgctcaccatctcctcgctgcaacccgaagatttcgccacctactactgccaacagttcaactcctacccctggactttcggacaaggcaccaaggtcgagattaag

SEQ ID NO: 153 - DNA, 1356 bp, synthetic construct
  misc_feature 1..1356: note = Heavy chain (nt) (P21)
  source 1..1356: mol_type = other DNA
caagtgcaactgcagcagtcaggccctggcctcgtaaagccttcggagactctgtccctgacgtgtaccgtcagcgggggatccatctccaactactactggacctggatcaggcagccgcctggaaagggtctggagtggatcggatacatcagcgactccgggtccaccaattacaacccctcgctgaagtcccgggtcaccatttccgtggacacctcaaagaaccagttcagcctgaagctgagctccgttaccgcggctgacacagccgtgtactactgcgcccgggaaaccatcgtcgtgcggggattctacttctacgtgatggacgtctggggccagggaaccactgtgactgtgtcctccgcatcaactaaggggccgtcagtgtttcctcttgccccgtcctccaagtcgacttctggaggaaccgctgcccttggctgcctggtcaaagactatttcccggaacccgtcacggtgtcctggaacagtggagcgctgaccagcggtgtccacaccttcccggctgtgctccagtcctccggactgtacagcctgagctcggtcgtgactgtgccgtcctcgtccctgggcacccagacatacatctgcaacgtgaaccataagccctcgaacaccaaggtcgataagagagtggagccgaagtcgtgcgataagactcacacttgcccaccgtgtccagcgccagaagcggaaggggcacctagcgtgttcctgttcccgcccaagccgaaggacaccctcatgattagccggacccccgaagtcacctgtgtggtggtcgacgtgtcccacgaagatcccgaagtcaagttcaattggtacgtggacggcgtggaagtgcacaacgccaagaccaagccgcgcgaggaacagtacaactccacctaccgcgtggtgtccgtgctcaccgtgttgcatcaggattggctgaacgggaaggagtacaagtgcaaagtgtccaacaaagcactgcctgcccccattgagaaaaccattagcaaggccaagggacagcctcgggagccacaagtgtacactctgcccccatcgagagaagaaatgaccaagaatcaagtgtcactgacgtgcctcgtgaagggattttacccctcggacatcgccgtggagtgggagtcgaacggccagcccgagaacaactacaagactaccccgcctgtgttggatagcgacggctcgttcttcctgtattccaagctcactgtggacaagtcacgctggcaacagggcaacgtgttctcctgctccgtgatgcacgaagccctgcataaccactacacccagaagtccctgtctctgagccctggt

SEQ ID NO: 154 - DNA, 642 bp, synthetic construct
  misc_feature 1..642: note = Light chain (nt) (P21)
  source 1..642: mol_type = other DNA
gccattcaactcacacagtccccgtcctctctgagcgcgtccgtgggtgatcgcgtgaccattacttgccgcgcctcacaaggcatcagctcggcgctcgcttggtaccagcagcggcctggaaaggcccctaagctgctgatctatgacgcctccaactgggagtcaggggtgccttcgaggttctccggaagcggatccggaactgacttcacgctcaccatctcctcgctgcaacccgaagatttcgccacctactactgccaacagttcaactcctacccctggactttcggacaaggcaccaaggtcgagattaagcggaccgtggctgcaccgtccgtgttcatcttcccaccctccgacgagcagctcaagagcggcaccgcaagcgtggtctgcctgctgaacaatttctacccgcgcgaagcgaaagtgcagtggaaggtcgataacgcgctgcagtcaggaaactcacaggaatccgtgactgaacaggactccaaggacagcacctactcgctgtcctccaccttgactctgtcgaaagccgactacgagaagcacaaggtctacgcctgtgaagtgacccatcagggtctgtcgagcccagtgaccaagtccttcaaccggggcgaatgc

SEQ ID NO: 155 - DNA, 369 bp, synthetic construct
  misc_feature 1..369: note = VH region (nt) (P21-V1)
  source 1..369: mol_type = other DNA
caggtgcagctgcaggagtcaggccctggcctcgtaaagccttcggagactctgtccctgacgtgtaccgtcagcgggggatccatctccaactactactggacctggatcaggcagccgcctggaaagggtctggagtggatcggatacatcagcgactccgggtccaccaattacaacccctcgctgaagtcccgggtcaccatttccgtggacacctcaaagaaccagttcagcctgaagctgagctccgttaccgcggctgacacagccgtgtactactgcgcccgggaaaccatcgtcgtgcggggattctacttctacgtgatggacgtctggggccagggaaccactgtgactgtgtcctcc

SEQ ID NO: 156 - DNA, 321 bp, synthetic construct
  misc_feature 1..321: note = VL region (nt) (P21-V1)
  source 1..321: mol_type = other DNA
gccattcaactcacacagtccccgtcctctctgagcgcgtccgtgggtgatcgcgtgaccattacttgccgcgcctcacaaggcatcagctcggcgctcgcttggtaccagcagaagcctggaaaggcccctaagctgctgatctatgaagcctccaactgggagtcaggggtgccttcgaggttctccggaagcggatccggaactgacttcacgctcaccatctcctcgctgcaacccgaagatttcgccacctactactgccaacagttcaactcctacccctggactttcggacaaggcaccaaggtcgagattaag

SEQ ID NO: 157 - DNA, 1356 bp, synthetic construct
  misc_feature 1..1356: note = Heavy chain (nt) (P21-V1)
  source 1..1356: mol_type = other DNA
caggtgcagctgcaggagtcaggccctggcctcgtaaagccttcggagactctgtccctgacgtgtaccgtcagcgggggatccatctccaactactactggacctggatcaggcagccgcctggaaagggtctggagtggatcggatacatcagcgactccgggtccaccaattacaacccctcgctgaagtcccgggtcaccatttccgtggacacctcaaagaaccagttcagcctgaagctgagctccgttaccgcggctgacacagccgtgtactactgcgcccgggaaaccatcgtcgtgcggggattctacttctacgtgatggacgtctggggccagggaaccactgtgactgtgtcctccgcatcaactaaggggccgtcagtgtttcctcttgccccgtcctccaagtcgacttctggaggaaccgctgcccttggctgcctggtcaaagactatttcccggaacccgtcacggtgtcctggaacagtggagcgctgaccagcggtgtccacaccttcccggctgtgctccagtcctccggactgtacagcctgagctcggtcgtgactgtgccgtcctcgtccctgggcacccagacatacatctgcaacgtgaaccataagccctcgaacaccaaggtcgataagagagtggagccgaagtcgtgcgataagactcacacttgcccaccgtgtccagcgccagaagcggaaggggcacctagcgtgttcctgttcccgcccaagccgaaggacaccctcatgattagccggacccccgaagtcacctgtgtggtggtcgacgtgtcccacgaagatcccgaagtcaagttcaattggtacgtggacggcgtggaagtgcacaacgccaagaccaagccgcgcgaggaacagtacaactccacctaccgcgtggtgtccgtgctcaccgtgttgcatcaggattggctgaacgggaaggagtacaagtgcaaagtgtccaacaaagcactgcctgcccccattgagaaaaccattagcaaggccaagggacagcctcgggagccacaagtgtacactctgcccccatcgagagaagaaatgaccaagaatcaagtgtcactgacgtgcctcgtgaagggattttacccctcggacatcgccgtggagtgggagtcgaacggccagcccgagaacaactacaagactaccccgcctgtgttggatagcgacggctcgttcttcctgtattccaagctcactgtggacaagtcacgctggcaacagggcaacgtgttctcctgctccgtgatgcacgaagccctgcataaccactacacccagaagtccctgtctctgagccctggt

SEQ ID NO: 158 - DNA, 642 bp, synthetic construct
  misc_feature 1..642: note = Light chain (nt) (P21-V1)
  source 1..642: mol_type = other DNA
gccattcaactcacacagtccccgtcctctctgagcgcgtccgtgggtgatcgcgtgaccattacttgccgcgcctcacaaggcatcagctcggcgctcgcttggtaccagcagaagcctggaaaggcccctaagctgctgatctatgaagcctccaactgggagtcaggggtgccttcgaggttctccggaagcggatccggaactgacttcacgctcaccatctcctcgctgcaacccgaagatttcgccacctactactgccaacagttcaactcctacccctggactttcggacaaggcaccaaggtcgagattaagcgcaccgtggccgcccctagcgtgtttatcttccctccctcggatgagcagcttaagtcaggcaccgcatccgtggtctgcctgctcaacaacttctacccgagggaagccaaagtgcagtggaaagtggacaacgcgctccagtcgggaaactcccaggagtccgtgaccgaacaggactccaaggacagcacttattccctgtcctccactctgacgctgtcaaaggccgactacgagaagcacaaggtctacgcctgcgaagtgacccatcaggggctttcctcgcccgtgactaagagcttcaatcggggcgaatgc

SEQ ID NO: 159 - protein, 453 aa, synthetic construct
  REGION 1..453: note = heavy chain with post-translational modification (aa) (P20-V1)
XVQLQESGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 124 aa, synthetic construct
  REGION 1..124: note = VH region with post-translational modification (aa) (P20-V1)
  VARIANT 1: note = Xaa = pyroglutamate
XVQLQESGPGLVKPSETLSLTCTVSGGSINNYYWSWIRQPPGKGLEWIGYIFYSGTTNYNPSLKSRVTISVDTSKNQFSLKLSSVTAADTAVYYCARDVGIAARNSYYYVMDVWGQGTTVTVSS

SEQ ID NO: 161 - protein, 6 aa, synthetic construct
  REGION 1..6: note = Burosumab heavy chain CDR3 (aa)
DIVDAF

SEQ ID NO: 162 - protein, 452 aa, synthetic construct
  REGION 1..452: note = heavy chain with post-translational modification (aa) (P8-V1)
  MOD_RES 1: note = X = pyroglutamate
XVQLVQSGAEVKKPGASVKVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 123 aa, synthetic construct
  REGION 1..123: note = VH region with post-translational modification (aa) (P8-V1)
  MOD_RES 1: note = X = pyroglutamate
XVQLVQSGAEVKKPGASVKVSCKASGYTFTSYYMHWVRQAPGQGLEWMGIINPRGGYTTYAQKFQGRVTMTRDTSTSTVYMELSSLRSEDTAVYYCAKEGRVSPTDYYGMDVWGQGTTVTVSS

(19) United States
(12) Patent Application Publication
(10) Pub. No.: US 2025/0230251 A1
(43) Pub. Date: Jul. 17, 2025

(54) ANTIBODIES TARGETING IL-18 RECEPTOR BETA (IL-18RB) AND RELATED METHODS

(71) Applicant: Bristol-Myers Squibb Company, Princeton, NJ (US)

(72) Inventors: Lin Hui SU, Arlington, MA (US); Gilles BUCHWALTER, Cambridge, MA (US); Burce Ergel GURBUZBALABAN, Princeton, NJ (US); Jason Robert PINCKNEY, Princeton, NJ (US); Maryline Michele ABRIAL, Princeton, NJ (US); Benjamin CAUSTON, Holliston, MA (US); David Atwater CRITTON, Princeton, NJ (US); Tahmid Rashid HASSAN, Princeton, NJ (US); Galina Pylypiv KOH, Princeton, NJ (US); Stephanie NGUYEN, Princeton, NJ (US); Samantha Elaine PACE, Ewing, NJ (US); David John ZAMMIT, Princeton, NJ (US); Devang Praful SHAH, Princeton, NJ (US); Anastacia PARTON, Randolph, NJ (US); Malinda AITKEN, Warrington, PA (US); Aisling O'HARA HALL, Crosswicks, NJ (US); Andrew J. PARIS, Merion Station, PA (US)

(73) Assignee: Bristol-Myers Squibb Company, Princeton, NJ (US)

(21) Appl. No.: 18/988,731

(22) Filed: Dec. 19, 2024

Related U.S. Application Data
(60) Provisional application No. 63/645,496, filed on May 10, 2024.
(60) Provisional application No. 63/612,973, filed on Dec. 20, 2023.

(51) Int. Cl. C07K 16/28; A61K 39/00; A61K 45/06; A61P 1/00; A61P 37/06

(52) U.S. Cl. CPC C07K 16/2866; A61K 45/06; A61P 1/00; A61P 37/06; A61K 2039/505; C07K 2317/31; C07K 2317/40; C07K 2317/52; C07K 2317/565; C07K 2317/76; C07K 2317/92; C07K 2317/94

(57) ABSTRACT

Provided are novel antibodies and antigen-binding fragments thereof that bind interleukin-18 receptor beta (IL-18Rβ), along with conjugates thereof, nucleic acids encoding the same, compositions comprising the same, and methods of producing and using the same, including in the treatment of various diseases and conditions, such as inflammatory bowel disease, and other immune-mediated diseases, autoimmune diseases, inflammatory diseases, cancers, and infectious diseases.

### CROSS-REFERENCE TO RELATED APPLICATIONS

This application is a U.S. non-provisional application which claims priority to U.S. Provisional Application No. 63/612,973, filed Dec. 20, 2023, and U.S. Provisional Application No. 63/645,496, filed May 10, 2024, each entitled “ANTIBODIES TARGETING IL-18 RECEPTOR BETA (IL-18Rβ) AND RELATED METHODS,” the contents of which is incorporated by reference in their entireties.

### REFERENCE TO AN ELECTRONIC SEQUENCE LISTING

The Sequence Listing is provided as a file entitled 735042028300SeqList.xml, created on Feb. 21, 2025, which is 247,764 bytes in size. The information in the electronic format of the Sequence Listing is incorporated by reference in its entirety.

### FIELD

The present disclosure provides novel antibodies and antigen-binding fragments thereof that bind interleukin-18 receptor beta (IL-18Rβ), along with conjugates thereof, nucleic acids encoding the same, compositions comprising the same, and methods of producing and using the same, including in the treatment of various diseases and conditions, such as inflammatory bowel disease.

### BACKGROUND

IL-18 is a member of the IL-1 family of cytokines, and plays an important role in the activation of immune responses, including both Th1 and Th2 responses, in a variety of diseases. Thus, there is a need for targeting the IL-18 signaling pathway for treatment of such diseases. However, current treatment options have not been met with success. For instance, a large proportion of inflammatory bowel disease patients do not achieve remission with standard-of-care therapies. Embodiments provided herein address these needs.

### SUMMARY

Provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 3 (CDR-L3) comprising the amino acid sequence of SEQ ID NO: 24.

Also provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 3 (CDR-L3) contained within the amino acid sequence of SEQ ID NO: 35.

Also provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 1 (CDR-L1) comprising the amino acid sequence of SEQ ID NO: 22, a light chain complementarity determining region 2 (CDR-L2) comprising the amino acid sequence of SEQ ID NO: 36, and a light chain complementarity determining region 3 (CDR-L3) comprising the amino acid sequence of SEQ ID NO: 24.

Also provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 35.

In some of any of such embodiments, the V_{H} region is or comprises an amino acid sequence having at least, or at least about 85% sequence identity to the amino acid sequence of SEQ ID NO: 34. In some of any of such embodiments, the V_{H} region comprises an amino acid sequence having at least, or at least about 90% sequence identity to the amino acid sequence of SEQ ID NO: 34. In some of any of such embodiments, the V_{H} region comprises an amino acid sequence having at least, or at least about 95% sequence identity to the amino acid sequence of SEQ ID NO: 34. In some of any of such embodiments, the V_{H} region comprises an amino acid sequence having at least, or at least about 97% sequence identity to the amino acid sequence of SEQ ID NO: 34. In some of any of such embodiments, the V_{H} region comprises an amino acid sequence having at least, or at least about 99% sequence identity to the amino acid sequence of SEQ ID NO: 34.

In some of any of such embodiments, the V_{L} region comprises an amino acid sequence having at least, or at least about 85% sequence identity to the amino acid sequence of SEQ ID NO: 35. In some of any of such embodiments, the V_{L} region comprises an amino acid sequence having at least, or at least about 90% sequence identity to the amino acid sequence of SEQ ID NO: 35. In some of any of such embodiments, the V_{L} region comprises an amino acid sequence having at least, or at least about 95% sequence identity to the amino acid sequence of SEQ ID NO: 35. In some of any of such embodiments, the V_{L} region comprises an amino acid sequence having at least, or at least about 97% sequence identity to the amino acid sequence of SEQ ID NO: 35. In some of any of such embodiments, the V_{L} region comprises an amino acid sequence having at least, or at least about 99% sequence identity to the amino acid sequence of SEQ ID NO: 35.

In some of any of such embodiments, the V_{H} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 34; and the V_{L} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 35.

In some of any of such embodiments, the V_{H} region comprises the amino acid sequence of SEQ ID NO: 34. In some of any of such embodiments, the V_{L} region comprises the amino acid sequence of SEQ ID NO: 35. In some of any of such embodiments, the V_{H} region comprises the amino acid sequence of SEQ ID NO: 34; and the V_{L} region comprises the amino acid sequence of SEQ ID NO: 35.

In some of any of such embodiments, the antibody or antigen-binding fragment thereof is an antibody comprising a constant domain. In some of any of such embodiments, the constant domain is a constant domain selected from the group consisting of an IgA1 constant domain, an IgA2 constant domain, an IgD constant domain, an IgE constant domain, an IgG1 constant domain, an IgG2 constant domain, an IgG3 constant domain, an IgG4 constant domain, and an IgM constant domain. In some of any of such embodiments, the constant domain is an IgG1 constant domain.

In some of any of such embodiments, the antibody comprises: a heavy chain constant domain comprising the amino acid sequence of SEQ ID NO: 30, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and a light chain constant domain comprising the amino acid sequence of SEQ ID NO: 31, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31. In some of any of such embodiments, the antibody comprises a heavy chain constant domain comprising the amino acid sequence of SEQ ID NO: 30, and a light chain constant domain comprising the amino acid sequence of SEQ ID NO: 31.

In some of any of such embodiments, the antibody comprises: a heavy chain comprising the amino acid sequence of SEQ ID NO: 32, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising the amino acid sequence of SEQ ID NO: 33, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 33. In some of any of such embodiments, the antibody comprises a heavy chain comprising the amino acid sequence of SEQ ID NO: 32, and a light chain comprising the amino acid sequence of SEQ ID NO: 33.

Also provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 52, a heavy chain complementarity determining region 2 (CDR-H2) comprising the amino acid sequence of SEQ ID NO: 53, and a heavy chain complementarity determining region 3 (CDR-H3) comprising the amino acid sequence of SEQ ID NO: 54; and the V_{L} region comprises a light chain complementarity determining region 1 (CDR-L1) comprising the amino acid sequence of SEQ ID NO: 66, a light chain complementarity determining region 2 (CDR-L2) comprising an amino acid sequence selected from the group consisting of SEQ ID NOs: 67, and a light chain complementarity determining region 3 (CDR-L3) comprising the amino acid sequence of SEQ ID NO: 68.

Also provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 75; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 76.

In some of any of such embodiments, the V_{H} region comprises an amino acid sequence having at least, or at least about 85% sequence identity to the amino acid sequence of SEQ ID NO: 75. In some of any of such embodiments, the V_{H} region comprises an amino acid sequence having at least, or at least about 90% sequence identity to the amino acid sequence of SEQ ID NO: 75. In some of any of such embodiments, the V_{H} region comprises an amino acid sequence having at least, or at least about 95% sequence identity to the amino acid sequence of SEQ ID NO: 75. In some of any of such embodiments, the V_{H} region comprises an amino acid sequence having at least, or at least about 97% sequence identity to the amino acid sequence of SEQ ID NO: 75. In some of any of such embodiments, the V_{H} region comprises an amino acid sequence having at least, or at least about 99% sequence identity to the amino acid sequence of SEQ ID NO: 75.

In some of any of such embodiments, the V_{L} region comprises an amino acid sequence having at least, or at least about 85% sequence identity to the amino acid sequence of SEQ ID NO: 76. In some of any of such embodiments, the V_{L} region comprises an amino acid sequence having at least, or at least about 90% sequence identity to the amino acid sequence of SEQ ID NO: 76. In some of any of such embodiments, the V_{L} region comprises an amino acid sequence having at least, or at least about 95% sequence identity to the amino acid sequence of SEQ ID NO: 76. In some of any of such embodiments, the V_{L} region comprises an amino acid sequence having at least, or at least about 97% sequence identity to the amino acid sequence of SEQ ID NO: 76. In some of any of such embodiments, the V_{L} region comprises an amino acid sequence having at least, or at least about 99% sequence identity to the amino acid sequence of SEQ ID NO: 76.

In some of any of such embodiments, the V_{H} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 75; and the V_{L} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 76.

In some of any of such embodiments, the V_{H} region comprises the amino acid sequence of SEQ ID NO: 75. In some of any of such embodiments, the V_{L} region comprises the amino acid sequence of SEQ ID NO: 76. In some of any of such embodiments, the V_{H} region comprises the amino acid sequence of SEQ ID NO: 75; and the V_{L} region comprises the amino acid sequence of SEQ ID NO: 76.

In some of any of such embodiments, the antibody or antigen-binding fragment thereof is an antibody comprising a constant domain. In some of any of such embodiments, the constant domain is a constant domain selected from the group consisting of an IgA1 constant domain, an IgA2 constant domain, an IgD constant domain, an IgE constant domain, an IgG1 constant domain, an IgG2 constant domain, an IgG3 constant domain, an IgG4 constant domain, and an IgM constant domain. In some of any of such embodiments, the constant domain is an IgG1 constant domain.

In some of any of such embodiments, the antibody comprises: a heavy chain constant domain comprising the amino acid sequence of SEQ ID NO: 30, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and a light chain constant domain comprising the amino acid sequence of SEQ ID NO: 31, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31. In some of any of such embodiments, the antibody comprises a heavy chain constant domain comprising the amino acid sequence of SEQ ID NO: 30, and a light chain constant domain comprising the amino acid sequence of SEQ ID NO: 31.

In some of any of such embodiments, the antibody comprises: a heavy chain comprising the amino acid sequence of SEQ ID NO: 73, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 73; and a light chain comprising the amino acid sequence of SEQ ID NO: 74, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 74.

In some of any of such embodiments, the antibody comprises a heavy chain comprising the amino acid sequence of SEQ ID NO: 73, and a light chain comprising the amino acid sequence of SEQ ID NO: 74.

In some of any of such embodiments, the antibody or antigen-binding fragment thereof is an antigen-binding fragment. In some of any of such embodiments, the antigen-binding fragment is selected from the group consisting of a single domain antibody, a single chain antibody, an unibody, a single chain variable fragment (scFv), a Fab fragment, and a F(ab′)₂ fragment.

In some of any of such embodiments, the antibody or antigen-binding fragment thereof is recombinant. In some of any of such embodiments, the antibody or antigen-binding fragment thereof is monoclonal. In some of any of such embodiments, the antibody or antigen-binding fragment thereof is human.

In some of any of such embodiments, the antibody or antigen-binding fragment thereof binds to human IL-18Rβ at pH 7.4 with an equilibrium dissociation constant (K_{D}) that is about 1.5 nM or is less than about 1.5 nM. In some of any of such embodiments, the antibody or antigen-binding fragment thereof binds to cynomolgus IL-18Rβ at pH 7.4 with an equilibrium dissociation constant (K_{D}) that is about 2 nM or is less than about 2 nM. In some of any of such embodiments, the antibody or antigen-binding fragment thereof binds to human IL-18Rβ at pH 7.4 with an equilibrium dissociation constant (K_{D}) that is, is about, is less than, or is less than about 2 nM. In some of any of such embodiments, the antibody or antigen-binding fragment thereof has a K_{D} ratio of K_{D} at pH 5 to K_{D} at pH 7 of 0.33 to 3.33, or about 0.33 to about 3.33. In some of any of such embodiments, the K_{D} ratio is 0.8 to 1.3, or about 0.8 to about 1.3.

In some of any of such embodiments, the antibody or antigen-binding fragment thereof has a K_{D} ratio of K_{D} at pH 5 to K_{D} at pH 7 of 0.25 to 1.00, or about 0.25 to about 1.00. In some of any of such embodiments, the K_{D} ratio is 0.25 to 0.5, or about 0.25 to about 0.5.

In some of any of such embodiments, the anti-IL-18Rβ antibody or antigen-binding fragment thereof binds to an epitope of human IL-18Rβ that comprises, consists essentially of, or consists of residues 242-248, 279, 281, 282, 312, 342, and 343, with residue numbering corresponding to the amino acid sequence of SEQ ID NO: 126.

In some of any of such embodiments, the human IL-18Rβ comprises, consists essentially of, or consists of the amino acid sequence of SEQ ID NO: 126.

In some of any of such embodiments, the anti-IL-18Rβ antibody or antigen-binding fragment thereof blocks IL-18Rβ binding to an IL-18/IL-18Rα complex.

In some of any of such embodiments, the anti-IL-18Rβ antibody or antigen-binding fragment thereof comprises at least one post-translational modification of the amino acid sequence, optionally wherein the at least one post-translational modification comprises a post-translational modification of a heavy chain N-terminal glutamine (Q) to a pyroglutamate.

Also provided herein is a multi-specific antibody, comprising a first antigen-binding domain that binds to IL-18Rβ and a second antigen-binding domain that binds to a second antigen, wherein the first antigen-binding domain comprises any anti-IL-18Rβ antibody or antigen-binding fragment thereof disclosed herein. In some embodiments, the multi-specific antibody is a bispecific antibody.

Also provided herein is a conjugate, comprising any anti-IL-18Rβ antibody or antigen-binding fragment thereof disclosed herein, or any multi-specific antibody disclosed herein, and a heterologous molecule or moiety.

Also provided herein is a polynucleotide comprising a nucleic acid(s) encoding any anti-IL-18Rβ antibody or antigen-binding fragment thereof disclosed herein, or a heavy chain or a light chain thereof.

Also provided herein is a nucleic acid(s) encoding any anti-IL-18Rβ antibody or antigen-binding fragment thereof disclosed herein, or a heavy chain or a light chain thereof.

In some embodiments, the nucleic acid(s) comprises a nucleic acid encoding a V_{H} region comprising the nucleotide sequence of SEQ ID NO: 127 or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 127; and a nucleic acid encoding a V_{L} region comprising the nucleotide sequence of SEQ ID NO: 128, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 128. In some embodiments, the nucleic acid(s) comprises a nucleic acid encoding a V_{H} region comprising the nucleotide sequence of SEQ ID NO: 127; and a nucleic acid encoding a V_{L} region comprising the nucleotide sequence of SEQ ID NO: 128. In some embodiments, the nucleic acid(s) comprises a nucleic acid encoding a heavy chain comprising the nucleotide sequence of SEQ ID NO: 129, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 129; and a nucleic acid encoding a light chain comprising the nucleotide sequence of SEQ ID NO: 130, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 130. In some embodiments, the nucleic acid(s) comprises a nucleic acid encoding a heavy chain comprising the nucleotide sequence of SEQ ID NO: 129; and a nucleic acid encoding a light chain comprising the nucleotide sequence of SEQ ID NO: 130.

In some embodiments, the nucleic acid(s) comprises a nucleic acid encoding a V_{H} region comprising the nucleotide sequence of SEQ ID NO: 131, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 131; and a nucleic acid encoding a V_{L} region comprising the nucleotide sequence of SEQ ID NO: 132, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 132. In some embodiments, the nucleic acid(s) comprises a nucleic acid encoding a V_{H} region comprising the nucleotide sequence of SEQ ID NO: 131; and a nucleic acid encoding a V_{L} region comprising the nucleotide sequence of SEQ ID NO: 132. In some embodiments, the nucleic acid(s) comprises a nucleic acid encoding a heavy chain comprising the nucleotide sequence of SEQ ID NO: 133, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 133; and a nucleic acid encoding a light chain comprising the nucleotide sequence of SEQ ID NO: 134, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 134. In some embodiments, the nucleic acid(s) comprises a nucleic acid encoding a heavy chain comprising the nucleotide sequence of SEQ ID NO: 133; and a nucleic acid encoding a light chain comprising the nucleotide sequence of SEQ ID NO: 134.

Also provided herein is a vector, comprising any polynucleotide or any nucleic acid(s) disclosed herein. In some embodiments, the vector is a viral vector. In some embodiments, the viral vector is a retroviral vector or a lentiviral vector.

Also provided herein is a host cell comprising any anti-IL-18Rβ antibody or antigen-binding fragment thereof disclosed herein, any polynucleotide disclosed herein, any nucleic acid(s) disclosed herein, or any vector disclosed herein.

Also provided herein is a method of producing an antibody or antigen-binding fragment thereof comprising culturing any host cell disclosed herein under a condition that produces the antibody or antigen-binding fragment thereof. In some embodiments, the method further comprises recovering the antibody or antigen-binding fragment thereof produced by the host cell.

Also provided herein is an antibody or antigen-binding fragment thereof produced by any method of production disclosed herein.

Also provided herein is a composition comprising any anti-IL-18Rβ antibody or antigen-binding fragment thereof disclosed herein, any multi-specific antibody disclosed herein, or any conjugate disclosed herein.

In some of embodiments, the composition further comprises a pharmaceutically acceptable excipient.

Also provided herein is a method of treatment, comprising administering any anti-IL-18Rβ antibody or antigen-binding fragment thereof disclosed herein, any multi-specific antibody disclosed herein, any conjugate disclosed herein, or any composition disclosed herein, to a subject having a disease or disorder.

In some embodiments, the method of treatment further comprises administering one or more additional therapeutic agents. In some embodiments, the one or more additional therapeutic agents comprises, consists essentially of, or consists of an aminosalicylate, a corticosteroid, a thiopurine, a methotrexate, a tumor necrosis factor (TNF) inhibitor, an α4β7 integrin inhibitor, a TNF-α inhibitor, an IL-23 inhibitor, a dual IL-12 and IL-23 inhibitor, a tumor necrosis factor-like cytokine 1A (TL1A) inhibitor, an IL-2-CD25 fusion protein, a bispecific anti-TL1A/anti-TNF-α binding protein, an E-type prostanoid receptor 4 (EP4) agonist, a TYK2 inhibitor, a sphingosine-1-phosphate receptor (S1PR) agonist, a Janus kinase (JAK) inhibitor, an interkeukin-1 receptor associated kinase 4 (IRAK-4) inhibitor, a toll-like receptor 7 and 8 (TLR7/8) inhibitor, or an IL-22 inhibitor.

In some of any of such embodiments, the one or more additional therapeutic agents is administered concurrently with the anti-IL-18Rβ antibody or antigen-binding fragment thereof, the multi-specific antibody, the conjugate, or the composition. In some of any of such embodiments, the one or more additional therapeutic agents is administered before or after the anti-IL-18Rβ antibody or antigen-binding fragment thereof, the multi-specific antibody, the conjugate, or the composition.

In some of any of such embodiments, the disease or disorder is an immune-mediated disease, an autoimmune disease, an inflammatory disease, a cancer, or an infectious disease. In some of any of such embodiments, the disease or disorder is associated with an increase in IL-18Rβ in a biological sample of the subject as compared to a subject not having the disease or disorder. In some embodiments, the biological sample is a serum sample.

In some of any of such embodiments, the disease or disorder is an immune-mediated disease. In some of any of such embodiments, the disease or disorder is an inflammatory disease. In some of any of such embodiments, the disease or disorder is an inflammatory bowel disease. In some embodiments, the inflammatory bowel disease is Crohn's disease. In some embodiments, the inflammatory bowel disease is ulcerative colitis. In some of any of such embodiments, the disease or disorder is chronic obstructive pulmonary disease (COPD). In some of any of such embodiments, the disease or disorder is an autoimmune disease. In some of any of such embodiments, the disease or disorder is a cancer. In some of any of such embodiments, the disease or disorder is an infectious disease.

In some of any of such embodiments, the disease or disorder is inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, Celiac disease, or age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).

In some of any of such embodiments: the inflammatory bowel disease is Crohn's disease or ulcerative colitis; the arthritis is rheumatoid arthritis, psoriatic arthritis, idiopathic arthritis, or giant cell arthritis; the psoriasis is plaque psoriasis; the organ transplant rejection is a kidney transplant rejection; the heart disease is ischemic heart disease; the MAS is infantile MAS; or the sarcoidosis is pulmonary sarcoidosis. In some of any of such embodiments: the rheumatoid arthritis is juvenile rheumatoid arthritis; or the idiopathic arthritis is juvenile idiopathic arthritis.

In some of any of such embodiments, the subject is a human.

Also provided herein is a use of any composition disclosed herein for the manufacture of a medicament for treatment of a disease or disorder.

Also provided herein is a use of any composition disclosed herein for treatment of a disease or disorder.

Also provided herein is any composition disclosed herein for use in treatment of a disease or disorder.

In some of any of such embodiments, the treatment further comprises administering one or more additional therapeutic agents. In some of any of such embodiments, the one or more additional therapeutic agents comprises, consists essentially of, or consists of an aminosalicylate, a corticosteroid, a thiopurine, a methotrexate, a tumor necrosis factor (TNF) inhibitor, an α4β7 integrin inhibitor, a TNF-α inhibitor, an IL-23 inhibitor, a dual IL-12 and IL-23 inhibitor, a tumor necrosis factor-like cytokine 1A (TL1A) inhibitor, an IL-2-CD25 fusion protein, a bispecific anti-TL1A/anti-TNF-α binding protein, an E-type prostanoid receptor 4 (EP4) agonist, a TYK2 inhibitor, a sphingosine-1-phosphate receptor (S1PR) agonist, a Janus kinase (JAK) inhibitor, an interkeukin-1 receptor associated kinase 4 (IRAK-4) inhibitor, a toll-like receptor 7 and 8 (TLR7/8) inhibitor, or an IL-22 inhibitor.

In some of any of such embodiments, the one or more additional therapeutic agents is to be administered concurrently with the anti-IL-18Rβ antibody or antigen-binding fragment thereof, the multi-specific antibody, the conjugate, or the composition. In some of any of such embodiments, the one or more additional therapeutic agents is to be administered sequentially with the anti-IL-18Rβ antibody or antigen-binding fragment thereof, the multi-specific antibody, the conjugate, or the composition.

In some of any of such embodiments, the disease or disorder is an immune-mediated disease, an autoimmune disease, an inflammatory disease, a cancer, or an infectious disease. In some of any of such embodiments, the disease or disorder is an immune-mediated disease. In some of any of such embodiments, the disease or disorder is an inflammatory disease. In some of any of such embodiments, the disease or disorder is an inflammatory bowel disease. In some of any of such embodiments, the inflammatory bowel disease is Crohn's disease. In some of any of such embodiments, the inflammatory bowel disease is ulcerative colitis. In some of any of such embodiments, the disease or disorder is chronic obstructive pulmonary disease (COPD). In some of any of such embodiments, the disease or disorder is an autoimmune disease. In some of any of such embodiments, the disease or disorder is a cancer. In some of any of such embodiments, the disease or disorder is an infectious disease.

In some of any of such embodiments, the disease or disorder is inflammatory bowel disease, allergy, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, Celiac disease, or age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).

In some of any of such embodiments: the inflammatory bowel disease is Crohn's disease or ulcerative colitis; the arthritis is rheumatoid arthritis, psoriatic arthritis, idiopathic arthritis, or giant cell arthritis; the psoriasis is plaque psoriasis; the organ transplant rejection is a kidney transplant rejection; the heart disease is ischemic heart disease; the MAS is infantile MAS; or the sarcoidosis is pulmonary sarcoidosis. In some of any of such embodiments: the rheumatoid arthritis is juvenile rheumatoid arthritis; or the idiopathic arthritis is juvenile idiopathic arthritis.

### BRIEF DESCRIPTION OF THE DRAWINGS

FIG. 1 shows a human IL-18Rβ LSA binding sensorgram overlay for one panel of candidate α-human IL-18Rβ antibodies.

FIG. 2 shows an isoaffinity plot for LSA screening of candidate α-IL-18Rβ antibodies.

FIG. 3 shows a human IL-18Rβ isoaffinity plot for Biacore 8K screening of candidate α-IL-18Rβ antibodies.

FIG. 4 shows a cynomolgus monkey IL-18Rβ isoaffinity plot for Biacore 8K screening of candidate α-IL-18Rβ antibodies.

FIG. 5 shows a representative sensorgram for an Octet IL-18Rβ blocking assay.

FIG. 6 with partial views FIG. 6A, FIG. 6B, and FIG. 6C shows rank-ordered binding responses of IL-18: IL-18Rα complex to antibody-blocked IL-18Rβ.

FIG. 7 with partial views FIG. 7A, FIG. 7B, and FIG. 7C shows a combined binary dendrogram for candidate α-human IL-18Rβ blocking antibodies included in a LSA epitope binning assay.

FIG. 8 shows a community node plot highlighting three main epitope bins for candidate α-huIL-18Rβ blocking antibodies.

FIG. 9A-FIG. 9D show multi-pH sensorgrams for two α-human IL-18Rβ antibodies binding to human IL-18Rβ. FIG. 9A shows sensorgrams for P20 and P8 at a pH of 7.4; FIG. 9B shows sensorgrams for P20 and P8 at a pH of 5.0; FIG. 9C shows sensorgrams for P20 and P8 at a pH of 5.5; and FIG. 9D shows sensorgrams for P20 and P8 at a pH of 6.0.

FIG. 10A shows a bar plot depicting the percent monomericity of antibody P20 at varying storage conditions using size exclusion chromatography.

FIG. 10B shows a bar plot depicting the percent monomericity of antibody P21 at varying storage conditions using size exclusion chromatography.

FIG. 10C shows a bar plot depicting the percent monomericity of antibody P8 at varying storage conditions using size exclusion chromatography.

FIG. 11 depicts the chemical stability of three α-huIL-18Rβ antibodies as a function of time and temperature.

FIG. 12 with partial views FIG. 12A and FIG. 12B shows a heat map for the light chain of antibody P20 assessing whether position D50 in the LCDR2 can be engineered to a different amino acid without losing activity, based on a deep mutational scan.

FIG. 13 shows a bar plot depicting the percent monomericity of antibodies P20-V1 and P21-V1 at varying storage conditions using size exclusion chromatography.

FIG. 14A shows the crystal structure of the IL-18Rβ+P20 fragment antibody binding (Fab) complex. Left, representation of the IL-18Rβ (molecular surface) epitope (dark grey) with the P20 Fab (cartoon; heavy chain, light grey; light chain, dark grey) paratope (sticks). Right, representation of the IL-18Rβ (cartoon) epitope (sticks) with the P20 Fab (molecular surface; heavy chain, light grey; light chain, medium grey) paratope (dark grey). Disordered internal loops represented as dashes.

FIG. 14B shows the crystal structure of the IL-18Rβ+P8 fragment antibody binding (Fab) complex. Left, representation of the IL-18Rβ (molecular surface) epitope (dark grey) with the P8 Fab (cartoon; heavy chain, light grey; light chain, dark grey) paratope (sticks). Right, representation of the IL-18Rβ (cartoon) epitope (sticks) with the P8 Fab (molecular surface: heavy chain, light grey; light chain, medium grey) paratope (dark grey). Disordered internal loops represented as dashes.

FIG. 14C shows crystal structures of different IL-18Rβ+anti-IL-18Rβ fragment antibody binding (Fab) complexes, where the complexes are IL-18Rβ+P20 Fab complex (left), IL-18Rβ+P20 Fab complex (middle), or IL-18Rβ+burosumab Fab complex (right). In each image, the HCDR3 of each anti-IL-18Rβ Fab is shown as a transparent surface and the LCDR2 of each anti-IL-18Rβ Fab is shown as dots.

FIG. 15 depicts the inhibition of interferon gamma (IFNg or IFNg) in KG-1 cells when treated with anti-human IL-18Rβ monoclonal antibody P20-V1 (average of 7 independent experiments).

FIG. 16 depicts the inhibition of interferon gamma (IFNg or IFNg) in human whole blood (LPS+IL-12 stimulation, n=15 donors) when treated with anti-human IL-18Rβ monoclonal antibody P20-V1.

FIG. 17 depicts the inhibition of interferon gamma (IFNg or IFNg) in human whole blood (IL-18+IL-12 stimulation, n=8 donors) when treated with anti-human IL-18Rβ monoclonal antibody P20-V1.

FIG. 18 depicts the inhibition of interferon gamma (IFNg or IFNg) in cynomolgus monkey whole blood (IL-18+IL-12 stimulation, n=14 donors) when treated with anti-human IL-18R monoclonal antibody P20-V1.

FIG. 19 depicts the dose-dependent exposure of anti-human IL-18Rβ monoclonal antibody P20-V1 in the serum of cynomolgus monkeys following P20-V1 administration in vivo.

FIG. 20 depicts the sustained inhibition of interferon gamma (IFNg or IFNg) in an ex vivo cynomolgus monkey (cyno) whole blood assay where whole blood taken from cyno was stimulated with human IL-18 and IL-12 to induce IFNg production following administration of anti-human IL-18Rβ monoclonal antibody P20-V1 at the indicated doses in vivo. The top panel shows IFNg concentration and the bottom panel shows IFNg inhibition for the indicated experimental conditions.

FIG. 21 depicts the lack of inhibition of interferon gamma (IFNg or IFNg) in wild-type mice whole blood stimulated with recombinant murine IL-18 and IL-12 when treated with anti-human IL-18Rβ monoclonal antibody P20-V1. The top panel shows IFNg concentration and the bottom panel shows IFNg inhibition for the indicated experimental conditions.

FIG. 22 depicts the inhibition of interferon gamma (IFNg or IFNg) in human IL-18Rβ knock-in mice whole blood stimulated with recombinant murine IL-18 and IL-12 when treated with anti-human IL-18Rβ monoclonal antibody P20-V1. The top panel shows IFNg concentration and the bottom panel shows IFNg inhibition for the indicated experimental conditions.

FIG. 23 depicts the lack of inhibition of interferon gamma (IFNg or IFNg) in wild-type mice splenocytes stimulated with recombinant murine IL-12 and LPS when treated with anti-human IL-18Rβ monoclonal antibody P20-V1. The top panel shows IFNg concentration and the bottom panel shows IFNg inhibition for the indicated experimental conditions.

FIG. 24 depicts the interferon gamma (IFNg or IFNg) concentration (top panel) and inhibition of IFNg (bottom panel) in human IL-18Rβ knock-in mice splenocytes that were unstimulated, or stimulated with recombinant murine IL-12 and LPS when treated with anti-human IL-18Rβ monoclonal antibody P20-V1 or isotype control.

FIG. 25 depicts IFNg (IFNg) concentration (left panel) and the inhibition of IFNg (right panel) in human IL-18Rβ knock-in mice whole blood that was unstimulated, or stimulated with IL-12 and IL-18 following in vivo administration of isotype control or anti-human IL-18Rβ monoclonal antibody P20-V1 at the indicated mg per kg (mpk) doses.

FIG. 26 depicts the dose-dependent exposure of anti-human IL-18Rβ monoclonal antibody P20-V1 in the serum of human IL-18Rβ knock-in mice following P20-V1 administration in vivo.

FIG. 27 depicts dose curve for ulcerative colitis (UC) lamina propria lymphocytes (LPLs) (n=7) pre-treated with P20-V1 or recombinant human IL-18 binding protein (IL-18BP) at various concentrations for 1 hour and stimulated with rhIL-12 (5 ng/ml) and rhIL-18 (6 ng/ml) for 24 hours. The top panel shows the IFNg (IFNg) concentration and the bottom panel shows percent inhibition of IFNg for the indicated experimental conditions.

FIG. 28 depicts dose curve for Crohn's disease (CD) lamina propria lymphocytes (LPLs) (n=8) pre-treated with P20-V1 or recombinant human IL-18 binding protein (IL-18BP) at various concentrations for 1 hour and stimulated with rhIL-12 (5 ng/ml) and rhIL-18 (6 ng/ml) for 24 hours. The top panel shows the IFNg (IFNg) concentration and the bottom panel shows percent inhibition of IFNg for the indicated experimental conditions.

FIG. 29 depicts dose curves for healthy, i.e., non-inflammatory bowel disease (IBD) lamina propria lymphocytes (LPLs) (n=6) pre-treated with P20-V1 or recombinant human IL-18 binding protein (IL-18BP) at various concentrations for 1 hour and stimulated with rhIL-12 (5 ng/ml) and rhIL-18 (6 ng/ml) for 24 hours. The top panel shows the IFNg (IFNg) concentration and the right panel shows percent inhibition of IFNg for the indicated experimental conditions.

FIGS. 30A-30C depict mRNA gene expression of IFNg (IFNG) (FIG. 30A), CXCL9 (FIG. 30B), and CXCL10 (FIG. 30C), respectively, of 3 donors of ulcerative colitis (UC) lamina propria lymphocytes (LPLs) pre-treated with P20-V1 at indicated concentrations for 1 hour and stimulated with rhIL-12 (5 ng/ml) and rhIL-18 (6 ng/ml) for 24 hours. Data was normalized to the GAPDH, ACTB, HPRT, RPLP0 and 18S house-keeping controls.

FIG. 31 depicts body weight in a healthy control mouse model (PBS), or in an IL-12/IL-18-induced colitis mice model following injection with a control isotype antibody (Isotype) or the indicated mg/kg (mpk) dose of anti-human IL-18Rβ monoclonal antibody P20-V1. Change in body weight over 4 days is shown in the left panel, and net change in body weight as quantified by area under the curve (AUC) is shown in the right panel.

FIG. 32 depicts total (left) and damage (right) histology scores in the colon of healthy control mice (PBS), and in IL-12/IL-18-induced colitis mice (IL-12/IL-18) following injection with a control isotype antibody (Ig) or with the indicated dose of anti-human IL-18Rβ monoclonal antibody P20-V1.

FIG. 33 depicts total (left) and damage (right) histology scores in the ileum of healthy control mice (PBS), and in IL-12/IL-18-induced colitis mice (IL-12/IL-18) following injection with a control isotype antibody (Ig) or with the indicated dose of anti-human IL-18Rβ monoclonal antibody P20-V1.

FIG. 34 depicts levels of anti-human IL-18Rβ monoclonal antibody P20-V1 in the serum (left panel), colon (middle panel), or ileum (right panel) in an IL-12/IL-18-induced colitis mouse model 120 hours post-administration of the indicated dose of P20-V1.

FIG. 35 depicts IFNg (IFNg) concentration (left panel) and the inhibition of IFNg (right panel) in serum from a healthy control mouse model (PBS), or from an IL-12/IL-18-induced colitis mouse model (IL-12/-18) following in vivo administration of isotype control (Isotype) or anti-human IL-18Rβ monoclonal antibody P20-V1 at the indicated mg per kg (mpk) doses.

FIG. 36 depicts IFNg (IFNg) concentration (left panel) and the inhibition of IFNg (right panel) in colons from a healthy control mouse model (PBS), or from an IL-12/IL-18-induced colitis mouse model (IL-12/-18) following in vivo administration of isotype control (Isotype) or anti-human IL-18Rβ monoclonal antibody P20-V1 at the indicated mg per kg (mpk) doses.

FIG. 37 depicts IFNg (IFNg) concentration (left panel) and the inhibition of IFNg (right panel) in ileums from a healthy control mouse model (PBS), or from an IL-12/IL-18-induced colitis mouse model (IL-12/-18) following in vivo administration of isotype control (Isotype) or anti-human IL-18Rβ monoclonal antibody P20-V1 at the indicated mg per kg (mpk) doses.

### DETAILED DESCRIPTION

The present disclosure provides anti-IL-18Rβ antibodies and antigen-binding fragments thereof, as well as multi-specific antibodies and conjugates comprising the same, and nucleic acids and vectors encoding the same. Also provided herein are methods of producing the anti-IL-18Rβ antibodies and antigen-binding fragments thereof, the multi-specific antibodies, and conjugates, and compositions, e.g., pharmaceutical compositions, comprising any of the foregoing. Also provided are methods of using any of the anti-IL-18Rβ antibodies and antigen-binding fragments thereof, multi-specific antibodies, conjugates, or compositions described herein, such as in the treatment of a disease or disorder, diagnosis, assessing IL-18Rβ expression or activity, or in monitoring the treatment of a disease or disorder.

IL-18 is a pro-inflammatory cytokine that is associated with a variety of diseases, including immune-mediated diseases, autoimmune diseases, inflammatory diseases, cancers, and infectious diseases.

For instance, IL-18 has been implicated in having a pathogenic role in diseases and disorders including inflammatory bowel disease, e.g., Crohn's disease and ulcerative colitis. The binding of IL-18Rβ to the IL-18/IL-18Rα complex results in formation of an IL-18 receptor-ligand complex and activation of the TIR domain of the IL-18 receptor, which leads to signal propagation, including the production of interferon gamma (IFN-γ). This complex signals through activation of the Toll/IL-1 (TIR) domain, which is responsible for propagation of downstream signaling. Such activation of the TIR domain of the IL-18 receptor is known to promote the initiation and progression of diseases, including immune-mediated diseases, autoimmune diseases, inflammatory diseases, cancers, and infectious diseases. Blocking the binding of the IL-18Rβ subunit to the IL-18/IL-18Rα complex can inhibit the downstream deleterious effects that occur through the IL-18 signaling pathway in such diseases and disorders, such as with regards to detrimental inflammatory responses. The anti-IL-18Rβ antibodies and antigen-binding fragments thereof provided herein advantageously block the binding of IL-18Rβ to the IL-18/IL-18Rα complex to inhibit IL-18 signaling, while exhibiting additional structural and/or functional features that give rise to additional advantageous effects. These additional advantageous effects include, in some embodiments, high affinity binding to human and cynomolgus monkey IL-18Rβ: potency in inhibition of IFNg production; pH-independent binding; low polyreactivity to extracellular matrix proteins; stability; a low immunogenicity risk; and/or being a human antibody, or any combination thereof. In some embodiments, provided herein are anti-IL-18Rβ antibodies and antigen-binding fragments thereof that block the binding of IL-18Rβ to the IL-18/IL-18Rα complex to inhibit IL-18 signaling, exhibit high affinity binding to human and cynomolgus monkey IL-18Rβ: exhibit potency in inhibition of IFNg production; exhibit pH-independent binding; exhibit low polyreactivity to extracellular matrix proteins; exhibit stability; exhibit a low immunogenicity risk; and are human.

Among the provided anti-IL-18Rβ antibodies and antigen-binding fragments thereof are those that exhibit high affinity binding to both human and cynomolgus monkey IL-18Rβ. This is advantageous, for instance, because such antibodies can be used in preclinical studies (e.g., toxicology studies) in cynomolgus monkey; this obviates the need to use a different, surrogate antibody for such studies.

In addition, in some embodiments, the provided anti-IL-18Rβ antibodies and antigen-binding fragments thereof exhibit potency in inhibition of IFNg production, including sustained inhibition over an extended period of time. This is advantageous because by blocking IL-18-induced IFNg production that is characteristic of many diseases and disorders mediated, at least in part, by IL-18 pathway signaling, such diseases and disorders can be treated.

In addition, in some embodiments, the provided anti-IL-18Rβ antibodies and antigen-binding fragments thereof exhibit pH-independent binding. This is advantageous because the anti-IL-18Rβ antibody or antigen-binding fragment thereof can bind to IL-18Rβ at physiological pH, while also binding to IL-18Rβ in more acidic environments, such as the gastrointestinal tract, e.g., for treatment of inflammatory bowel disease, or in tumor microenvironments, e.g., for treatment of cancers, among other acidic environments.

In addition, in some embodiments, the provided anti-IL-18Rβ antibodies and antigen-binding fragments thereof exhibit low polyreactivity to extracellular matrix (ECM) proteins. This is advantageous because low polyreactivity with ECM proteins can reduce off-target effects caused by the antibody binding to ECM proteins, and can reduce or avoid the negative effects that polyreactivity can have on pharmacokinetics and bioavailability of the antibody.

In addition, in some embodiments, the provided anti-IL-18Rβ antibodies and antigen-binding fragments thereof exhibit stability as evidenced by, e.g., low surface hydrophobicity and low self-interaction propensity (thereby reducing the risk of self-aggregation), low monomericity, low charge heterogeneity, and/or low (or absent) chemical liability risk. Among the provided anti-IL-18Rβ antibodies and antigen-binding fragments thereof include those in which a chemical liability risk is reduced or eliminated by introducing an amino acid mutation. (e.g., a mutation of D50 in the light chain variable region, such as, e.g., a D50E or D50G mutation in the light chain variable region) to substitute the amino acid identified as posing a chemical liability risk. In some embodiments, the provided anti-IL-18Rβ antibodies and antigen-binding fragments thereof include those in which, advantageously, a chemical instability risk is eliminated, e.g., by a D50 mutation (e.g., a D50E or D50G mutation) in the light chain variable region, without negatively impacting its: (i) binding to human and cynomolgus monkey IL-18Rβ, including pH-independent binding, (ii) potency in inhibiting IFNg production; and/or (iii) low immunogenicity risk, as compared to the parental antibody that has the chemical instability risk.

In addition, in some embodiments, the provided anti-IL-18Rβ antibodies and antigen-binding fragments thereof exhibit a low immunogenicity risk, as determined, e.g., by a low response rate observed in donors. In some embodiments, the antibody or antigen-binding fragment thereof is human, thereby contributing to a low immunogenicity risk.

In some embodiments, the provided anti-IL-18Rβ antibodies or antigen binding fragments include one or more mutations (e.g., framework revertant mutations) disclosed herein. In some embodiments, the antibodies or antigen binding fragments include Q6E in the heavy chain variable region. In some embodiments, the antibodies or antigen binding fragments do not include F46L in the light chain variable region.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Anti-IL-18Rβ Antibodies and Antigen-Binding Fragments Thereof

Provided herein are anti-IL-18Rβ antibodies and antigen-binding fragments thereof. Among the provided anti-IL-18Rβ antibodies and antigen-binding fragments thereof are those that exhibit one or more (e.g., at least two, three, four, five, or all) of the following advantageous technical effects: high affinity binding to human and cynomolgus monkey IL-18Rβ: potency, for instance, in inhibition of IFNg production; pH-independent binding; low polyreactivity; blocking of IL-18Rβ interaction with the IL-18/IL-18Rα complex; and a low immunogenicity risk.

Among the provided embodiments of anti-IL-18Rβ antibodies and antigen-binding fragments thereof are variants of the anti-IL-18Rβ antibodies and antigen-binding fragments thereof disclosed herein, such as variants having at least 85%, 90%, or 95% sequence identity to a reference antibody. In some embodiments, the variants are variants of antibody P20 or antibody P8. In some embodiments, the variants are variants of antibody P20 and include P20-V1, P20-V2, and P20-V3. In some embodiments, the variants are variants of antibody P8 and include P8-V1.

Among the provided embodiments of anti-IL-18Rβ antibodies and antigen-binding fragments thereof are multi-specific antibodies, such as bispecific antibodies, that comprise a first antigen-binding domain comprising any of the anti-IL-18Rβ antibodies and antigen-binding fragments thereof disclosed herein, and a second antigen-binding domain that binds to a second antigen.

Also provided herein are conjugates comprising an anti-IL-18Rβ antibody or antigen-binding fragment thereof disclosed herein and a heterologous molecule or moiety.

### A. Exemplary Antibodies and Antigen-Binding Fragments Thereof

Provided herein are anti-IL-18Rβ antibodies and antigen-binding fragments thereof, including those having the sequences as described herein, such as those as described in Tables provided in the Examples (e.g., Tables E12A and E16A). In some embodiments, the anti-IL-18Rβ antibody or antigen-binding fragment thereof comprises a V_{H} region that comprises a CDR-H1, a CDR-H2, and a CDR-H3 as described, and comprises a V_{L} region that comprises a CDR-L1, a CDR-L2, and a CDR-L3 as described. In the present disclosure, it is understood that reference to a sequence (such as, but not limited to, sequences for a V_{H} region or heavy chain) by a SEQ ID NO includes the corresponding amino acid sequence or the corresponding amino acid sequence comprising a post-translational modification. Post-translational modifications can include, e.g., glycosylation, phosphorylation, citrullination, isomerization, ubiquitination, acetylation, hydroxylation, methylation, AMPylation, prenylation, deamidation, eliminylation, carbamylation and carbamoylation.

In some embodiments, the post-translational modification is the modification of an amino acid side chain, such as conversion of an N-terminal amino acid (e.g., glutamate or glutamine) to pyroglutamate. In some such embodiments, this modification occurs at the N-terminus of the heavy chain. In a particular example, an antibody or antigen binding fragment thereof that comprises the amino acid sequence SEQ ID NO: 32 may include a post-translational modification to SEQ ID NO: 32, such that the N-terminal glutamine (Q) residue of SEQ ID NO: 32 is post-translationally modified to pyroglutamate as set forth in SEQ ID NO: 159, wherein X is a pyroglutamate. In another particular example, an antibody or antigen binding fragment thereof that comprises the amino acid sequence SEQ ID NO: 34 may include a post-translational modification to SEQ ID NO: 34, such that the N-terminal glutamine (Q) residue of SEQ ID NO: 34 is post-translationally modified to pyroglutamate as set forth in SEQ ID NO:160, wherein X is a pyroglutamate. In another particular example, an antibody or antigen binding fragment thereof that comprises the amino acid sequence SEQ ID NO: 73 may include a post-translational modification to SEQ ID NO: 73, such that the N-terminal glutamine (Q) residue of SEQ ID NO: 73 is post-translationally modified to pyroglutamate as set forth in SEQ ID NO: 162, wherein X is a pyroglutamate. In another particular example, an antibody or antigen binding fragment thereof that comprises the amino acid sequence SEQ ID NO: 75 may include a post-translational modification to SEQ ID NO: 75, such that the N-terminal glutamine (Q) residue of SEQ ID NO: 75 is post-translationally modified to pyroglutamate as set forth in SEQ ID NO:163, wherein X is a pyroglutamate. In some embodiments, in a composition of an antibody or antigen-binding fragment thereof as disclosed herein, such a post-translational modification to pyroglutamate (e.g., at the N-terminus of the heavy chain, e.g., at the N-terminus of SEQ ID NO: 34) is a dominant post-translational modification; in some such embodiments, the antibody or antigen binding fragment thereof in which an N-terminal pyroglutamate is present represents greater than 50%, greater than 60%, greater than 70%, greater than 80%, greater than 90%, or greater than 95% (w/w) of the antibody or antigen binding fragment thereof in the composition.

In some embodiments, the post-translational modification is the cleavage of one or more, particularly one or two, amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a heavy chain constant domain or an anti-IL-18Rβ antibody may include a heavy chain constant domain with both a C-terminal glycine and lysine, without the C-terminal lysine, or without both the C-terminal glycine and lysine. In some embodiments, in a composition of an antibody or antigen-binding fragment thereof as disclosed herein, glycine cleavage is not a dominant post-translational modification; in some such embodiments, the antibody or antigen binding fragment thereof in which the C-terminal lysine is cleaved from the heavy chain constant domain represents less than 10%, less than 5%, or less than 4% (w/w) of the antibody or antigen binding fragment thereof in the composition.

Provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 3 (CDR-L3) contained within the amino acid sequence of SEQ ID NO: 35.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 35.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 8, 11, 13, 15, and 18, a heavy chain complementarity determining region 2 (CDR-H2) comprising an amino acid sequence selected from the group consisting of SEQ ID NOs: 9, 12, 14, 16, and 19, and a heavy chain complementarity determining region 3 (CDR-H3) comprising an amino acid sequence selected from the group consisting of SEQ ID NOs: 10, 17, and 20; and the V_{L} region comprises a light chain complementarity determining region 1 (CDR-L1) comprising an amino acid sequence selected from the group consisting of SEQ ID NOs: 22, 25, and 28, a light chain complementarity determining region 2 (CDR-L2) comprising an amino acid sequence selected from the group consisting of SEQ ID NOs: 36, 37, and 38, and a light chain complementarity determining region 3 (CDR-L3) comprising the amino acid sequence of SEQ ID NO: 24 or 27.

In some embodiments, the V_{H} region comprises a CDR-H1, a CDR-H2, and a CDR-H3 comprising the amino acid sequences of SEQ ID NOs: 8, 9, and 10, respectively; and the V_{L} region comprises a CDR-L1, a CDR-L2, and a CDR-L3 comprising the amino acid sequences of SEQ ID NOs: 22, 36, and 24, respectively; wherein the numbering is according to Kabat numbering.

In some embodiments, the V_{H} region comprises a CDR-H1, a CDR-H2, and a CDR-H3 comprising the amino acid sequences of SEQ ID NOs: 11, 12, and 10, respectively; and the V_{L} region comprises a CDR-L1, a CDR-L2, and a CDR-L3 comprising the amino acid sequences of SEQ ID NOS: 22, 36, and 24, respectively; wherein the numbering is according to Chothia numbering.

In some embodiments, the V_{H} region comprises a CDR-H1, a CDR-H2, and a CDR-H3 comprising the amino acid sequences of SEQ ID NOs: 13, 14, and 10, respectively; and the V_{L} region comprises a CDR-L1, a CDR-L2, and a CDR-L3 comprising the amino acid sequences of SEQ ID NOS: 22, 36, and 24, respectively; wherein the numbering is according to AbM numbering.

In some embodiments, the V_{H} region comprises a CDR-H1, a CDR-H2, and a CDR-H3 comprising the amino acid sequences of SEQ ID NOs: 15, 16, and 17, respectively; and the V_{L} region comprises a CDR-L1, a CDR-L2, and a CDR-L3 comprising the amino acid sequences of SEQ ID NOS: 25, 37, and 27, respectively; wherein the numbering is according to Contact numbering.

In some embodiments, the V_{H} region comprises a CDR-H1, a CDR-H2, and a CDR-H3 comprising the amino acid sequences of SEQ ID NOs: 18, 19, and 20, respectively; and the V_{L} region comprises a CDR-L1, a CDR-L2, and a CDR-L3 comprising the amino acid sequences of SEQ ID NOs: 28, 38, and 24, respectively; wherein the numbering is according to IMGT numbering.

In some embodiments, provided herein is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 3 (CDR-L3) comprising the amino acid sequence of SEQ ID NO: 24. In some embodiments, the V_{L} region further comprises a light chain complementarity determining region 1 (CDR-L1) comprising the amino acid sequence of SEQ ID NO: 22, and a light chain complementarity determining region 2 (CDR-L2) comprising the amino acid sequence of SEQ ID NO: 36.

In some embodiments, provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 1 (CDR-L1) comprising the amino acid sequence of SEQ ID NO: 22, a light chain complementarity determining region 2 (CDR-L2) comprising the amino acid sequence of SEQ ID NO: 36, and a light chain complementarity determining region 3 (CDR-L3) comprising the amino acid sequence of SEQ ID NO: 24.

In some embodiments, the V_{H} region comprises, consists essentially of, or consists of an amino acid sequence having at least, or at least about, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34. In some embodiments, the V_{H} region comprises, consists essentially of, or consists of an amino acid sequence having at least, or at least about 85% sequence identity to the amino acid sequence of SEQ ID NO: 34. In some embodiments, the V_{H} region comprises, consists essentially of, or consists of an amino acid sequence having at least, or at least about 90% sequence identity to the amino acid sequence of SEQ ID NO: 34. In some embodiments, the V_{H} region comprises, consists essentially of, or consists of an amino acid sequence having at least, or at least about 95% sequence identity to the amino acid sequence of SEQ ID NO: 34. In some embodiments, the V_{H} region comprises, consists essentially of, or consists of an amino acid sequence having at least, or at least about 96% sequence identity to the amino acid sequence of SEQ ID NO: 34. In some embodiments, the V_{H} region comprises, consists essentially of, or consists of an amino acid sequence having at least, or at least about 97% sequence identity to the amino acid sequence of SEQ ID NO: 34. In some embodiments, the V_{H} region comprises, consists essentially of, or consists of an amino acid sequence having at least, or at least about 98% sequence identity to the amino acid sequence of SEQ ID NO: 34. In some embodiments, the V_{H} region comprises, consists essentially of, or consists of an amino acid sequence having at least, or at least about 99% sequence identity to the amino acid sequence of SEQ ID NO: 34.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 35. In some embodiments, the V_{L} region comprises, consists essentially of, or consists of an amino acid sequence having at least, or at least about 85% sequence identity to the amino acid sequence of SEQ ID NO: 35. In some embodiments, the V_{L} region comprises, consists essentially of, or consists of an amino acid sequence having at least, or at least about 90% sequence identity to the amino acid sequence of SEQ ID NO: 35. In some embodiments, the V_{L} region comprises, consists essentially of, or consists of an amino acid sequence having at least, or at least about 95% sequence identity to the amino acid sequence of SEQ ID NO: 35. In some embodiments, the V_{L} region comprises, consists essentially of, or consists of an amino acid sequence having at least, or at least about 96% sequence identity to the amino acid sequence of SEQ ID NO: 35. In some embodiments, the V_{L} region comprises, consists essentially of, or consists of an amino acid sequence having at least, or at least about 97% sequence identity to the amino acid sequence of SEQ ID NO: 35. In some embodiments, the V_{L} region comprises, consists essentially of, or consists of an amino acid sequence having at least, or at least about 98% sequence identity to the amino acid sequence of SEQ ID NO: 35. In some embodiments, the V_{L} region comprises, consists essentially of, or consists of an amino acid sequence having at least, or at least about 99% sequence identity to the amino acid sequence of SEQ ID NO: 35. In some embodiments, the V_{L} region does not comprise D at position 50. In some such embodiments, the V_{L} region comprises E or G at position 50. In some such embodiments, the V_{L} region comprises E at position 50. In some embodiments, the V_{L} region comprises E at position 50 and G at position 51. In some embodiments, the V_{L} region comprises F at position 46. In some embodiments, the V_{L} region comprises F at position 46 and D at position 50. In some embodiments, the V_{L} region does not comprise L at position 46.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 35. In some embodiments, the V_{H} region comprises, consists essentially of, or consists of an amino acid sequence having at least, or at least about 85% sequence identity to the amino acid sequence of SEQ ID NO: 34; and the V_{L} region comprises, consists essentially of, or consists of an amino acid sequence having at least, or at least about 85% sequence identity to the amino acid sequence of SEQ ID NO: 35. In some embodiments, the V_{H} region comprises, consists essentially of, or consists of an amino acid sequence having at least, or at least about 90% sequence identity to the amino acid sequence of SEQ ID NO: 34; and the V_{L} region comprises, consists essentially of, or consists of an amino acid sequence having at least, or at least about 90% sequence identity to the amino acid sequence of SEQ ID NO: 35. In some embodiments, the V_{H} region comprises, consists essentially of, or consists of an amino acid sequence having at least, or at least about 95% sequence identity to the amino acid sequence of SEQ ID NO: 34; and the V_{L} region comprises, consists essentially of, or consists of an amino acid sequence having at least, or at least about 95% sequence identity to the amino acid sequence of SEQ ID NO: 35. In some embodiments, the V_{H} region comprises, consists essentially of, or consists of an amino acid sequence having at least, or at least about 96% sequence identity to the amino acid sequence of SEQ ID NO: 34; and the V_{L} region comprises, consists essentially of, or consists of an amino acid sequence having at least, or at least about 96% sequence identity to the amino acid sequence of SEQ ID NO: 35. In some embodiments, the V_{H} region comprises, consists essentially of, or consists of an amino acid sequence having at least, or at least about 97% sequence identity to the amino acid sequence of SEQ ID NO: 34; and the V_{L} region comprises, consists essentially of, or consists of an amino acid sequence having at least, or at least about 97% sequence identity to the amino acid sequence of SEQ ID NO: 35. In some embodiments, the V_{H} region comprises, consists essentially of, or consists of an amino acid sequence having at least, or at least about 98% sequence identity to the amino acid sequence of SEQ ID NO: 34; and the V_{L} region comprises, consists essentially of, or consists of an amino acid sequence having at least, or at least about 98% sequence identity to the amino acid sequence of SEQ ID NO: 35. In some embodiments, the V_{H} region comprises, consists essentially of, or consists of an amino acid sequence having at least, or at least about 99% sequence identity to the amino acid sequence of SEQ ID NO: 34; and the V_{L} region comprises, consists essentially of, or consists of an amino acid sequence having at least, or at least about 99% sequence identity to the amino acid sequence of SEQ ID NO: 35.

In some embodiments, the V_{H} region comprises, consists essentially of, or consists of the amino acid sequence of SEQ ID NO: 34. In some embodiments, the V_{H} region comprises the amino acid sequence of SEQ ID NO: 34, wherein the N-terminal glutamine (Q) residue of SEQ ID NO: 34 is post-translationally modified to pyroglutamine. In some embodiments, the V_{H} region comprises, consists essentially of, or consists of the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 35.

In some embodiments, the V_{L} region comprises, consists essentially of, or consists of the amino acid sequence of SEQ ID NO: 35. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of the amino acid sequence of SEQ ID NO: 35.

In some embodiments, the V_{H} region comprises, consists essentially of, or consists of the amino acid sequence of SEQ ID NO: 34; and the V_{L} region comprises, consists essentially of, or consists of the amino acid sequence of SEQ ID NO: 35.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32. In some embodiments, the heavy chain comprises an amino acid sequence having at least, or at least about 85% sequence identity to the amino acid sequence of SEQ ID NO: 32. In some embodiments, the heavy chain comprises an amino acid sequence having at least, or at least about 90% sequence identity to the amino acid sequence of SEQ ID NO: 32. In some embodiments, the heavy chain comprises an amino acid sequence having at least, or at least about 95% sequence identity to the amino acid sequence of SEQ ID NO: 32. In some embodiments, the heavy chain comprises an amino acid sequence having at least, or at least about 96% sequence identity to the amino acid sequence of SEQ ID NO: 32. In some embodiments, the heavy chain comprises an amino acid sequence having at least, or at least about 97% sequence identity to the amino acid sequence of SEQ ID NO: 32. In some embodiments, the heavy chain comprises an amino acid sequence having at least, or at least about 98% sequence identity to the amino acid sequence of SEQ ID NO: 32. In some embodiments, the heavy chain comprises an amino acid sequence having at least, or at least about 99% sequence identity to the amino acid sequence of SEQ ID NO: 32.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 33. In some embodiments, the light chain comprises an amino acid sequence having at least, or at least about 85% sequence identity to the amino acid sequence of SEQ ID NO: 33. In some embodiments, the light chain comprises an amino acid sequence having at least, or at least about 90% sequence identity to the amino acid sequence of SEQ ID NO: 33. In some embodiments, the light chain comprises an amino acid sequence having at least, or at least about 95% sequence identity to the amino acid sequence of SEQ ID NO: 33. In some embodiments, the light chain comprises an amino acid sequence having at least, or at least about 96% sequence identity to the amino acid sequence of SEQ ID NO: 33. In some embodiments, the light chain comprises an amino acid sequence having at least, or at least about 97% sequence identity to the amino acid sequence of SEQ ID NO: 33. In some embodiments, the light chain comprises an amino acid sequence having at least, or at least about 98% sequence identity to the amino acid sequence of SEQ ID NO: 33. In some embodiments, the light chain comprises an amino acid sequence having at least, or at least about 99% sequence identity to the amino acid sequence of SEQ ID NO: 33.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 33.

In some embodiments, the heavy chain comprises an amino acid sequence having at least, or at least about 85% sequence identity to the amino acid sequence of SEQ ID NO: 32; and the light chain comprises an amino acid sequence having at least, or at least about 85% sequence identity to the amino acid sequence of SEQ ID NO: 33. In some embodiments, the heavy chain comprises an amino acid sequence having at least, or at least about 90% sequence identity to the amino acid sequence of SEQ ID NO: 32; and the light chain comprises an amino acid sequence having at least, or at least about 90% sequence identity to the amino acid sequence of SEQ ID NO: 33. In some embodiments, the heavy chain comprises an amino acid sequence having at least, or at least about 95% sequence identity to the amino acid sequence of SEQ ID NO: 32; and the light chain comprises an amino acid sequence having at least, or at least about 95% sequence identity to the amino acid sequence of SEQ ID NO: 33. In some embodiments, the heavy chain comprises an amino acid sequence having at least, or at least about 96% sequence identity to the amino acid sequence of SEQ ID NO: 32; and the light chain comprises an amino acid sequence having at least, or at least about 96% sequence identity to the amino acid sequence of SEQ ID NO: 33. In some embodiments, the heavy chain comprises an amino acid sequence having at least, or at least about 97% sequence identity to the amino acid sequence of SEQ ID NO: 32; and the light chain comprises an amino acid sequence having at least, or at least about 97% sequence identity to the amino acid sequence of SEQ ID NO: 33. In some embodiments, the heavy chain comprises an amino acid sequence having at least, or at least about 98% sequence identity to the amino acid sequence of SEQ ID NO: 32; and the light chain comprises an amino acid sequence having at least, or at least about 98% sequence identity to the amino acid sequence of SEQ ID NO: 33. In some embodiments, the heavy chain comprises an amino acid sequence having at least, or at least about 99% sequence identity to the amino acid sequence of SEQ ID NO: 32; and the light chain comprises an amino acid sequence having at least, or at least about 99% sequence identity to the amino acid sequence of SEQ ID NO: 33.

In some embodiments, the anti-IL-18Rβ antibody or antigen-binding fragment thereof comprises a heavy chain comprising the amino acid sequence of SEQ ID NO: 32. In some embodiments, the anti-IL-18Rβ antibody or antigen-binding fragment thereof comprises a heavy chain comprising the amino acid sequence of SEQ ID NO: 32, and at least one post-translational modification of the amino acid sequence of SEQ ID NO:32 is present. In some such embodiments, the at least one post-translational modification includes a post-translational modification of the N-terminal glutamine (Q) residue of SEQ ID NO: 32 to a pyroglutamine. In some embodiments, the anti-IL-18Rβ antibody or antigen-binding fragment thereof comprises a heavy chain comprising the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 33.

In some embodiments, the anti-IL-18Rβ antibody or antigen-binding fragment thereof comprises a light chain comprising the amino acid sequence of SEQ ID NO: 33. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising the amino acid sequence of SEQ ID NO: 33.

In some embodiments, the anti-IL-18Rβ antibody or antigen-binding fragment thereof comprises a heavy chain comprising the amino acid sequence of SEQ ID NO: 32, and a light chain comprising the amino acid sequence of SEQ ID NO: 33.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a V_{H} region that comprises, consists essentially of, or consists of (a) an amino acid sequence having at least, or at least about, 90%, 91%, 92%, 93%, 94%, 95%, 96%, 97%, 98%, or 99% sequence identity to the amino acid sequence of SEQ ID NO: 7 or SEQ ID NO: 34 and (b) a V_{L} region that comprises, consists essentially of, or consists of an amino acid sequence having at least, or at least about, 90%, 91%, 92%, 93%, 94%, 95%, 96%, 97%, 98%, or 99% sequence identity to the amino acid sequence of SEQ ID NO: 21 or SEQ ID NO: 35. In some embodiments, the V_{H} region comprises, consists essentially of, or consists of an amino acid sequence having at least, or at least about, 90% sequence identity to the amino acid sequence of SEQ ID NO: 34. In some embodiments, the V_{H} region comprises, consists essentially of, or consists of an amino acid sequence having at least, or at least about, 95% sequence identity to the amino acid sequence of SEQ ID NO: 34. In some embodiments, the V_{H} region comprises, consists essentially of, or consists of an amino acid sequence having at least, or at least about, 96% sequence identity to the amino acid sequence of SEQ ID NO: 34. In some embodiments, the V_{H} region comprises, consists essentially of, or consists of an amino acid sequence having at least, or at least about, 97% sequence identity to the amino acid sequence of SEQ ID NO: 34. In some embodiments, the V_{H} region comprises, consists essentially of, or consists of an amino acid sequence having at least, or at least about, 98% sequence identity to the amino acid sequence of SEQ ID NO: 34. In some embodiments, the V_{H} region comprises, consists essentially of, or consists of an amino acid sequence having at least, or at least about, 99% sequence identity to the amino acid sequence of SEQ ID NO: 34. In some embodiments, V_{H} region comprises E at position 6. In some embodiments, the V_{L} region comprises, consists essentially of, or consists of an amino acid sequence having at least, or at least about, 90% sequence identity to the amino acid sequence of SEQ ID NO: 35. In some embodiments, the V_{L} region comprises, consists essentially of, or consists of an amino acid sequence having at least, or at least about, 95% sequence identity to the amino acid sequence of SEQ ID NO: 35. In some embodiments, the V_{L} region comprises, consists essentially of, or consists of an amino acid sequence having at least, or at least about, 96% sequence identity to the amino acid sequence of SEQ ID NO: 35. In some embodiments, the V_{L} region comprises, consists essentially of, or consists of an amino acid sequence having at least, or at least about, 97% sequence identity to the amino acid sequence of SEQ ID NO: 35. In some embodiments, the V_{L} region comprises, consists essentially of, or consists of an amino acid sequence having at least, or at least about, 98% sequence identity to the amino acid sequence of SEQ ID NO: 35. In some embodiments, the V_{L} region comprises, consists essentially of, or consists of an amino acid sequence having at least, or at least about, 99% sequence identity to the amino acid sequence of SEQ ID NO: 35. In some embodiments, the V_{L} region does not comprise D at position 50. In some such embodiments, the V_{L} region comprises G or E at position 50. In some embodiments, the V_{L} region comprises E at position 50 and G at position 51. In some embodiments, the V_{L} region comprises F at position 46. In some embodiments, the V_{L} region comprises F at position 46 and D at position 50. In some embodiments, the V_{L} region does not comprise L at position 46.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 51; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 65.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 52, 55, 57, 59, and 62, a heavy chain complementarity determining region 2 (CDR-H2) comprising an amino acid sequence selected from the group consisting of SEQ ID NOs: 53, 56, 58, 60, and 63, and a heavy chain complementarity determining region 3 (CDR-H3) comprising an amino acid sequence selected from the group consisting of SEQ ID NOs: 54, 61, and 64; and the V_{L} region comprises a light chain complementarity determining region 1 (CDR-L1) comprising an amino acid sequence selected from the group consisting of SEQ ID NOs: 66, 69, and 72, a light chain complementarity determining region 2 (CDR-L2) comprising an amino acid sequence selected from the group consisting of SEQ ID NOs: 67, 70, and 29, and a light chain complementarity determining region 3 (CDR-L3) comprising the amino acid sequence of SEQ ID NO: 68 or 71.

In some embodiments, the V_{H} region comprises a CDR-H1, a CDR-H2, and a CDR-H3 comprising the amino acid sequences of SEQ ID NOs: 52, 53, and 54, respectively; and the V_{L} region comprises a CDR-L1, a CDR-L2, and a CDR-L3 comprising the amino acid sequences of SEQ ID NOs: 66, 67, and 68, respectively; wherein the numbering is according to Kabat numbering.

In some embodiments, the V_{H} region comprises a CDR-H1, a CDR-H2, and a CDR-H3 comprising the amino acid sequences of SEQ ID NOs: 55, 56, and 54, respectively; and the V_{L} region comprises a CDR-L1, a CDR-L2, and a CDR-L3 comprising the amino acid sequences of SEQ ID NOs: 66, 67, and 68, respectively; wherein the numbering is according to Chothia numbering.

In some embodiments, the V_{H} region comprises a CDR-H1, a CDR-H2, and a CDR-H3 comprising the amino acid sequences of SEQ ID NOs: 57, 58, and 54, respectively; and the V_{L} region comprises a CDR-L1, a CDR-L2, and a CDR-L3 comprising the amino acid sequences of SEQ ID NOS: 66, 67, and 68, respectively; wherein the numbering is according to AbM numbering.

In some embodiments, the V_{H} region comprises a CDR-H1, a CDR-H2, and a CDR-H3 comprising the amino acid sequences of SEQ ID NOs: 59, 60, and 61, respectively; and the V_{L} region comprises a CDR-L1, a CDR-L2, and a CDR-L3 comprising the amino acid sequences of SEQ ID NOs: 69, 70, and 71, respectively; wherein the numbering is according to Contact numbering.

In some embodiments, the V_{H} region comprises a CDR-H1, a CDR-H2, and a CDR-H3 comprising the amino acid sequences of SEQ ID NOs: 62, 63, and 64, respectively; and the V_{L} region comprises a CDR-L1, a CDR-L2, and a CDR-L3 comprising the amino acid sequences of SEQ ID NOs: 72, 29, and 68, respectively; wherein the numbering is according to IMGT numbering.

In some embodiments, provided herein is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 52, a heavy chain complementarity determining region 2 (CDR-H2) comprising the amino acid sequence of SEQ ID NO: 53, and a heavy chain complementarity determining region 3 (CDR-H3) comprising the amino acid sequence of SEQ ID NO: 54; and the V_{L} region comprises a light chain complementarity determining region 1 (CDR-L1) comprising the amino acid sequence of SEQ ID NO: 66, a light chain complementarity determining region 2 (CDR-L2) comprising an amino acid sequence selected from the group consisting of SEQ ID NOs: 67, and a light chain complementarity determining region 3 (CDR-L3) comprising the amino acid sequence of SEQ ID NO: 68.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 51. In some embodiments, the V_{H} region comprises, consists essentially of, or consists of an amino acid sequence having at least, or at least about 85% sequence identity to the amino acid sequence of SEQ ID NO: 51. In some embodiments, the V_{H} region comprises, consists essentially of, or consists of an amino acid sequence having at least, or at least about 90% sequence identity to the amino acid sequence of SEQ ID NO: 51. In some embodiments, the V_{H} region comprises, consists essentially of, or consists of an amino acid sequence having at least, or at least about 95% sequence identity to the amino acid sequence of SEQ ID NO: 51. In some embodiments, the V_{H} region comprises, consists essentially of, or consists of an amino acid sequence having at least, or at least about 96% sequence identity to the amino acid sequence of SEQ ID NO: 51. In some embodiments, the V_{H} region comprises, consists essentially of, or consists of an amino acid sequence having at least, or at least about 97% sequence identity to the amino acid sequence of SEQ ID NO: 51. In some embodiments, the V_{H} region comprises, consists essentially of, or consists of an amino acid sequence having at least, or at least about 98% sequence identity to the amino acid sequence of SEQ ID NO: 51. In some embodiments, the V_{H} region comprises, consists essentially of, or consists of an amino acid sequence having at least, or at least about 99% sequence identity to the amino acid sequence of SEQ ID NO: 51.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 65. In some embodiments, the V_{L} region comprises, consists essentially of, or consists of an amino acid sequence having at least, or at least about 85% sequence identity to the amino acid sequence of SEQ ID NO: 65. In some embodiments, the V_{L} region comprises, consists essentially of, or consists of an amino acid sequence having at least, or at least about 90% sequence identity to the amino acid sequence of SEQ ID NO: 65. In some embodiments, the V_{L} region comprises, consists essentially of, or consists of an amino acid sequence having at least, or at least about 95% sequence identity to the amino acid sequence of SEQ ID NO: 65. In some embodiments, the V_{L} region comprises, consists essentially of, or consists of an amino acid sequence having at least, or at least about 96% sequence identity to the amino acid sequence of SEQ ID NO: 65. In some embodiments, the V_{L} region comprises, consists essentially of, or consists of an amino acid sequence having at least, or at least about 97% sequence identity to the amino acid sequence of SEQ ID NO: 65. In some embodiments, the V_{L} region comprises, consists essentially of, or consists of an amino acid sequence having at least, or at least about 98% sequence identity to the amino acid sequence of SEQ ID NO: 65. In some embodiments, the V_{L} region comprises, consists essentially of, or consists of an amino acid sequence having at least, or at least about 99% sequence identity to the amino acid sequence of SEQ ID NO: 65.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 51; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 65. In some embodiments, the V_{H} region comprises, consists essentially of, or consists of an amino acid sequence having at least, or at least about 85% sequence identity to the amino acid sequence of SEQ ID NO: 51; and the V_{L} region comprises, consists essentially of, or consists of an amino acid sequence having at least, or at least about 85% sequence identity to the amino acid sequence of SEQ ID NO: 65. In some embodiments, the V_{H} region comprises, consists essentially of, or consists of an amino acid sequence having at least, or at least about 90% sequence identity to the amino acid sequence of SEQ ID NO: 51; and the V_{L} region comprises, consists essentially of, or consists of an amino acid sequence having at least, or at least about 90% sequence identity to the amino acid sequence of SEQ ID NO: 65. In some embodiments, the V_{H} region comprises, consists essentially of, or consists of an amino acid sequence having at least, or at least about 95% sequence identity to the amino acid sequence of SEQ ID NO: 51; and the V_{L} region comprises, consists essentially of, or consists of an amino acid sequence having at least, or at least about 95% sequence identity to the amino acid sequence of SEQ ID NO: 65. In some embodiments, the V_{H} region comprises, consists essentially of, or consists of an amino acid sequence having at least, or at least about 96% sequence identity to the amino acid sequence of SEQ ID NO: 51; and the V_{L} region comprises, consists essentially of, or consists of an amino acid sequence having at least, or at least about 96% sequence identity to the amino acid sequence of SEQ ID NO: 65. In some embodiments, the V_{H} region comprises, consists essentially of, or consists of an amino acid sequence having at least, or at least about 97% sequence identity to the amino acid sequence of SEQ ID NO: 51; and the V_{L} region comprises, consists essentially of, or consists of an amino acid sequence having at least, or at least about 97% sequence identity to the amino acid sequence of SEQ ID NO: 65. In some embodiments, the V_{H} region comprises, consists essentially of, or consists of an amino acid sequence having at least, or at least about 98% sequence identity to the amino acid sequence of SEQ ID NO: 51; and the V_{L} region comprises, consists essentially of, or consists of an amino acid sequence having at least, or at least about 98% sequence identity to the amino acid sequence of SEQ ID NO: 65. In some embodiments, the V_{H} region comprises, consists essentially of, or consists of an amino acid sequence having at least, or at least about 99% sequence identity to the amino acid sequence of SEQ ID NO: 51; and the V_{L} region comprises, consists essentially of, or consists of an amino acid sequence having at least, or at least about 99% sequence identity to the amino acid sequence of SEQ ID NO: 65.

In some embodiments, the V_{H} region comprises, consists essentially of, or consists of the amino acid sequence of SEQ ID NO: 51. In some embodiments, the V_{H} region comprises, consists essentially of, or consists of the amino acid sequence of SEQ ID NO: 51; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 65.

In some embodiments, the V_{L} region comprises, consists essentially of, or consists of the amino acid sequence of SEQ ID NO: 65. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 51; and the V_{L} region comprises, consists essentially of, or consists of the amino acid sequence of SEQ ID NO: 65.

In some embodiments, the V_{H} region comprises, consists essentially of, or consists of the amino acid sequence of SEQ ID NO: 51; and the V_{L} region comprises, consists essentially of, or consists of the amino acid sequence of SEQ ID NO: 65.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 49. In some embodiments, the heavy chain comprises an amino acid sequence having at least, or at least about 85% sequence identity to the amino acid sequence of SEQ ID NO: 49. In some embodiments, the heavy chain comprises an amino acid sequence having at least, or at least about 90% sequence identity to the amino acid sequence of SEQ ID NO: 49. In some embodiments, the heavy chain comprises an amino acid sequence having at least, or at least about 95% sequence identity to the amino acid sequence of SEQ ID NO: 49. In some embodiments, the heavy chain comprises an amino acid sequence having at least, or at least about 96% sequence identity to the amino acid sequence of SEQ ID NO: 49. In some embodiments, the heavy chain comprises an amino acid sequence having at least, or at least about 97% sequence identity to the amino acid sequence of SEQ ID NO: 49. In some embodiments, the heavy chain comprises an amino acid sequence having at least, or at least about 98% sequence identity to the amino acid sequence of SEQ ID NO: 49. In some embodiments, the heavy chain comprises an amino acid sequence having at least, or at least about 99% sequence identity to the amino acid sequence of SEQ ID NO: 49.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50. In some embodiments, the light chain comprises an amino acid sequence having at least, or at least about 85% sequence identity to the amino acid sequence of SEQ ID NO: 50. In some embodiments, the light chain comprises an amino acid sequence having at least, or at least about 90% sequence identity to the amino acid sequence of SEQ ID NO: 50. In some embodiments, the light chain comprises an amino acid sequence having at least, or at least about 95% sequence identity to the amino acid sequence of SEQ ID NO: 50. In some embodiments, the light chain comprises an amino acid sequence having at least, or at least about 96% sequence identity to the amino acid sequence of SEQ ID NO: 50. In some embodiments, the light chain comprises an amino acid sequence having at least, or at least about 97% sequence identity to the amino acid sequence of SEQ ID NO: 50. In some embodiments, the light chain comprises an amino acid sequence having at least, or at least about 98% sequence identity to the amino acid sequence of SEQ ID NO: 50. In some embodiments, the light chain comprises an amino acid sequence having at least, or at least about 99% sequence identity to the amino acid sequence of SEQ ID NO: 50.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 49; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50.

In some embodiments, the heavy chain comprises an amino acid sequence having at least, or at least about 85% sequence identity to the amino acid sequence of SEQ ID NO: 49; and the light chain comprises an amino acid sequence having at least, or at least about 85% sequence identity to the amino acid sequence of SEQ ID NO: 50. In some embodiments, the heavy chain comprises an amino acid sequence having at least, or at least about 90% sequence identity to the amino acid sequence of SEQ ID NO: 49; and the light chain comprises an amino acid sequence having at least, or at least about 90% sequence identity to the amino acid sequence of SEQ ID NO: 50. In some embodiments, the heavy chain comprises an amino acid sequence having at least, or at least about 95% sequence identity to the amino acid sequence of SEQ ID NO: 49; and the light chain comprises an amino acid sequence having at least, or at least about 95% sequence identity to the amino acid sequence of SEQ ID NO: 50. In some embodiments, the heavy chain comprises an amino acid sequence having at least, or at least about 96% sequence identity to the amino acid sequence of SEQ ID NO: 49; and the light chain comprises an amino acid sequence having at least, or at least about 96% sequence identity to the amino acid sequence of SEQ ID NO: 50. In some embodiments, the heavy chain comprises an amino acid sequence having at least, or at least about 97% sequence identity to the amino acid sequence of SEQ ID NO: 49; and the light chain comprises an amino acid sequence having at least, or at least about 97% sequence identity to the amino acid sequence of SEQ ID NO: 50. In some embodiments, the heavy chain comprises an amino acid sequence having at least, or at least about 98% sequence identity to the amino acid sequence of SEQ ID NO: 49; and the light chain comprises an amino acid sequence having at least, or at least about 98% sequence identity to the amino acid sequence of SEQ ID NO: 50. In some embodiments, the heavy chain comprises an amino acid sequence having at least, or at least about 99% sequence identity to the amino acid sequence of SEQ ID NO: 49; and the light chain comprises an amino acid sequence having at least, or at least about 99% sequence identity to the amino acid sequence of SEQ ID NO: 50.

In some embodiments, the anti-IL-18Rβ antibody or antigen-binding fragment thereof comprises a heavy chain comprising the amino acid sequence of SEQ ID NO: 49. In some embodiments, the anti-IL-18Rβ antibody or antigen-binding fragment thereof comprises a heavy chain comprising the amino acid sequence of SEQ ID NO: 49; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50.

In some embodiments, the anti-IL-18Rβ antibody or antigen-binding fragment thereof comprises a light chain comprising the amino acid sequence of SEQ ID NO: 50. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 49; and a light chain comprising the amino acid sequence of SEQ ID NO: 50.

In some embodiments, the anti-IL-18Rβ antibody or antigen-binding fragment thereof comprises a heavy chain comprising the amino acid sequence of SEQ ID NO: 49, and a light chain comprising the amino acid sequence of SEQ ID NO: 50.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 7; and the V_{L} region comprises a light chain complementarity determining region 3 (CDR-L3) contained within the amino acid sequence of SEQ ID NO: 21.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 8, 11, 13, 15, and 18, a heavy chain complementarity determining region 2 (CDR-H2) comprising an amino acid sequence selected from the group consisting of SEQ ID NOs: 9, 12, 14, 16, and 19, and a heavy chain complementarity determining region 3 (CDR-H3) comprising an amino acid sequence selected from the group consisting of SEQ ID NOs: 10, 17, and 20; and the V_{L} region comprises a light chain complementarity determining region 1 (CDR-L1) comprising an amino acid sequence selected from the group consisting of SEQ ID NOs: 22, 25, and 28, a light chain complementarity determining region 2 (CDR-L2) comprising an amino acid sequence selected from the group consisting of SEQ ID NOs: 23. 26, and 29, and a light chain complementarity determining region 3 (CDR-L3) comprising the amino acid sequence of SEQ ID NO: 24 or 27.

In some embodiments, the V_{H} region comprises a CDR-H1, a CDR-H2, and a CDR-H3 comprising the amino acid sequences of SEQ ID NOs: 8, 9, and 10, respectively; and the V_{L} region comprises a CDR-L1, a CDR-L2, and a CDR-L3 comprising the amino acid sequences of SEQ ID NOS: 22, 23, and 24, respectively; wherein the numbering is according to Kabat numbering.

In some embodiments, the V_{H} region comprises a CDR-H1, a CDR-H2, and a CDR-H3 comprising the amino acid sequences of SEQ ID NOs: 11, 12, and 10, respectively; and the V_{L} region comprises a CDR-L1, a CDR-L2, and a CDR-L3 comprising the amino acid sequences of SEQ ID NOS: 22, 23, and 24, respectively; wherein the numbering is according to Chothia numbering.

In some embodiments, the V_{H} region comprises a CDR-H1, a CDR-H2, and a CDR-H3 comprising the amino acid sequences of SEQ ID NOs: 13, 14, and 10, respectively; and the V_{L} region comprises a CDR-L1, a CDR-L2, and a CDR-L3 comprising the amino acid sequences of SEQ ID NOs: 22, 23, and 24, respectively; wherein the numbering is according to AbM numbering.

In some embodiments, the V_{H} region comprises a CDR-H1, a CDR-H2, and a CDR-H3 comprising the amino acid sequences of SEQ ID NOs: 15, 16, and 17, respectively; and the V_{L} region comprises a CDR-L1, a CDR-L2, and a CDR-L3 comprising the amino acid sequences of SEQ ID NOS: 25, 26, and 27, respectively; wherein the numbering is according to Contact numbering.

In some embodiments, the V_{H} region comprises a CDR-H1, a CDR-H2, and a CDR-H3 comprising the amino acid sequences of SEQ ID NOs: 18, 19, and 20, respectively; and the V_{L} region comprises a CDR-L1, a CDR-L2, and a CDR-L3 comprising the amino acid sequences of SEQ ID NOS: 28, 29, and 24, respectively; wherein the numbering is according to IMGT numbering.

In some embodiments, provided herein is an anti-interleukin 18 receptor beta (IL-18R) antibody or antigen-binding fragment thereof, comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 1 (CDR-L1) comprising the amino acid sequence of SEQ ID NO: 22, a light chain complementarity determining region 2 (CDR-L2) comprising the amino acid sequence of SEQ ID NO: 23, and a light chain complementarity determining region 3 (CDR-L3) comprising the amino acid sequence of SEQ ID NO: 24.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 7. In some embodiments, the V_{H} region comprises, consists essentially of, or consists of an amino acid sequence having at least, or at least about 85% sequence identity to the amino acid sequence of SEQ ID NO: 7. In some embodiments, the V_{H} region comprises, consists essentially of, or consists of an amino acid sequence having at least, or at least about 90% sequence identity to the amino acid sequence of SEQ ID NO: 7. In some embodiments, the V_{H} region comprises, consists essentially of, or consists of an amino acid sequence having at least, or at least about 95% sequence identity to the amino acid sequence of SEQ ID NO: 7. In some embodiments, the V_{H} region comprises, consists essentially of, or consists of an amino acid sequence having at least, or at least about 96% sequence identity to the amino acid sequence of SEQ ID NO: 7. In some embodiments, the V_{H} region comprises, consists essentially of, or consists of an amino acid sequence having at least, or at least about 97% sequence identity to the amino acid sequence of SEQ ID NO: 7. In some embodiments, the V_{H} region comprises, consists essentially of, or consists of an amino acid sequence having at least, or at least about 98% sequence identity to the amino acid sequence of SEQ ID NO: 7. In some embodiments, the V_{H} region comprises, consists essentially of, or consists of an amino acid sequence having at least, or at least about 99% sequence identity to the amino acid sequence of SEQ ID NO: 7.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 21. In some embodiments, the V_{L} region comprises, consists essentially of, or consists of an amino acid sequence having at least, or at least about 85% sequence identity to the amino acid sequence of SEQ ID NO: 21. In some embodiments, the V_{L} region comprises, consists essentially of, or consists of an amino acid sequence having at least, or at least about 90% sequence identity to the amino acid sequence of SEQ ID NO: 21. In some embodiments, the V_{L} region comprises, consists essentially of, or consists of an amino acid sequence having at least, or at least about 95% sequence identity to the amino acid sequence of SEQ ID NO: 21. In some embodiments, the V_{L} region comprises, consists essentially of, or consists of an amino acid sequence having at least, or at least about 96% sequence identity to the amino acid sequence of SEQ ID NO: 21. In some embodiments, the V_{L} region comprises, consists essentially of, or consists of an amino acid sequence having at least, or at least about 97% sequence identity to the amino acid sequence of SEQ ID NO: 21. In some embodiments, the V_{L} region comprises, consists essentially of, or consists of an amino acid sequence having at least, or at least about 98% sequence identity to the amino acid sequence of SEQ ID NO: 21. In some embodiments, the V_{L} region comprises, consists essentially of, or consists of an amino acid sequence having at least, or at least about 99% sequence identity to the amino acid sequence of SEQ ID NO: 21.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 21. In some embodiments, the V_{H} region comprises, consists essentially of, or consists of an amino acid sequence having at least, or at least about 85% sequence identity to the amino acid sequence of SEQ ID NO: 7; and the V_{L} region comprises, consists essentially of, or consists of an amino acid sequence having at least, or at least about 85% sequence identity to the amino acid sequence of SEQ ID NO: 21. In some embodiments, the V_{H} region comprises, consists essentially of, or consists of an amino acid sequence having at least, or at least about 90% sequence identity to the amino acid sequence of SEQ ID NO: 7; and the V_{L} region comprises, consists essentially of, or consists of an amino acid sequence having at least, or at least about 90% sequence identity to the amino acid sequence of SEQ ID NO: 21. In some embodiments, the V_{H} region comprises, consists essentially of, or consists of an amino acid sequence having at least, or at least about 95% sequence identity to the amino acid sequence of SEQ ID NO: 7; and the V_{L} region comprises, consists essentially of, or consists of an amino acid sequence having at least, or at least about 95% sequence identity to the amino acid sequence of SEQ ID NO: 21. In some embodiments, the V_{H} region comprises, consists essentially of, or consists of an amino acid sequence having at least, or at least about 96% sequence identity to the amino acid sequence of SEQ ID NO: 7; and the V_{L} region comprises, consists essentially of, or consists of an amino acid sequence having at least, or at least about 96% sequence identity to the amino acid sequence of SEQ ID NO: 21. In some embodiments, the V_{H} region comprises, consists essentially of, or consists of an amino acid sequence having at least, or at least about 97% sequence identity to the amino acid sequence of SEQ ID NO: 7; and the V_{L} region comprises, consists essentially of, or consists of an amino acid sequence having at least, or at least about 97% sequence identity to the amino acid sequence of SEQ ID NO: 21. In some embodiments, the V_{H} region comprises, consists essentially of, or consists of an amino acid sequence having at least, or at least about 98% sequence identity to the amino acid sequence of SEQ ID NO: 7; and the V_{L} region comprises, consists essentially of, or consists of an amino acid sequence having at least, or at least about 98% sequence identity to the amino acid sequence of SEQ ID NO: 21. In some embodiments, the V_{H} region comprises, consists essentially of, or consists of an amino acid sequence having at least, or at least about 99% sequence identity to the amino acid sequence of SEQ ID NO: 7; and the V_{L} region comprises, consists essentially of, or consists of an amino acid sequence having at least, or at least about 99% sequence identity to the amino acid sequence of SEQ ID NO: 21.

In some embodiments, the V_{H} region comprises, consists essentially of, or consists of the amino acid sequence of SEQ ID NO: 7. In some embodiments, the V_{H} region comprises, consists essentially of, or consists of the amino acid sequence of SEQ ID NO: 7; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 21.

In some embodiments, the V_{L} region comprises, consists essentially of, or consists of the amino acid sequence of SEQ ID NO: 21. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the V_{L} region comprises, consists essentially of, or consists of the amino acid sequence of SEQ ID NO: 21.

In some embodiments, the V_{H} region comprises, consists essentially of, or consists of the amino acid sequence of SEQ ID NO: 7; and the V_{L} region comprises, consists essentially of, or consists of the amino acid sequence of SEQ ID NO: 21.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 5. In some embodiments, the heavy chain comprises an amino acid sequence having at least, or at least about 85% sequence identity to the amino acid sequence of SEQ ID NO: 5. In some embodiments, the heavy chain comprises an amino acid sequence having at least, or at least about 90% sequence identity to the amino acid sequence of SEQ ID NO: 5. In some embodiments, the heavy chain comprises an amino acid sequence having at least, or at least about 95% sequence identity to the amino acid sequence of SEQ ID NO: 5. In some embodiments, the heavy chain comprises an amino acid sequence having at least, or at least about 96% sequence identity to the amino acid sequence of SEQ ID NO: 5. In some embodiments, the heavy chain comprises an amino acid sequence having at least, or at least about 97% sequence identity to the amino acid sequence of SEQ ID NO: 5. In some embodiments, the heavy chain comprises an amino acid sequence having at least, or at least about 98% sequence identity to the amino acid sequence of SEQ ID NO: 5. In some embodiments, the heavy chain comprises an amino acid sequence having at least, or at least about 99% sequence identity to the amino acid sequence of SEQ ID NO: 5.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 6. In some embodiments, the light chain comprises an amino acid sequence having at least, or at least about 85% sequence identity to the amino acid sequence of SEQ ID NO: 6. In some embodiments, the light chain comprises an amino acid sequence having at least, or at least about 90% sequence identity to the amino acid sequence of SEQ ID NO: 6. In some embodiments, the light chain comprises an amino acid sequence having at least, or at least about 95% sequence identity to the amino acid sequence of SEQ ID NO: 6. In some embodiments, the light chain comprises an amino acid sequence having at least, or at least about 96% sequence identity to the amino acid sequence of SEQ ID NO: 6. In some embodiments, the light chain comprises an amino acid sequence having at least, or at least about 97% sequence identity to the amino acid sequence of SEQ ID NO: 6. In some embodiments, the light chain comprises an amino acid sequence having at least, or at least about 98% sequence identity to the amino acid sequence of SEQ ID NO: 6. In some embodiments, the light chain comprises an amino acid sequence having at least, or at least about 99% sequence identity to the amino acid sequence of SEQ ID NO: 6.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 5; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 6.

In some embodiments, the heavy chain comprises an amino acid sequence having at least, or at least about 85% sequence identity to the amino acid sequence of SEQ ID NO: 5; and the light chain comprises an amino acid sequence having at least, or at least about 85% sequence identity to the amino acid sequence of SEQ ID NO: 6. In some embodiments, the heavy chain comprises an amino acid sequence having at least, or at least about 90% sequence identity to the amino acid sequence of SEQ ID NO: 5; and the light chain comprises an amino acid sequence having at least, or at least about 90% sequence identity to the amino acid sequence of SEQ ID NO: 6. In some embodiments, the heavy chain comprises an amino acid sequence having at least, or at least about 95% sequence identity to the amino acid sequence of SEQ ID NO: 5; and the light chain comprises an amino acid sequence having at least, or at least about 95% sequence identity to the amino acid sequence of SEQ ID NO: 6. In some embodiments, the heavy chain comprises an amino acid sequence having at least, or at least about 96% sequence identity to the amino acid sequence of SEQ ID NO: 5; and the light chain comprises an amino acid sequence having at least, or at least about 96% sequence identity to the amino acid sequence of SEQ ID NO: 6. In some embodiments, the heavy chain comprises an amino acid sequence having at least, or at least about 97% sequence identity to the amino acid sequence of SEQ ID NO: 5; and the light chain comprises an amino acid sequence having at least, or at least about 97% sequence identity to the amino acid sequence of SEQ ID NO: 6. In some embodiments, the heavy chain comprises an amino acid sequence having at least, or at least about 98% sequence identity to the amino acid sequence of SEQ ID NO: 5; and the light chain comprises an amino acid sequence having at least, or at least about 98% sequence identity to the amino acid sequence of SEQ ID NO: 6. In some embodiments, the heavy chain comprises an amino acid sequence having at least, or at least about 99% sequence identity to the amino acid sequence of SEQ ID NO: 5; and the light chain comprises an amino acid sequence having at least, or at least about 99% sequence identity to the amino acid sequence of SEQ ID NO: 6.

In some embodiments, the anti-IL-18Rβ antibody or antigen-binding fragment thereof comprises a heavy chain comprising the amino acid sequence of SEQ ID NO: 5. In some embodiments, the anti-IL-18Rβ antibody or antigen-binding fragment thereof comprises a heavy chain comprising the amino acid sequence of SEQ ID NO: 5; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 6.

In some embodiments, the anti-IL-18Rβ antibody or antigen-binding fragment thereof comprises a light chain comprising the amino acid sequence of SEQ ID NO: 6. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 5; and a light chain comprising the amino acid sequence of SEQ ID NO: 6.

In some embodiments, the anti-IL-18Rβ antibody or antigen-binding fragment thereof comprises a heavy chain comprising the amino acid sequence of SEQ ID NO: 5, and a light chain comprising the amino acid sequence of SEQ ID NO: 6.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 40 or 45.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 8, 11, 13, 15, and 18, a heavy chain complementarity determining region 2 (CDR-H2) comprising an amino acid sequence selected from the group consisting of SEQ ID NOs: 9, 12, 14, 16, and 19, and a heavy chain complementarity determining region 3 (CDR-H3) comprising an amino acid sequence selected from the group consisting of SEQ ID NOs: 10, 17, and 20; and the V_{L} region comprises a light chain complementarity determining region 1 (CDR-L1) comprising an amino acid sequence selected from the group consisting of SEQ ID NOs: 22, 25, and 28, a light chain complementarity determining region 2 (CDR-L2) comprising an amino acid sequence selected from the group consisting of SEQ ID NOs: 41-43 and 46-48, and a light chain complementarity determining region 3 (CDR-L3) comprising the amino acid sequence of SEQ ID NO: 24 or 27.

In some embodiments, the V_{H} region comprises a CDR-H1, a CDR-H2, and a CDR-H3 comprising the amino acid sequences of SEQ ID NOs: 8, 9, and 10, respectively; and the V_{L} region comprises a CDR-L1 comprising the amino acid sequence of SEQ ID NO: 22, a CDR-L2 comprising the amino acid sequence of SEQ ID NO: 41 or 46, and a CDR-L3 comprising the amino acid sequence of SEQ ID NO: 24; wherein the numbering is according to Kabat numbering.

In some embodiments, the V_{H} region comprises a CDR-H1, a CDR-H2, and a CDR-H3 comprising the amino acid sequences of SEQ ID NOs: 11, 12, and 10, respectively; and the V_{L} region comprises a CDR-L1 comprising the amino acid sequence of SEQ ID NO: 22, a CDR-L2 comprising the amino acid sequence of SEQ ID NO: 41 or 46, and a CDR-L3 comprising the amino acid sequence of SEQ ID NO: 24; wherein the numbering is according to Chothia numbering.

In some embodiments, the V_{H} region comprises a CDR-H1, a CDR-H2, and a CDR-H3 comprising the amino acid sequences of SEQ ID NOs: 13, 14, and 10, respectively; and the V_{L} region comprises a CDR-L1 comprising the amino acid sequence of SEQ ID NO: 22, a CDR-L2 comprising the amino acid sequence of SEQ ID NO: 41 or 46, and a CDR-L3 comprising the amino acid sequence of SEQ ID NO: 24; wherein the numbering is according to AbM numbering.

In some embodiments, the V_{H} region comprises a CDR-H1, a CDR-H2, and a CDR-H3 comprising the amino acid sequences of SEQ ID NOs: 15, 16, and 17, respectively; and the V_{L} region comprises a CDR-L1 comprising the amino acid sequence of SEQ ID NO: 25, a CDR-L2 comprising the amino acid sequence of SEQ ID NO: 42 or 47, and a CDR-L3 comprising the amino acid sequence of SEQ ID NO: 27; wherein the numbering is according to Contact numbering.

In some embodiments, the V_{H} region comprises a CDR-H1, a CDR-H2, and a CDR-H3 comprising the amino acid sequences of SEQ ID NOs: 18, 19, and 20, respectively; and the V_{L} region comprises a CDR-L1 comprising the amino acid sequence of SEQ ID NO: 28, a CDR-L2 comprising the amino acid sequence of SEQ ID NO: 43 or 48, and a CDR-L3 comprising the amino acid sequence of SEQ ID NO: 24; wherein the numbering is according to IMGT numbering.

In some embodiments, provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a CDR-H1, a CDR-H2, and a CDR-H3 comprising the amino acid sequences of SEQ ID NOs: 8, 9, and 10, respectively; and the V_{L} region comprises a CDR-L1, a CDR-L2, and a CDR-L3 comprising the amino acid sequences of SEQ ID NOs: 22, 41, and 24, respectively.

In some embodiments, provided herein is an anti-interleukin 18 receptor beta (IL-18Rβ) antibody or antigen-binding fragment thereof, comprising a heavy chain variable (V_{H}) region and a light chain variable (V_{L}) region, wherein: the V_{H} region comprises a CDR-H1, a CDR-H2, and a CDR-H3 comprising the amino acid sequences of SEQ ID NOs: 8, 9, and 10, respectively; and the V_{L} region comprises a CDR-L1, a CDR-L2, and a CDR-L3 comprising the amino acid sequences of SEQ ID NOS: 22, 46, and 24, respectively.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34. In some embodiments, the V_{H} region comprises, consists essentially of, or consists of an amino acid sequence having at least, or at least about 85% sequence identity to the amino acid sequence of SEQ ID NO: 34. In some embodiments, the V_{H} region comprises, consists essentially of, or consists of an amino acid sequence having at least, or at least about 90% sequence identity to the amino acid sequence of SEQ ID NO: 34. In some embodiments, the V_{H} region comprises, consists essentially of, or consists of an amino acid sequence having at least, or at least about 95% sequence identity to the amino acid sequence of SEQ ID NO: 34. In some embodiments, the V_{H} region comprises, consists essentially of, or consists of an amino acid sequence having at least, or at least about 96% sequence identity to the amino acid sequence of SEQ ID NO: 34. In some embodiments, the V_{H} region comprises, consists essentially of, or consists of an amino acid sequence having at least, or at least about 97% sequence identity to the amino acid sequence of SEQ ID NO: 34. In some embodiments, the V_{H} region comprises, consists essentially of, or consists of an amino acid sequence having at least, or at least about 98% sequence identity to the amino acid sequence of SEQ ID NO: 34. In some embodiments, the V_{H} region comprises, consists essentially of, or consists of an amino acid sequence having at least, or at least about 99% sequence identity to the amino acid sequence of SEQ ID NO: 34.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 40 or 45. In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 40. In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 45. In some embodiments, the V_{L} region comprises, consists essentially of, or consists of an amino acid sequence having at least, or at least about 85% sequence identity to the amino acid sequence of SEQ ID NO: 40 or 45. In some embodiments, the V_{L} region comprises, consists essentially of, or consists of an amino acid sequence having at least, or at least about 90% sequence identity to the amino acid sequence of SEQ ID NO: 40 or 45. In some embodiments, the V_{L} region comprises, consists essentially of, or consists of an amino acid sequence having at least, or at least about 95% sequence identity to the amino acid sequence of SEQ ID NO: 40 or 45. In some embodiments, the V_{L} region comprises, consists essentially of, or consists of an amino acid sequence having at least, or at least about 96% sequence identity to the amino acid sequence of SEQ ID NO: 40 or 45. In some embodiments, the V_{L} region comprises, consists essentially of, or consists of an amino acid sequence having at least, or at least about 97% sequence identity to the amino acid sequence of SEQ ID NO: 40 or 45. In some embodiments, the V_{L} region comprises, consists essentially of, or consists of an amino acid sequence having at least, or at least about 98% sequence identity to the amino acid sequence of SEQ ID NO: 40 or 45. In some embodiments, the V_{L} region comprises, consists essentially of, or consists of an amino acid sequence having at least, or at least about 99% sequence identity to the amino acid sequence of SEQ ID NO: 40 or 45.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 40 or 45. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 40. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 45.

In some embodiments, the V_{H} region comprises, consists essentially of, or consists of an amino acid sequence having at least, or at least about 85% sequence identity to the amino acid sequence of SEQ ID NO: 34; and the V_{L} region comprises, consists essentially of, or consists of an amino acid sequence having at least, or at least about 85% sequence identity to the amino acid sequence of SEQ ID NO: 40 or 45. In some embodiments, the V_{H} region comprises, consists essentially of, or consists of an amino acid sequence having at least, or at least about 90% sequence identity to the amino acid sequence of SEQ ID NO: 34; and the V_{L} region comprises, consists essentially of, or consists of an amino acid sequence having at least, or at least about 90% sequence identity to the amino acid sequence of SEQ ID NO: 40 or 45. In some embodiments, the V_{H} region comprises, consists essentially of, or consists of an amino acid sequence having at least, or at least about 95% sequence identity to the amino acid sequence of SEQ ID NO: 34; and the V_{L} region comprises, consists essentially of, or consists of an amino acid sequence having at least, or at least about 95% sequence identity to the amino acid sequence of SEQ ID NO: 40 or 45. In some embodiments, the V_{H} region comprises, consists essentially of, or consists of an amino acid sequence having at least, or at least about 96% sequence identity to the amino acid sequence of SEQ ID NO: 34; and the V_{L} region comprises, consists essentially of, or consists of an amino acid sequence having at least, or at least about 96% sequence identity to the amino acid sequence of SEQ ID NO: 40 or 45. In some embodiments, the V_{H} region comprises, consists essentially of, or consists of an amino acid sequence having at least, or at least about 97% sequence identity to the amino acid sequence of SEQ ID NO: 34; and the V_{L} region comprises, consists essentially of, or consists of an amino acid sequence having at least, or at least about 97% sequence identity to the amino acid sequence of SEQ ID NO: 40 or 45. In some embodiments, the V_{H} region comprises, consists essentially of, or consists of an amino acid sequence having at least, or at least about 98% sequence identity to the amino acid sequence of SEQ ID NO: 34; and the V_{L} region comprises, consists essentially of, or consists of an amino acid sequence having at least, or at least about 98% sequence identity to the amino acid sequence of SEQ ID NO: 40 or 45. In some embodiments, the V_{H} region comprises, consists essentially of, or consists of an amino acid sequence having at least, or at least about 99% sequence identity to the amino acid sequence of SEQ ID NO: 34; and the V_{L} region comprises, consists essentially of, or consists of an amino acid sequence having at least, or at least about 99% sequence identity to the amino acid sequence of SEQ ID NO: 40 or 45.

In some embodiments, the V_{H} region comprises, consists essentially of, or consists of the amino acid sequence of SEQ ID NO: 34. In some embodiments, the V_{H} region comprises, consists essentially of, or consists of the amino acid sequence of SEQ ID NO: 34; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 40 or 45.

In some embodiments, the V_{L} region comprises, consists essentially of, or consists of the amino acid sequence of SEQ ID NO: 40 or 45. In some embodiments, the V_{L} region comprises, consists essentially of, or consists of the amino acid sequence of SEQ ID NO: 40. In some embodiments, the V_{L} region comprises, consists essentially of, or consists of the amino acid sequence of SEQ ID NO: 45. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 34; and the V_{L} region comprises, consists essentially of, or consists of the amino acid sequence of SEQ ID NO: 40 or 45.

In some embodiments, the V_{H} region comprises, consists essentially of, or consists of the amino acid sequence of SEQ ID NO: 34; and the V_{L} region comprises, consists essentially of, or consists of the amino acid sequence of SEQ ID NO: 40 or 45. In some embodiments, the V_{H} region comprises, consists essentially of, or consists of the amino acid sequence of SEQ ID NO: 34; and the V_{L} region comprises, consists essentially of, or consists of the amino acid sequence of SEQ ID NO: 40. In some embodiments, the V_{H} region comprises, consists essentially of, or consists of the amino acid sequence of SEQ ID NO: 34; and the V_{L} region comprises, consists essentially of, or consists of the amino acid sequence of SEQ ID NO: 45.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32. In some embodiments, the heavy chain comprises an amino acid sequence having at least, or at least about 85% sequence identity to the amino acid sequence of SEQ ID NO: 32. In some embodiments, the heavy chain comprises an amino acid sequence having at least, or at least about 90% sequence identity to the amino acid sequence of SEQ ID NO: 32. In some embodiments, the heavy chain comprises an amino acid sequence having at least, or at least about 95% sequence identity to the amino acid sequence of SEQ ID NO: 32. In some embodiments, the heavy chain comprises an amino acid sequence having at least, or at least about 96% sequence identity to the amino acid sequence of SEQ ID NO: 32. In some embodiments, the heavy chain comprises an amino acid sequence having at least, or at least about 97% sequence identity to the amino acid sequence of SEQ ID NO: 32. In some embodiments, the heavy chain comprises an amino acid sequence having at least, or at least about 98% sequence identity to the amino acid sequence of SEQ ID NO: 32. In some embodiments, the heavy chain comprises an amino acid sequence having at least, or at least about 99% sequence identity to the amino acid sequence of SEQ ID NO: 32.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 39 or 44. In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 39. In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 44. In some embodiments, the light chain comprises an amino acid sequence having at least, or at least about 85% sequence identity to the amino acid sequence of SEQ ID NO: 39 or 44. In some embodiments, the light chain comprises an amino acid sequence having at least, or at least about 90% sequence identity to the amino acid sequence of SEQ ID NO: 39 or 44. In some embodiments, the light chain comprises an amino acid sequence having at least, or at least about 95% sequence identity to the amino acid sequence of SEQ ID NO: 39 or 44. In some embodiments, the light chain comprises an amino acid sequence having at least, or at least about 96% sequence identity to the amino acid sequence of SEQ ID NO: 39 or 44. In some embodiments, the light chain comprises an amino acid sequence having at least, or at least about 97% sequence identity to the amino acid sequence of SEQ ID NO: 39 or 44. In some embodiments, the light chain comprises an amino acid sequence having at least, or at least about 98% sequence identity to the amino acid sequence of SEQ ID NO: 39 or 44. In some embodiments, the light chain comprises an amino acid sequence having at least, or at least about 99% sequence identity to the amino acid sequence of SEQ ID NO: 39 or 44.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 39 or 44. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 39. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 44.

In some embodiments, the heavy chain comprises an amino acid sequence having at least, or at least about 85% sequence identity to the amino acid sequence of SEQ ID NO: 32; and the light chain comprises an amino acid sequence having at least, or at least about 85% sequence identity to the amino acid sequence of SEQ ID NO: 39 or 44. In some embodiments, the heavy chain comprises an amino acid sequence having at least, or at least about 90% sequence identity to the amino acid sequence of SEQ ID NO: 32; and the light chain comprises an amino acid sequence having at least, or at least about 90% sequence identity to the amino acid sequence of SEQ ID NO: 39 or 44. In some embodiments, the heavy chain comprises an amino acid sequence having at least, or at least about 95% sequence identity to the amino acid sequence of SEQ ID NO: 32; and the light chain comprises an amino acid sequence having at least, or at least about 95% sequence identity to the amino acid sequence of SEQ ID NO: 39 or 44. In some embodiments, the heavy chain comprises an amino acid sequence having at least, or at least about 96% sequence identity to the amino acid sequence of SEQ ID NO: 32; and the light chain comprises an amino acid sequence having at least, or at least about 96% sequence identity to the amino acid sequence of SEQ ID NO: 39 or 44. In some embodiments, the heavy chain comprises an amino acid sequence having at least, or at least about 97% sequence identity to the amino acid sequence of SEQ ID NO: 32; and the light chain comprises an amino acid sequence having at least, or at least about 97% sequence identity to the amino acid sequence of SEQ ID NO: 39 or 44. In some embodiments, the heavy chain comprises an amino acid sequence having at least, or at least about 98% sequence identity to the amino acid sequence of SEQ ID NO: 32; and the light chain comprises an amino acid sequence having at least, or at least about 98% sequence identity to the amino acid sequence of SEQ ID NO: 39 or 44. In some embodiments, the heavy chain comprises an amino acid sequence having at least, or at least about 99% sequence identity to the amino acid sequence of SEQ ID NO: 32; and the light chain comprises an amino acid sequence having at least, or at least about 99% sequence identity to the amino acid sequence of SEQ ID NO: 39 or 44.

In some embodiments, the anti-IL-18Rβ antibody or antigen-binding fragment thereof comprises a heavy chain comprising the amino acid sequence of SEQ ID NO: 32. In some embodiments, the anti-IL-18Rβ antibody or antigen-binding fragment thereof comprises a heavy chain comprising the amino acid sequence of SEQ ID NO: 32; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 39 or 44.

In some embodiments, the anti-IL-18Rβ antibody or antigen-binding fragment thereof comprises a light chain comprising the amino acid sequence of SEQ ID NO: 39 or 44. In some embodiments, the anti-IL-18Rβ antibody or antigen-binding fragment thereof comprises a light chain comprising the amino acid sequence of SEQ ID NO: 39. In some embodiments, the anti-IL-18Rβ antibody or antigen-binding fragment thereof comprises a light chain comprising the amino acid sequence of SEQ ID NO: 44. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising the amino acid sequence of SEQ ID NO: 39 or 44.

In some embodiments, the anti-IL-18Rβ antibody or antigen-binding fragment thereof comprises a heavy chain comprising the amino acid sequence of SEQ ID NO: 32, and a light chain comprising the amino acid sequence of SEQ ID NO: 39 or 44. In some embodiments, the anti-IL-18Rβ antibody or antigen-binding fragment thereof comprises a heavy chain comprising the amino acid sequence of SEQ ID NO: 32, and a light chain comprising the amino acid sequence of SEQ ID NO: 39. In some embodiments, the anti-IL-18Rβ antibody or antigen-binding fragment thereof comprises a heavy chain comprising the amino acid sequence of SEQ ID NO: 32, and a light chain comprising the amino acid sequence of SEQ ID NO: 44.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 75; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 76.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 52, 55, 57, 59, and 62, a heavy chain complementarity determining region 2 (CDR-H2) comprising an amino acid sequence selected from the group consisting of SEQ ID NOs: 53, 56, 58, 60, and 63, and a heavy chain complementarity determining region 3 (CDR-H3) comprising an amino acid sequence selected from the group consisting of SEQ ID NOs: 54, 61, and 64; and the V_{L} region comprises a light chain complementarity determining region 1 (CDR-L1) comprising an amino acid sequence selected from the group consisting of SEQ ID NOs: 66, 69, and 72, a light chain complementarity determining region 2 (CDR-L2) comprising an amino acid sequence selected from the group consisting of SEQ ID NOs: 67, 70, and 29, and a light chain complementarity determining region 3 (CDR-L3) comprising the amino acid sequence of SEQ ID NO: 68 or 71.

In some embodiments, the V_{H} region comprises a CDR-H1, a CDR-H2, and a CDR-H3 comprising the amino acid sequences of SEQ ID NOs: 52, 53, and 54, respectively; and the V_{L} region comprises a CDR-L1, a CDR-L2, and a CDR-L3 comprising the amino acid sequences of SEQ ID NOS: 66, 67, and 68, respectively; wherein the numbering is according to Kabat numbering.

In some embodiments, the V_{H} region comprises a CDR-H1, a CDR-H2, and a CDR-H3 comprising the amino acid sequences of SEQ ID NOs: 55, 56, and 54, respectively; and the V_{L} region comprises a CDR-L1, a CDR-L2, and a CDR-L3 comprising the amino acid sequences of SEQ ID NOS: 66, 67, and 68, respectively; wherein the numbering is according to Chothia numbering.

In some embodiments, the V_{H} region comprises a CDR-H1, a CDR-H2, and a CDR-H3 comprising the amino acid sequences of SEQ ID NOs: 57, 58, and 54, respectively; and the V_{L} region comprises a CDR-L1, a CDR-L2, and a CDR-L3 comprising the amino acid sequences of SEQ ID NOS: 66, 67, and 68, respectively; wherein the numbering is according to AbM numbering.

In some embodiments, the V_{H} region comprises a CDR-H1, a CDR-H2, and a CDR-H3 comprising the amino acid sequences of SEQ ID NOs: 59, 60, and 61, respectively; and the V_{L} region comprises a CDR-L1, a CDR-L2, and a CDR-L3 comprising the amino acid sequences of SEQ ID NOS: 69, 70, and 71, respectively; wherein the numbering is according to Contact numbering.

In some embodiments, the V_{H} region comprises a CDR-H1, a CDR-H2, and a CDR-H3 comprising the amino acid sequences of SEQ ID NOs: 62, 63, and 64, respectively; and the V_{L} region comprises a CDR-L1, a CDR-L2, and a CDR-L3 comprising the amino acid sequences of SEQ ID NOs: 72, 29, and 68, respectively; wherein the numbering is according to IMGT numbering.

In some embodiments, provided herein is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 52, a heavy chain complementarity determining region 2 (CDR-H2) comprising the amino acid sequence of SEQ ID NO: 53, and a heavy chain complementarity determining region 3 (CDR-H3) comprising the amino acid sequence of SEQ ID NO: 54; and the V_{L} region comprises a light chain complementarity determining region 1 (CDR-L1) comprising the amino acid sequence of SEQ ID NO: 66, a light chain complementarity determining region 2 (CDR-L2) comprising an amino acid sequence selected from the group consisting of SEQ ID NOs: 67, and a light chain complementarity determining region 3 (CDR-L3) comprising the amino acid sequence of SEQ ID NO: 68.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 75. In some embodiments, the V_{H} region comprises, consists essentially of, or consists of an amino acid sequence having at least, or at least about 85% sequence identity to the amino acid sequence of SEQ ID NO: 75. In some embodiments, the V_{H} region comprises, consists essentially of, or consists of an amino acid sequence having at least, or at least about 90% sequence identity to the amino acid sequence of SEQ ID NO: 75. In some embodiments, the V_{H} region comprises, consists essentially of, or consists of an amino acid sequence having at least, or at least about 95% sequence identity to the amino acid sequence of SEQ ID NO: 75. In some embodiments, the V_{H} region comprises, consists essentially of, or consists of an amino acid sequence having at least, or at least about 96% sequence identity to the amino acid sequence of SEQ ID NO: 75. In some embodiments, the V_{H} region comprises, consists essentially of, or consists of an amino acid sequence having at least, or at least about 97% sequence identity to the amino acid sequence of SEQ ID NO: 75. In some embodiments, the V_{H} region comprises, consists essentially of, or consists of an amino acid sequence having at least, or at least about 98% sequence identity to the amino acid sequence of SEQ ID NO: 75. In some embodiments, the V_{H} region comprises, consists essentially of, or consists of an amino acid sequence having at least, or at least about 99% sequence identity to the amino acid sequence of SEQ ID NO: 75.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 76. In some embodiments, the V_{L} region comprises, consists essentially of, or consists of an amino acid sequence having at least, or at least about 85% sequence identity to the amino acid sequence of SEQ ID NO: 76. In some embodiments, the V_{L} region comprises, consists essentially of, or consists of an amino acid sequence having at least, or at least about 90% sequence identity to the amino acid sequence of SEQ ID NO: 76. In some embodiments, the V_{L} region comprises, consists essentially of, or consists of an amino acid sequence having at least, or at least about 95% sequence identity to the amino acid sequence of SEQ ID NO: 76. In some embodiments, the V_{L} region comprises, consists essentially of, or consists of an amino acid sequence having at least, or at least about 96% sequence identity to the amino acid sequence of SEQ ID NO: 76. In some embodiments, the V_{L} region comprises, consists essentially of, or consists of an amino acid sequence having at least, or at least about 97% sequence identity to the amino acid sequence of SEQ ID NO: 76. In some embodiments, the V_{L} region comprises, consists essentially of, or consists of an amino acid sequence having at least, or at least about 98% sequence identity to the amino acid sequence of SEQ ID NO: 76. In some embodiments, the V_{L} region comprises, consists essentially of, or consists of an amino acid sequence having at least, or at least about 99% sequence identity to the amino acid sequence of SEQ ID NO: 76.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 75; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 76. In some embodiments, the V_{H} region comprises, consists essentially of, or consists of an amino acid sequence having at least, or at least about 85% sequence identity to the amino acid sequence of SEQ ID NO: 75; and the V_{L} region comprises, consists essentially of, or consists of an amino acid sequence having at least, or at least about 85% sequence identity to the amino acid sequence of SEQ ID NO: 76. In some embodiments, the V_{H} region comprises, consists essentially of, or consists of an amino acid sequence having at least, or at least about 90% sequence identity to the amino acid sequence of SEQ ID NO: 75; and the V_{L} region comprises, consists essentially of, or consists of an amino acid sequence having at least, or at least about 90% sequence identity to the amino acid sequence of SEQ ID NO: 76. In some embodiments, the V_{H} region comprises, consists essentially of, or consists of an amino acid sequence having at least, or at least about 95% sequence identity to the amino acid sequence of SEQ ID NO: 75; and the V_{L} region comprises, consists essentially of, or consists of an amino acid sequence having at least, or at least about 95% sequence identity to the amino acid sequence of SEQ ID NO: 76. In some embodiments, the V_{H} region comprises, consists essentially of, or consists of an amino acid sequence having at least, or at least about 96% sequence identity to the amino acid sequence of SEQ ID NO: 75; and the V_{L} region comprises, consists essentially of, or consists of an amino acid sequence having at least, or at least about 96% sequence identity to the amino acid sequence of SEQ ID NO: 76. In some embodiments, the V_{H} region comprises, consists essentially of, or consists of an amino acid sequence having at least, or at least about 97% sequence identity to the amino acid sequence of SEQ ID NO: 75; and the V_{L} region comprises, consists essentially of, or consists of an amino acid sequence having at least, or at least about 97% sequence identity to the amino acid sequence of SEQ ID NO: 76. In some embodiments, the V_{H} region comprises, consists essentially of, or consists of an amino acid sequence having at least, or at least about 98% sequence identity to the amino acid sequence of SEQ ID NO: 75; and the V_{L} region comprises, consists essentially of, or consists of an amino acid sequence having at least, or at least about 98% sequence identity to the amino acid sequence of SEQ ID NO: 76. In some embodiments, the V_{H} region comprises, consists essentially of, or consists of an amino acid sequence having at least, or at least about 99% sequence identity to the amino acid sequence of SEQ ID NO: 75; and the V_{L} region comprises, consists essentially of, or consists of an amino acid sequence having at least, or at least about 99% sequence identity to the amino acid sequence of SEQ ID NO: 76.

In some embodiments, the V_{H} region comprises, consists essentially of, or consists of the amino acid sequence of SEQ ID NO: 75. In some embodiments, the V_{H} region comprises, consists essentially of, or consists of the amino acid sequence of SEQ ID NO: 75; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 76.

In some embodiments, the V_{L} region comprises, consists essentially of, or consists of the amino acid sequence of SEQ ID NO: 76. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 75; and the V_{L} region comprises, consists essentially of, or consists of the amino acid sequence of SEQ ID NO: 76.

In some embodiments, the V_{H} region comprises, consists essentially of, or consists of the amino acid sequence of SEQ ID NO: 75; and the V_{L} region comprises, consists essentially of, or consists of the amino acid sequence of SEQ ID NO: 76.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 73. In some embodiments, the heavy chain comprises an amino acid sequence having at least, or at least about 85% sequence identity to the amino acid sequence of SEQ ID NO: 73. In some embodiments, the heavy chain comprises an amino acid sequence having at least, or at least about 90% sequence identity to the amino acid sequence of SEQ ID NO: 73. In some embodiments, the heavy chain comprises an amino acid sequence having at least, or at least about 95% sequence identity to the amino acid sequence of SEQ ID NO: 73. In some embodiments, the heavy chain comprises an amino acid sequence having at least, or at least about 96% sequence identity to the amino acid sequence of SEQ ID NO: 73. In some embodiments, the heavy chain comprises an amino acid sequence having at least, or at least about 97% sequence identity to the amino acid sequence of SEQ ID NO: 73. In some embodiments, the heavy chain comprises an amino acid sequence having at least, or at least about 98% sequence identity to the amino acid sequence of SEQ ID NO: 73. In some embodiments, the heavy chain comprises an amino acid sequence having at least, or at least about 99% sequence identity to the amino acid sequence of SEQ ID NO: 73.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 74. In some embodiments, the light chain comprises an amino acid sequence having at least, or at least about 85% sequence identity to the amino acid sequence of SEQ ID NO: 74. In some embodiments, the light chain comprises an amino acid sequence having at least, or at least about 90% sequence identity to the amino acid sequence of SEQ ID NO: 74. In some embodiments, the light chain comprises an amino acid sequence having at least, or at least about 95% sequence identity to the amino acid sequence of SEQ ID NO: 74. In some embodiments, the light chain comprises an amino acid sequence having at least, or at least about 96% sequence identity to the amino acid sequence of SEQ ID NO: 74. In some embodiments, the light chain comprises an amino acid sequence having at least, or at least about 97% sequence identity to the amino acid sequence of SEQ ID NO: 74. In some embodiments, the light chain comprises an amino acid sequence having at least, or at least about 98% sequence identity to the amino acid sequence of SEQ ID NO: 74. In some embodiments, the light chain comprises an amino acid sequence having at least, or at least about 99% sequence identity to the amino acid sequence of SEQ ID NO: 74.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 73; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 74.

In some embodiments, the heavy chain comprises an amino acid sequence having at least, or at least about 85% sequence identity to the amino acid sequence of SEQ ID NO: 73; and the light chain comprises an amino acid sequence having at least, or at least about 85% sequence identity to the amino acid sequence of SEQ ID NO: 74. In some embodiments, the heavy chain comprises an amino acid sequence having at least, or at least about 90% sequence identity to the amino acid sequence of SEQ ID NO: 73; and the light chain comprises an amino acid sequence having at least, or at least about 90% sequence identity to the amino acid sequence of SEQ ID NO: 74. In some embodiments, the heavy chain comprises an amino acid sequence having at least, or at least about 95% sequence identity to the amino acid sequence of SEQ ID NO: 73; and the light chain comprises an amino acid sequence having at least, or at least about 95% sequence identity to the amino acid sequence of SEQ ID NO: 74. In some embodiments, the heavy chain comprises an amino acid sequence having at least, or at least about 96% sequence identity to the amino acid sequence of SEQ ID NO: 73; and the light chain comprises an amino acid sequence having at least, or at least about 96% sequence identity to the amino acid sequence of SEQ ID NO: 74. In some embodiments, the heavy chain comprises an amino acid sequence having at least, or at least about 97% sequence identity to the amino acid sequence of SEQ ID NO: 73; and the light chain comprises an amino acid sequence having at least, or at least about 97% sequence identity to the amino acid sequence of SEQ ID NO: 74. In some embodiments, the heavy chain comprises an amino acid sequence having at least, or at least about 98% sequence identity to the amino acid sequence of SEQ ID NO: 73; and the light chain comprises an amino acid sequence having at least, or at least about 98% sequence identity to the amino acid sequence of SEQ ID NO: 74. In some embodiments, the heavy chain comprises an amino acid sequence having at least, or at least about 99% sequence identity to the amino acid sequence of SEQ ID NO: 73; and the light chain comprises an amino acid sequence having at least, or at least about 99% sequence identity to the amino acid sequence of SEQ ID NO: 74.

In some embodiments, the anti-IL-18Rβ antibody or antigen-binding fragment thereof comprises a heavy chain comprising the amino acid sequence of SEQ ID NO: 73. In some embodiments, the anti-IL-18Rβ antibody or antigen-binding fragment thereof comprises a heavy chain comprising the amino acid sequence of SEQ ID NO: 73; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 74.

In some embodiments, the anti-IL-18Rβ antibody or antigen-binding fragment thereof comprises a light chain comprising the amino acid sequence of SEQ ID NO: 74. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 73; and a light chain comprising the amino acid sequence of SEQ ID NO: 74.

In some embodiments, the anti-IL-18Rβ antibody or antigen-binding fragment thereof comprises a heavy chain comprising the amino acid sequence of SEQ ID NO: 73, and a light chain comprising the amino acid sequence of SEQ ID NO: 74.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 79; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 93.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 80, 83, 85, 87, and 90, a heavy chain complementarity determining region 2 (CDR-H2) comprising an amino acid sequence selected from the group consisting of SEQ ID NOs: 81, 84, 86, 88, and 91, and a heavy chain complementarity determining region 3 (CDR-H3) comprising an amino acid sequence selected from the group consisting of SEQ ID NOs: 82, 89, and 92; and the V_{L} region comprises a light chain complementarity determining region 1 (CDR-L1) comprising an amino acid sequence selected from the group consisting of SEQ ID NOs: 22, 25, and 28, a light chain complementarity determining region 2 (CDR-L2) comprising an amino acid sequence selected from the group consisting of SEQ ID NOs: 94, 96, and 29, and a light chain complementarity determining region 3 (CDR-L3) comprising the amino acid sequence of SEQ ID NO: 95 or 97.

In some embodiments, the V_{H} region comprises a CDR-H1, a CDR-H2, and a CDR-H3 comprising the amino acid sequences of SEQ ID NOs: 80, 81, and 82, respectively; and the V_{L} region comprises a CDR-L1, a CDR-L2, and a CDR-L3 comprising the amino acid sequences of SEQ ID NOS: 22, 94, and 95, respectively; wherein the numbering is according to Kabat numbering.

In some embodiments, the V_{H} region comprises a CDR-H1, a CDR-H2, and a CDR-H3 comprising the amino acid sequences of SEQ ID NOs: 83, 84, and 82, respectively; and the V_{L} region comprises a CDR-L1, a CDR-L2, and a CDR-L3 comprising the amino acid sequences of SEQ ID NOs: 22, 94, and 95, respectively; wherein the numbering is according to Chothia numbering.

In some embodiments, the V_{H} region comprises a CDR-H1, a CDR-H2, and a CDR-H3 comprising the amino acid sequences of SEQ ID NOs: 85, 86, and 82, respectively; and the V_{L} region comprises a CDR-L1, a CDR-L2, and a CDR-L3 comprising the amino acid sequences of SEQ ID NOS: 22, 94, and 95, respectively; wherein the numbering is according to AbM numbering.

In some embodiments, the V_{H} region comprises a CDR-H1, a CDR-H2, and a CDR-H3 comprising the amino acid sequences of SEQ ID NOs: 87, 88, and 89, respectively; and the V_{L} region comprises a CDR-L1, a CDR-L2, and a CDR-L3 comprising the amino acid sequences of SEQ ID NOs: 25, 96, and 97, respectively; wherein the numbering is according to Contact numbering.

In some embodiments, the V_{H} region comprises a CDR-H1, a CDR-H2, and a CDR-H3 comprising the amino acid sequences of SEQ ID NOs: 90, 91, and 92, respectively; and the V_{L} region comprises a CDR-L1, a CDR-L2, and a CDR-L3 comprising the amino acid sequences of SEQ ID NOs: 28, 29, and 95, respectively; wherein the numbering is according to IMGT numbering.

In some embodiments, provided herein is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 80, a heavy chain complementarity determining region 2 (CDR-H2) comprising the amino acid sequence of SEQ ID NO: 81, and a heavy chain complementarity determining region 3 (CDR-H3) comprising the amino acid sequence of SEQ ID NO: 82; and the V_{L} region comprises a light chain complementarity determining region 1 (CDR-L1) comprising the amino acid sequence of SEQ ID NO: 22, a light chain complementarity determining region 2 (CDR-L2) comprising an amino acid sequence selected from the group consisting of SEQ ID NOs: 94, and a light chain complementarity determining region 3 (CDR-L3) comprising the amino acid sequence of SEQ ID NO: 95.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 79. In some embodiments, the V_{H} region comprises, consists essentially of, or consists of an amino acid sequence having at least, or at least about 85% sequence identity to the amino acid sequence of SEQ ID NO: 79. In some embodiments, the V_{H} region comprises, consists essentially of, or consists of an amino acid sequence having at least, or at least about 90% sequence identity to the amino acid sequence of SEQ ID NO: 79. In some embodiments, the V_{H} region comprises, consists essentially of, or consists of an amino acid sequence having at least, or at least about 95% sequence identity to the amino acid sequence of SEQ ID NO: 79. In some embodiments, the V_{H} region comprises, consists essentially of, or consists of an amino acid sequence having at least, or at least about 96% sequence identity to the amino acid sequence of SEQ ID NO: 79. In some embodiments, the V_{H} region comprises, consists essentially of, or consists of an amino acid sequence having at least, or at least about 97% sequence identity to the amino acid sequence of SEQ ID NO: 79. In some embodiments, the V_{H} region comprises, consists essentially of, or consists of an amino acid sequence having at least, or at least about 98% sequence identity to the amino acid sequence of SEQ ID NO: 79. In some embodiments, the V_{H} region comprises, consists essentially of, or consists of an amino acid sequence having at least, or at least about 99% sequence identity to the amino acid sequence of SEQ ID NO: 79.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 93. In some embodiments, the V_{L} region comprises, consists essentially of, or consists of an amino acid sequence having at least, or at least about 85% sequence identity to the amino acid sequence of SEQ ID NO: 93. In some embodiments, the V_{L} region comprises, consists essentially of, or consists of an amino acid sequence having at least, or at least about 90% sequence identity to the amino acid sequence of SEQ ID NO: 93. In some embodiments, the V_{L} region comprises, consists essentially of, or consists of an amino acid sequence having at least, or at least about 95% sequence identity to the amino acid sequence of SEQ ID NO: 93. In some embodiments, the V_{L} region comprises, consists essentially of, or consists of an amino acid sequence having at least, or at least about 96% sequence identity to the amino acid sequence of SEQ ID NO: 93. In some embodiments, the V_{L} region comprises, consists essentially of, or consists of an amino acid sequence having at least, or at least about 97% sequence identity to the amino acid sequence of SEQ ID NO: 93. In some embodiments, the V_{L} region comprises, consists essentially of, or consists of an amino acid sequence having at least, or at least about 98% sequence identity to the amino acid sequence of SEQ ID NO: 93. In some embodiments, the V_{L} region comprises, consists essentially of, or consists of an amino acid sequence having at least, or at least about 99% sequence identity to the amino acid sequence of SEQ ID NO: 93.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 79; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 93. In some embodiments, the V_{H} region comprises, consists essentially of, or consists of an amino acid sequence having at least, or at least about 85% sequence identity to the amino acid sequence of SEQ ID NO: 79; and the V_{L} region comprises, consists essentially of, or consists of an amino acid sequence having at least, or at least about 85% sequence identity to the amino acid sequence of SEQ ID NO: 93. In some embodiments, the V_{H} region comprises, consists essentially of, or consists of an amino acid sequence having at least, or at least about 90% sequence identity to the amino acid sequence of SEQ ID NO: 79; and the V_{L} region comprises, consists essentially of, or consists of an amino acid sequence having at least, or at least about 90% sequence identity to the amino acid sequence of SEQ ID NO: 93. In some embodiments, the V_{H} region comprises, consists essentially of, or consists of an amino acid sequence having at least, or at least about 95% sequence identity to the amino acid sequence of SEQ ID NO: 79; and the V_{L} region comprises, consists essentially of, or consists of an amino acid sequence having at least, or at least about 95% sequence identity to the amino acid sequence of SEQ ID NO: 93. In some embodiments, the V_{H} region comprises, consists essentially of, or consists of an amino acid sequence having at least, or at least about 96% sequence identity to the amino acid sequence of SEQ ID NO: 79; and the V_{L} region comprises, consists essentially of, or consists of an amino acid sequence having at least, or at least about 96% sequence identity to the amino acid sequence of SEQ ID NO: 93. In some embodiments, the V_{H} region comprises, consists essentially of, or consists of an amino acid sequence having at least, or at least about 97% sequence identity to the amino acid sequence of SEQ ID NO: 79; and the V_{L} region comprises, consists essentially of, or consists of an amino acid sequence having at least, or at least about 97% sequence identity to the amino acid sequence of SEQ ID NO: 93. In some embodiments, the V_{H} region comprises, consists essentially of, or consists of an amino acid sequence having at least, or at least about 98% sequence identity to the amino acid sequence of SEQ ID NO: 79; and the V_{L} region comprises, consists essentially of, or consists of an amino acid sequence having at least, or at least about 98% sequence identity to the amino acid sequence of SEQ ID NO: 93. In some embodiments, the V_{H} region comprises, consists essentially of, or consists of an amino acid sequence having at least, or at least about 99% sequence identity to the amino acid sequence of SEQ ID NO: 79; and the V_{L} region comprises, consists essentially of, or consists of an amino acid sequence having at least, or at least about 99% sequence identity to the amino acid sequence of SEQ ID NO: 93.

In some embodiments, the V_{H} region comprises, consists essentially of, or consists of the amino acid sequence of SEQ ID NO: 79. In some embodiments, the V_{H} region comprises, consists essentially of, or consists of the amino acid sequence of SEQ ID NO: 79; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 93.

In some embodiments, the V_{L} region comprises, consists essentially of, or consists of the amino acid sequence of SEQ ID NO: 93. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 79; and the V_{L} region comprises, consists essentially of, or consists of the amino acid sequence of SEQ ID NO: 93.

In some embodiments, the V_{H} region comprises, consists essentially of, or consists of the amino acid sequence of SEQ ID NO: 79; and the V_{L} region comprises, consists essentially of, or consists of the amino acid sequence of SEQ ID NO: 93.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 77. In some embodiments, the heavy chain comprises an amino acid sequence having at least, or at least about 85% sequence identity to the amino acid sequence of SEQ ID NO: 77. In some embodiments, the heavy chain comprises an amino acid sequence having at least, or at least about 90% sequence identity to the amino acid sequence of SEQ ID NO: 77. In some embodiments, the heavy chain comprises an amino acid sequence having at least, or at least about 95% sequence identity to the amino acid sequence of SEQ ID NO: 77. In some embodiments, the heavy chain comprises an amino acid sequence having at least, or at least about 96% sequence identity to the amino acid sequence of SEQ ID NO: 77. In some embodiments, the heavy chain comprises an amino acid sequence having at least, or at least about 97% sequence identity to the amino acid sequence of SEQ ID NO: 77. In some embodiments, the heavy chain comprises an amino acid sequence having at least, or at least about 98% sequence identity to the amino acid sequence of SEQ ID NO: 77. In some embodiments, the heavy chain comprises an amino acid sequence having at least, or at least about 99% sequence identity to the amino acid sequence of SEQ ID NO: 77.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 78. In some embodiments, the light chain comprises an amino acid sequence having at least, or at least about 85% sequence identity to the amino acid sequence of SEQ ID NO: 78. In some embodiments, the light chain comprises an amino acid sequence having at least, or at least about 90% sequence identity to the amino acid sequence of SEQ ID NO: 78. In some embodiments, the light chain comprises an amino acid sequence having at least, or at least about 95% sequence identity to the amino acid sequence of SEQ ID NO: 78. In some embodiments, the light chain comprises an amino acid sequence having at least, or at least about 96% sequence identity to the amino acid sequence of SEQ ID NO: 78. In some embodiments, the light chain comprises an amino acid sequence having at least, or at least about 97% sequence identity to the amino acid sequence of SEQ ID NO: 78. In some embodiments, the light chain comprises an amino acid sequence having at least, or at least about 98% sequence identity to the amino acid sequence of SEQ ID NO: 78. In some embodiments, the light chain comprises an amino acid sequence having at least, or at least about 99% sequence identity to the amino acid sequence of SEQ ID NO: 78.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 77; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 78.

In some embodiments, the heavy chain comprises an amino acid sequence having at least, or at least about 85% sequence identity to the amino acid sequence of SEQ ID NO: 77; and the light chain comprises an amino acid sequence having at least, or at least about 85% sequence identity to the amino acid sequence of SEQ ID NO: 78. In some embodiments, the heavy chain comprises an amino acid sequence having at least, or at least about 90% sequence identity to the amino acid sequence of SEQ ID NO: 77; and the light chain comprises an amino acid sequence having at least, or at least about 90% sequence identity to the amino acid sequence of SEQ ID NO: 78. In some embodiments, the heavy chain comprises an amino acid sequence having at least, or at least about 95% sequence identity to the amino acid sequence of SEQ ID NO: 77; and the light chain comprises an amino acid sequence having at least, or at least about 95% sequence identity to the amino acid sequence of SEQ ID NO: 78. In some embodiments, the heavy chain comprises an amino acid sequence having at least, or at least about 96% sequence identity to the amino acid sequence of SEQ ID NO: 77; and the light chain comprises an amino acid sequence having at least, or at least about 96% sequence identity to the amino acid sequence of SEQ ID NO: 78. In some embodiments, the heavy chain comprises an amino acid sequence having at least, or at least about 97% sequence identity to the amino acid sequence of SEQ ID NO: 77; and the light chain comprises an amino acid sequence having at least, or at least about 97% sequence identity to the amino acid sequence of SEQ ID NO: 78. In some embodiments, the heavy chain comprises an amino acid sequence having at least, or at least about 98% sequence identity to the amino acid sequence of SEQ ID NO: 77; and the light chain comprises an amino acid sequence having at least, or at least about 98% sequence identity to the amino acid sequence of SEQ ID NO: 78. In some embodiments, the heavy chain comprises an amino acid sequence having at least, or at least about 99% sequence identity to the amino acid sequence of SEQ ID NO: 77; and the light chain comprises an amino acid sequence having at least, or at least about 99% sequence identity to the amino acid sequence of SEQ ID NO: 78.

In some embodiments, the anti-IL-18Rβ antibody or antigen-binding fragment thereof comprises a heavy chain comprising the amino acid sequence of SEQ ID NO: 77. In some embodiments, the anti-IL-18Rβ antibody or antigen-binding fragment thereof comprises a heavy chain comprising the amino acid sequence of SEQ ID NO: 77; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 78.

In some embodiments, the anti-IL-18Rβ antibody or antigen-binding fragment thereof comprises a light chain comprising the amino acid sequence of SEQ ID NO: 78. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 77; and a light chain comprising the amino acid sequence of SEQ ID NO: 78.

In some embodiments, the anti-IL-18Rβ antibody or antigen-binding fragment thereof comprises a heavy chain comprising the amino acid sequence of SEQ ID NO: 77, and a light chain comprising the amino acid sequence of SEQ ID NO: 78.

In some embodiments, the anti-IL-18Rβ antibody or antigen-binding fragment thereof is an antigen-binding fragment thereof, such as any of those as described herein.

Also provided herein, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 100; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 101.

Also provided, in some embodiments, is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising an amino acid sequence selected from the group consisting of SEQ ID NOs: 80, 83, 85, 87, and 90, a heavy chain complementarity determining region 2 (CDR-H2) comprising an amino acid sequence selected from the group consisting of SEQ ID NOs: 81, 84, 86, 88, and 91, and a heavy chain complementarity determining region 3 (CDR-H3) comprising an amino acid sequence selected from the group consisting of SEQ ID NOs: 82, 89, and 92; and the V_{L} region comprises a light chain complementarity determining region 1 (CDR-L1) comprising an amino acid sequence selected from the group consisting of SEQ ID NOs: 22, 25, and 28, a light chain complementarity determining region 2 (CDR-L2) comprising an amino acid sequence selected from the group consisting of SEQ ID NOs: 102, 103, and 38, and a light chain complementarity determining region 3 (CDR-L3) comprising the amino acid sequence of SEQ ID NO: 95 or 97.

In some embodiments, the V_{H} region comprises a CDR-H1, a CDR-H2, and a CDR-H3 comprising the amino acid sequences of SEQ ID NOs: 80, 81, and 82, respectively; and the V_{L} region comprises a CDR-L1, a CDR-L2, and a CDR-L3 comprising the amino acid sequences of SEQ ID NOS: 22, 102, and 95, respectively; wherein the numbering is according to Kabat numbering.

In some embodiments, the V_{H} region comprises a CDR-H1, a CDR-H2, and a CDR-H3 comprising the amino acid sequences of SEQ ID NOs: 83, 84, and 82, respectively; and the V_{L} region comprises a CDR-L1, a CDR-L2, and a CDR-L3 comprising the amino acid sequences of SEQ ID NOS: 22, 102, and 95, respectively; wherein the numbering is according to Chothia numbering.

In some embodiments, the V_{H} region comprises a CDR-H1, a CDR-H2, and a CDR-H3 comprising the amino acid sequences of SEQ ID NOs: 85, 86, and 82, respectively; and the V_{L} region comprises a CDR-L1, a CDR-L2, and a CDR-L3 comprising the amino acid sequences of SEQ ID NOS: 22, 102, and 95, respectively; wherein the numbering is according to AbM numbering.

In some embodiments, the V_{H} region comprises a CDR-H1, a CDR-H2, and a CDR-H3 comprising the amino acid sequences of SEQ ID NOs: 87, 88, and 89, respectively; and the V_{L} region comprises a CDR-L1, a CDR-L2, and a CDR-L3 comprising the amino acid sequences of SEQ ID NOS: 25, 103, and 97, respectively; wherein the numbering is according to Contact numbering.

In some embodiments, the V_{H} region comprises a CDR-H1, a CDR-H2, and a CDR-H3 comprising the amino acid sequences of SEQ ID NOs: 90, 91, and 92, respectively; and the V_{L} region comprises a CDR-L1, a CDR-L2, and a CDR-L3 comprising the amino acid sequences of SEQ ID NOS: 28, 38, and 95, respectively; wherein the numbering is according to IMGT numbering.

In some embodiments, provided herein is an anti-IL-18Rβ antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 80, a heavy chain complementarity determining region 2 (CDR-H2) comprising the amino acid sequence of SEQ ID NO: 81, and a heavy chain complementarity determining region 3 (CDR-H3) comprising the amino acid sequence of SEQ ID NO: 82; and the V_{L} region comprises a light chain complementarity determining region 1 (CDR-L1) comprising the amino acid sequence of SEQ ID NO: 22, a light chain complementarity determining region 2 (CDR-L2) comprising an amino acid sequence selected from the group consisting of SEQ ID NOs: 102, and a light chain complementarity determining region 3 (CDR-L3) comprising the amino acid sequence of SEQ ID NO: 95.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 100. In some embodiments, the V_{H} region comprises, consists essentially of, or consists of an amino acid sequence having at least, or at least about 85% sequence identity to the amino acid sequence of SEQ ID NO: 100. In some embodiments, the V_{H} region comprises, consists essentially of, or consists of an amino acid sequence having at least, or at least about 90% sequence identity to the amino acid sequence of SEQ ID NO: 100. In some embodiments, the V_{H} region comprises, consists essentially of, or consists of an amino acid sequence having at least, or at least about 95% sequence identity to the amino acid sequence of SEQ ID NO: 100. In some embodiments, the V_{H} region comprises, consists essentially of, or consists of an amino acid sequence having at least, or at least about 96% sequence identity to the amino acid sequence of SEQ ID NO: 100. In some embodiments, the V_{H} region comprises, consists essentially of, or consists of an amino acid sequence having at least, or at least about 97% sequence identity to the amino acid sequence of SEQ ID NO: 100. In some embodiments, the V_{H} region comprises, consists essentially of, or consists of an amino acid sequence having at least, or at least about 98% sequence identity to the amino acid sequence of SEQ ID NO: 100. In some embodiments, the V_{H} region comprises, consists essentially of, or consists of an amino acid sequence having at least, or at least about 99% sequence identity to the amino acid sequence of SEQ ID NO: 100.

In some embodiments, the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 101. In some embodiments, the V_{L} region comprises, consists essentially of, or consists of an amino acid sequence having at least, or at least about 85% sequence identity to the amino acid sequence of SEQ ID NO: 101. In some embodiments, the V_{L} region comprises, consists essentially of, or consists of an amino acid sequence having at least, or at least about 90% sequence identity to the amino acid sequence of SEQ ID NO: 101. In some embodiments, the V_{L} region comprises, consists essentially of, or consists of an amino acid sequence having at least, or at least about 95% sequence identity to the amino acid sequence of SEQ ID NO: 101. In some embodiments, the V_{L} region comprises, consists essentially of, or consists of an amino acid sequence having at least, or at least about 96% sequence identity to the amino acid sequence of SEQ ID NO: 101. In some embodiments, the V_{L} region comprises, consists essentially of, or consists of an amino acid sequence having at least, or at least about 97% sequence identity to the amino acid sequence of SEQ ID NO: 101. In some embodiments, the V_{L} region comprises, consists essentially of, or consists of an amino acid sequence having at least, or at least about 98% sequence identity to the amino acid sequence of SEQ ID NO: 101. In some embodiments, the V_{L} region comprises, consists essentially of, or consists of an amino acid sequence having at least, or at least about 99% sequence identity to the amino acid sequence of SEQ ID NO: 101.

In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 100; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 101. In some embodiments, the V_{H} region comprises, consists essentially of, or consists of an amino acid sequence having at least, or at least about 85% sequence identity to the amino acid sequence of SEQ ID NO: 100; and the V_{L} region comprises, consists essentially of, or consists of an amino acid sequence having at least, or at least about 85% sequence identity to the amino acid sequence of SEQ ID NO: 101. In some embodiments, the V_{H} region comprises, consists essentially of, or consists of an amino acid sequence having at least, or at least about 90% sequence identity to the amino acid sequence of SEQ ID NO: 100; and the V_{L} region comprises, consists essentially of, or consists of an amino acid sequence having at least, or at least about 90% sequence identity to the amino acid sequence of SEQ ID NO: 101. In some embodiments, the V_{H} region comprises, consists essentially of, or consists of an amino acid sequence having at least, or at least about 95% sequence identity to the amino acid sequence of SEQ ID NO: 100; and the V_{L} region comprises, consists essentially of, or consists of an amino acid sequence having at least, or at least about 95% sequence identity to the amino acid sequence of SEQ ID NO: 101. In some embodiments, the V_{H} region comprises, consists essentially of, or consists of an amino acid sequence having at least, or at least about 96% sequence identity to the amino acid sequence of SEQ ID NO: 100; and the V_{L} region comprises, consists essentially of, or consists of an amino acid sequence having at least, or at least about 96% sequence identity to the amino acid sequence of SEQ ID NO: 101. In some embodiments, the V_{H} region comprises, consists essentially of, or consists of an amino acid sequence having at least, or at least about 97% sequence identity to the amino acid sequence of SEQ ID NO: 100; and the V_{L} region comprises, consists essentially of, or consists of an amino acid sequence having at least, or at least about 97% sequence identity to the amino acid sequence of SEQ ID NO: 101. In some embodiments, the V_{H} region comprises, consists essentially of, or consists of an amino acid sequence having at least, or at least about 98% sequence identity to the amino acid sequence of SEQ ID NO: 100; and the V_{L} region comprises, consists essentially of, or consists of an amino acid sequence having at least, or at least about 98% sequence identity to the amino acid sequence of SEQ ID NO: 101. In some embodiments, the V_{H} region comprises, consists essentially of, or consists of an amino acid sequence having at least, or at least about 99% sequence identity to the amino acid sequence of SEQ ID NO: 100; and the V_{L} region comprises, consists essentially of, or consists of an amino acid sequence having at least, or at least about 99% sequence identity to the amino acid sequence of SEQ ID NO: 101.

In some embodiments, the V_{H} region comprises, consists essentially of, or consists of the amino acid sequence of SEQ ID NO: 100. In some embodiments, the V_{H} region comprises, consists essentially of, or consists of the amino acid sequence of SEQ ID NO: 100; and the V_{L} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 101.

In some embodiments, the V_{L} region comprises, consists essentially of, or consists of the amino acid sequence of SEQ ID NO: 101. In some embodiments, the V_{H} region comprises, consists essentially of, or consists of an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 100; and the V_{L} region comprises, consists essentially of, or consists of the amino acid sequence of SEQ ID NO: 101.

In some embodiments, the V_{H} region comprises, consists essentially of, or consists of the amino acid sequence of SEQ ID NO: 100; and the V_{L} region comprises, consists essentially of, or consists of the amino acid sequence of SEQ ID NO: 101.

In some embodiments, the anti-IL-18Rβ antibody or antigen-binding fragment thereof is an antibody comprising a constant domain, such as any constant domain as described herein. In some embodiments, the anti-IL-18Rβ antibody or antigen-binding fragment thereof comprises a heavy chain and a light chain. In some embodiments, the anti-IL-18Rβ antibody or antigen-binding fragment thereof is an antibody, and the V_{H} region is comprised within a heavy chain and the V_{L} region is comprised within a light chain.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 98. In some embodiments, the heavy chain comprises an amino acid sequence having at least, or at least about 85% sequence identity to the amino acid sequence of SEQ ID NO: 98. In some embodiments, the heavy chain comprises an amino acid sequence having at least, or at least about 90% sequence identity to the amino acid sequence of SEQ ID NO: 98. In some embodiments, the heavy chain comprises an amino acid sequence having at least, or at least about 95% sequence identity to the amino acid sequence of SEQ ID NO: 98. In some embodiments, the heavy chain comprises an amino acid sequence having at least, or at least about 96% sequence identity to the amino acid sequence of SEQ ID NO: 98. In some embodiments, the heavy chain comprises an amino acid sequence having at least, or at least about 97% sequence identity to the amino acid sequence of SEQ ID NO: 98. In some embodiments, the heavy chain comprises an amino acid sequence having at least, or at least about 98% sequence identity to the amino acid sequence of SEQ ID NO: 98. In some embodiments, the heavy chain comprises an amino acid sequence having at least, or at least about 99% sequence identity to the amino acid sequence of SEQ ID NO: 98.

In some embodiments, the anti-IL-18Rβ antibody or antigen-binding fragment thereof comprises a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 99. In some embodiments, the light chain comprises an amino acid sequence having at least, or at least about 85% sequence identity to the amino acid sequence of SEQ ID NO: 99. In some embodiments, the light chain comprises an amino acid sequence having at least, or at least about 90% sequence identity to the amino acid sequence of SEQ ID NO: 99. In some embodiments, the light chain comprises an amino acid sequence having at least, or at least about 95% sequence identity to the amino acid sequence of SEQ ID NO: 99. In some embodiments, the light chain comprises an amino acid sequence having at least, or at least about 96% sequence identity to the amino acid sequence of SEQ ID NO: 99. In some embodiments, the light chain comprises an amino acid sequence having at least, or at least about 97% sequence identity to the amino acid sequence of SEQ ID NO: 99. In some embodiments, the light chain comprises an amino acid sequence having at least, or at least about 98% sequence identity to the amino acid sequence of SEQ ID NO: 99. In some embodiments, the light chain comprises an amino acid sequence having at least, or at least about 99% sequence identity to the amino acid sequence of SEQ ID NO: 99.

In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 98; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 99.

In some embodiments, the heavy chain comprises an amino acid sequence having at least, or at least about 85% sequence identity to the amino acid sequence of SEQ ID NO: 98; and the light chain comprises an amino acid sequence having at least, or at least about 85% sequence identity to the amino acid sequence of SEQ ID NO: 99. In some embodiments, the heavy chain comprises an amino acid sequence having at least, or at least about 90% sequence identity to the amino acid sequence of SEQ ID NO: 98; and the light chain comprises an amino acid sequence having at least, or at least about 90% sequence identity to the amino acid sequence of SEQ ID NO: 99. In some embodiments, the heavy chain comprises an amino acid sequence having at least, or at least about 95% sequence identity to the amino acid sequence of SEQ ID NO: 98; and the light chain comprises an amino acid sequence having at least, or at least about 95% sequence identity to the amino acid sequence of SEQ ID NO: 99. In some embodiments, the heavy chain comprises an amino acid sequence having at least, or at least about 96% sequence identity to the amino acid sequence of SEQ ID NO: 98; and the light chain comprises an amino acid sequence having at least, or at least about 96% sequence identity to the amino acid sequence of SEQ ID NO: 99. In some embodiments, the heavy chain comprises an amino acid sequence having at least, or at least about 97% sequence identity to the amino acid sequence of SEQ ID NO: 98; and the light chain comprises an amino acid sequence having at least, or at least about 97% sequence identity to the amino acid sequence of SEQ ID NO: 99. In some embodiments, the heavy chain comprises an amino acid sequence having at least, or at least about 98% sequence identity to the amino acid sequence of SEQ ID NO: 98; and the light chain comprises an amino acid sequence having at least, or at least about 98% sequence identity to the amino acid sequence of SEQ ID NO: 99. In some embodiments, the heavy chain comprises an amino acid sequence having at least, or at least about 99% sequence identity to the amino acid sequence of SEQ ID NO: 98; and the light chain comprises an amino acid sequence having at least, or at least about 99% sequence identity to the amino acid sequence of SEQ ID NO: 99.

In some embodiments, the anti-IL-18Rβ antibody or antigen-binding fragment thereof comprises a heavy chain comprising the amino acid sequence of SEQ ID NO: 98. In some embodiments, the anti-IL-18Rβ antibody or antigen-binding fragment thereof comprises a heavy chain comprising the amino acid sequence of SEQ ID NO: 98; and a light chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 99.

In some embodiments, the anti-IL-18Rβ antibody or antigen-binding fragment thereof comprises a light chain comprising the amino acid sequence of SEQ ID NO: 99. In some embodiments, the anti-IL-18Rβ antibody or antigen-binding fragment thereof comprises a heavy chain comprising an amino acid sequence having at least, or at least about 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 98; and a light chain comprising the amino acid sequence of SEQ ID NO: 99.

In some embodiments, the anti-IL-18Rβ antibody or antigen-binding fragment thereof comprises a heavy chain comprising the amino acid sequence of SEQ ID NO: 98, and a light chain comprising the amino acid sequence of SEQ ID NO: 99.

In some embodiments, the anti-IL-18Rβ antibody or antigen-binding fragment thereof is an antigen-binding fragment thereof, such as any of those as described herein.

In some of any of the embodiments provided herein, the anti-IL-18Rβ antibody or antigen-binding fragment thereof comprises a constant domain. In some of any of the embodiments provided herein, the anti-IL-18Rβ antibody or antigen-binding fragment thereof is an antibody comprising a constant domain.

In certain embodiments, antibody variable domains as described are fused to immunoglobulin constant domain sequences. In certain embodiments, the fusion is with an Ig heavy chain constant domain, comprising at least part of the hinge, CH2, and CH3 regions. In some of any of the embodiments herein, the antibody further comprises a heavy chain constant domain and/or a light chain constant domain. In some embodiments, the heavy chain and/or light constant domain is human.

In general, the “constant region” or “constant domain” contains a sequence of amino acids that is comparatively more conserved among antibodies than the variable region domain. The constant regions of immunoglobulins show less sequence diversity than the variable regions, and are responsible for binding a number of natural proteins to elicit important biochemical events. Each light chain has a single light chain constant region (CL) domain, which is either of the kappa or lambda type. Each heavy chain contains one or more heavy chain constant region (CH) domains that can be assigned to different classes or isotypes. There are five classes of immunoglobulins: IgA, IgD, IgE, IgG and IgM, having heavy chains designated ┘, ┘, ┘ and ┘, respectively. Full-length IgA, IgD and IgG isotypes contain CH1, CH2, CH3 and a hinge region, while IgE and IgM contain CH1, CH2, CH3 and CH4. The ┘ and ┘ classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant, Fc regions, although subtler differences may exist in the V region. An antibody constant region can include an Fc portion. Although the boundaries of the Fc region of an immunoglobulin heavy chain might vary, the human IgG heavy-chain Fc region usually includes the region containing the CH2 and CH3 domains and the hinge region, such as an amino acid residue at position Cys226, or from Pro230, to the carboxyl-terminus thereof.

In some embodiments, the constant domain is a constant domain selected from the group consisting of an IgA1 constant domain, an IgA2 constant domain, an IgD constant domain, an IgE constant domain, a IgG1 constant domain, an IgG2 constant domain, an IgG3 constant domain, an IgG4 constant domain, and an IgM constant domain. In some embodiments, the constant domain is a human constant domain, e.g., a human constant domain selected from the group consisting of an IgA1 constant domain, an IgA2 constant domain, an IgD constant domain, an IgE constant domain, an IgG1 constant domain, an IgG2 constant domain, an IgG3 constant domain, an IgG4 constant domain, and an IgM constant domain. In some embodiments, the constant domain is an IgG1 constant domain. In some embodiments, the constant domain is a human IgG1 constant domain.

In certain embodiments, a provided antibody contains an Ig heavy chain constant domain comprising at least part of the hinge, CH2, and CH3 regions. Certain embodiments have the first heavy-chain constant region (CH1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired fusion antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In some embodiments, the antibody or antigen-binding fragment thereof, may contain at least a portion of an immunoglobulin constant region, such as one or more constant region domain. In some embodiments, the constant regions include a light chain constant region and/or a heavy chain constant region 1 (CH1). In some embodiments, the antibody includes at least a portion of a hinge region or a variant thereof. In some embodiments, the antibody includes a CH2 and/or CH3 domain, such as an Fc region. In some embodiments, the Fc region is an Fc region from an IgA1, IgA2, IgD. IgE, IgG1, IgG2, IgG3, IgG4, or IgM. In some embodiments, the constant domain of the antibody comprises CH2 and CH3 domains from one or more of the different Ig classes.

In some embodiments, the heavy chain constant sequence includes an IgG4 containing the S228P mutation, which has been shown to prevent recombination between a therapeutic antibody and an endogenous IgG4 by Fab-arm exchange (see e.g. Labrijin et al. (2009) Nat. Biotechnol., 27 (8): 767-71.)

In some embodiments, the constant domain of the anti-IL-18Rβ antibody or antigen-binding fragment thereof comprises a heavy chain constant domain and a light chain constant domain. In some embodiments, the antibody comprises a heavy chain constant domain and a light chain constant domain.

In some embodiments, the heavy chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30. In some embodiments, the heavy chain constant domain comprises an amino acid sequence having at least, or at least about 85% sequence identity to the amino acid sequence of SEQ ID NO: 30. In some embodiments, the heavy chain constant domain comprises an amino acid sequence having at least, or at least about 90% sequence identity to the amino acid sequence of SEQ ID NO: 30. In some embodiments, the heavy chain constant domain comprises an amino acid sequence having at least, or at least about 95% sequence identity to the amino acid sequence of SEQ ID NO: 30. In some embodiments, the heavy chain constant domain comprises an amino acid sequence having at least, or at least about 96% sequence identity to the amino acid sequence of SEQ ID NO: 30. In some embodiments, the heavy chain constant domain comprises an amino acid sequence having at least, or at least about 97% sequence identity to the amino acid sequence of SEQ ID NO: 30. In some embodiments, the heavy chain constant domain comprises an amino acid sequence having at least, or at least about 98% sequence identity to the amino acid sequence of SEQ ID NO: 30. In some embodiments, the heavy chain constant domain comprises an amino acid sequence having at least, or at least about 99% sequence identity to the amino acid sequence of SEQ ID NO: 30.

In some embodiments, the light chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31. In some embodiments, the light chain constant domain comprises an amino acid sequence having at least, or at least about 85% sequence identity to the amino acid sequence of SEQ ID NO: 31. In some embodiments, the light chain constant domain comprises an amino acid sequence having at least, or at least about 90% sequence identity to the amino acid sequence of SEQ ID NO: 31. In some embodiments, the light chain constant domain comprises an amino acid sequence having at least, or at least about 95% sequence identity to the amino acid sequence of SEQ ID NO: 31. In some embodiments, the light chain constant domain comprises an amino acid sequence having at least, or at least about 96% sequence identity to the amino acid sequence of SEQ ID NO: 31. In some embodiments, the light chain constant domain comprises an amino acid sequence having at least, or at least about 97% sequence identity to the amino acid sequence of SEQ ID NO: 31. In some embodiments, the light chain constant domain comprises an amino acid sequence having at least, or at least about 98% sequence identity to the amino acid sequence of SEQ ID NO: 31. In some embodiments, the light chain constant domain comprises an amino acid sequence having at least, or at least about 99% sequence identity to the amino acid sequence of SEQ ID NO: 31.

In some embodiments, the heavy chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and the light chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31.

In some embodiments, the heavy chain constant domain comprises the amino acid sequence of SEQ ID NO: 30. In some embodiments, the light chain constant domain comprises the amino acid sequence of SEQ ID NO: 31.

In some embodiments, the heavy chain constant domain comprises an amino acid sequence selected from the group consisting of SEQ ID NOs: 30, 104, 106, 108, 110, 112, 114, 116, 118, 120, and 122, or an amino acid sequence having at least, or at least about 85%, 86%, 87%, 88%, 89%, 90%, 91%, 92%, 93%, 94%, 95%, 96%, 97%, 98% or 99% sequence identity to an amino acid sequence selected from the group consisting of SEQ ID NOs: 30, 104, 106, 108, 110, 112, 114, 116, 118, 120, and 122.

In some embodiments, the heavy chain constant domain comprises an amino acid sequence selected from the group consisting of SEQ ID NOs: 30, 104, 106, 108, 110, 112, 114, 116, 118, 120, and 122. In some embodiments, the heavy chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to an amino acid sequence selected from the group consisting of SEQ ID NOS: 30, 104, 106, 108, 110, 112, 114, 116, 118, 120, and 122.

In some embodiments, the heavy chain constant domain comprises the amino acid sequence of SEQ ID NO: 104, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 104. In some embodiments, the heavy chain constant domain comprises the amino acid sequence of SEQ ID NO: 106, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 106. In some embodiments, the heavy chain constant domain comprises the amino acid sequence of SEQ ID NO: 108, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 108. In some embodiments, the heavy chain constant domain comprises the amino acid sequence of SEQ ID NO: 110, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 110. In some embodiments, the heavy chain constant domain comprises the amino acid sequence of SEQ ID NO: 112, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 112. In some embodiments, the heavy chain constant domain comprises the amino acid sequence of SEQ ID NO: 114, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 114. In some embodiments, the heavy chain constant domain comprises the amino acid sequence of SEQ ID NO: 116, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 116. In some embodiments, the heavy chain constant domain comprises the amino acid sequence of SEQ ID NO: 118, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 118. In some embodiments, the heavy chain constant domain comprises the amino acid sequence of SEQ ID NO: 120, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 120. In some embodiments, the heavy chain constant domain comprises the amino acid sequence of SEQ ID NO: 122, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 122.

In some embodiments, the heavy chain constant domain comprises the amino acid sequence of SEQ ID NO: 104. In some embodiments, the heavy chain constant domain comprises the amino acid sequence of SEQ ID NO: 106. In some embodiments, the heavy chain constant domain comprises the amino acid sequence of SEQ ID NO: 108. In some embodiments, the heavy chain constant domain comprises the amino acid sequence of SEQ ID NO: 110. In some embodiments, the heavy chain constant domain comprises the amino acid sequence of SEQ ID NO: 112. In some embodiments, the heavy chain constant domain comprises the amino acid sequence of SEQ ID NO: 114. In some embodiments, the heavy chain constant domain comprises the amino acid sequence of SEQ ID NO: 116. In some embodiments, the heavy chain constant domain comprises the amino acid sequence of SEQ ID NO: 118. In some embodiments, the heavy chain constant domain comprises the amino acid sequence of SEQ ID NO: 120. In some embodiments, the heavy chain constant domain comprises the amino acid sequence of SEQ ID NO: 122.

In some embodiments, the heavy chain constant domain is encoded by a nucleic acid sequence selected from the group consisting of SEQ ID NOs: 105, 107, 109, 111, 113, 115, 117, 119, 121, and 123, or a nucleic acid having at least, or at least about 85%, 86%, 87%, 88%, 89%, 90%, 91%, 92%, 93%, 94%, 95%, 96%, 97%, 98% or 99% sequence identity to a nucleic acid sequence selected from the group consisting of SEQ ID NOs: 105, 107, 109, 111, 113, 115, 117, 119, 121, and 123.

In some embodiments, the heavy chain constant domain is encoded by the nucleic acid sequence of SEQ ID NO: 105, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 105. In some embodiments, the heavy chain constant domain is encoded by the nucleic acid sequence of SEQ ID NO: 107, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 107. In some embodiments, the heavy chain constant domain is encoded by the nucleic acid sequence of SEQ ID NO: 109, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 109. In some embodiments, the heavy chain constant domain is encoded by the nucleic acid sequence of SEQ ID NO: 111, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 111. In some embodiments, the heavy chain constant domain is encoded by the nucleic acid sequence of SEQ ID NO: 113, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 113. In some embodiments, the heavy chain constant domain is encoded by the nucleic acid sequence of SEQ ID NO: 115, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 115. In some embodiments, the heavy chain constant domain is encoded by the nucleic acid sequence of SEQ ID NO: 117, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 117. In some embodiments, the heavy chain constant domain is encoded by the nucleic acid sequence of SEQ ID NO: 119, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 119. In some embodiments, the heavy chain constant domain is encoded by the nucleic acid sequence of SEQ ID NO: 121, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 121. In some embodiments, the heavy chain constant domain is encoded by the nucleic acid sequence of SEQ ID NO: 123, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 123.

In some embodiments, the light chain constant domain comprises the amino acid sequence of SEQ ID NO: 31 or 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31 or 124.

In some embodiments, the light chain constant domain comprises the amino acid sequence of SEQ ID NO: 31 or 124. In some embodiments, the light chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31 or 124.

In some embodiments, the light chain constant domain comprises the amino acid sequence of SEQ ID NO: 124. In some embodiments, the light chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the light chain constant domain is encoded by the nucleic acid sequence of SEQ ID NO: 125, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 125.

In some embodiments, the heavy chain constant domain is encoded by a nucleic acid sequence selected from the group consisting of SEQ ID NOs: 105, 107, 109, 111, 113, 115, 117, 119, 121, and 123, or a nucleic acid having at least, or at least about 85%, 86%, 87%, 88%, 89%, 90%, 91%, 92%, 93%, 94%, 95%, 96%, 97%, 98% or 99% sequence identity to a nucleic acid sequence selected from the group consisting of SEQ ID NOs: 105, 107, 109, 111, 113, 115, 117, 119, 121, and 123; and the light chain constant domain is encoded by the nucleic acid sequence of SEQ ID NO: 125, or a nucleic acid having at least, or at least about 85%, 86%, 87%, 88%, 89%, 90%, 91%, 92%, 93%, 94%, 95%, 96%, 97%, 98% or 99% sequence identity to the nucleic acid sequence of SEQ ID NO: 125.

In some embodiments, the heavy chain constant domain comprises an amino acid sequence selected from the group consisting of SEQ ID NOs: 30, 104, 106, 108, 110, 112, 114, 116, 118, 120, and 122, or an amino acid sequence having at least, or at least about 85%, 86%, 87%, 88%, 89%, 90%, 91%, 92%, 93%, 94%, 95%, 96%, 97%, 98% or 99% sequence identity to an amino acid sequence selected from the group consisting of SEQ ID NOs: 30, 104, 106, 108, 110, 112, 114, 116, 118, 120, and 122; and the light chain constant domain comprises the amino acid sequence of SEQ ID NO: 31 or 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31 or 124.

In some embodiments, the heavy chain constant domain comprises an amino acid sequence selected from the group consisting of SEQ ID NOs: 30, 104, 106, 108, 110, 112, 114, 116, 118, 120, and 122; and the light chain constant domain comprises the amino acid sequence of SEQ ID NO: 31 or 124.

In some embodiments, the heavy chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to an amino acid sequence selected from the group consisting of SEQ ID NOs: 30, 104, 106, 108, 110, 112, 114, 116, 118, 120, and 122; and the light chain constant domain comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31 or 124.

In some embodiments, the heavy chain constant domain comprises the amino acid sequence of SEQ ID NO: 30, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 104, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 104; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 106, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 106; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 108, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 108; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 110, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 110; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 112, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 112; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 114, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 114; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 116, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 116; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 118, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 118; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 120, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 120; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

In some embodiments, the heavy chain constant domain comprises the amino acid sequence of SEQ ID NO: 122, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 122; and the light chain constant domain comprises the amino acid sequence of SEQ ID NO: 124, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 124.

Sequences of anti-IL-18Rβ antibodies and antigen-binding fragments thereof disclosed herein, in some embodiments, are provided in Table E12A and Table E16A in the Examples.

In some embodiments, the provided anti-IL-18Rβ antibodies or antigen-binding fragments thereof are human antibodies, or antibodies or antigen-binding fragments thereof that are modified from or variants of human antibodies. The anti-IL-18Rβ antibodies or antigen-binding fragments thereof include isolated antibodies.

In some embodiments, the anti-IL-18Rβ antibody or antigen-binding fragment thereof is an antigen-binding fragment. In some embodiments, the antigen-binding fragment is selected from the group consisting of fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody, V_{HH}) fragments.

In some embodiments, the anti-IL-18Rβ antibodies or antigen-binding fragments thereof include genetically engineered and/or otherwise modified forms of immunoglobulins, such as intrabodies, peptibodies, chimeric antibodies, fully human antibodies, humanized antibodies, and heteroconjugate antibodies, multi-specific antibodies, e.g., bispecific or trispecific antibodies, diabodies, triabodies, and tetrabodies, tandem di-scFv, tandem tri-scFv. In some embodiments, the anti-IL-18Rβ antibody or antigen-binding fragment thereof is an intact or full-length antibody. In some embodiments, the anti-IL-18Rβ antibody or antigen-binding fragment thereof is an intact or full-length antibody comprising a constant domain. In some embodiments, the anti-IL-18Rβ antibody or antigen-binding fragment thereof is an antibody or antigen-binding fragment thereof of any class or sub-class, including IgG and sub-classes thereof, IgM, IgE, IgA, and IgD.

In general, antibodies and antigen-binding fragments thereof include complementarity determining regions (CDRs) and framework regions (FRs) in their variable regions. CDRs are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3), which are separated by FRs that refer to the non-CDR portions of the variable regions of the heavy and light chains. In general, there are four FRs in each full-length heavy chain variable region (FR-H1, FR-H2. FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme): Al-Lazikani et al., J Mol Biol, 1997; 273 (4): 927-48 (“Chothia” numbering scheme); MacCallum et al., J. Mol. Biol, 1996:262:732-745.” (“Contact” numbering scheme); Lefranc M P et al., Dev Comp Immunol, 2003; 27 (1): 55-77 (“IMGT” numbering scheme); Honegger A and Plückthun A, J Mol Biol, 2001; 309 (3): 657-70, (“Aho” numbering scheme); Martin et al., PNAS, 1989; 86 (23): 9268-9272, (“AbM” numbering scheme); and Ye et al., Nucleic Acids Res. 2013; 41 (Web Server issue): W34-40, (“IgBLAST numbering scheme). Details regarding various numbering schemes are also described in, for example, Jarasch et al., Proteins, 2017; 85 (1): 65-71; Martin et al., Bioinformatics tools for antibody engineering. In: Dübel, S. (editor) Handbook of Therapeutic Antibodies, Vol. 1. Wiley-VCH, Weinheim, Germany; Martin, A. C. R. (2010). Protein Sequence and Structure Analysis of Antibody Variable Domains. In: Kontermann, R., Dübel, S. (eds) Antibody Engineering. Springer Protocols Handbooks. Springer, Berlin, Heidelberg; and Martin, A C R. Antibody Information: How to identify the CDRs by looking at a sequence [online] bioinf.org.uk/abs/info.html, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V. D and J genes, and can be determined using National Center for Biotechnology Information (NCBI)'s IgBLAST tool.

In some embodiments, Kabat numbering can be determined by known sequence rules as described in, for example, Kabat et al. (1991), “Sequences of Proteins of Immunological Interest,” 5th Ed. Public Health Service, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y. L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by typically V, but also can be I or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue—the first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some cases, according to the Chothia numbering scheme, exact boundary positions of certain CDRs can differ based on different definitions for the CDRs (See e.g., Martin, A C R, Antibody Information: How to identify the CDRs by looking at a sequence [online] bioinf.org.uk/abs/info.html). For example, in some instances, the boundary positions for CDR-L1 according to Chothia numbering can be L26-L32 (Chothia et al., Science, 1986; 233 (4765): 755-8 and Chothia C. and Lesk A. M. J Mol Biol, 1987; 196 (4): 901-17). In some instances, the boundary positions for CDR-L1 can be L25-L32 (Al-Lazikani et al., J Mol Biol, 1997; 273 (4): 927-48). In some instances, the boundary positions for CDR-L2 can be L50-L52 and for CDR-L3 can be L91-L96 (Chothia et al., Science, 1986; 233 (4765): 755-8; Chothia C. and Lesk A. M. J Mol Biol, 1987; 196 (4): 901-17; and Al-Lazikani et al., J Mol Biol, 1997; 273 (4): 927-48). In some instances, the boundary positions for CDR-H1 according to Chothia numbering can be H26-H32 (Chothia et al., Science, 1986; 233 (4765): 755-8; Chothia C. and Lesk A. M. J Mol Biol, 1987; 196 (4): 901-17; and Al-Lazikani et al., J Mol Biol, 1997; 273 (4): 927-48). In some instances, the boundary positions for CDR-H2 can be H53-H55 (Chothia et al., Science, 1986; 233 (4765): 755-8 and Chothia C. and Lesk A. M. J Mol Biol, 1987, 196 (4): 901-17); H52a-H55 (Tramontano et al., J Mol Biol, 1990, 215 (1): 175-82), or H52-H56 (Al-Lazikani et al., J Mol Biol., 1997; 273 (4): 927-48). In some instances, the boundary positions for CDR-H3 can be H96-H101 (Chothia et al., Science, 1986; 233 (4765): 755-8 and Chothia C. and Lesk A. M. J Mol Biol., 1987; 196 (4): 901-17). In some instances, the boundary positions for CDR-H3 can be H92-H104 (Morea et al., Biophys Chem, 1997; 68 (1-3): 9-16 and Morea et al., J Mol Biol., 1998; 275 (2): 269-94).

Table 1, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2. FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 | H26--H35B | H30--H35B |
| (Kabat | | . . . 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., J Mol Biol., 1997; 273(4): 927-48).

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given V_{H} region amino acid sequence and a CDR-L1, a CDR-L2, and a CDR-L3 as contained within a given V_{L} region amino acid sequence, the CDRs can be defined by any of the aforementioned schemes (e.g., each CDR can be independently defined according to any one of the aforementioned schemes, or all CDRs can be defined according to a single scheme), such as Kabat, Chothia, AbM, IgBLAST, IMGT, or Contact method, or other known scheme. In some embodiments, specific CDR sequences are specified. Exemplary CDR sequences of provided antibodies or antigen binding fragments thereof are described using various numbering schemes, although it is understood that a provided antibody or antigen binding fragment thereof can include CDRs as described according to any of the other aforementioned numbering schemes or other known numbering schemes. In some embodiments, the CDRs are defined by less than six CDRs, such as four CDRs, e.g., CDR-H1, CDR-H2, CDR-H3, and CDR-L3, that include the paratope.

In some embodiments, an antibody or antigen binding fragment thereof provided herein comprises CDRs in which the specific amino acids that are identified herein as the paratope are present and one or more amino acids at other positions (e.g., at up to 1, 2, 3, 4, 5, 6, 7, 8, 9, 10, 11 or 12 positions) are mutated relative to the CDRs of the antibody for which the applicable paratope was identified. In some embodiments, the V_{H} and/or V_{L} sequences of such an antibody or antigen binding fragment thereof have at least 80%, 85%, 90%, 95%, 97%, 98%, or 99% identity to the respective V_{H} and V_{L} sequences of the parent antibody (e.g., P20) for which the applicable paratope is provided herein.

Likewise, unless otherwise specified, a FR or individual specified FR(s) (e.g., FR-H1, FR-H2, FR-H3, FR-H4, FR-L1, FR-L2. FR-L3, and/or FR-L4), of a given antibody or region thereof, such as a variable region thereof, should be understood to encompass a (or the specific) framework region as defined by any of the known schemes. In some instances, the scheme for identification of a particular CDR. FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given V_{H} region amino acid sequence and a FR-L1, a FR-L2, a FR-L3, and a FR-L4 as contained within a given V_{L} region amino acid sequence, the FRs can be defined by any of the aforementioned schemes, such as Kabat, Chothia, AbM, IgBLAST, IMGT, or Contact method, or other known scheme.

In some embodiments, the anti-IL-18Rβ antibodies or antigen-binding fragments thereof are defined by their variable region or variable domain, which refers to the domain of an antibody heavy or light chain that is involved in binding the antibody to antigen. In general, variable regions of the heavy chain and light chain (V_{H} and V_{L}, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). In some embodiments, a single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

In some embodiments, the anti-IL-18Rβ antibody or antigen-binding fragment thereof is a human antibody or antigen-binding fragment thereof. In some embodiments, the human antibody or antigen-binding fragment thereof comprises an amino acid sequence corresponding to that of an antibody produced by a human or a human cell, or a non-human source, e.g., mouse, that utilizes human antibody repertoires or other human antibody-encoding sequences, including human antibody libraries. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

In some embodiments, the anti-IL-18Rβ antibody or antigen-binding fragment thereof is a chimeric antibody or antigen-binding fragment thereof. In some embodiments, the chimeric antibody is created by fusing the variable regions of an antibody from one species (e.g., human) to the constant domain of an antibody from another species (e.g., non-human). In some embodiments, the constant domain of the chimeric antibody is from a different Ig class from an IL-18Rβ antibody described herein, including IgA (including subclasses IgA1 and IgA2), IgD, IgE, IgG (including subclasses IgG1, IgG2, IgG3, and IgG4), and IgM. In some embodiments, the constant domain of the chimeric antibody comprises CH2 and CH3 domains from one or more of the different Ig classes.

Among the provided anti-IL-18Rβ antibodies or antigen-binding fragments thereof are antibody fragments. Typically, an antigen binding fragment comprises all CDRs of a variable heavy chain (V_{H}) and variable light chain (V_{L}) sequence from antibodies and fragments thereof that bind IL-18Rβ set forth herein. However, in some embodiments, an antigen-binding fragment comprises less than all six CDRs, such as CDR-H1, CDR-H2, CDR-H3, and CDR-L3, from antibodies and fragments thereof that bind IL-18Rβ set forth herein. In some embodiments, the anti-IL-18Rβ antibody or antigen-binding fragment thereof is an antigen-binding fragment selected from the group consisting of Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multi-specific antibodies formed from antibody fragments. In some embodiments, the antigen-binding fragment is a Fab fragment. In particular embodiments, the antibodies are single-chain antibody fragments comprising a heavy chain variable (V_{H}) region and/or a light chain variable (V_{L}) region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the antigen-binding fragment is a F(ab) comprising a covalent heterodimer of the VH chain and VL chain.

In some embodiments, the antigen-binding fragment is an F(ab)₂ comprising two F(ab) fragments.

In some embodiments, the antigen-binding fragment is an Fv fragment. In some embodiments, the Fv fragment comprises a non-covalent VH::VL heterodimer. The VH:VL heterodimer comprises an antigen-binding site which retains much of the antigen recognition and binding capabilities of the native antibody molecule, but lacking the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, the antibody or antigen-binding fragment thereof is a single chain Fv (scFv) antibody. In some embodiments, the scFv comprising the VH region and VL region of any of the embodiments disclosed herein. In some embodiments, the scFv comprises one or more linkers joining two antibody domains or regions, such as a VH region and a VL region. The linker typically is a peptide linker, e.g., a flexible and/or soluble peptide linker. In some embodiments, the linker is rich in glycine and serine and/or, in some cases, threonine. In some embodiments, the linker further comprises charged residues such as lysine and/or glutamate, which can improve solubility. In some embodiments, the linker further comprises one or more proline.

ScFv antibodies may be prepared using standard molecular biology techniques following the teachings of the present application. An scFv polypeptide is a covalently linked VH::VL heterodimer which is expressed from a gene fusion including VH- and VL-encoding genes linked by a peptide-encoding linker. Huston et al. (1988) Proc. Nat. Acad. Sci. USA 85 (16): 5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, the antibodies described herein may be provided in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies or antigen-binding fragments thereof of the present disclosure may be “non-naturally occurring” antibodies. Non-naturally occurring antibodies can refer to antibodies that comprise one or more amino acid modifications, such that the resultant antibody is substantially non-naturally occurring (e.g., does not exists in nature). These amino acid modifications can include point mutations, wherein a naturally occurring amino acid is substituted for another naturally occurring amino acid. In some embodiments, the amino acid modifications can include point mutations wherein a non-naturally occurring amino acid is substituted for a naturally occurring amino acid. Non-naturally occurring antibodies can also refer to antibodies that are conjugated to a heterologous protein or compound, such as a detectable marker.

Among the provided antibodies or antigen-binding fragments thereof are monoclonal antibodies, including monoclonal antibody fragments. A monoclonal antibody may be made by a variety of techniques, including but not limited to generation from a hybridoma, recombinant DNA methods, phage-display and other antibody display methods.

In some aspects, the antibody or the antigen-binding fragments of the antibody is isolated.

The present disclosure contemplates variants of any of the antibodies disclosed herein, including any variants as disclosed herein that comprise any modifications as described herein.

Tables E12A and E16A provide the SEQ ID NOs: of exemplary provided anti-IL-18Rβ antibodies or antigen-binding fragments thereof. In some embodiments, the anti-IL-18Rβ antibody or antigen-binding fragment thereof comprises a V_{H} region that comprises the CDR-H1, the CDR-H2, and the CDR-H3 sequence, and a V_{L} region that comprises the CDR-L3 sequence, as set forth in the SEQ ID NOs: listed in any row of Table E12A or E16A (by any of the listed numbering schemes, such as Kabat). In some embodiments, the anti-IL-18Rβ antibody or antigen-binding fragment thereof comprises a V_{H} region that comprises the CDR-H1, the CDR-H2, and the CDR-H3 sequence, and a V_{L} region that comprises the CDR-L1, the CDR-L2, and the CDR-L3 sequence, as set forth in the SEQ ID NOs: listed in any row of Table E12A or E16A (by any of the listed numbering schemes, such as, e.g., Kabat). In some embodiments, the anti-IL-18Rβ antibody or antigen-binding fragment thereof comprises a Vu region sequence and a V_{L} region sequence set forth in the SEQ ID NOs: listed in any row of Table E12A or E16A. In some embodiments, the anti-IL-18Rβ antibody or antigen-binding fragment thereof comprises a V_{H} region amino acid sequence and a V_{L} region amino acid sequence that has at least 80%, 85%, 90%, 91%, 92%, 93%, 94%, 95%, 96%, 97%, 98%, or 99% sequence identity to the respective V_{H} region sequence and V_{L} region sequence set forth in the SEQ ID NOs: listed in any row of Table E12A or E16A. In some embodiments, the anti-IL-18Rβ antibody or antigen-binding fragment thereof comprises a V_{H} region sequence and a V_{L} region sequence set forth in the SEQ ID NOs: listed in any row of Table E12A or E16A.

### B. Exemplary Features

In some aspects, the provided anti-IL-18Rβ antibodies or antigen-binding fragments thereof have one or more specified structural and/or functional features that provide advantageous technical effects, such as high affinity binding to human and cynomolgus monkey IL-18Rβ; potency, for instance, in inhibition of IFNg production; pH-independent binding; low polyreactivity; stability; blocking of IL-18Rβ interaction with the IL-18/IL-18Rα complex; a low immunogenicity risk; and being human.

In some embodiments, the provided antibodies or antigen-binding fragment thereof specifically bind to an IL-18Rβ protein. In some embodiments, the provided antibodies and antigen-binding fragments thereof specifically bind to human IL-18Rβ. In some embodiments, the provided antibodies and antigen-binding fragments thereof specifically bind to human IL-18Rβ and cynomolgus monkey IL-18Rβ. In some embodiments, the human IL-18Rβ comprises, consists essentially of, or consists of the amino acid sequence of SEQ ID NO: 126. Methods to determine such specific or preferential binding are also well known in the art, e.g., an immunoassay. Antibodies or antigen-binding fragments thereof that exhibit cross-reactive binding to both human IL-18Rβ and cynomolgus monkey IL-18Rβ are advantageous because it allows for testing the same antibody or antigen-binding fragment thereof in preclinical animal studies (using cynomolgus monkeys) that will later be used in humans, such as in the clinic for human studies. In other words, a surrogate antibody or antigen-binding fragment thereof would not be necessary when the antibody or antigen-binding fragment thereof of interest for use in humans binds to both human IL-18Rβ and cynomolgus monkey IL-18Rβ.

In some embodiments, the provided antibodies or antigen-binding fragment thereof exhibits allosteric blocking of IL-18Rβ interacting with the IL-18/IL-18Rα complex. Without being bound to a theory, in some embodiments, the anti-IL-18Rβ antibody or antigen-binding fragment thereof, such as those that comprise a CDR-H1, CDR-H2, and CDR-3 comprising the amino acid sequences of SEQ ID NOs: 8, 9, and 10, respectively, and a CDR-L3 comprising the amino acid sequence of SEQ ID NO: 24; or a CDR-H1, CDR-H2, and CDR-3 comprising SEQ ID NOs: 8, 9, and 10, and a CDR-L1, CDR-L2, and CDR-L3 comprising the amino acid sequences of SEQ ID NOs: 22, 23, and 24, respectively, exhibit allosteric blocking of IL-18Rβ interacting with the IL-18/IL-18Rα complex.

In some embodiments, the anti-IL-18Rβ antibody or antigen-binding fragment thereof, such as those that comprise a CDR-H1, CDR-H2, and CDR-3 comprising the amino acid sequences of SEQ ID NOs: 8, 9, and 10, respectively, and a CDR-L3 comprising the amino acid sequence of SEQ ID NO: 24; or a CDR-H1, CDR-H2, and CDR-3 comprising SEQ ID NOs: 8, 9, and 10, and a CDR-L1, CDR-L2, and CDR-L3 comprising the amino acid sequences of SEQ ID NOs: 22, 23, and 24, respectively, binds to an epitope of human IL-18Rβ that comprises, consists essentially of, or consists of residues 242-248, 279, 281, 282, 312, 342, and 343 of human IL-18Rβ, with residue numbering corresponding to the amino acid sequence of SEQ ID NO: 126. In some embodiments, the anti-IL-18Rβ antibody or antigen-binding fragment thereof, such as those that comprise a CDR-H1, CDR-H2, and CDR-3 comprising the amino acid sequences of SEQ ID NOs: 8, 9, and 10, respectively, and a CDR-L3 comprising the amino acid sequence of SEQ ID NO: 24; or a CDR-H1, CDR-H2, and CDR-3 comprising SEQ ID NOs: 8, 9, and 10, and a CDR-L1, CDR-L2, and CDR-L3 comprising the amino acid sequences of SEQ ID NOs: 22, 23, and 24, respectively, binds to an epitope of human IL-18Rβ that comprises, consists essentially of, or consists of residues 242-248, Phe 279, Arg 281, Val 282, Leu 312, Ser 342, and Ile 343 of human IL-18Rβ, wherein numbering of the residues corresponds to the numbering in SEQ ID NO:126. In some embodiments, the human IL-18Rβ comprises, consists essentially of, or consists of the amino acid sequence of SEQ ID NO: 126 or an isoform thereof. Isoforms of the human IL-18Rβ sequence include, for example, UniProt sequence 095256-1 (SEQ ID NO: 126), as well as other isoforms such as, e.g., UniProt sequences 095256-2, 095256-3, and 095256-4. In some embodiments, the human IL-18Rβ comprises, consists essentially of, or consists of the mature extracellular domain of a human IL-18Rβ or a domain of human IL-18 Rβ corresponding to amino acid residues 20 to 356 of SEQ ID NO: 126. In some embodiments, the human IL-18Rβ comprises, consists essentially of, or consists of the amino acid sequence of SEQ ID NO: 126.

In some embodiments, the anti-IL-18Rβ antibody or antigen-binding fragment thereof comprises a paratope disclosed herein. In some embodiments, the paratope of the anti-IL-18Rβ antibody or antigen-binding fragment thereof includes heavy chain CDR1 residues Asn 31, Tyr 32, and Tyr 33; heavy chain CDR2 residues Tyr 50, Phe 52, Tyr 53, Ser 54, Thr 56, and Asn 58; heavy chain CDR3 residues Asp 98, Val 99, Gly 100, Ile 101, Ala 102, Ala 103, Asn 105, Tyr 107, and Tyr 109; and light chain CDR3 residues Phe 91, Tyr 94, and Ile 96. In some such embodiments, the anti-IL-18Rβ antibody or antigen-binding fragment thereof comprises a CDR-H1, CDR-H2, and CDR-3 comprising the amino acid sequences of SEQ ID NOs: 8, 9, and 10, respectively, and a CDR-L3 comprising the amino acid sequence of SEQ ID NO: 24; or a CDR-H1, CDR-H2, and CDR-3 comprising SEQ ID NOs: 8, 9, and 10, and a CDR-L1, CDR-L2, and CDR-L3 comprising the amino acid sequences of SEQ ID NOs: 22, 23, and 24, respectively.

In some embodiments, the provided antibodies or antigen-binding fragment thereof exhibit potency, such as potency in inhibition of IFNg production. For instance, in some embodiments, the anti-IL-18Rβ antibody or antigen-binding fragment thereof exhibits inhibition of IFNg production. The production of IFNg following activation of the IL-18 receptor complex mediates inflammatory effects that are associated with a variety of diseases and disorders. As such, potent inhibition of IFNg production is an advantageous technical effect for an anti-IL-18Rβ antibody or antigen-binding fragment thereof to have.

Assessing inhibition of IFNg production can be performed by various assays, such by stimulating human whole blood with IL-18 and/or LPS and IL-12 in the presence of the anti-IL-18Rβ antibody or antigen-binding fragment thereof or an isotype control antibody. In some embodiments, inhibition of IFNg production is assessed based on stimulating whole human blood with LPS and IL-12 in the presence of the anti-IL-18Rβ antibody or antigen-binding fragment thereof. In some embodiments, inhibition of IFNg production is assessed based on stimulating whole human blood with IL-18 and IL-12 in the presence of the anti-IL-18Rβ antibody or antigen-binding fragment thereof.

In some embodiments, the anti-IL-18Rβ antibody or antigen-binding fragment thereof exhibits at least 85%, 86%, 87%, 88%, 89%, 90%, 91%, 92%, 93%, 94%, 95%, 96%, 97%, 98%, or 99% inhibition of IFNg production, e.g., in human whole blood cells or in a cell line. In some embodiments, the anti-IL-18Rβ antibody or antigen-binding fragment thereof inhibits IFNg production with an IC₅₀ at or below 5 nM, 4 nM, 3 nM, or 2 nM. In some embodiments, the anti-IL-18Rβ antibody or antigen-binding fragment thereof inhibits IFNg production with an IC₅₀ below 3 nM. In some embodiments, the anti-IL-18Rβ antibody or antigen-binding fragment thereof inhibits IFNg production with an IC₅₀ from about 0.25 nM to about 2.5 nM, or from about 0.5 nM to about 2.5 nM, or from about 1 nM to about 2 nM, optionally wherein inhibition of IFNg production is assessed based on stimulating whole human blood with LPS and IL-12 in the presence of the anti-IL-18Rβ antibody or antigen-binding fragment thereof. In some embodiments, the anti-IL-18Rβ antibody or antigen-binding fragment thereof inhibits IFNg production with an IC₅₀ from about 0.25 nM to about 5 nM, from about 1 nM to about 5 nM, from about 1 nM to about 4 nM, from about 1 nM to about 3 nM, or from about 2 nM to about 3 nM, optionally wherein inhibition of IFNg production is assessed based on stimulating whole human blood with IL-18 and IL-12 in the presence of the anti-IL-18Rβ antibody or antigen-binding fragment thereof.

In some embodiments, the anti-IL-18Rβ antibody or antigen-binding fragment thereof exhibits sustained inhibition of IFN production of at least 90% for at least one week, two weeks, or three weeks in an ex vivo assay of cynomolgus monkey whole blood assay.

In some embodiments, the provided anti-IL-18Rβ antibodies or antigen-binding fragments thereof exhibit pH-independent binding. This can be advantageous by allowing the anti-IL-18Rβ antibody or antigen-binding fragment thereof to specifically bind to IL-18Rβ at both physiological pH, and in more acidic environments, such as the gastrointestinal tract or tumor microenvironments. For instance, normal tissues and cells typically exhibit a physiological pH that is a more neutral pH 7.35 to 7.45, such as pH about 7.4, whereas the pH of a tumor microenvironment or the gastrointestinal tract is typically more acidic, sometimes between about pH 5.6 and 6.8, such as about pH 6.0. The ability to bind IL-18Rβ at both physiological pH and a more acidic pH would, thus, be advantageous for treating a variety of diseases and conditions, including, e.g., inflammatory bowel disease, e.g., Crohn's disease or ulcerative colitis, and cancers.

In some embodiments, the antibody or antigen-binding fragment thereof has a KD ratio of KD at pH 5 to KD at pH 7 of 0.33 to 3.33, or about 0.33 to about 3.33. In some embodiments, the antibody or antigen-binding fragment thereof has a KD ratio of KD at pH 5 to KD at pH 7 of about 0.33 to about 3.33, about 0.33 to about 3.00, about 0.33 to about 2.5, about 0.33 to about 2, about 0.33 to about 1.75, about 0.33 to about 1.5, about 0.33 to about 1.3, about 0.5 to about 3.33, about 0.5 to about 3.00, about 0.5 to about 2.5, about 0.5 to about 2, about 0.5 to about 1.75, about 0.5 to about 1.5, about 0.5 to about 1.3, about 0.8 to about 3.33, about 0.8 to about 3.00, about 0.8 to about 2.5, about 0.8 to about 2, about 0.8 to about 1.75, about 0.8 to about 1.5, or about 0.8 to about 1.3. In some embodiments, the anti-IL-18Rβ antibody or antigen-binding fragment thereof has a KD ratio of KD at pH 5 to KD at pH 7 of 0.8 to 1.3, or about 0.8 to about 1.3. In some embodiments, the anti-IL-18Rβ antibody or antigen-binding fragment thereof has a KD ratio of KD at pH 5 to KD at pH 7 of about 0.9 to about 1.2. In some embodiments, the anti-IL-18Rβ antibody or antigen-binding fragment thereof has a KD ratio of KD at pH 5 to KD at pH 7 of about 1.0 to about 1.2.

In some embodiments, the antibody or antigen-binding fragment thereof has a KD ratio of KD at pH 5 to KD at pH 7 of 0.2 to 1.00, or about 0.2 to about 1.00. In some embodiments, the antibody or antigen-binding fragment thereof has a KD ratio of KD at pH 5 to KD at pH 7 of about 0.2 to about 1.00, about 0.2 to about 0.8, about 0.2 to about 0.6, about 0.2 to about 0.5, about 0.25 to about 1.00, about 0.25 to about 0.8, about 0.25 to about 0.6, or about or 0.25 to about 0.5. In some embodiments, the antibody or antigen-binding fragment thereof has a KD ratio of KD at pH 5 to KD at pH 7 of about 0.25 to about 0.5. In some embodiments, the antibody or antigen-binding fragment thereof has a KD ratio of KD at pH 5 to KD at pH 7 of about 0.3 to about 0.4.

In some embodiments, the provided anti-IL-18Rβ antibodies or antigen-binding fragments thereof low polyreactivity. As used herein, an antibody or antigen-binding fragment thereof that exhibits “low polyreactivity” exhibits low non-specific binding to extracellular matrix proteins. Accordingly, in some embodiments, the anti-IL-18Rβ antibody or antigen-binding fragment thereof exhibits low non-specific binding to extracellular matrix proteins, such as by having an extracellular matrix (ECM) score of less than 6. Low polyreactivity is advantageous because it can, e.g., reduce off-target effects caused by the antibody binding to extracellular matrix proteins, for instance; and it can also reduce or avoid the negative effects that polyreactivity can have on pharmacokinetics and bioavailability of the therapeutic antibody. Polyreactivity can be assessed by, for instance, reference to an ECM score that is calculated based on an ECM ELISA assay, where the ECM score is calculated by dividing the absorbance value of an ECM-coated sample well containing the antibody or antigen-binding fragment thereof by the absorbance of an ECM-coated sample well that does not contain the antibody or antigen-binding fragment thereof. In some embodiments, an ECM score of 6 at a concentration of 1 M antibody or antigen-binding fragment thereof is used to determine whether an antibody or antigen-binding fragment thereof has a high or low ECM score, with a high ECM score being at or higher than 6, and a low ECM score being less than 6. In some embodiments, the anti-IL-18Rβ antibody or antigen-binding fragment thereof has an ECM score of less than 6, less than 5, less than 4, or less than 3.5. In some embodiments, the anti-IL-18Rβ antibody or antigen-binding fragment thereof has an ECM score of less than 4.

In some embodiments, the provided anti-IL-18Rβ antibodies or antigen-binding fragments thereof are capable of binding IL-18Rβ, such as human IL-18Rβ, with at least a certain affinity, as measured by any of a number of known methods. In some embodiments, the affinity is represented by an equilibrium dissociation constant (K_{D}).

A variety of assays are known for assessing binding affinity, equilibrium dissociation constant (K_{D}), equilibrium association constant (K_{A}), EC50, on-rate (association rate constant; kₒₙ or kₐ; units of 1/Ms or M⁻¹s⁻¹) and the off-rate (dissociation rate constant; k_{off} or k_{d}; units of 1/s or s⁻¹) and/or determining whether a binding molecule (e.g., an antibody or fragment thereof) specifically binds to a particular ligand (e.g., an antigen, such as an IL-18Rβ protein). One can determine the binding affinity of a binding molecule, e.g., an anti-IL-18Rβ antibodies or antigen-binding fragments thereof, by using any of a number of binding assays that are well known. For example, in some embodiments, a BIAcore® instrument can be used to determine the binding kinetics and constants of a complex between two proteins (e.g., an anti-IL-18Rβ antibody or antigen-binding fragment thereof, and an antigen, such as an IL-18Rβ protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation rate constant (K_{off} or k_{d}), the association rate constant (kₒₙ or kₐ) and/or equilibrium dissociation constant (K_{D}) and/or equilibrium association constant (K_{A}) for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing, genetic reporter assays, flow cytometry, and other methods for detection of expressed nucleic acids or binding of proteins.

In some binding can be determined under conditions that mimic those that exist in natural environments in the body of a subject. For instance, the pH of a tumor microenvironment or the gastrointestinal tract is typically more acidic, sometimes between about pH 5.6 and 6.8, such as about pH 6.0. In contrast, physiological pH is a more neutral pH 7.35 to 7.45, such as pH about 7.4 and more closely mimics the environment of normal tissues or cells in the blood.

In some embodiments, the antibody or antigen-binding fragment thereof binds to human IL-18Rβ at pH 7.4 with an equilibrium dissociation constant (K_{D}) that is about or less than about 10 nM, about or less than about 5 nM, about or less than about 4 nM, about or less than about 3 nM, about or less than about 2 nM, about or less than about 1.5 nM, about or less than about 1.4 nM, about or less than about 1.3 nM, about or less than about 1.2 nM, about or less than about 1.1 nM, or about or less than about 1.0 nM. In some embodiments, the antibody or antigen-binding fragment thereof binds to human IL-18Rβ at pH 7.4 with a K_{D} that is, is about, is less than, or is less than about 1.3 nM. In some embodiments, the antibody or antigen-binding fragment thereof binds to human IL-18Rβ at pH 7.4 with a K_{D} that is or is about 0.8 nM, 0.9 nM, 1.0 nM. 1.1 nM. 1.2 nM, or 1.3 nM. In some embodiments, the antibody or antigen-binding fragment thereof binds to human IL-18Rβ at pH 7.4 with a K_{D} that is or is about 1 nM.

In some embodiments, the antibody or antigen-binding fragment thereof binds to cynomolgus IL-18Rβ at pH 7.4 with an equilibrium dissociation constant (K_{D}) that is about or less than about 10 nM, about or less than about 5 nM, about or less than about 4 nM, about or less than about 3 nM, about or less than about 2 nM, or about or less than about 1.5 nM. In some embodiments, the antibody or antigen-binding fragment thereof binds to cynomolgus IL-18Rβ at pH 7.4 with a K_{D} that is, is about, less than, or less than about 2 nM. In some embodiments, the antibody or antigen-binding fragment thereof binds to cynomolgus IL-18Rβ at pH 7.4 with a K_{D} that is or is about 1 nM, 1.1 nM, 1.2 nM, 1.3 nM, 1.4 nM, 1.5 nM. 1.6 nM, or 1.7 nM. In some embodiments, the antibody or antigen-binding fragment thereof binds to cynomolgus IL-18Rβ at pH 7.4 with a K_{D} that is or is about 1.3 nM.

In some embodiments, the antibody or antigen-binding fragment thereof binds to human IL-18Rβ at pH 7.4 with a K_{D} that is, is about, less than, or less than about 1.3 nM, and binds to cynomolgus IL-18Rβ at pH 7.4 with a K_{D} that is, is about, less than, or less than about 2 nM.

In some embodiments, the provided anti-IL-18Rβ antibodies or antigen-binding fragments thereof exhibit a low immunogenicity risk, e.g., a low immunogenicity risk when administered to human subjects. Immunogenicity risk can be assessed by, for instance, determining whether a candidate therapeutic, e.g., an antibody or antigen-binding fragment thereof, contains a functional T cell epitope(s) based on the candidate therapeutic's ability to stimulate antigen-specific CD4+ T cells in vitro. A low immunogenicity risk, e.g., in human subjects, for an antibody or antigen-binding fragment thereof is advantageous because it reduces the likelihood that the administered antibody or antigen-binding fragment thereof would trigger an unwanted immune response against the antibody or antigen-binding fragment thereof itself.

Typically, protein therapeutics that induce less than a 20% response in donors are considered to have low immunogenicity risk. In some embodiments, the antibody or antigen-binding fragment thereof exhibits a low immunogenicity risk that is characterized by less than a 20% response in donors.

In some embodiments, the provided anti-IL-18Rβ antibodies or antigen-binding fragments thereof are human antibodies. An antibody or antigen-binding fragment thereof that is human typically exhibits a lower immunogenicity risk than humanized antibodies, thereby contributing to a low immunogenicity risk.

In some embodiments, the provided anti-IL-18Rβ antibodies or antigen-binding fragments thereof exhibit low surface hydrophobicity. Surface hydrophobicity can be assessed, for instance, by hydrophobic interaction chromatography (HIC) to determine a HIC retention time. HIC is an assay that can be used to quantify the hydrophobicity of an antibody. Slower retention times in an HIC assay are associated with, e.g., a tendency for the antibody to precipitate. In some embodiments, the HIC retention time is less than 13 minutes, less than 12 minutes, or less than 11 minutes. In some embodiments, a HIC retention time of 11 min is used as a cutoff for determining whether an antibody or antigen-binding fragment thereof exhibits low surface hydrophobicity, since ˜75% of 48 clinically approved monoclonal antibodies were found in Example 2 to have an HIC retention time of lower than 11 minutes. Accordingly, in some embodiments, the provided anti-IL-18Rβ antibodies or antigen-binding fragments thereof exhibit low surface hydrophobicity, as determined by having a HIC retention time of less than 11 minutes.

In some embodiments, the provided anti-IL-18Rβ antibodies or antigen-binding fragments thereof exhibit a low self-interaction propensity. Self-interaction of a therapeutic antibody can result in undesirable effects, such as aggregation, low solubility, or high viscosity. As such, a therapeutic antibody or antigen-binding fragment thereof having a low self-interaction propensity is advantageous for the developability of the therapeutic antibody or antigen-binding fragment thereof. The self-interaction propensity of an antibody can, for instance, be measured using a clone self-interaction (CSI) using bio-layer interferometry (BLI) assay (CSI-BLI assay), such as described in Sun et al. (2013) MAbs 2 (6): 838-41, or a modified version thereof. Lower CSI-BLI values are generally desirable because they indicate that the antibody has less propensity to self-interact. In some embodiments, the provided anti-IL-18Rβ antibodies or antigen-binding fragments thereof exhibit a low self-interaction propensity, as determined by having a CSI-BLI value of less than 0.25, less than 0.20, less than 0.15, less than 0.10, less than 0.5, or less than 0.3. In some embodiments, the CSI-BLI value is about −0.1 to about 0.1. In some embodiments, the CSI-BLI value is about −0.05 to about 0.05. In some embodiments, the CSI-BLI value is about −0.05 to about 0.05, about −0.04 to about 0.03, or about −0.03 to about 0.02. In some embodiments, the CSI-BLI value is or is about −0.03, is or is about −0.02, is or is about −0.01, is or is about 0.00, is or is about 0.01, or is or is about 0.02. The self-interaction propensity of an antibody can, alternatively, be measured using a self-interaction chromatography (SIC) or cross-interaction chromatography (CIC) assay, which measure the retention time of an antibody, such as described in Sun et al., supra.

In some embodiments, the provided anti-IL-18Rβ antibodies or antigen-binding fragments thereof exhibit stability, such as stability based on measurements of monomericity, charge heterogeneity, chemical stability, and/or binding to IL-18Rβ, e.g., human IL-18Rβ. In some embodiments, the provided anti-IL-18Rβ antibodies or antigen-binding fragments thereof exhibit stability based on measurements of monomericity, charge heterogeneity, chemical stability, and binding to IL-18Rβ. e.g., human IL-18Rβ. Stability can be assessed, for instance, by measurements of monomericity, charge heterogeneity, chemical stability, and binding to IL-18Rβ, e.g., human IL-18Rβ. In some embodiments, such measurements are taken following one or more freeze-thaw cycles; and/or at different temperatures, pH, and time points.

In some embodiments, the provided anti-IL-18Rβ antibodies or antigen-binding fragments thereof exhibit stability based on retention of a monomeric peak. Retention of a monomeric peak can be assessed, e.g., by a high performance liquid chromatography (HPLC) instrument. In some embodiments, stability is based on a change in monomeric peak exhibiting less than a threshold percentage over a period of four weeks following one or more, e.g., one, two, three, four, or five, freeze-thaw cycles. In some embodiments, stability is assessed following five freeze-thaw cycles. In some embodiments, the provided anti-IL-18Rβ antibodies or antigen-binding fragments thereof exhibit less than a 10% loss, less than a 9% loss, less than a 8% loss, less than a 7% loss, less than a 6% loss, or less than a 5% loss, of monomeric peak over the course of four weeks following five freeze-thaw cycles. In some embodiments, the provided anti-IL-18Rβ antibodies or antigen-binding fragments thereof exhibit less than a 5% loss of monomeric peak over the course of four weeks following five freeze-thaw cycles.

In some embodiments, the provided anti-IL-18Rβ antibodies or antigen-binding fragments thereof exhibit stability based on a change in charge heterogeneity of the main peak exhibiting less than a threshold percentage after 4 weeks of storage, e.g., at 4° C. A change in charge heterogeneity for a therapeutic antibody can be caused by chemical degradation over time, such as when in storage. A large change in charge heterogeneity can negatively impact a therapeutic antibody in a number of ways, including stability, pharmacokinetics, potency, and binding affinity. In some embodiments, the provided anti-IL-18Rβ antibodies or antigen-binding fragments thereof exhibit stability based on a change in charge heterogeneity of the main peak exhibiting less a 10% change, less than a 5% change, less than a 4% change, or less than 3% change in charge heterogeneity after 4 weeks of storage at 4° C. In some embodiments, the provided anti-IL-18Rβ antibodies or antigen-binding fragments thereof exhibit stability based on a change in charge heterogeneity of the main peak exhibiting less a less than a 5% change in charge heterogeneity after 4 weeks of storage at 4° C. In some embodiments, the provided anti-IL-18Rβ antibodies or antigen-binding fragments thereof exhibit stability based on a change in charge heterogeneity of the main peak exhibiting less a less than a 3% change in charge heterogeneity after 4 weeks of storage at 4° C.

In some embodiments, the provided anti-IL-18Rβ antibodies or antigen-binding fragments thereof exhibit stability based on chemical stability. The presence of a chemical liability in a therapeutic antibody can result in negative consequences, such as increased degradation, and can make certain formulations, e.g., liquid formulation, challenging. Chemical stability can be assessed, e.g., by monitoring the oxidation, deamidation, and isomerization levels over time, e.g., over the course of 1 month of storage at 4° C., 25° C., or 40° C. In some embodiments, the anti-IL-18Rβ antibody or antigen-binding fragment thereof exhibit does not exhibit a chemical liability based on oxidation, deamidation, and isomerization monitoring over the course of 1 month of storage at 4° C., 25° C., or 40° C.

In some embodiments, the provided anti-IL-18Rβ antibodies or antigen-binding fragments thereof include anti-IL-18Rβ antibodies or antigen-binding fragments thereof that comprise a substitution of a D50 amino acid residue of the light chain of a parental antibody, e.g., to eliminate a chemical liability. In some embodiments, the D50 amino acid residue of the light chain is contained within the CDR-2 of the light chain. In some embodiments, the D50 amino acid residue of the light chain of the parental antibody exhibits a chemical liability that is an aspartate isomerization liability. In some embodiments, the substitution is a D50E substitution in the light chain. In some embodiments, the provided anti-IL-18Rβ antibodies or antigen-binding fragments thereof include anti-IL-18Rβ antibodies or antigen-binding fragments thereof that comprise a glutamic acid (E) residue at amino acid position 50 of the light chain. In some embodiments, comprising a glutamic acid (E) residue at amino acid position 50 of the light chain eliminates an aspartate isomerization liability of the parental antibody, thereby improving stability.

In some embodiments, the anti-IL-18Rβ antibody or antigen-binding fragment thereof provided herein comprises a glutamic acid (E) residue at amino acid position 50 of the light chain, and advantageously exhibits comparable affinity and potency of a parent antibody that comprises an aspartic acid (D) at amino acid position 50 of the light chain, while maintaining low immunogenicity risk and eliminating the aspartate isomerization liability of the parental antibody.

### C. Multi-Specific Antibodies

Provided herein, in some embodiments, are multi-specific antibodies. In some embodiments, the multi-specific antibodies comprises a first antigen-binding domain that binds to IL-18Rβ and a second antigen-binding domain that binds to a second antigen, wherein the first antigen-binding domain comprises any anti-IL-18Rβ antibody or antigen-binding fragment thereof described herein, such as any of those disclosed in Section I.A.

In some embodiments, the multi-specific antibody further comprises one or more additional antigen-binding domains that each bind to a different additional antigen.

In some embodiments, the multi-specific antibody is a bispecific antibody. In some embodiments, the bispecific antibody comprises a first antigen-binding domain that binds to IL-18Rβ and a second antigen-binding domain that binds to a second antigen, wherein the first antigen-binding domain comprises any anti-IL-18Rβ antibody or antigen-binding fragment thereof described herein.

Typically, the second antigen-binding domain and/or the one or more additional domains bind to an antigen or protein other than IL-18Rβ, such as any antigen or protein other than IL-18Rβ that is associated with the IL-18 pathway and/or is associated any disease or disorder as described herein. However, in some embodiments, the second antigen-binding domain and/or the one or more additional domains bind to a different epitope of IL-18Rβ and/or comprises a different paratope. In some embodiments, the second antigen-binding domain and/or the one or more additional domains bind to an antigen that is associated with inflammatory bowel disease.

In some embodiments, a provided binding molecule is a bispecific T cell engager that is composed of an anti-IL-18Rβ antibody or antigen-binding fragment as described herein and at least one additional binding molecule capable of binding to a surface molecule expressed on a T cell. In some embodiments, the surface molecule is an activating component of a T cell, such as a component of the T cell receptor complex. In particular aspects, the surface molecule is an activating T cell antigen that is expressed on a T cell and is capable of inducing T cell activation upon interaction with an antigen binding molecule. For example, in some aspects, interaction of an antigen binding molecule with an activating T cell antigen may induce T cell activation by triggering the signaling cascade of the T cell receptor complex. Suitable assays to measure T cell activation are known, and include any assay to measure or assess proliferation, differentiation, cytokine secretion, cytotoxic activity and/or expression of one or more activation marker. In some embodiments, the simultaneous or near simultaneous binding of such a multi-specific antibody to both of its targets, e.g., IL-18Rβ expressed on a target cell and a T cell molecule expressed on a T cell, e.g. activating T cell antigen, can result in a temporary interaction between the target cell and T cell, thereby resulting in activation, e.g. cytotoxic activity, of the T cell and subsequent lysis of the target cell, such as when used for the treatment of cancer.

Among bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). Similar formats of any of the above molecules can be generated using any of the anti-PD-1 antibodies or antigen binding fragments provided herein.

In some embodiments, the second or additional binding domain is an antigen-binding fragment selected from a Fab fragment, a F(ab′)₂ fragment, an Fv fragment, a scFv, disulfide stabilized Fv fragment (dsFv), a scAb, a dAb, a single domain heavy chain antibody (VHH), or a single domain light chain antibody.

### D. Variants and Modifications

Amino acid sequence modification(s) of the anti-IL-18Rβ antibodies and antigen-binding fragments thereof described herein are contemplated. For example, it may be desirable to improve the binding affinity and/or other biological properties of the antibody or antigen-binding fragment thereof. For example, amino acid sequence variants of an antibody may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final antibody, provided that the final construct possesses the desired characteristics (e.g., high affinity binding to IL-18Rβ, reduced immunogenicity, increased stability, etc.). The amino acid changes may alter post-translational processes of the antibody, such as changing the number or position of glycosylation sites. Any of the variations and modifications described above for polypeptides of the present invention may be included in antibodies of the present invention.

In certain embodiments, the antibodies or antigen-binding fragments thereof include one or more amino acid variations, e.g., substitutions, deletions, insertions, and/or mutations, compared to the sequence of an antibody or antigen-binding fragment thereof described herein. Exemplary variants include those designed to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from, and/or insertions into and/or substitutions of residues within the amino acid sequences of the antibody or antigen-binding fragment thereof. Any combination of deletion, insertion, and substitution can be made to arrive at the final construct, provided that the final construct possesses the desired characteristics, e.g., antigen-binding.

In certain embodiments, the antibodies or antigen-binding fragments thereof include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC). Exemplary amino acid substitutions include those as shown, e.g., in Table 2, but also includes an amino acid that is substituted with an isosteric non-natural amino acid.

In some embodiments, one or more residues within a CDR of a parent antibody (e.g., a human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant V_{H} and V_{L} sequences provided above, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In some embodiments, the anti-IL-18Rβ antibody or antigen-binding fragment thereof is modified to remove one or more glycosylation sites and/or is modified by pegylation.

In certain embodiments, the antibody or antigen-binding fragment thereof is altered to increase or decrease the extent to which the antibody is glycosylated, for example, by removing or inserting one or more glycosylation sites by altering the amino acid sequence and/or by modifying the oligosaccharide(s) attached to the glycosylation sites, e.g., using certain cell lines.

In some embodiments, an N-linked glycosylation, which is a glycosylation that occurs at asparagines at a glycosylation site characterized by the consensus sequence-Asn-Xaa-Ser/Thr, is removed or inserted. In some embodiments, one or more asparagines are replaced with another amino acid to remove the glycosylation site.

In some embodiments, the anti-IL-18Rβ antibody or antigen-binding fragment thereof is an aglycosylated antibody or antigen-binding fragment thereof, which is an antibody or antigen-binding fragment thereof that lacks glycosylation. In some embodiments, the anti-IL-18Rβ antibody or antigen-binding fragment thereof comprises one or more modifications, e.g., amino acid substitutions, that eliminates glycosylation at that amino acid position. Modifying an antibody to alter glycosylation can, for instance, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can, in some embodiments, increase the affinity of the antibody for antigen. Such an approach is described in further detail in U.S. Pat. Nos. 5,714,350 and 6,350,861 by Co et al.

Glycosylation of the constant region on N297 can, for instance, be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation, such as in methods for antibody production as described herein. For example EP 1,176,195 by Hanai et al. describes a cell line with a functionally disrupted FUT8 gene, which encodes a fucosyl transferase, such that antibodies expressed in such a cell line exhibit hypofucosylation. WO 03/035835 describes a variant CHO cell line, Led 3 cells, with reduced ability to attach fucose to Asn (297)-linked carbohydrates, also resulting in hypofucosylation of antibodies expressed in that host cell (see also Shields, R. L. et al. (2002) J. Biol. Chem. 277:26733-26740). WO 99/54342 describes cell lines engineered to express glycoprotein-modifying glycosyl transferases (e.g., beta (1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

In some embodiments, the anti-IL-18Rβ antibodies or antigen-binding fragments thereof are modified by pegylation. Accordingly, in some embodiments, the anti-IL-18Rβ antibodies or antigen-binding fragments thereof is pegylated. An antibody can be pegylated to, for example, increase the biological (e.g., serum) half-life of the antibody. In some embodiments, the antibody or antigen-binding fragment thereof is pegylated by reacting it with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See, e.g., EP 0 154 316 by Nishimura et al. and EP 0 401 384 by Ishikawa et al.

Exemplary modifications, variants, and cell lines are described, e.g., in Patent Publication Nos. US 2003/0157108. US 2004/0093621, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng, 87:614 (2004). Ripka et al. Arch. Biochem. Biophys. 249:533-545 (1986); US Pat Appl No US 2003/0157108 A1, Presta, L; and WO 2004/056312 A1, Yamane-Ohnuki et al. Biotech. Bioeng, 87:614 (2004); Kanda, Y. et al., Biotechnol. Bioeng., 94 (4): 680-688 (2006); and WO2003/085107); WO 2003/011878 (Jean-Mairet et al.); U.S. Pat. No. 6,602,684 (Umana et al.); and US 2005/0123546 (Umana et al.); WO 1997/30087 (Patel et al.); WO 1998/58964 (Raju, S.); and WO 1999/22764 (Raju. S.).

Among the modified antibodies and antigen-binding fragments thereof are those having one or more amino acid modifications in the Fc region, such as those having a human Fe region sequence or other portion of a constant region (e.g., a human IgG1, IgG2, IgG3 or IgG4 Fc region) comprising an amino acid modification (e.g. a substitution) at one or more amino acid positions. In some embodiments, the anti-IL-18Rβ antibody or antigen-binding fragment thereof comprises one or more amino acid modifications in the heavy chain constant domain and/or comprises one or more amino acid modifications in the light chain constant domain.

Such modifications can be made, e.g., to improve half-life, alter binding to one or more types of Fc receptors, and/or alter (e.g., reduce) effector functions.

Also among the variants are cysteine engineered antibodies such as “thioMAbs” and other cysteine engineered variants, in which one or more residues of an antibody are substituted with cysteine residues, in order to generate reactive thiol groups at accessible sites, e.g., for use in conjugation of agents and linker-agents, to produce immunoconjugates. Cysteine engineered antibodies are described, e.g., in U.S. Pat. Nos. 7,855,275 and 7,521.541.

In some embodiments, the antibodies or antigen-binding fragments thereof are modified to contain additional nonproteinaceous moieties, including water soluble polymers. Exemplary polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Antibodies and antigen-binding fragments and variants thereof of the present disclosure may also be modified to include a detectable label, e.g., an epitope tag or label, e.g., for use in purification or diagnostic applications, such as described in Section I.E. These may be conjugated to the antibody or antigen-binding fragment thereof as a fusion protein or conjugate, e.g., using a linker or linking group. There are many linking groups known in the art for making antibody conjugates, including, for example, those disclosed in U.S. Pat. No. 5,208,020 or EP Patent 0 425 235 B1, and Chari et al., Cancer Research 52:127-131 (1992). Linking groups include disulfide groups, thioester groups, acid labile groups, photolabile groups, peptidase labile groups, or esterase labile groups, as disclosed in the above-identified patents. Examples of tags and/or labels can include, but are not limited to, FLAG tags, poly-histidine tags (e.g, 6×His), cMyc tags, glutathione-S-transferase tags, avidin, fluorescent labels, polymer particles, metal particles, haptens, enzyme labels, luminescent labels, electrochemiluminescent labels, bioluminescent labels, radioisotopes, or oligonucleotides.

In some embodiments, the anti-IL-18Rβ antibody or antigen-binding fragment thereof disclosed herein comprises one or more substitutions that lower, or may lower, the immunogenicity risk of the anti-IL-18Rβ antibody or antigen-binding fragment thereof to a human subject. In some embodiments, the anti-IL-18Rβ antibody or antigen-binding fragment thereof disclosed herein comprises a Q6E and/or R19K substitution in the heavy chain. In some embodiments, the anti-IL-18Rβ antibody or antigen-binding fragment thereof disclosed herein comprises a Q6E substitution in the heavy chain. In some embodiments, the anti-IL-18Rβ antibody or antigen-binding fragment thereof disclosed herein comprises a R19K substitution in the heavy chain. In some embodiments, the anti-IL-18Rβ antibody or antigen-binding fragment thereof disclosed herein comprises a R3Q and/or R39K and/or M75I substitution in the light chain. In some embodiments, the anti-IL-18Rβ antibody or antigen-binding fragment thereof disclosed herein comprises a R3Q substitution in the light chain. In some embodiments, the anti-IL-18Rβ antibody or antigen-binding fragment thereof disclosed herein comprises a R39K substitution in the light chain. In some embodiments, the anti-IL-18Rβ antibody or antigen-binding fragment thereof disclosed herein comprises a M75I substitution in the light chain. In some embodiments, the anti-IL-18Rβ antibody or antigen-binding fragment thereof disclosed herein comprises a Q6E and/or R19K substitution in the heavy chain; and a R3Q and/or R39K substitution in the light chain. In some embodiments, the anti-IL-18Rβ antibody or antigen-binding fragment thereof disclosed herein comprises a R19K substitution in the heavy chain; and a R3Q and M75I substitution in the light chain. In some embodiments, the anti-IL-18Rβ antibody or antigen-binding fragment thereof disclosed herein comprises a Q6E substitution in the heavy chain; and a R39K substitution in the light chain.

In some embodiments, the anti-IL-18Rβ antibody or antigen-binding fragment thereof disclosed herein comprises one or more substitutions that improve chemical stability. In some embodiments, the anti-IL-18Rβ antibody or antigen-binding fragment thereof comprises one or more of the following amino acid substitutions: D50E, D50G, and A51E in the light chain. In some embodiments, the anti-IL-18Rβ antibody or antigen-binding fragment thereof comprises a D50E substitution in the light chain. In some embodiments, the anti-IL-18Rβ antibody or antigen-binding fragment thereof comprises a D50E and A51G substitution in the light chain. In some embodiments, the anti-IL-18Rβ antibody or antigen-binding fragment thereof comprises a D50G substitution in the light chain.

In some embodiments, the anti-IL-18Rβ antibody or antigen-binding fragment thereof disclosed herein comprises a Q6E substitution in the heavy chain; and a D50E substitution in the light chain.

In some embodiments, the anti-IL-18Rβ antibody or antigen-binding fragment thereof disclosed herein comprises a Q6E substitution in the heavy chain; and a D50E and A51G substitution in the light chain.

In some embodiments, the anti-IL-18Rβ antibody or antigen-binding fragment thereof disclosed herein comprises a Q6E substitution in the heavy chain; and a D50G substitution in the light chain.

In some embodiments, the anti-IL-18Rβ antibody or antigen-binding fragment thereof disclosed herein comprises a R19K substitution in the heavy chain; and a R3Q and M75I substitution in the light chain.

In some embodiments, the anti-IL-18Rβ antibody or antigen-binding fragment thereof disclosed herein comprises a Q6E substitution in the heavy chain; and a R39K and D50E substitution in the light chain.

In some embodiments, the anti-IL-18Rβ antibody or antigen-binding fragment thereof disclosed herein comprises at least one post-translational modification of the amino acid sequence. In some embodiments, the modification is a modification of a N-terminal glutamate or glutamine to a pyroglutamate. In some embodiments, the anti-IL-18Rβ antibody or antigen-binding fragment thereof disclosed herein comprises a Q1X substitution, wherein X is pyroglutamate, in the V_{H} region or in the heavy chain.

### E. Conjugates

Also provided herein, in some embodiments, are conjugates. In some embodiments, provided herein is a conjugate comprising any anti-IL-18Rβ antibody or antigen-binding fragment thereof disclosed herein, or any multi-specific antibody disclosed herein, and a heterologous molecule or moiety. In some embodiments, the conjugate comprises any anti-IL-18Rβ antibody or antigen-binding fragment thereof disclosed herein and a heterologous molecule or moiety. In some embodiments, the conjugate comprises any multi-specific antibody disclosed herein and a heterologous molecule or moiety.

In some embodiments, the heterologous molecule or moiety can be any substance having biological or detectable activity, for example, therapeutic agents, detectable labels, binding agents, or prodrugs, which are metabolized to an active agent in vivo. In some embodiments, the heterologous molecule or moiety can be a targeting moiety, a small molecule drug (e.g., non-polypeptide drug of less than 500 Daltons molar mass), a toxin, a cytostatic agent, a cytotoxic agent, an immunosuppressive agent, a radioactive agent suitable for diagnostic purposes, a radioactive metal ion for therapeutic purposes, a prodrug-activating enzyme, an agent that increases biological half-life, or a diagnostic or detectable agent. In some embodiments, the heterologous molecule or moiety is a protein, peptide, or a nucleic acid molecule, which can be synthesized or produced by recombinant means. In some embodiments, the heterologous molecule or moiety is a drug moiety, which may be synthesized artificially or purified from a natural source.

In some embodiments, the heterologous molecule or moiety is a therapeutic agent. The therapeutic agent can be any therapeutic agent suitable for conjugation to the anti-IL-18Rβ antibody or antigen-binding fragment thereof for any of the uses as described herein, e.g., for the treatment of a disease or disorder, such as inflammatory bowel disease.

In some embodiments, the therapeutic agent is an anti-inflammatory agent. In some embodiments, the anti-inflammatory agent is an analgesic, a corticosteroid, an antirheumatic agent, an aminosalicylate, or a gastrointestinal agent. In some embodiments, the analgesic is indomethacin, naproxen, ibuprofen, choline magnesium trisalicylate, ketorolac tromethamine, or rofecoxib. In some embodiments, the corticosteroid is clobetasol, dexamethasone, dexamethasone phosphate, hydrocortisone, hydrocortisone sodium phosphate, or methylprednisolone acetate. In some embodiments, the antirheumatic agent is dexamethasone, cyclosporine, sulfasalazine, valdecoxib, or penicillamine. In some embodiments, the gastrointestinal agent is mesalamine, olsalazine sodium, balsalazide disodium. In some embodiments, the therapeutic agent is an aminosalicylate, such as mesalamine, olsalazine, or balsalazide. In some embodiments, the therapeutic agent is an immune system suppressor. In some embodiments, the immune system suppressor is azathioprine, methotrexate, mercaptopurine, tofacitinib, ozanimod, or upadacitinib.

In some embodiments, the heterologous molecule or moiety is a cytotoxic agent. In some embodiments, the cytotoxic agent is selected from the group consisting of chemotherapeutic agents or drugs, growth inhibitory agents, enzymes, toxins, and radioactive isotopes.

In some embodiments, the cytotoxic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP. MMAF, and MMAE. The synthesis and structure of exemplary auristatins are described in U.S. Pat. Nos. 6,884,869, 7,098,308, 7,256,257, 7,423,116, 7,498,298 and 7,745,394, each of which is incorporated by reference herein in its entirety and for all purposes.

Auristatins have been shown to interfere with microtubule dynamics and nuclear and cellular division and have anticancer activity. Auristatins of the present invention bind tubulin and can exert a cytotoxic or cytostatic effect on a 5T4 expressing cell or cell line. There are a number of different assays, known in the art, that can be used for determining whether an auristatin or resultant antibody-drug conjugate exerts a cytostatic or cytotoxic effect on a desired cell or cell line. Methods for determining whether a compound binds tubulin are known in the art. See, for example, Muller et al., Anal. Chem 2006, 78, 4390-4397; Hamel et al., Molecular Pharmacology, 1995 47:965-976; and Hamel et al., The Journal of Biological Chemistry, 1990 265:28, 17141-17149.

In some embodiments, the cytotoxic agent is an anti-cancer agent. Exemplary anti-cancer agents include, but are not limited to, cytostatics, enzyme inhibitors, gene regulators, cytotoxic nucleosides, tubulin binding agents or tubulin inhibitors, proteasome inhibitors, hormones and hormone antagonists, anti-angiogenesis agents, and the like. Exemplary cytostatic anti-cancer agents include alkylating agents such as the anthracycline family of drugs (e.g. adriamycin, carminomycin, cyclosporin-A, chloroquine, methopterin, mithramycin, porfiromycin, streptonigrin, porfiromycin, anthracenediones, and aziridines). Other cytostatic anti-cancer agents include DNA synthesis inhibitors (e.g., methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C), DNA-intercalators or cross-linkers (e.g., bleomycin, carboplatin, carmustine, chlorambucil, cyclophosphamide, cis-diammineplatinum (II) dichloride (cisplatin), melphalan, mitoxantrone, and oxaliplatin), and DNA-RNA transcription regulators (e.g., actinomycin D, daunorubicin, doxorubicin, homoharringtonine, and idarubicin). Other exemplary cytostatic agents that are compatible with the present disclosure include ansamycin benzoquinones, quinonoid derivatives (e.g. quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g. carmustine, lomustine, semustine).

In some embodiments, the cytotoxic agent comprises, consists essentially of, or consists of a toxin. In some embodiments, the toxin comprises a polypeptide having ribosome-inactivating activity including, for example, gelonin, bouganin, saporin, ricin, ricin A chain, bryodin, diphtheria toxin, restrictocin, Pseudomonas exotoxin A and variants thereof.

In some embodiments, the cytotoxic agent comprises, consists essentially of, or consists of a radioactive isotope, a radionuclide (e.g., a peptide receptor radionuclide), or a radiolabel. Exemplary high-energy radioactive isotopes or radionuclides include: 90Y, 125I, 131I, 123I, 111In, 105Rh, 153Sm, 67Cu, 67Ga. 166Ho, 177Lu, 186Re and 188Re. These isotopes typically produce high energy α- or β-particles which have a short path length. Such radioactive isotopes or radionuclides kill cells to which they are in close proximity, for example neoplastic cells to which the conjugate has attached or has entered. They have little or no effect on non-localized cells and are essentially non-immunogenic. Alternatively, high-energy isotopes may be generated by thermal irradiation of an otherwise stable isotope, for example as in boron neutron-capture therapy (Guan et al., PNAS, 95:13206-10, 1998). Accordingly, in some embodiments, the cytotoxic agent is a radioactive isotope selected from the group consisting of 90Y, 125I, 131I, 123I, 111 In, 105Rh, 153Sm, 67Cu, 67Ga, 166Ho, 177Lu, 186Re and 188Re.

In some embodiments, the anti-IL-18Rβ antibody or antigen-binding fragment thereof is conjugated to a radioisotope, wherein the radioisotope is a metal or non-metal radioisotope. In some embodiments, the anti-IL-18Rβ antibody or antigen-binding fragment thereof is conjugated to a radioisotope, wherein the radioisotope is selected from ¹¹C, ¹⁸F, ⁷⁶Br, ¹²⁴I, ⁶⁸Ga, ⁴⁴Sc, ⁶⁴Cu, ⁸⁶Y, ⁵⁵Co, ⁷²As, ⁸⁹Zr, ¹²³I, ¹³¹I, ^{99m}Tc, ¹¹¹In, ⁶⁷Ga, and ¹⁷⁷Lu. In some embodiments, the anti-IL-18Rβ antibody or antigen-binding fragment thereof is conjugated to an MRI contrast agent. In some embodiments, the MRI contrast agent is selected from paramagnetic metal complexes (e.g. complexes containing gadolinium (III), dysprosium (III), or manganese (II)) or superparamagnetic agents (e.g. iron oxide nanoparticles (SPION) or ultrasmall superparamagnetic iron oxide (USPIO)), In some embodiments, the anti-IL-18Rβ antibody or antigen-binding fragment thereof is conjugated to a CT contrast agent (e.g. iodinated compounds).

In some embodiments, the heterologous molecule or moiety, e.g., drug moiety, has an intracellular activity. In some embodiments, the anti-IL-18Rβ antibody or antigen-binding fragment of the conjugate is internalized and the heterologous molecule or moiety, e.g., drug moiety, is a cytotoxin that blocks the protein synthesis of the cell, therein leading to cell death. The conjugate can be used for inhibiting the multiplication of a tumor cell or cancer cell, causing apoptosis in a tumor or cancer cell, or for treating cancer in a patient. The conjugate can be used accordingly in a variety of settings for the treatment of animal cancers. The conjugate can be used to deliver a drug to a tumor cell or cancer cell. In some embodiments, upon binding to IL-18Rβ on a tumor cell, the conjugate and/or drug can be taken up inside a tumor cell or cancer cell through receptor-mediated endocytosis. In some embodiments, the conjugate can be used for targeting infected cells in a subject having an infectious disease.

In some embodiments, the conjugate is an antibody drug conjugate (ADC, also called immunoconjugates) containing an anti-IL-18Rβ antibody or antigen-binding fragment provided herein conjugated to a drug moiety that acts as a therapeutic agent. The therapeutic agent can be any therapeutic agent suitable for treating the disease or disorder. In some embodiments, the therapeutic agent is an anti-inflammatory agent or an anti-cancer agent. In some embodiments, the therapeutic agent is an anti-inflammatory agent that inhibits or otherwise lessens an inflammatory response. In some embodiments, the therapeutic agent is either cytotoxic, cytostatic, or otherwise provides some therapeutic benefit in the treatment of a cancer. In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In some embodiments, provided antibody drug conjugates of the present disclosure allow targeted-delivery of the drug moiety to tumors. In some cases, this can result in targeted killing of the tumor cell.

Examples of drugs or payloads as drug moieties are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-(9C1)-L-valinamide), mc-MMAF (maleimidocaproyl-monomethylauristatin For N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino) heptanoyl-(αR,βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[4-[N-[6-(2.5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-methyl-L-valyl-N-[(1S,2R)-4-[(2S)-2-[(1R,2R)-3-[[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives. The preferred payloads of the present invention are selected from the group consisting of mc-MMAF and mc-Val-Cit-PABA-MMAE.

In some embodiments, the heterologous molecule or moiety is an imaging agent or a detectable moiety.

In some embodiments, the imaging agent or detectable moiety comprises, consists essentially of, or consists of a label, which can generate a detectable signal, indirectly or directly. These conjugates can be used for research or diagnostic applications, such as for the in vivo detection of a disease or disorder, e.g., cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin: an enzyme, such as alkaline phosphatase, β-galactosidase or horseradish peroxidase; an imaging agent; or a metal ion. In some embodiments, the label is a radioactive atom for scintigraphic studies, for example 99Tc or 123I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The conjugates may be prepared using any methods known in the art. See, e.g., WO 2009/067800, WO 2011/133886, and U.S. Patent Application Publication No. 2014322129, incorporated by reference herein in their entirety.

In some embodiments, the linkage of an antibody or antigen binding fragment to a heterologous molecule or moiety, such as a drug or toxin or other heterologous molecule or moiety, is a direct or indirect linkage. In some embodiments, the attachment can be covalent or non-covalent, e.g., via a biotin-streptavidin non-covalent interaction. For example, a moiety can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols. In some embodiments, the attachment of a heterologous molecule or moiety, such as a therapeutic agent, can be by chemical conjugation and linkage methods known in the art. In some embodiments, the moiety can be linked to an antibody by a linker. In some embodiments, linkers such as peptide linkers, cleavable linkers, non-cleavable linkers or linkers that aid in the conjugation reaction, can be used to link or conjugate the effector moieties to the antibody or antigen-binding fragment. Attachment of a linker to an antibody or an antigen-binding fragment thereof can be accomplished in a variety of ways, such as through surface lysines, reductive-coupling to oxidized carbohydrates, and through cysteine residues liberated by reducing interchain disulfide linkages. A variety of antibody drug conjugate linkage systems are known in the art, including hydrazone-, disulfide- and peptide-based linkages.

In some embodiments, an anti-IL-18Rβ antibody or antigen-binding fragment thereof provided herein is conjugated to one or more moieties, e.g. about 1 to about 20 drug moieties, through a linker (L). In some embodiments, the conjugate comprises the following components: (antibody or antigen-binding fragment), (L) q and (moiety) m, wherein the antibody or antigen-binding fragment is any anti-IL-18Rβ antibody or antigen-binding fragment thereof as described herein; L is a linker for linking the protein or polypeptide to the moiety; m is at least 1; q is 0 or more; and the resulting conjugate binds to IL-18Rβ. In particular embodiments, m is 1 to 4 and q is 0 to 8.

The linker may be composed of one or more linker components. For covalent attachment of the antibody and the drug moiety the linker typically has two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzymes, for example, a tumor-associated protease, cathepsin B, C and D, at a plasmin protease.

Exemplary linker components include 6-maleimidocaproyl (“MC”), maleimidopropanoyl (“MP”), valine-citrulline (“val-cit”), a alanine-phenylalanine (“ala-phe”), p-aminobenzyloxycarbonyl (“PAB”), N-Succinimidyl 4-(2-pyridylthio) pentanoate (“SPP”), N-Succinimidyl 4-(N-maleimidomethyl) cyclohexane-I carboxylate (“SMCC”), and N-Succinimidyl (4-iodo-acetyl) aminobenzoate (“SIAB”).

Conjugates of an anti-IL-18Rβ antibody or antigen-binding fragment provided herein and therapeutic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl substrate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The antibody drug conjugate can be prepared by a variety of methods, such as organic chemistry reactions, conditions, and reagents known to those skilled in the art. Alternatively, a fusion protein containing an antibody or antigen-binding fragment and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### F. Nucleic Acids and Expression Vectors and Methods of Producing Antibodies

Also provided herein, in some embodiments, are polynucleotides comprising nucleic acid(s) encoding any anti-IL-18Rβ antibody or antigen-binding fragment thereof disclosed herein, or any heavy chain or light chain thereof (or variable region thereof); or any multi-specific antibody disclosed herein.

Also provided herein, in some embodiments, are nucleic acid(s) encoding any anti-IL-18Rβ antibody or antigen-binding fragment thereof disclosed herein, or any heavy chain or light chain thereof (or variable region thereof); or any multi-specific antibody disclosed herein.

The polypeptide sequences may be used to determine appropriate nucleic acid sequences encoding the particular antibody or antigen-binding fragment thereof disclosed thereby. The nucleic acid sequence may be optimized to reflect particular codon “preferences” for various expression systems according to standard methods well known to those of skill in the art.

In some embodiments, the nucleic acid(s) encode any anti-IL-18Rβ antibody or antigen-binding fragment thereof disclosed herein.

In some embodiments, the nucleic acid(s) encode the VH region of any anti-IL-18Rβ antibody or antigen-binding fragment thereof disclosed herein. In some embodiments, the nucleic acid(s) encode the VL region of any anti-IL-18Rβ antibody or antigen-binding fragment thereof disclosed herein. In some embodiments, the nucleic acid(s) encode the VH region and VL region of any anti-IL-18Rβ antibody or antigen-binding fragment thereof disclosed herein. In some embodiments, the nucleic acid(s) encode a VH region comprising the CDR-H1, CDR-H2, and CDR-H3 of any anti-IL-18Rβ antibody or antigen-binding fragment thereof disclosed herein; and/or a VL region comprising the CDR-L1, CDR-L2, and CDR-L3 of any anti-IL-18Rβ antibody or antigen-binding fragment thereof disclosed herein. In some embodiments, the nucleic acid(s) encode a VH region comprising the CDR-H1, CDR-H2, and CDR-H3 of any anti-IL-18Rβ antibody or antigen-binding fragment thereof disclosed herein; and/or a VL region comprising the CDR-L3 of any anti-IL-18Rβ antibody or antigen-binding fragment thereof disclosed herein.

In some embodiments, the nucleic acid(s) comprises a nucleic acid encoding a VH region comprising any one of the nucleotide sequences set forth in SEQ ID NOs: 127, 131, 135, 139, 143, 147, 151, and 155, or a nucleotide sequence having at least, or at least about 85%, 86%, 87%, 88%, 89%, 90%, 91%, 92%, 93%, 94%, 95%, 96%, 97%, 98% or 99% sequence identity to any of the foregoing; and a nucleic acid encoding a VL region comprising any one of the nucleotide sequence set forth in SEQ ID NOs: 128, 132, 136, 140, 144, 148, 152, and 156, or a nucleotide sequence having at least, or at least about 85%, 86%, 87%, 88%, 89%, 90%, 91%, 92%, 93%, 94%, 95%, 96%, 97%, 98% or 99% sequence identity to any of the foregoing.

In some embodiments, the nucleic acid(s) comprises a nucleic acid encoding a VH region comprising the nucleotide sequence of SEQ ID NO: 127 or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 127; and a nucleic acid encoding a VL region comprising the nucleotide sequence of SEQ ID NO: 128, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 128. In some embodiments, the nucleic acid(s) comprises a nucleic acid encoding a VH region comprising the nucleotide sequence of SEQ ID NO: 127; and a nucleic acid encoding a VL region comprising the nucleotide sequence of SEQ ID NO: 128.

In some embodiments, the nucleic acid(s) comprises a nucleic acid encoding a VH region comprising the nucleotide sequence of SEQ ID NO: 131, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 131; and a nucleic acid encoding a VL region comprising the nucleotide sequence of SEQ ID NO: 132, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 132. In some embodiments, the nucleic acid(s) comprises a nucleic acid encoding a VH region comprising the nucleotide sequence of SEQ ID NO: 131; and a nucleic acid encoding a VL region comprising the nucleotide sequence of SEQ ID NO: 132.

In some embodiments, the nucleic acid(s) comprises a nucleic acid encoding a VH region comprising the nucleotide sequence of SEQ ID NO: 135, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 135; and a nucleic acid encoding a VL region comprising the nucleotide sequence of SEQ ID NO: 136, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 136. In some embodiments, the nucleic acid(s) comprises a nucleic acid encoding a VH region comprising the nucleotide sequence of SEQ ID NO: 135; and a nucleic acid encoding a VL region comprising the nucleotide sequence of SEQ ID NO: 136.

In some embodiments, the nucleic acid(s) comprises a nucleic acid encoding a VH region comprising the nucleotide sequence of SEQ ID NO: 139, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 139; and a nucleic acid encoding a VL region comprising the nucleotide sequence of SEQ ID NO: 140, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 140. In some embodiments, the nucleic acid(s) comprises a nucleic acid encoding a VH region comprising the nucleotide sequence of SEQ ID NO: 139; and a nucleic acid encoding a VL region comprising the nucleotide sequence of SEQ ID NO: 140.

In some embodiments, the nucleic acid(s) comprises a nucleic acid encoding a VH region comprising the nucleotide sequence of SEQ ID NO: 143, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 143; and a nucleic acid encoding a VL region comprising the nucleotide sequence of SEQ ID NO: 144, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 144. In some embodiments, the nucleic acid(s) comprises a nucleic acid encoding a VH region comprising the nucleotide sequence of SEQ ID NO: 143; and a nucleic acid encoding a VL region comprising the nucleotide sequence of SEQ ID NO: 144.

In some embodiments, the nucleic acid(s) comprises a nucleic acid encoding a VH region comprising the nucleotide sequence of SEQ ID NO: 147, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 147; and a nucleic acid encoding a VL region comprising the nucleotide sequence of SEQ ID NO: 148, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 148. In some embodiments, the nucleic acid(s) comprises a nucleic acid encoding a VH region comprising the nucleotide sequence of SEQ ID NO: 147; and a nucleic acid encoding a VL region comprising the nucleotide sequence of SEQ ID NO: 148.

In some embodiments, the nucleic acid(s) comprises a nucleic acid encoding a VH region comprising the nucleotide sequence of SEQ ID NO: 151, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 151; and a nucleic acid encoding a VL region comprising the nucleotide sequence of SEQ ID NO: 152, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 152. In some embodiments, the nucleic acid(s) comprises a nucleic acid encoding a VH region comprising the nucleotide sequence of SEQ ID NO: 151; and a nucleic acid encoding a VL region comprising the nucleotide sequence of SEQ ID NO: 152.

In some embodiments, the nucleic acid(s) comprises a nucleic acid encoding a VH region comprising the nucleotide sequence of SEQ ID NO: 155, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 155; and a nucleic acid encoding a VL region comprising the nucleotide sequence of SEQ ID NO: 156, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 156. In some embodiments, the nucleic acid(s) comprises a nucleic acid encoding a VH region comprising the nucleotide sequence of SEQ ID NO: 155; and a nucleic acid encoding a VL region comprising the nucleotide sequence of SEQ ID NO: 156.

In some embodiments, the nucleic acid(s) encode the heavy chain of any anti-IL-18Rβ antibody or antigen-binding fragment thereof disclosed herein. In some embodiments, the nucleic acid(s) encode the light chain of any anti-IL-18Rβ antibody or antigen-binding fragment thereof disclosed herein. In some embodiments, the nucleic acid(s) encode the heavy chain of any anti-IL-18Rβ antibody or antigen-binding fragment thereof disclosed herein; and the light chain of any anti-IL-18Rβ antibody or antigen-binding fragment thereof disclosed herein.

In some embodiments, the nucleic acid(s) comprises a nucleic acid encoding a heavy chain comprising any one of the nucleotide sequences set forth in SEQ ID NOs: 129, 133, 137, 141, 145, 149, 153, and 157, or a nucleotide sequence having at least, or at least about 85%, 86%, 87%, 88%, 89%, 90%, 91%, 92%, 93%, 94%, 95%, 96%, 97%, 98% or 99% sequence identity to any of the foregoing; and a nucleic acid encoding a light chain comprising any one of the nucleotide sequence set forth in SEQ ID NOs: 130, 134, 138, 142, 146, 150, 154, and 158, or a nucleotide sequence having at least, or at least about 85%, 86%, 87%, 88%, 89%, 90%, 91%, 92%, 93%, 94%, 95%, 96%, 97%, 98% or 99% sequence identity to any of the foregoing.

In some embodiments, the nucleic acid(s) comprises a nucleic acid encoding a heavy chain comprising the nucleotide sequence of SEQ ID NO: 129, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 129; and a nucleic acid encoding a light chain comprising the nucleotide sequence of SEQ ID NO: 130, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 130. In some embodiments, the nucleic acid(s) comprises a nucleic acid encoding a heavy chain comprising the nucleotide sequence of SEQ ID NO: 129; and a nucleic acid encoding a light chain comprising the nucleotide sequence of SEQ ID NO: 130.

In some embodiments, the nucleic acid(s) comprises a nucleic acid encoding a heavy chain comprising the nucleotide sequence of SEQ ID NO: 133, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 133; and a nucleic acid encoding a light chain comprising the nucleotide sequence of SEQ ID NO: 134, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 134. In some embodiments, the nucleic acid(s) comprises a nucleic acid encoding a heavy chain comprising the nucleotide sequence of SEQ ID NO: 133; and a nucleic acid encoding a light chain comprising the nucleotide sequence of SEQ ID NO: 134.

In some embodiments, the nucleic acid(s) comprises a nucleic acid encoding a heavy chain comprising the nucleotide sequence of SEQ ID NO: 137, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 137; and a nucleic acid encoding a light chain comprising the nucleotide sequence of SEQ ID NO: 138, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 138. In some embodiments, the nucleic acid(s) comprises a nucleic acid encoding a heavy chain comprising the nucleotide sequence of SEQ ID NO: 137; and a nucleic acid encoding a light chain comprising the nucleotide sequence of SEQ ID NO: 138.

In some embodiments, the nucleic acid(s) comprises a nucleic acid encoding a heavy chain comprising the nucleotide sequence of SEQ ID NO: 141, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 141; and a nucleic acid encoding a light chain comprising the nucleotide sequence of SEQ ID NO: 142, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 142. In some embodiments, the nucleic acid(s) comprises a nucleic acid encoding a heavy chain comprising the nucleotide sequence of SEQ ID NO: 141; and a nucleic acid encoding a light chain comprising the nucleotide sequence of SEQ ID NO: 142.

In some embodiments, the nucleic acid(s) comprises a nucleic acid encoding a heavy chain comprising the nucleotide sequence of SEQ ID NO: 145, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 145; and a nucleic acid encoding a light chain comprising the nucleotide sequence of SEQ ID NO: 146, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 146. In some embodiments, the nucleic acid(s) comprises a nucleic acid encoding a heavy chain comprising the nucleotide sequence of SEQ ID NO: 145; and a nucleic acid encoding a light chain comprising the nucleotide sequence of SEQ ID NO: 146.

In some embodiments, the nucleic acid(s) comprises a nucleic acid encoding a heavy chain comprising the nucleotide sequence of SEQ ID NO: 149, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 149; and a nucleic acid encoding a light chain comprising the nucleotide sequence of SEQ ID NO: 150, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 150. In some embodiments, the nucleic acid(s) comprises a nucleic acid encoding a heavy chain comprising the nucleotide sequence of SEQ ID NO: 149; and a nucleic acid encoding a light chain comprising the nucleotide sequence of SEQ ID NO: 150.

In some embodiments, the nucleic acid(s) comprises a nucleic acid encoding a heavy chain comprising the nucleotide sequence of SEQ ID NO: 153, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 153; and a nucleic acid encoding a light chain comprising the nucleotide sequence of SEQ ID NO: 154, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 154. In some embodiments, the nucleic acid(s) comprises a nucleic acid encoding a heavy chain comprising the nucleotide sequence of SEQ ID NO: 153; and a nucleic acid encoding a light chain comprising the nucleotide sequence of SEQ ID NO: 154.

In some embodiments, the nucleic acid(s) comprises a nucleic acid encoding a heavy chain comprising the nucleotide sequence of SEQ ID NO: 157, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 157; and a nucleic acid encoding a light chain comprising the nucleotide sequence of SEQ ID NO: 158, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 158. In some embodiments, the nucleic acid(s) comprises a nucleic acid encoding a heavy chain comprising the nucleotide sequence of SEQ ID NO: 157; and a nucleic acid encoding a light chain comprising the nucleotide sequence of SEQ ID NO: 158.

In some embodiments, the nucleic acid(s) is an isolated nucleic acid(s). The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain variable domain fragment and a light chain variable domain fragment linked together.

This disclosure is not intended to be limited with regard to the source of the antibody or the manner in which it is made. Various procedures known within the art may be used for the production of monoclonal antibodies directed against IL-18Rβ (See, for example, Antibodies: A Laboratory Manual, Harlow E, and Lane D, 1988, Cold Spring Harbor Laboratory Press, Cold Spring Harbor, NY, incorporated herein by reference). In some embodiments, the anti-IL-18Rβ antibodies, and antigen binding fragments thereof, are recombinantly expressed and produced.

In some embodiments, a nucleic acid of the present disclosure may be operably linked to a transcriptional control element, e.g., a promoter, and enhancer, etc. Suitable promoter and enhancer elements are known to those of skill in the art.

In some embodiments, a polynucleotide encoding a heavy chain or light chain of an anti-IL-18Rβ antibody or antigen-binding fragment thereof comprises a nucleotide sequence that encodes a leader sequence, which, when translated, is located at the N terminus of the heavy chain or light chain. The leader sequence may be the native heavy or light chain leader sequence, or may be another heterologous leader sequence.

Nucleic acid molecules may be constructed using conventional recombinant DNA techniques well-known in the art. In some embodiments, a nucleic acid molecule is an expression vector that is suitable for expression in a selected host cell.

Also provided herein are vectors comprising polynucleotides that encode any of the anti-IL-18Rβ antibodies disclosed herein, or any of the heavy chains and/or light chains thereof that are disclosed herein.

Also provided herein are vectors comprising nucleic acid(s) that encode any of the anti-IL-18Rβ antibodies disclosed herein, or any of the heavy chains and/or light chains thereof that are disclosed herein.

In certain embodiments, the vector is a DNA vector, an RNA vector, a plasmid, a lentiviral vector, an adenoviral vector, an adeno-associated viral vector, and a retroviral vector. In some embodiments, the vector is a viral vector. In some embodiments, the viral vector is a retroviral vector or a lentiviral vector. In some embodiments, the viral vector is a retroviral vector. In some embodiments, the viral vector is a lentiviral vector.

In certain embodiments, the vector is an expression vector. A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain variable domain fragment and light chain variable domain fragment are expressed as part of a single polypeptide, such as, for example, when the antibody is an scFv.

In some embodiments, a first vector comprises a polynucleotide that encodes a heavy chain, such as any heavy chain or variable region thereof disclosed herein, and a second vector comprises a polynucleotide that encodes a light chain, such as any light chain or variable region thereof disclosed herein. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Also provided is a host cell comprising any of the vectors, nucleic acids, or polynucleotides disclosed herein, or any anti-IL-18Rβ antibody or antigen-binding fragment thereof disclosed herein. The host cell may be of eukaryotic, prokaryotic, mammalian, or bacterial origin. In various embodiments, anti-IL-18Rβ heavy chains and/or anti-IL-18Rβ light chains, such as any of those described herein, may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells (Crucell); and NSO cells. In some embodiments, anti-IL-18Rβ heavy chains and/or anti-IL-18Rβ light chains may be expressed in yeast. See, e.g., U.S. Publication No. US 2006/0270045 A1. In some embodiments, a particular eukaryotic host cell is selected based on its ability to make desired post-translational modifications to the anti-IL-18Rβ heavy chains and/or anti-IL-18Rβ light chains. For example, in some embodiments, CHO cells produce polypeptides that have a higher level of sialylation than the same polypeptide produced in HEK293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, the host cells are capable of over-expressing heterologous polynucleotides or nucleic acids, which can be used for the purpose of isolating the encoded antibody or antigen-binding fragment thereof, of heavy chain and/or light chain thereof. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisiae, S. pombe; or K. lactis).

Also provided herein is a method of producing an anti-IL-18Rβ antibody or antigen-binding fragment thereof that comprises culturing any host cell disclosed herein, e.g., any host cell that comprises any vector, polynucleotide, or nucleic acid(s) disclosed herein, under a condition that produces the antibody or antigen-binding fragment thereof.

In some embodiments, a vector, polynucleotide, or nucleic acid(s) encoding any anti-IL-18Rβ antibody or antigen-binding fragment thereof disclosed herein, is introduced directly into a host cell, and the cell incubated under conditions sufficient to induce expression of the encoded antibody or antigen-binding fragment thereof. Expression may conveniently be achieved by culturing under appropriate conditions recombinant host cells containing the nucleic acid(s). Following production by expression, an antibody or antigen-binding fragment thereof, may be recovered, isolated, and/or purified using any suitable technique, and then used as desired. In some embodiments, the method further comprises recovering the anti-IL-18Rβ antibody or antigen-binding fragment thereof that is produced.

Anti-IL-18Rβ antibodies and antigen-binding fragments thereof may be purified by any suitable method. Such methods include, but are not limited to, the use of affinity matrices or hydrophobic interaction chromatography. Suitable affinity ligands such as ligands that bind antibody constant regions. For example, a Protein A, Protein G, Protein A/G, or an antibody affinity column may be used to bind the constant region and to purify an anti-IL-18Rβ antibody or antigen-binding fragment thereof. Hydrophobic interactive chromatography, for example, a butyl or phenyl column, may also suitable for purifying some polypeptides such as antibodies. Ion exchange chromatography (e.g., anion exchange chromatography and/or cation exchange chromatography) may also suitable for purifying some polypeptides such as antibodies. Mixed-mode chromatography (e.g., reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

Anti-IL-18Rβ antibodies and antigen-binding fragments thereof as provided herein, and encoding nucleic acid molecules, polynucleotides, and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function.

### II. Pharmaceutical Compositions

Provided herein are compositions containing any of the anti-IL-18Rβ antibodies or antigen-binding fragments thereof as described herein, or any of the multi-specific antibodies or conjugates as described herein. In some embodiments, the composition, e.g., pharmaceutical composition, further comprise a pharmaceutically acceptable excipient. In some embodiments, the composition is for use in detection or imaging. In some embodiments, the composition, e.g., pharmaceutical composition, is for treatment of a disease or disorder. In some embodiments, the composition is a pharmaceutical composition.

Also provided herein are pharmaceutical compositions comprising any of the anti-IL-18Rβ antibodies or antigen-binding fragments thereof as described herein, or any of the multi-specific antibodies or conjugates as described herein; and a pharmaceutically acceptable excipient.

In some embodiments, the composition, e.g., pharmaceutical composition, can comprise one or more excipients for modifying, maintaining or preserving, for example, the pH, osmolarity, viscosity, clarity, color, isotonicity, odor, sterility, stability, rate of dissolution or release, adsorption, or penetration of the composition. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the pharmaceutical composition is a solid, such as a powder, capsule, or tablet. In some instances, the components of the pharmaceutical composition can be lyophilized. In some embodiments, the pharmaceutical composition is a lyophilized composition. In some embodiments, the solid pharmaceutical composition (e.g., the lyophilized composition) is reconstituted or dissolved in a liquid prior to administration.

In some embodiments, the pharmaceutical composition is a liquid, for example a provided protein dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

A pharmaceutically acceptable carrier may be a pharmaceutically acceptable material, composition, or vehicle. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is for administration to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

In some embodiments, long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some embodiments, the pharmaceutical composition is for administration to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the pharmaceutical composition is for administration intravenously (IV) and/or subcutaneously (SC).

A provided pharmaceutical composition may, in some embodiments, be in a form suitable for intravenous infusion.

In some embodiments, the pharmaceutical composition is formulated in one or more dosages for one or more administrations, such as any of the dosing frequencies and/or amounts described herein.

### III. Methods of Use

Provided herein are methods of using any anti-IL-18Rβ antibodies and antigen-binding fragments thereof, any multi-specific antibody, any conjugate, or any composition, e.g., pharmaceutical composition, described herein. The anti-IL-18Rβ antibodies and antigen-binding fragments thereof described herein, and the multi-specific antibodies and conjugates containing the anti-IL-18Rβ antibodies and antigen-binding fragments thereof described herein, including pharmaceutical compositions thereof, can be used in a variety of therapeutic applications, such as the treatment of a disease or disorder in a subject having the disease or disorder, and/or in diagnostic, detection, and imaging applications, such as in subjects having a disease or disorder or in a sample from such a subject.

Also provided herein are uses of any anti-IL-18Rβ antibody or antigen-binding fragment thereof described herein, any multi-specific antibody described herein, any conjugate described herein, or any composition, e.g., pharmaceutical composition, described herein, for one or more of treatment of a disease or disorder, diagnosis of a disease or disorder, assessment of IL-18Rβ expression or activity, or monitoring of treatment of a disease or disorder.

Also provided herein are uses of any anti-IL-18Rβ antibody or antigen-binding fragment thereof described herein, any multi-specific antibody described herein, any conjugate described herein, or any composition, e.g., pharmaceutical composition, described herein, in the manufacture of a medicament for one or more of treatment of a disease or disorder, diagnosis of a disease or disorder, assessment of IL-18Rβ expression or activity, or monitoring of treatment of a disease or disorder.

Also provided herein is any anti-IL-18Rβ antibody or antigen-binding fragment thereof described herein, any multi-specific antibody described herein, any conjugate described herein, or any composition, e.g., pharmaceutical composition, described herein, for use in treatment of a disease or disorder, diagnosis of a disease or disorder, assessment of IL-18Rβ expression or activity, or monitoring of treatment of a disease or disorder.

### A. Methods of Treatment

Provided herein are methods of treatment that comprise administering any anti-IL-18Rβ antibody or antigen-binding fragment thereof described herein, any multi-specific antibody described herein, any conjugate described herein, or any composition, e.g., pharmaceutical composition, described herein, to a subject having a disease or disorder. In some embodiments, provided herein is a method of treatment comprising administering any anti-IL-18Rβ antibody or antigen-binding fragment thereof described herein to a subject having a disease or disorder. In some embodiments, provided herein is a method of treatment comprising administering any multi-specific antibody described herein to a subject having a disease or disorder. In some embodiments, provided herein is a method of treatment comprising administering any conjugate described herein to a subject having a disease or disorder. In some embodiments, provided herein is a method of treatment comprising administering any composition, e.g., pharmaceutical composition, described herein to a subject having a disease or disorder.

Also provided herein are uses of any anti-IL-18Rβ antibody or antigen-binding fragment thereof described herein, any multi-specific antibody described herein, any conjugate described herein, or any composition, e.g., pharmaceutical composition, described herein, for treatment of a disease or disorder.

Also provided herein are uses of any anti-IL-18Rβ antibody or antigen-binding fragment thereof described herein, any multi-specific antibody described herein, any conjugate described herein, or any composition, e.g., pharmaceutical composition, described herein, for the manufacture of a medicament for treatment of a disease or disorder.

Also provided herein is any anti-IL-18Rβ antibody or antigen-binding fragment thereof described herein, any multi-specific antibody described herein, any conjugate described herein, or any composition, e.g., pharmaceutical composition, described herein, for use in treatment of a disease or disorder.

Generally, treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition of this disclosure can be used to prevent the onset or reoccurrence of the disease or disorder in the subject.

In some embodiments, the anti-IL-18Rβ antibody or antigen-binding fragment thereof, multi-specific antibody, conjugate, or composition, e.g., pharmaceutical composition, can be used to inhibit growth of mammalian cancer cells, such as human cancer cells. In some embodiments, such a method of treating cancer can include administering an effective amount of any of the pharmaceutical compositions described herein to a subject with a cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the pharmaceutical composition to a subject having the cancer.

In some embodiments, the anti-IL-18Rβ antibody or antigen-binding fragment there, multi-specific antibody, conjugate, or composition, e.g., pharmaceutical composition, are useful in treating, alleviating a symptom of, ameliorating and/or delaying the progression of a disease or disorder that is a cancer or other neoplastic condition.

In some embodiments, the disease or disorder is an autoimmune disease, an inflammatory disease, a cancer, or an infectious disease.

In some embodiments, the disease or disorder is associated with an increase in IL-18Rβ in a biological sample of the subject as compared to a subject not having the disease or disorder. In some embodiments, the disease or disorder is associated with an increase in activity of the IL-18 signaling pathway in a biological sample of the subject as compared to a subject not having the disease or disorder. In some embodiments, the biological sample comprises, consists essentially of, or consists of body fluids, such as blood, plasma, serum, cerebrospinal fluid, synovial fluid, urine or sweat; tissue or organ samples, including processed samples derived therefrom. In some embodiments, the biological sample is a serum sample. In some embodiments, the biological sample is a tissue sample.

In some embodiments, the disease or disorder is an inflammatory disease. The inflammatory disease can, in some embodiments, be any inflammatory disease, such as any inflammatory disease associated with an increase in levels of IL-18Rβ and/or an increase in activity of the IL-18 signaling pathway, as compared to a subject not having the inflammatory disease.

In some embodiments, the disease or disorder is an autoimmune disease. The inflammatory disease can, in some embodiments, be any autoimmune disease, such as any autoimmune disease associated with an increase in levels of IL-18Rβ and/or an increase in activity of the IL-18 signaling pathway, as compared to a subject not having the autoimmune disease.

In some embodiments, the disease or disorder is a cancer. The cancer can, in some embodiments, be any cancer, such as any cancer associated with an increase in levels of IL-18Rβ and/or an increase in activity of the IL-18 signaling pathway, as compared to a subject not having the cancer.

In some embodiments, the disease or disorder is an infectious disease. The infectious disease can, in some embodiments, be any autoimmune disease, such as any infectious disease associated with an increase in levels of IL-18Rβ and/or an increase in activity of the IL-18 signaling pathway, as compared to a subject not having the infectious disease. In some embodiments, the infectious disease is a disease caused by a viral infection. In some embodiments, the infectious disease is caused by acute respiratory syndrome coronavirus (SARS-CoV) or acute respiratory syndrome coronavirus 2 (SARS-CoV-2). In some embodiments, the infectious disease is caused by SARS-CoV. In some embodiments, the infectious disease is caused by SARS-CoV-2. In some embodiments, the disease or disorder is COVID-19.

In some embodiments, the disease or disorder is inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome (SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, monogenic enterocolitis, Celiac disease, age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).

In some embodiments, the disease or disorder is inflammatory bowel disease (IBD). In some embodiments, the inflammatory bowel disease is Crohn's disease or ulcerative colitis. In some embodiments, the inflammatory bowel disease is Crohn's disease. In some embodiments, the inflammatory bowel disease is ulcerative colitis. Accordingly, in some embodiments, the disease or disorder is Crohn's disease. In some embodiments, the disease or disorder is ulcerative colitis.

In some embodiments, the IBD is monogenic IBD.

In some embodiments, the IBD is very early onset IBD.

In some embodiments, the disease or disorder is a genetic alteration associated with a gain of function of NLRC4. In some embodiments, the genetic alteration is a gain of function mutation. In some embodiments, the genetic alteration is associated with very early onset IBD.

In some embodiments, the disease or disorder is a genetic alteration associated with a loss of function of XIAP. In some embodiments, the genetic alteration is associated with very early onset IBD.

In some embodiments, the disease or disorder is COPD. In some embodiments, the disease or disorder is type 1 diabetes or type 2 diabetes. In some embodiments, the disease or disorder is liver disease. In some embodiments, the disease or disorder is AOSD. In some embodiments, the disease or disorder is HLH. In some embodiments, the disease or disorder is an ocular inflammatory disease. In some embodiments, the disease or disorder is DED. In some embodiments, the disease or disorder is CRS. In some embodiments, the disease or disorder is SIRS. In some embodiments, the disease or disorder is AIFEC. In some embodiments, the disease or disorder is XIAP deficiency. In some embodiments, the disease or disorder is NLRC4 gain of function. In some embodiments, the disease or disorder is atopic dermatitis. In some embodiments, the disease or disorder is Behçet's disease. In some embodiments, the disease or disorder is SLE. In some embodiments, the disease or disorder is acute coronary syndrome. In some embodiments, the disease or disorder is an allergy. In some embodiments, the disease or disorder is ALS. In some embodiments, the disease or disorder is asthma. In some embodiments, the disease or disorder is Celiac disease. In some embodiments, the idiopathic arthritis is multiple sclerosis.

In some embodiments, the disease or disorder is Parkinson's disease. In some embodiments, the disease or disorder is Grave's disease. In some embodiments, the disease or disorder is Hashimoto's thyroiditis. In some embodiments, the disease or disorder is Addison's disease. In some embodiments, the disease or disorder is dermatomyositis. In some embodiments, the disease or disorder is myasthenia gravis. In some embodiments, the disease or disorder is pernicious anemia. In some embodiments, the disease or disorder is Sjogren syndrome. In some embodiments, the disease or disorder is vasculitis. In some embodiments, the disease or disorder is uveitis. In some embodiments, the disease or disorder is sepsis. In some embodiments, the disease or disorder is atherosclerosis. In some embodiments, the disease or disorder is ankylosing spondylitis. In some embodiments, the disease or disorder is arthritis. In some embodiments, the arthritis is rheumatoid arthritis, psoriatic arthritis, or idiopathic arthritis. In some embodiments, the arthritis is rheumatoid arthritis. In some embodiments, the rheumatoid arthritis is juvenile rheumatoid arthritis. In some embodiments, the arthritis is idiopathic arthritis. In some embodiments, the idiopathic arthritis is juvenile idiopathic arthritis. In some embodiments, the idiopathic arthritis is systemic juvenile idiopathic arthritis (SJIA). In some embodiments, the arthritis is giant cell arthritis (GCA). In some embodiments, the disease or disorder is psoriasis. In some embodiments, the psoriasis is plaque psoriasis. In some embodiments, the disease or disorder is an organ transplant rejection. In some embodiments, the organ transplant rejection is a kidney transplant rejection. In some embodiments, the disease or disorder is heart disease. In some embodiments, the heart disease is ischemic heart disease. In some embodiments, the disease or disorder is MAS. In some embodiments, the MAS is infantile MAS. In some embodiments, the disease or disorder is sarcoidosis. In some embodiments, the sarcoidosis is pulmonary sarcoidosis (e.g., chronic pulmonary sarcoidosis). In some embodiments, the disease or disorder is AMD. In some embodiments, the AMD is geographic atrophy or atrophic AMD.

In some embodiments, the disease or disorder is osteoporosis.

In some embodiments, the disease or disorder is a cancer. In some embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In certain embodiments, the cancer is a metastatic cancer, refractory cancer, or recurrent cancer.

Also provided herein are methods of treatment that comprise administering any anti-IL-18Rβ antibody or antigen-binding fragment thereof described herein, any multi-specific antibody described herein, any conjugate described herein, or any composition, e.g., pharmaceutical composition, described herein, to a subject having a disease or disorder, wherein the subject has received one or more prior therapy. In some embodiments, the subject has received one or more prior therapy, such as one prior therapy, two prior therapies, three prior therapies, or four prior therapies.

In some embodiments, the subject has relapsed or is refractory to treatment after one or more prior therapy, such as one prior therapy, two prior therapies, three prior therapies, or four prior therapies. In some embodiments, the disease or disorder is a relapsed or refractory disease or disorder. In some embodiments, a prior therapy has not worked (refractory to treatment) or the disease or disorder has returned after the prior therapy (relapsed).

The one or more prior therapy can, in some embodiments, be any therapeutic agent suitable for treatment of the disease or disorder disclosed herein. In some embodiments, the one or more additional therapeutic agents is an aminosalicylate, a corticosteroid (e.g., prednisone, methylprednisolone, hydrocortisone), a thiopurine, a methotrexate, a tumor necrosis factor (TNF) inhibitor, an α4β7 integrin inhibitor (e.g., vedolizumab), a TNF-α inhibitor (e.g., infliximab, adalimumab, certolizumab pegol, or golimumab), an IL-23 inhibitor (e.g., Risankizumab-rzaa), a dual IL-12 and IL-23 inhibitor (e.g., ustekinumab), a tumor necrosis factor-like cytokine 1A (TL1A) inhibitor (e.g., PRA023, PF-06480605, RVT-3101, or TEV-48574), an IL-2-CD25 fusion protein, a bispecific anti-TL1A/anti-TNF-α binding protein, an E-type prostanoid receptor 4 (EP4) agonist, a TYK2 inhibitor (e.g., deucravacitinib), a sphingosine-1-phosphate receptor (S1PR) agonist (e.g., ozanimod), a Janus kinase (JAK) inhibitor (e.g., abrocitinib, baricitinib, delgocitinib, fedratinib, filgotinib, oclacitinib, pacritinib, peficitinib, ruxolitinib, tofacitinib, or upadacitinib), an interkeukin-1 receptor associated kinase 4 (IRAK-4) inhibitor (e.g., KT-474, BAY1834845 (zabedosertib), BAY1830839, emavusertib, GLPG2534), a toll-like receptor 7 and 8 (TLR7/8) inhibitor (e.g., afimetoran), an IL-22 inhibitor, a beta agonist, muscarinic antagonist, systemic steroid, azithromycin, roflumilast, or dupilumab. In some embodiments, the IRAK-4 inhibitor is an IRAK-4 degrader. In some embodiments, the IRAK-4 inhibitor is an inhibitor disclosed in WO2024/020084.

Also provided herein are methods of treatment that comprise administering any anti-IL-18Rβ antibody or antigen-binding fragment thereof described herein, any multi-specific antibody described herein, any conjugate described herein, or any composition, e.g., pharmaceutical composition, described herein, to a subject having inflammatory bowel disorder, e.g., Crohn's disease or ulcerative colitis.

Also provided herein are uses of any anti-IL-18Rβ antibody or antigen-binding fragment thereof described herein, any multi-specific antibody described herein, any conjugate described herein, or any composition, e.g., pharmaceutical composition, described herein, for treatment of inflammatory bowel disorder, e.g., Crohn's disease or ulcerative colitis.

Compositions of the invention can be administered in dosages and routes and at times to be determined in appropriate pre-clinical and clinical experimentation and trials. Compositions may be administered multiple times at dosages within these ranges. Administration of the compositions may be combined with other methods useful to treat the desired disease or disorder as determined by those of skill in the art. Typically, precise amount of the compositions of the present disclosure to be administered can be determined by a physician with consideration of individual differences in age, weight, tumor size, extent of infection or metastasis, and condition of the patient (subject).

In some embodiments, a therapeutically effective dose may be, by way of nonlimiting example, from about 0.01 μg/kg body weight to about 10 mg/kg body weight. In some embodiments, the therapeutically effective dose may be, by way of nonlimiting example, from about 0.01 mg/kg body weight to about 5-10 mg/kg body weight. In some embodiments, the therapeutically effective dose may be, by way of nonlimiting example, from about 3 mg to about 1800 mg body weight. In some embodiments, the therapeutically effective dose may be, by way of nonlimiting example, at or greater than 900 mg. Common dosing frequencies may range, for example, from multiple daily administrations to one administration per week, in some embodiments.

In some embodiments, the dosage of the pharmaceutical composition is a single dose or a repeated dose. In some embodiments, the doses are given to a subject once per day, twice per day, three times per day, or four or more times per day. In some embodiments, one dose of the pharmaceutical composition is administered per day. In some embodiments, two doses of the pharmaceutical composition are administered per day (B.I.D.). In some embodiments, three doses of the pharmaceutical composition are administered per day. In some embodiments, four or more doses of the pharmaceutical composition are administered per day.

In some embodiments, the one or more doses of the pharmaceutical composition are administered on one or more days per week. In some embodiments, the one or more doses of the pharmaceutical composition are administered on one day per week. In some embodiments, the one or more doses of the pharmaceutical composition are administered on two days per week. In some embodiments, the one or more doses of the pharmaceutical composition are administered on three days per week. In some embodiments, the one or more doses of the pharmaceutical composition are administered on four days per week. In some embodiments, the one or more doses of the pharmaceutical composition are administered on five days per week. In some embodiments, the one or more doses of the pharmaceutical composition are administered on six days per week. In some embodiments, the one or more doses of the pharmaceutical composition are administered on seven days per week.

In some embodiments, about 1 or more (such as about 2 or more, about 3 or more, about 4 or more, about 5 or more, about 6 or more, or about 7 or more) doses are given in a week. In some embodiments, one dose of the pharmaceutical composition is administered per week. In some embodiments, two doses of the pharmaceutical composition is administered per week. In some embodiments, three dose of the pharmaceutical composition is administered per week (T.I.W.). In some embodiments, four doses of the pharmaceutical composition is administered per week. In some embodiments, five doses of the pharmaceutical composition is administered per week. In some embodiments, six doses of the pharmaceutical composition is administered per week. In some embodiments, seven doses of the pharmaceutical composition is administered per week.

In some embodiments, the one or more doses of the pharmaceutical composition are administered once per week, once every 2 weeks, once every 3 weeks, once every 4 weeks (i.e., once every month), once every 8 weeks (i.e., once every 2 months), or once every 12 weeks (i.e., once every 3 months).

In some embodiments, multiple doses of the pharmaceutical composition are administered over the course of days, weeks, months, or years. In some embodiments, multiple doses of the pharmaceutical composition are administered over the course of one or more weeks. In some embodiments, multiple doses of the pharmaceutical composition are administered over the course of at least four weeks. In some embodiments, multiple doses of the pharmaceutical composition are administered over the course of at least one month. In some embodiments, multiple doses of the pharmaceutical composition are administered over the course of at or at least 2, 3, 4, 5, 6, 9, 12, 18, or 24 months. In some embodiments, multiple doses of the pharmaceutical compositions are administered for up to 6 months, 12 months, 24 months, or 36 months. In some embodiments, multiple doses of the pharmaceutical compositions are administered indefinitely, e.g., chronically. In some embodiments, multiple doses of the pharmaceutical compositions are administered chronically, such as for at least 2 years, 5 years, or 10 years.

In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the pharmaceutical composition is about 0.01 μg of protein per kg subject body mass or more (such as about 0.1 μg of protein per kg subject body mass or more, 0.5 μg of protein per kg subject body mass or more, 1 μg of protein per kg subject body mass or more, 2 μg of protein per kg subject body mass or more, about 5 μg of protein per kg subject body mass or more, about 10 μg of protein per kg subject body mass or more, about 25 μg of protein per kg subject body mass or more, about 50 μg of protein per kg subject body mass or more, about 100 μg of protein per kg subject body mass or more, about 250 μg of protein per kg subject body mass or more, about 500 μg of protein per kg subject body mass or more, about 1 mg of protein per kg subject body mass or more, about 2 mg of protein per kg subject body mass or more, or about 5 mg of protein per kg subject body mass or more).

In some embodiments, an administered dose of the pharmaceutical composition is or is about 0.1 μg to or to about 250 μg, is or is about 0.1 μg to or to about 200 μg, is or is about 0.1 μg to or to about 150 μg, is or is about 0.1 μg to or to about 100 μg, is or is about 0.1 μg to or to about 75 μg, is or is about 0.1 μg to or to about 50 μg, is or is about 0.1 μg to or to about 25 μg, is or is about 0.1 μg to or to about 20 μg, is or is about 0.1 μg to or to about 15 μg, is or is about 1 μg to or to about 250 μg, is or is about 1 μg to or to about 200 μg, is or is about 1 μg to or to about 150 μg, is or is about 1 μg to or to about 100 μg, is or is about 1 μg to or to about 75 μg, is or is about 1 μg to or to about 50 μg, is or is about 1 μg to or to about 25 μg, is or is about 1 μg to or to about 20 μg, is or is about 1 μg to or to about 15 μg, is or is about 5 μg to or to about 250 μg, is or is about 5 μg to or to about 200 μg, is or is about 5 μg to or to about 150 μg, is or is about 5 μg to or to about 100 μg, is or is about 5 μg to or to about 75 μg, is or is about 5 μg to or to about 50 μg, is or is about 5 μg to or to about 25 μg, is or is about 5 μg to or to about 20 μg, is or is about 5 μg to or to about 15 μg, is or is about 10 μg to or to about 250 μg, is or is about 10 μg to or to about 200 μg, is or is about 10 μg to or to about 150 μg, is or is about 10 μg to or to about 100 μg, is or is about 10 μg to or to about 75 μg, is or is about 10 μg to or to about 50 μg, is or is about 10 μg to or to about 25 μg, or is or is about 10 μg to or to about 20 μg, of the antibody or antigen-binding fragment thereof per kg subject body mass.

In some embodiments, an administered dose of the pharmaceutical composition is or is about 1 μg to or to about 100 μg of the antibody or antigen-binding fragment thereof per kg subject body mass. In some embodiments, an administered dose of the pharmaceutical composition is or is about 1 μg to or to about 75 μg of the antibody or antigen-binding fragment thereof per kg subject body mass. In some embodiments, an administered dose of the pharmaceutical composition is or is about 1 μg to or to about 50 μg of the antibody or antigen-binding fragment thereof per kg subject body mass. In some embodiments, an administered dose of the pharmaceutical composition is or is about 1 μg to or to about 40 μg of the antibody or antigen-binding fragment thereof per kg subject body mass. In some embodiments, an administered dose of the pharmaceutical composition is or is about 1 μg to or to about 30 μg of the antibody or antigen-binding fragment thereof per kg subject body mass. In some embodiments, an administered dose of the pharmaceutical composition is or is about 1 μg to or to about 25 μg of the antibody or antigen-binding fragment thereof per kg subject body mass. In some embodiments, an administered dose of the pharmaceutical composition is or is about 1 μg to or to about 20 μg of the antibody or antigen-binding fragment thereof per kg subject body mass. In some embodiments, an administered dose of the pharmaceutical composition is or is about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, or 25 μg of the antibody or antigen-binding fragment thereof per kg subject body mass.

In some embodiments, an administered dose of the pharmaceutical composition is or is about 3 mg to or to about 1800 mg, is or is about 3 mg to or to about 1600 mg, is or is about 3 mg to or to about 1400 mg, is or is about 3 mg to or to about 1200 mg, is or is about 3 mg to or to about 1000 mg, is or is about 3 mg to or to about 900 mg, is or is about 3 mg to or to about 800 mg, is or is about 3 mg to or to about 700 mg, is or is about 3 mg to or to about 600 mg, is or is about 3 mg to or to about 500 mg, is or is about 3 mg to or to about 400 mg, is or is about 3 mg to or to about 300 mg, is or is about 3 mg to or to about 200 mg, is or is about 3 mg to or to about 100 mg, is or is about 3 mg to or to about 50 mg, is or is about 3 mg to or to about 10 mg, is or is about 3 mg to or to about 5 mg, is or is about 10 mg to or to about 1800 mg, is or is about 10 mg to or to about 1600 mg, is or is about 10 mg to or to about 1400 mg, is or is about 10 mg to or to about 1200 mg, is or is about 10 mg to or to about 1000 mg, is or is about 10 mg to or to about 900 mg, is or is about 10 mg to or to about 800 mg, is or is about 10 mg to or to about 700 mg, is or is about 10 mg to or to about 600 mg, is or is about 10 mg to or to about 500 mg, is or is about 10 mg to or to about 400 mg, is or is about 10 mg to or to about 300 mg, is or is about 10 mg to or to about 200 mg, is or is about 10 mg to or to about 100 mg, is or is about 10 mg to or to about 50 mg, is or is about 50 mg to or to about 1800 mg, is or is about 50 mg to or to about 1600 mg, is or is about 50 mg to or to about 1400 mg, is or is about 50 mg to or to about 1200 mg, is or is about 50 mg to or to about 1000 mg, is or is about 50 mg to or to about 900 mg, is or is about 50 mg to or to about 800 mg, is or is about 50 mg to or to about 700 mg, is or is about 50 mg to or to about 600 mg, is or is about 50 mg to or to about 500 mg, is or is about 50 mg to or to about 400 mg, is or is about 50 mg to or to about 300 mg, is or is about 50 mg to or to about 200 mg, is or is about 50 mg to or to about 100 mg, is or is about 100 mg to or to about 1800 mg, is or is about 100 mg to or to about 1600 mg, is or is about 100 mg to or to about 1400 mg, is or is about 100 mg to or to about 1200 mg, is or is about 100 mg to or to about 1000 mg, is or is about 100 mg to or to about 900 mg, is or is about 100 mg to or to about 800 mg, is or is about 100 mg to or to about 700 mg, is or is about 100 mg to or to about 600 mg, is or is about 100 mg to or to about 500 mg, is or is about 100 mg to or to about 400 mg, is or is about 100 mg to or to about 300 mg, is or is about 100 mg to or to about 200 mg, is or is about 500 mg to or to about 1800 mg, is or is about 500 mg to or to about 1600 mg, is or is about 500 mg to or to about 1400 mg, is or is about 500 mg to or to about 1200 mg, is or is about 500 mg to or to about 1000 mg, is or is about 500 mg to or to about 900 mg, is or is about 500 mg to or to about 800 mg, is or is about 500 mg to or to about 700 mg, is or is about 500 mg to or to about 600 mg, is or is about 800 mg to or to about 1800 mg, is or is about 800 mg to or to about 1600 mg, is or is about 800 mg to or to about 1400 mg, is or is about 800 mg to or to about 1200 mg, is or is about 800 mg to or to about 1000 mg, is or is about 800 mg to or to about 900 mg, is or is about 900 mg to or to about 1800 mg, is or is about 900 mg to or to about 1600 mg, is or is about 900 mg to or to about 1400 mg, is or is about 900 mg to or to about 1200 mg, is or is about 900 mg to or to about 1000 mg, is or is about 1000 mg to or to about 1800 mg, is or is about 1000 mg to or to about 1600 mg, is or is about 1000 mg to or to about 1400 mg, is or is about 1000 mg to or to about 1200 mg, is or is about 1200 mg to or to about 1800 mg, is or is about 1200 mg to or to about 1600 mg, is or is about 1200 mg to or to about 1400 mg, is or is about 1400 mg to or to about 1800 mg, is or is about 1400 mg to or to about 1600 mg, is or is about 1600 mg to or to about 1800 mg, of the antibody or antigen-binding fragment thereof.

In some embodiments, an administered dose of the pharmaceutical composition is or is about 3 mg to or to about 1800 mg of the antibody or antigen-binding fragment thereof. In some embodiments, an administered dose of the pharmaceutical composition is or is about 3 mg, 5 mg, 10 mg, 50 mg, 100 mg, 200 mg, 300 mg, 400 mg, 500 mg, 600 mg, 700 mg, 800 mg, 900 mg, 1000 mg, 1200 mg, 1400 mg, 1600 mg, or 1800 mg of the antibody or antigen-binding fragment thereof.

In some embodiments, the pharmaceutical composition is administered intravenously (IV). In some embodiments, an intravenously administered dose of the pharmaceutical composition is or is about 3 mg to or to about 1800 mg of the antibody or antigen-binding fragment thereof. In some embodiments, an intravenously administered dose of the pharmaceutical composition is or is about 3 mg, 5 mg, 10 mg, 50 mg, 100 mg, 200 mg, 300 mg, 400 mg, 500 mg, 600 mg, 700 mg, 800 mg, 900 mg, 1000 mg, 1200 mg, 1400 mg, 1600 mg, or 1800 mg of the antibody or antigen-binding fragment thereof.

In some embodiments, the pharmaceutical composition is administered subcutaneously (SC). In some embodiments, a subcutaneously administered dose of the pharmaceutical composition is at or greater than 900 mg of the antibody or antigen-binding fragment thereof. In some embodiments, a subcutaneously administered dose of the pharmaceutical composition is at or is greater than about 900 mg, 1000 mg, 1200 mg, 1400 mg, 1600 mg, or 1800 mg of the antibody or antigen-binding fragment thereof.

In some embodiments, the pharmaceutical composition is administered intravenously (IV) for one or more initial doses. In some embodiments, the pharmaceutical composition is administered subcutaneously (SC) for one or more maintenance doses following the one or more initial doses. In some embodiments, the pharmaceutical composition is administered intravenously (IV) for one or more initial doses, and is administered subcutaneously (SC) for one or more maintenance doses following the one or more initial doses. In some embodiments, the pharmaceutical composition is administered intravenously (IV) for one or more initial doses, wherein the one or more initial doses each is from is or is about 3 mg to or to about 1800 mg of the antibody or antigen-binding fragment thereof. In some embodiments, following the one or more initial doses, the pharmaceutical composition is administered subcutaneously (SC) for one or more maintenance doses following the one or more initial doses, wherein the one or more maintenance doses each is or is greater than 900 mg of the antibody or antigen-binding fragment thereof. In some embodiments, the one or more initial doses is at least one, two, three, four, five, or more doses. In some embodiments, the one or more maintenance doses are administered indefinitely, e.g., chronically.

In some embodiments, a therapeutically effective amount of the composition of the present disclosure is administered. The optimal dosage and treatment regime for a particular patient can readily be determined and/or adjusted by one skilled in the art of medicine, such as based on the weight, age, health, and/or sex of the subject, and/or by the severity of the disease or disorder, and/or by monitoring the subject for signs of the disease or disorder and adjusting the treatment accordingly.

In some embodiments, the anti-IL-18Rβ antibody or antigen-binding fragment thereof, multi-specific antibody, conjugate, or composition, e.g., pharmaceutical composition, can be administered alone or in combination with other modes of treatment, such as other agents suitable for treatment of the disease or disorder. They can be provided before, substantially contemporaneous with, or after other modes of treatment. In some embodiments, they are administered concurrently (e.g., simultaneously or sequentially). In some embodiments, the anti-IL-18Rβ antibody or antigen-binding fragment thereof, multi-specific antibody, conjugate, or composition, e.g., pharmaceutical composition, is administered concurrently with other modes of treatment, such as other agents suitable for treatment of the disease or disorder (e.g., one or more additional therapeutic agents). As used herein, the term “concurrently” in the context of the anti-IL-18Rβ antibody or antigen-binding fragment thereof, multi-specific antibody, conjugate, or composition, e.g., pharmaceutical composition, means that the one or more additional therapeutic agents or treatments and the anti-IL-18Rβ antibody or antigen-binding fragment thereof, multi-specific antibody, conjugate, or composition, e.g., pharmaceutical composition are administered such that the anti-IL-18Rβ antibody or antigen-binding fragment thereof, multi-specific antibody, conjugate, or composition and the one or more additional therapeutic agents are present in the body at the same time. Typically, the anti-IL-18Rβ antibody or antigen-binding fragment thereof, multi-specific antibody, conjugate, or composition and the one or more additional therapeutic agents are administered such that the combination of agents administered is therapeutically effective. In some such embodiments, the anti-IL-18Rβ antibody or antigen-binding fragment thereof, multi-specific antibody, conjugate, or composition and the one or more additional therapeutic agents are administered such that they are present in the body in jointly therapeutically effective amounts. In some embodiments, the anti-IL-18Rβ antibody or antigen-binding fragment thereof, multi-specific antibody, conjugate, or composition and one or more (e.g., each of) the one or more additional therapeutic agents are administered at the same time (e.g., administered simultaneously in a single composition or substantially contemporaneously in separate compositions). In some embodiments, the anti-IL-18Rβ antibody or antigen-binding fragment thereof, multi-specific antibody, conjugate, or composition and one or more (e.g., each of) the one or more additional therapeutic agents are administered sequentially. As used herein “sequentially” means that the anti-IL-18Rβ antibody or antigen-binding fragment thereof, multi-specific antibody, conjugate, or composition and one or more (e.g., each of) the one or more additional therapeutic agents are administered before or after each other and not simultaneously or substantially contemporaneously. In some embodiments, the anti-IL-18Rβ antibody or antigen-binding fragment thereof, multi-specific antibody, conjugate, or composition and one or more (e.g., each of) the one or more additional therapeutic agents are administered within 4 hours, 6 hours, 12 hours, 1 day, 2 days, 1 week, 2 weeks, 3 weeks, 4 weeks, 6 weeks, 1 month or 2 months of each other.

In some embodiments, the anti-IL-18Rβ antibody or antigen-binding fragment thereof, multi-specific antibody, conjugate, or composition, e.g., pharmaceutical composition, is present in the body at the same time as other agents suitable for treatment of the disease or disorder (e.g., one or more additional therapeutic agents), such that they are present in the body at the same time in amounts that are therapeutically effective (e.g., in amounts that are jointly therapeutically effective).

Accordingly, in some embodiments, provided herein is a method of treatment that comprises administering any anti-IL-18Rβ antibody or antigen-binding fragment thereof described herein, any multi-specific antibody described herein, any conjugate described herein, or any composition, e.g., pharmaceutical composition, described herein; and one or more additional therapeutic agents, to a subject having a disease or disorder. The disease or disorder can be any disease or disorder described herein.

Also provided herein, in some embodiments, is a method of treatment that comprises administering any anti-IL-18Rβ antibody or antigen-binding fragment thereof described herein, any multi-specific antibody described herein, any conjugate described herein, or any composition, e.g., pharmaceutical composition, described herein; and one or more additional therapeutic agents, to a subject having inflammatory bowel disease, e.g., Crohn's disease or ulcerative colitis.

The one or more additional therapeutic agents can, in some embodiments, be any therapeutic agent suitable for treatment of the disease or disorder, e.g., inflammatory bowel disease. In some embodiments, the one or more additional therapeutic agents is an aminosalicylate, a corticosteroid (e.g., prednisone, methylprednisolone, hydrocortisone), a thiopurine, a methotrexate, a tumor necrosis factor (TNF) inhibitor, an α4β7 integrin inhibitor (e.g., vedolizumab), a TNF-α inhibitor (e.g., infliximab, adalimumab, certolizumab pegol, or golimumab), an IL-23 inhibitor (e.g., Risankizumab-rzaa), a dual IL-12 and IL-23 inhibitor (e.g., ustekinumab), a tumor necrosis factor-like cytokine 1A (TL1A) inhibitor (e.g., PRA023, PF-06480605, RVT-3101, or TEV-48574), an IL-2-CD25 fusion protein, a bispecific anti-TL1A/anti-TNF-α binding protein, an E-type prostanoid receptor 4 (EP4) agonist, a TYK2 inhibitor (e.g., deucravacitinib), a sphingosine-1-phosphate receptor (S1PR) agonist (e.g., ozanimod), a Janus kinase (JAK) inhibitor (e.g., abrocitinib, baricitinib, delgocitinib, fedratinib, filgotinib, oclacitinib, pacritinib, peficitinib, ruxolitinib, tofacitinib, or upadacitinib), an interkeukin-1 receptor associated kinase 4 (IRAK-4) inhibitor (e.g., KT-474), a toll-like receptor 7 and 8 (TLR7/8) inhibitor (e.g., afimetoran), or an IL-22 inhibitor. In some embodiments, the one or more additional therapeutic agents is a beta agonist, muscarinic antagonist, systemic steroid, azithromycin, roflumilast, or dupilumab. In some embodiments, the one or more additional therapeutic agents is a topical mesalamine, a steroid (e.g., a topical steroid, an oral steroid, and an intravenous steroid), an 5-ASA agent (e.g., mesalamine and sulfasalazine), an integrin inhibitor, a surgical intervention (e.g., bullectomy), an acetaminophen, a COX-2 selective nonsteroidal anti-inflammatory drug (NSAID), a salicylate, a proprionic acid, an oxicam, an acetic acid, a fenamate, an IL-6 targeting agent, an anti-CD20 antibody, a CTLA4 inhibitor, an IL-17 inhibitor, PDE4 inhibitor, hydroxychloroquine, sulfasalazine, methotrexate, leflunomide, minocycline, azathioprine, an emollient, UV light therapy, tralokinumab, antibiotics, a phosphodiesterase 3 & 4 inhibitor, or a minimally invasive intervention.

Also provided herein, in some embodiments, is a method of treatment that comprises administering any anti-IL-18Rβ antibody or antigen-binding fragment thereof described herein, any multi-specific antibody described herein, any conjugate described herein, or any composition, e.g., pharmaceutical composition, described herein; and one or more additional therapeutic agents, to a subject having inflammatory bowel disease, e.g., Crohn's disease or ulcerative colitis, wherein the one or more additional therapeutic agents is an aminosalicylate, a corticosteroid (e.g., prednisone, methylprednisolone, hydrocortisone), a thiopurine, a methotrexate, a tumor necrosis factor (TNF) inhibitor, an α4β7 integrin inhibitor (e.g., vedolizumab), a TNF-α inhibitor (e.g., infliximab, adalimumab, certolizumab pegol, or golimumab), an IL-23 inhibitor (e.g., Risankizumab-rzaa), a dual IL-12 and IL-23 inhibitor (e.g., ustekinumab), a tumor necrosis factor-like cytokine 1A (TL1A) inhibitor (e.g., PRA023, PF-06480605, RVT-3101, or TEV-48574), an IL-2-CD25 fusion protein, a bispecific anti-TL1A/anti-TNF-α binding protein, an E-type prostanoid receptor 4 (EP4) agonist, a TYK2 inhibitor (e.g., deucravacitinib), a sphingosine-1-phosphate receptor (S1PR) agonist (e.g., ozanimod), a Janus kinase (JAK) inhibitor (e.g., abrocitinib, baricitinib, delgocitinib, fedratinib, filgotinib, oclacitinib, pacritinib, peficitinib, ruxolitinib, tofacitinib, or upadacitinib), an interkeukin-1 receptor associated kinase 4 (IRAK-4) inhibitor (e.g., KT-474, BAY1834845 (zabedosertib), BAY1830839, emavusertib, GLPG2534), a toll-like receptor 7 and 8 (TLR7/8) inhibitor (e.g., afimetoran), an IL-22 inhibitor, a beta agonist, muscarinic antagonist, systemic steroid, azithromycin, roflumilast, or dupilumab.

In some embodiments, the one or more additional therapeutic agents comprises, consists essentially of, or consists of an aminosalicylate.

In some embodiments, the one or more additional therapeutic agents comprises, consists essentially of, or consists of a corticosteroid. In some embodiments, the corticosteroid is prednisone, methylprednisolone, or hydrocortisone.

In some embodiments, the one or more additional therapeutic agents comprises, consists essentially of, or consists of a thiopurine.

In some embodiments, the one or more additional therapeutic agents comprises, consists essentially of, or consists of a methotrexate.

In some embodiments, the one or more additional therapeutic agents comprises, consists essentially of, or consists of a TNF inhibitor. In some embodiments, the TNF inhibitor is an anti-TNF antibody or antigen-binding fragment thereof.

In some embodiments, the one or more additional therapeutic agents comprises, consists essentially of, or consists of an additional therapeutic agent as disclosed in, e.g., Cai Z, Wang S and Li J (2021), Treatment of Inflammatory Bowel Disease: A Comprehensive Review, Front. Med. 8:765474.

In some embodiments, the one or more additional therapeutic agents comprises, consists essentially of, or consists of an α4β7 integrin inhibitor. In some embodiments, the α4β7 integrin inhibitor is an anti-α4β7 integrin antibody or antigen-binding fragment thereof. In some embodiments, the anti-α4β7 integrin antibody is vedolizumab or an antigen-binding fragment thereof. In some embodiments, the anti-α4β7 integrin antibody is vedolizumab (ENTYVIO®).

In some embodiments, the one or more additional therapeutic agents comprises, consists essentially of, or consists of an TNF-α inhibitor. In some embodiments, the TNF-α inhibitor is an anti-TNF-α antibody or antigen-binding fragment thereof. In some embodiments, the anti-TNF-α antibody or antigen-binding fragment thereof is infliximab, adalimumab, certolizumab pegol, or golimumab, or an antigen-binding fragment of any of the foregoing. In some embodiments, the anti-TNF-α antibody or antigen-binding fragment thereof is infliximab or an antigen-binding fragment thereof. In some embodiments, the anti-TNF-α antibody or antigen-binding fragment thereof is infliximab. Infliximab is marketed as, e.g., REMICADE®, or as a biosimilar thereof, such as Inflectra®, Renflexis®, or Avsola®. In some embodiments, the anti-TNF-α antibody or antigen-binding fragment thereof is adalimumab or an antigen-binding fragment thereof. In some embodiments, the anti-TNF-α antibody or antigen-binding fragment thereof is adalimumab. Adalimumab is marketed as, e.g., Humira®, or as a biosimilar thereof, such as Amgevita®, Cyltezo®, Hefia®, Hulio®, Hyrimoz®, Idacio®, Imraldi®, Kromeya®, Solymbic, or Yuflyma®. In some embodiments, the anti-TNF-α antibody or antigen-binding fragment thereof is certolizumab pegol. Certolizumab pegol is marketed as, e.g., CIMZIA®. In some embodiments, the anti-TNF-α antibody or antigen-binding fragment thereof is golimumab or an antigen-binding fragment thereof. In some embodiments, the anti-TNF-α antibody or antigen-binding fragment thereof is golimumab. Golimumab is marketed as, e.g., SIMPONI ARIA®.

In some embodiments, the one or more additional therapeutic agents comprises, consists essentially of, or consists of an IL-23 inhibitor. In some embodiments, the IL-23 inhibitor is an anti-IL-23 antibody or antigen-binding fragment thereof. In some embodiments, the anti-IL-23 antibody or antigen-binding fragment thereof is risankizumab-rzaa or an antigen-binding fragment thereof. In some embodiments, the anti-IL-23 antibody or antigen-binding fragment thereof is risankizumab-rzaa. Risankizumab-rzaa is marketed as, e.g., SKYRIZI®. In some embodiments, the anti-IL-23 antibody or antigen-binding fragment thereof is mirikizumab-mrkz. Mirikizumab-mrkz is marketed as, e.g., OMVOH®. In some embodiments, the anti-IL-23 antibody or antigen-binding fragment thereof is guselkumab. Guselkumab is marketed as, e.g., TREMFYA®.

In some embodiments, the one or more additional therapeutic agents comprises, consists essentially of, or consists of a dual IL-12 and IL-23 inhibitor. A dual IL-12 and IL-23 inhibitor is an inhibitor that inhibits both IL-12 and IL-23, such as by targeting a subunit common to both cytokines, e.g., p40. In some embodiments, the dual IL-12 and IL-23 inhibitor is a p40 inhibitor. p40 is a subunit that is common to both IL-12 and IL-23, thereby allowing for dual inhibition of both IL-12 and IL-23 by an inhibitor of p40. Accordingly, in some embodiments, the one or more additional therapeutic agents comprises, consists essentially of, or consists of a p40 inhibitor. In some embodiments, the dual IL-12 and IL-23 inhibitor or the p40 inhibitor is an anti-p40 antibody or antigen-binding fragment thereof. In some embodiments, anti-p40 antibody or antigen-binding fragment thereof is ustekinumab or an antigen-binding fragment thereof. In some embodiments, anti-p40 antibody or antigen-binding fragment thereof is ustekinumab. Ustekinumab is marketed as, e.g., STELARA®.

In some embodiments, the one or more additional therapeutic agents comprises, consists essentially of, or consists of a TL1A inhibitor. In some embodiments, the TL1A inhibitor is an anti-TL1A antibody or antigen-binding fragment thereof. Exemplary anti-TL1A antibodies or antigen-binding fragments thereof include those as disclosed in, e.g., WO2012064682A1, U.S. Pat. No. 9,556,277B2, WO2015073580A1. WO2014106602A1, WO2017049024A1, WO2017196663A1, U.S. Pat. No. 10,968,279B2, U.S. Pat. No. 11,767,364B2, WO2019209995A2, WO2021206577A2, WO2018081074A1, WO2019/20995A2, U.S. Pat. No. 11,292,848B2, WO2023009545A1, and Clarke A W, et al. Mabs, 2018 May/June; 10 (4): 664-677, each of which is incorporated by reference herein in its entirety and for all purposes. In some embodiments, the anti-TL1A antibody or antigen-binding fragment thereof is PRA023, PF-06480605, RVT-3101, or TEV-48574, or an antigen-binding fragment of any of the foregoing. In some embodiments, the anti-TL1A antibody or antigen-binding fragment thereof is PRA023 or an antigen-binding fragment thereof. In some embodiments, the anti-TL1A antibody or antigen-binding fragment thereof is PRA023. In some embodiments, the anti-TL1A antibody or antigen-binding fragment thereof is PF-06480605 or an antigen-binding fragment thereof. In some embodiments, the anti-TL1A antibody or antigen-binding fragment thereof is PF-06480605. In some embodiments, the anti-TL1A antibody or antigen-binding fragment thereof is RVT-3101 or an antigen-binding fragment thereof. In some embodiments, the anti-TL1A antibody or antigen-binding fragment thereof is RVT-3101. In some embodiments, the anti-TL1A antibody or antigen-binding fragment thereof is TEV-48574 or an antigen-binding fragment thereof. In some embodiments, the anti-TL1A antibody or antigen-binding fragment thereof is TEV-48574.

In some embodiments, the one or more additional therapeutic agents comprises, consists essentially of, or consists of a TL1A inhibitor, such as any of the TL1A inhibitors described herein, and an IL-23 inhibitor, such as any IL-23 inhibitor described herein. In some embodiments, the one or more additional therapeutic agents comprises, consists essentially of, or consists of a TL1A inhibitor and an IL-23 inhibitor, such as disclosed in WO2023133538A2, which is incorporated by reference herein in its entirety and for all purposes.

In some embodiments, the one or more additional therapeutic agents comprises, consists essentially of, or consists of an IL-2-CD25 fusion protein. In some embodiments, the IL-2-CD25 fusion protein comprises a first polypeptide comprising IL-2, and a second polypeptide comprising an extracellular domain of CD25 (also known as IL-2 receptor alpha), wherein the first polypeptide is fused to the second polypeptide. In some embodiments, the IL-2 and/or the CD25 of the IL-2-CD25 fusion protein comprises at least one fewer glycosylation sites compared to native IL-2 or native CD25. In some embodiments, the IL-2-CD25 fusion protein is an IL-2-CD25 fusion protein as disclosed in U.S. Pat. No. 10,787,494 or U.S. Pat. No. 11,359,000, each of which is incorporated by reference herein in its entirety and for all purposes.

In some embodiments, the one or more additional therapeutic agents comprises, consists essentially of, or consists of a bispecific anti-TL1A/anti-TNF-α binding protein. In some embodiments, the bispecific anti-TL1A/anti-TNF-α binding protein, is a bispecific anti-TL1A/anti-TNF-α antibody, or antigen-binding fragment thereof. In some embodiments, the bispecific anti-TL1A/anti-TNF-α antibody, or antigen-binding fragment thereof, is a bispecific anti-TL1A/anti-TNF-α antibody, or antigen-binding fragment thereof, as disclosed in WO2017106383A1, which is incorporated by reference herein in its entirety and for all purposes.

In some embodiments, the one or more additional therapeutic agents comprises, consists essentially of, or consists of an EP4 agonist. Exemplary EP4 agonists include those as disclosed in, e.g., Konya et al., Pharmacol. Ther., 2013, 138 (3): 485-502; and Markovic et al., Drug Discovery Today, 2017, 22 (1): 57-71, each of which is incorporated by reference herein in its entirety and for all purposes.

In some embodiments, the one or more additional therapeutic agents comprises, consists essentially of, or consists of a TYK2 inhibitor. Exemplary TYK2 inhibitors include those as disclosed in, e.g., WO2012074661 and WO2019103952, each of which is incorporated by reference herein in its entirety and for all purposes. In some embodiments, the TYK2 inhibitor is deucravacitinib.

In some embodiments, the one or more additional therapeutic agents comprises, consists essentially of, or consists of an S1PR agonist. In some embodiments, the S1PR agonist is an S1PR agonist as disclosed in, e.g., WO2009151529A or WO2011060392A1, each of which is incorporated by reference herein in its entirety and for all purposes. In some embodiments, the S1PR agonist disclosed in WO2009151529A and WO2011060392A1 is ozanimod. Accordingly, in some embodiments, the S1PR agonist is ozanimod.

In some embodiments, the one or more additional therapeutic agents comprises, consists essentially of, or consists of a JAK inhibitor. Exemplary JAK inhibitors include those as disclosed in, e.g., Shawky et al., Pharmaceutics, 2022 May; 14 (5): 1001, which is incorporated by reference herein in its entirety and for all purposes. In some embodiments, the JAK inhibitor is an inhibitor of one or more of JAK1, JAK2, JAK3, or TYK2. In some embodiments, the JAK inhibitor is an inhibitor of JAK1. In some embodiments, the JAK inhibitor is an inhibitor of JAK2. In some embodiments, the JAK inhibitor is an inhibitor of JAK3. In some embodiments, the JAK inhibitor is an inhibitor of TYK2. In some embodiments, the JAK inhibitor is an inhibitor of JAK1 and JAK2. In some embodiments, the JAK inhibitor is an inhibitor of JAK1, JAK2, and JAK3. In some embodiments, the JAK inhibitor is an inhibitor of JAK1, JAK2, JAK3, and TYK2. In some embodiments, the JAK inhibitor is abrocitinib, baricitinib, delgocitinib, fedratinib, filgotinib, oclacitinib, pacritinib, peficitinib, ruxolitinib, tofacitinib, or upadacitinib. In some embodiments, the JAK inhibitor is upadacitinib.

In some embodiments, the one or more additional therapeutic agents comprises, consists essentially of, or consists of an IRAK-4 inhibitor. Exemplary IRAK-4 inhibitors include those as disclosed in, e.g., WO2023201274, WO2023147594A2, WO2019133531, WO2020113233, WO2020264490, WO2021127283, and WO2021011868, each of which is incorporated by reference herein in its entirety and for all purposes. In some embodiments, the IRAK-4 inhibitor is KT-474 and/or is an IRAK-4 inhibitor disclosed in WO2023201274 or WO2023147594A2. Accordingly, in some embodiments, the IRAK-4 inhibitor is KT-474.

In some embodiments, the one or more additional therapeutic agents comprises, consists essentially of, or consists of a TLR7/8 inhibitor. Exemplary TLR7/8 inhibitors include those as disclosed in, e.g., WO2018/005586, WO2021252718, and WO2021262561, each of which is incorporated by reference herein in its entirety and for all purposes. In some embodiments, the TLR7/8 inhibitor disclosed in WO2018/005586 is afimetoran (BMS-986256). Accordingly, in some embodiments, the TLR7/8 inhibitor is afimetoran (BMS-986256).

In some embodiments, the one or more additional therapeutic agents comprises, consists essentially of, or consists of an IL-22 inhibitor. In some embodiments, the IL-22 inhibitor is an anti-IL-22 antibody or antigen-binding fragment thereof.

In some embodiments, the one or more additional therapeutic agents comprises, consists essentially of, or consists of one or more anti-inflammatory agents and/or one or more immune system suppressors, including any of those as disclosed herein.

In some embodiments, the one or more additional therapeutic agents comprises, consists essentially of, or consists of an IL-10 targeting agent. In some embodiments, the IL-10 targeting agent is engineered to selectively target myeloid activation without stimulating and/or activating T cell or B cells. In some embodiments, the IL-10 targeting agent is an IL-10 fusion protein. Exemplary IL-10 fusion proteins include those as disclosed in, e.g., WO2012/045334, WO2014/023673, WO2015/117930, WO2020/192648, WO2022/124950 and WO2023/078245, each of which is incorporated herein by reference herein in its entirety and for all purposes.

In some embodiments, the one or more additional therapeutic agents comprises, consists essentially of, or consists of a B cell targeting T cell engager (TCE).

In some embodiments, the B cell targeting T cell engager (TCE) is a CD19×CD3 bispecific antibody or antigen-binding fragment thereof. Exemplary CD19×CD3 bispecific antibodies or antigen-binding fragments thereof include those as disclosed in, e.g., WO1995/004440, WO2005/052004, WO2007/068354, WO2010/052013, WO2010/052014, WO2011/051307, U.S. Pat. Nos. 7,112,324, 7,575,923, U.S. Pat. No. 10,000,574, WO2006/114115, U.S. Pat. Nos. 10,071,158, 10,576,149, US2021/015917, US2023/310597, U.S. Pat. Nos. 8,709,421, 11,154,617, 8,007,796, 8,007,796, 11,597,766, 10,662,243, 8,840,888, WO2021/255155, U.S. Pat. No. 11,780,920, and WO2024/094741, each of which is incorporated by reference herein in its entirety and for all purposes. In some embodiments, the CD19×CD3 bispecific antibody or antigen-binding fragment thereof disclosed in WO1995/004440, WO2005/052004, WO2007/068354, WO2010/052013, WO2010/052014, WO2011/051307, U.S. Pat. Nos. 7,112,324, 7,575,923, 10,000,574, WO2006/114115, U.S. Pat. Nos. 10,071,158, 10,576,149, 8,709,421, 11,154,617, 8,007,796, 8,007,796, 11,597,766, 10,662,243, and 8,840,888 is blinatumomab. Accordingly, in some embodiments, the CD19×CD3 bispecific antibody or antigen-binding fragment thereof is blinatumomab. Blinatumomab is marketed as, e.g., BLINCYTO®. In some embodiments, the CD19×CD3 bispecific antibody or antigen-binding fragment thereof disclosed in WO2021/255155, U.S. Pat. No. 11,780,920, and WO2024/094741 is RO7507062. Accordingly, in some embodiments, the CD19×CD3 bispecific antibody or antigen-binding fragment thereof is RO7507062.

In some embodiments, the B cell targeting T cell engager (TCE) is a BCMA×CD3 bispecific antibody or antigen-binding fragment thereof. Exemplary BCMA×CD3 bispecific antibodies or antigen-binding fragments thereof include those as disclosed in, e.g., WO 2017/021450, which is incorporated by reference herein in its entirety and for all purposes. In some embodiments, the BCMA×CD3 bispecific antibody or antigen-binding fragment thereof disclosed in WO 2017/021450 is alnuctamab (BMS-986349). Accordingly, in some embodiments, the BCMA×CD3 bispecific antibody or antigen-binding fragment thereof is alnuctamab (BMS-986349).

In some embodiments, the B cell targeting T cell engager (TCE) is a CD20×CD3 bispecific antibody or antigen-binding fragment thereof. Exemplary CD20×CD3 bispecific antibodies or antigen-binding fragments thereof include those as disclosed in, e.g., WO2015/095392, U.S. Pat. Nos. 10,174,124, 10,640,572, 10,865,251, 11,186,650, 11,530,275, 11,732,054, WO2016/141303, U.S. Pat. No. 10,787,520, and WO2024/073700, each of which is incorporated by reference herein in its entirety and for all purposes. In some embodiments, the CD20×CD3 bispecific antibody or antigen-binding fragment thereof disclosed in WO2015/095392, U.S. Pat. Nos. 10,174,124, 10,640,572, 10,865,251, 11,186,650, 11,530,275, and 11,732,054 is mosunetuzumab. Accordingly, in some embodiments, the CD20 ×CD3 bispecific antibody is mosunetuzumab. In some embodiments, the CD20×CD3 bispecific antibody or antigen-binding fragment thereof disclosed in, e.g., WO2016/141303, U.S. Pat. No. 10,787,520, and WO2024/073700 is imvotamab. Accordingly, in some embodiments, the CD20×CD3 bispecific antibody or antigen-binding fragment thereof is imvotamab.

In some embodiments, the B cell targeting T cell engager (TCE) is a CD19×CD3×CD2 trispecific antibody or antigen-binding fragment thereof. Exemplary CD19×CD3×CD2 trispecific antibodies or antigen-binding fragments thereof are disclosed in, e.g., WO2023/199235, which is incorporated by reference herein in its entirety and for all purposes. In some embodiments, the CD19 ×CD3×CD2 trispecific antibody or antigen-binding fragment thereof disclosed in WO2023/199235 is PIT565. Accordingly, in some embodiments, the CD19×CD3×CD2 trispecific antibody or antigen-binding fragment thereof is PIT565.

In some embodiments, the one or more additional therapeutic agents comprises, consists essentially of, or consists of a CTLA4-Ig. Exemplary CTLA4-Ig include those as disclosed in, e.g., U.S. Pat. No. 5,851,795, which is incorporated by reference herein in its entirety and for all purposes. In some embodiments, the CTLA4-Ig disclosed in U.S. Pat. No. 5,851,795 is abatacept. Accordingly, in some embodiments, the CTLA4-Ig is abatacept. Abatacept is marketed as, e.g., ORENCIA®.

In some embodiments, the one or more additional therapeutic agents comprises, consists essentially of, or consists of a dual IL-4 and IL-23 inhibitor. Exemplary dual IL-4 and IL-23 inhibitors include those as disclosed in, e.g., WO2019/002340, U.S. Pat. Nos. 7,186,809, 7,465,450, 8,679,487, 9,587,026, WO2008/054606, WO2010/053751, U.S. Pat. Nos. 7,605,237, 7,608,693, 7,608,693, 7,794,717, 8,075,887, 8,092,802, 8,337,839, 8,338,135, 8,735,095, WO2012/047954, U.S. Pat. Nos. 10,435,473, 11,059,896, 11,926,670, 8,945,559, 9,238,692, WO2014/031610, U.S. Pat. Nos. 11,845,800, 9,574,004, WO2014/039461, WO2014/205365, U.S. Pat. No. 10,059,771, WO2015/006571, U.S. Pat. Nos. 10,730,948, 11,421,036, 9,290,574, WO2015/127229, U.S. Pat. No. 10,137,193, WO2018/045130, U.S. Pat. No. 11,771,743, WO2018/057776, U.S. Pat. Nos. 10,485,844, and 11,167,004, each of which is incorporated by reference herein in its entirety and for all purposes. In some embodiments, the dual IL-4 and IL-23 inhibitor disclosed in WO2019/002340, U.S. Pat. Nos. 7,186,809, 7,465,450, 8,679,487, 9,587,026, WO2008/054606, WO2010/053751, U.S. Pat. Nos. 7,605,237, 7,608,693, 7,608,693, 7,794,717, 8,075,887, 8,092,802, 8,337,839, 8,338,135, 8,735,095, WO2012/047954, U.S. Pat. Nos. 10,435,473, 11,059,896, 11,926,670, 8,945,559, 9,238,692, WO2014/031610, U.S. Pat. Nos. 11,845,800, 9,574,004, WO2014/039461, WO2014/205365, U.S. Pat. No. 10,059,771, WO2015/006571, U.S. Pat. Nos. 10,730,948, 11,421,036, 9,290,574, WO2015/127229, U.S. Pat. No. 10,137,193, WO2018/045130, U.S. Pat. No. 11,771,743, WO2018/057776, U.S. Pat. Nos. 10,485,844, and 11,167,004 is dupilumab. Accordingly, in some embodiments, the dual IL-4 and IL-23 inhibitor is dupilumab. Dupilumab is marketed as, e.g., DUPIXENT®.

In some embodiments, the one or more additional therapeutic agents comprises, consists essentially of, or consists of an IL-13 targeting agent. In some embodiments, the IL-13 targeting agent is an anti-IL-13 antibody or antigen-binding fragment thereof. Exemplary anti-IL-13 antibodies or antigen-binding fragments thereof include those as disclosed in, e.g., U.S. Pat. No. 7,915,388, WO2005/062967, WO2005/062972. U.S. Pat. No. 11,434,287. U.S. Pat. Nos. 7,674,459, 8,067,199, 8,088,618, 8,318,160, 8,734,797, 8,734,801, 9,067,994, 9,605,065, WO2013/066866, U.S. Pat. Nos. 10,000,562, 10,947,307, WO2015/038888, U.S. Pat. Nos. 10,494,429, 10,597,446, 10,597,447, 10,822,404, 11,667,706, 9,920,120, WO2018/057849, and U.S. Pat. No. 11,434,286, each of which is incorporated by reference herein in its entirety and for all purposes. In some embodiments, the anti-IL-13 antibody or antigen-binding fragment thereof disclosed in U.S. Pat. No. 7,915,388 is cendakimab. Accordingly, in some embodiments, the anti-IL-13 antibody or antigen-binding fragment thereof is cendakimab. In some embodiments, the anti-IL-13 antibody or antigen-binding fragment thereof disclosed in WO2005/062967, WO2005/062972. U.S. Pat. No. 11,434,287. U.S. Pat. Nos. 7,674,459, 8,067,199, 8,088,618, 8,318,160, 8,734,797, 8,734,801, 9,067,994, 9,605,065, WO2013/066866, U.S. Pat. Nos. 10,000,562, 10,947,307, WO2015/038888, U.S. Pat. Nos. 10,494,429, 10,597,446, 10,597,447, 10,822,404, 11,667,706, 9,920,120, WO2018/057849, and U.S. Pat. No. 11,434,286, is lebrikizumab. Accordingly, in some embodiments, the anti-IL-13 antibody or antigen-binding fragment thereof is lebrikizumab. Lebrikizumab is marketed, e.g., EBGLYSS®.

In some embodiments, the one or more additional therapeutic agents comprises, consists essentially of, or consists of a STAT6 inhibitor or degrader. In some embodiments, the STAT6 inhibitor or degrader is a STAT6 inhibitor. In some embodiments, the STAT6 inhibitor or degrader is a STAT6 degrader. Exemplary STAT6 degraders include those as disclosed in, e.g., WO2024/064080, WO2019/140380, U.S. Pat. Nos. 11,485,743, 12,006,329, WO2020/206424, U.S. Pat. Nos. 11,485,750, 11,746,120, and 12,077,555, each of which is incorporated by reference herein in its entirety and for all purposes. In some embodiments, the STAT6 degrader is disclosed in WO2024/064080, WO2019/140380, U.S. Pat. Nos. 11,485,743, 12,006,329, WO2020/206424, U.S. Pat. Nos. 11,485,750, 11,746,120, and 12,077,555 is Kymera KT-621. Accordingly, in some embodiments, the STAT6 degrader is Kymera KT-621.

In some embodiments, the one or more additional therapeutic agents comprises, consists essentially of, or consists of an eIF2b activator. Exemplary eIF2b activators include those as disclosed in, e.g., WO2020/216766, which is incorporated by reference herein in its entirety and for all purposes. In some embodiments, the eIF2b activator is disclosed in WO2020/216766.

In some embodiments, the one or more additional therapeutic agents comprises, consists essentially of, or consists of an IL-5 targeting agent. In some embodiments, the IL-5 targeting agent is an anti-IL-5 antibody or antigen-binding fragment thereof. Exemplary anti-IL-5 antibodies or antigen-binding fragments thereof include those as disclosed in, e.g., WO1993/016184, U.S. Pat. Nos. 5,683,892, 5,693,323, 5,783,184, 5,851,525, 6,129,913, 5,096,071, 6,056,957, 6,451,982, 7,422,742 and 7,141,653, each of which is incorporated by reference herein in its entirety and for all purposes. In some embodiments, the anti-IL-5 antibody or antigen-binding fragment thereof disclosed in WO1993/016184 and U.S. Pat. No. 6,451,982 is reslizumab. Accordingly, in some embodiments, the anti-IL-5 antibody or antigen-binding fragment thereof is reslizumab. Reslizumab is marketed as, e.g., CINQAIR® and CINQAERO®.

In some embodiments, the one or more additional therapeutic agents comprises, consists essentially of, or consists of an IL-33 targeting agent. In some embodiments, the IL-33 targeting agent is an anti-IL-33 antibody or antigen-binding fragment thereof. Exemplary anti-IL-33 antibodies or antigen-binding fragments thereof include those as disclosed in, e.g., WO2014/164959, WO2021/204707, and WO2022/063281, each of which is incorporated by reference herein in its entirety and for all purposes. In some embodiments, the anti-IL-33 antibody or antigen-binding fragment thereof is tozorakimab (AstraZeneca). In some embodiments, the anti-IL-33 antibody or antigen-binding fragment thereof is itepekimab (Regeneron).

In some embodiments, the one or more additional therapeutic agents comprises, consists essentially of, or consists of an IL-31 targeting agent. In some embodiments, the IL-31 targeting agent is an anti-IL-31 antibody or antigen-binding fragment thereof. Exemplary anti-IL-31 antibodies or antigen-binding fragments thereof include those as disclosed in, e.g., WO2006/122079, which is incorporated by reference herein in its entirety and for all purposes. In some embodiments, the anti-IL-31 antibody or antigen-binding fragment thereof is BMS-981164.

In some embodiments, the one or more additional therapeutic agents comprises, consists essentially of, or consists of a thymic stromal lymphopoietin (TSLP) targeting agent. In some embodiments, the TSLP targeting agent is an anti-TSLP antibody or antigen-binding fragment thereof. Exemplary anti-TSLP antibodies or antigen-binding fragments thereof include those as disclosed in, e.g., WO2017/042701, which is incorporated by reference herein in its entirety and for all purposes. In some embodiments, the anti-TSLP antibody or antigen-binding fragment thereof is tezepelumab.

In some embodiments, the one or more additional therapeutic agents comprises, consists essentially of, or consists of an IL-6 targeting agent. In some embodiments, an IL-6 targeting agent is an anti-IL-6 antibody or antigen-binding fragment thereof. Exemplary anti-IL-6 antibodies or antigen-binding fragments thereof include those as disclosed in, e.g., U.S. Pat. No. 7,479,453, which is incorporated by reference herein in its entirety and for all purposes. In some embodiments, the anti-IL-6 antibody or antigen-binding fragment thereof disclosed in U.S. Pat. No. 7,479,453 is tocilizumab. Accordingly, in some embodiments, the anti-IL-6 antibody or antigen-binding fragment thereof is tocilizumab. Tocilizumab is marketed as, e.g., ACTEMRA®.

In some embodiments, the one or more additional therapeutic agents comprises, consists essentially of, or consists of a PAD4 inhibitor. In some embodiments, a PAD4 inhibitor is an anti-PAD4 antibody or antigen-binding fragment thereof. Exemplary anti-PAD4 antibodies or antigen-binding fragments thereof include those as disclosed in, e.g., WO2024020579, U.S. Pat. No. 11,447,569, WO2022176970, and WO2024133161, each of which is incorporated by reference herein in its entirety and for all purposes.

In some embodiments in which the disease or disorder is inflammatory bowel disease, the one or more additional therapeutic agents comprises, consists essentially of, or consists of one or more anti-inflammatory agents and/or one or more immune system suppressors, including any of those as disclosed herein. In some embodiments in which the disease or disorder is inflammatory bowel disease, the one or more additional therapeutic agents comprises, consists essentially of, or consists of an aminosalicylate, a corticosteroid, a thiopurine, a methotrexate, a TNF inhibitor, or an S1PR agonist. In some embodiments in which the disease or disorder is inflammatory bowel disease, the one or more additional therapeutic agents comprises, consists essentially of, or consists of a topical mesalamine, a steroid (e.g., a topical steroid, an oral steroid, and an intravenous steroid), a 5-ASA agent (e.g., mesalamine and sulfasalazine), an IL-23 inhibitor (e.g., Risankizumab-rzaa), a dual IL-12 and IL-23 inhibitor (e.g., ustekinumab), a Janus kinase (JAK) inhibitor (e.g., abrocitinib, baricitinib, delgocitinib, fedratinib, filgotinib, oclacitinib, pacritinib, peficitinib, ruxolitinib, tofacitinib, or upadacitinib), an integrin inhibitor, or a surgical intervention.

In some embodiments in which the disease or disorder is psoriatic arthritis, the one or more additional therapeutic agents comprises, consists essentially of, or consists of one or more anti-inflammatory agents and/or one or more immune system suppressors, including any of those as disclosed herein. In some embodiments in which the disease or disorder is psoriatic arthritis, the one or more additional therapeutic agents comprises, consists essentially of, or consists of an acetaminophen, a COX-2 selective NSAID, a salicylate, a propionic acid, an oxicam, an acetic acid, a fenamate, a TNF inhibitor, an IL-6 targeting agent (e.g., an IL-6 inhibitor), an anti-CD20 antibody, a CLTA4 inhibitor, a dual IL-12 and IL-23 inhibitor (e.g., ustekinumab), an IL-17 inhibitor, an IL-23 inhibitor (e.g., Risankizumab-rzaa), a Janus kinase (JAK) inhibitor (e.g., abrocitinib, baricitinib, delgocitinib, fedratinib, filgotinib, oclacitinib, pacritinib, peficitinib, ruxolitinib, tofacitinib, or upadacitinib), a PDE4 inhibitor, a steroid, a hydroxychloroquine, a sulfasalazine, a methotrexate, a leflunomide, a minocycline, an azathioprine, or a TYK2 inhibitor (e.g., deucravacitinib).

In some embodiments in which the disease or disorder is atopic dermatitis, the one or more additional therapeutic agents comprises, consists essentially of, or consists of one or more anti-inflammatory agents and/or one or more immune system suppressors, including any of those as disclosed herein. In some embodiments in which the disease or disorder is atopic dermatitis, the one or more additional therapeutic agents comprises, consists essentially of, or consists of an emollient, a steroid (e.g., a topical steroid and an oral steroid), UV light therapy, a Janus kinase (JAK) inhibitor (such as an oral JAK inhibitor), dupilumab, tralokinumab, or an antibiotic. In some embodiments in which the disease or disorder is impetigo, the one or one or more additional therapeutic agents comprises, consists essentially of, or consists of one or more antibiotics.

In some embodiments in which the disease or disorder is chronic obstructive pulmonary disease (COPD), the one or more additional therapeutic agents comprises, consists essentially of, or consists of one or more anti-inflammatory agents and/or one or more immune system suppressors, including any of those as disclosed herein. In some embodiments in which the disease or disorder is chronic obstructive pulmonary disease (COPD), the one or more additional therapeutic agents comprises, consists essentially of, or consists of one or more antibiotics and/or one or more anti-viral agents. In some embodiments in which the disease or disorder is chronic obstructive pulmonary disease (COPD), the one or more additional therapeutic agents comprises, consists essentially of, or consists of a beta agonist, muscarinic antagonist, systemic steroid, azithromycin, roflumilast, or dupilumab. In some embodiments in which the disease or disorder is chronic obstructive pulmonary disease (COPD), the one or more additional therapeutic agents comprises, consists essentially of, or consists of an inhaled beta agonist, an inhaled muscarinic antagonic, an inhaled steroid, a phosphodiesterase 3 & 4 inhibitor, a steroid (e.g., an oral steroid), an oral azithromycin, an oral PDE4 inhibitor, a surgical invention (e.g., bullectomy), or a minimally invasive intervention (e.g., endobronchial valves).

In some embodiments in which the disease or disorder is sarcoidosis, the one or more additional therapeutic agents comprises, consists essentially of, or consists of one or more anti-inflammatory agents and/or one or more immune system suppressors, including any of those as disclosed herein. In some embodiments in which the disease or disorder is sarcoidosis, the one or more additional therapeutic agents comprises, consists essentially of, or consists of a steroid (e.g., an inhaled or oral steroid), a methotrexate, an azathioprine, or a leflunomide.

In some embodiments, the anti-inflammatory agent is an analgesic, a corticosteroid, an antirheumatic agent, an aminosalicylate, or a gastrointestinal agent. In some embodiments, the analgesic is indomethacin, naproxen, ibuprofen, choline magnesium trisalicylate, ketorolac tromethamine, or rofecoxib. In some embodiments, the corticosteroid is clobetasol, dexamethasone, dexamethasone phosphate, hydrocortisone, hydrocortisone sodium phosphate, or methylprednisolone acetate. In some embodiments, the antirheumatic agent is dexamethasone, cyclosporine, sulfasalazine, valdecoxib, or penicillamine. In some embodiments, the gastrointestinal agent is mesalamine, olsalazine sodium, balsalazide disodium. In some embodiments, the anti-inflammatory agent is an aminosalicylate, such as mesalamine, olsalazine, or balsalazide. In some embodiments, the anti-inflammatory agent is a corticosteroid. In some embodiments, the therapeutic agent is an immune system suppressor. In some embodiments, the immune system suppressor is azathioprine, methotrexate, mercaptopurine, tofacitinib, ozanimod, or upadacitinib.

In some embodiments in which the disease or disorder is an infectious disease, the one or more additional therapeutic agents comprises, consists essentially of, or consists of one or more antibiotics and/or one or more anti-viral agents.

In some embodiments in which the disease or disorder is a cancer, the method of treatment or use described herein can further include administering one or more of: radiation therapy, surgery, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, a fusion protein, or receptor extracellular domain-Fc fusion, including any of those as disclosed herein.

In some embodiments, the one or more additional therapeutic agents is administered for acute treatment of the disease or disorder, and/or for maintenance treatment of the disease or disorder. In some embodiments, the one or more additional therapeutic agents is administered over a period of at or at least one week, two weeks, three weeks, four weeks, one month, two months, three months, six months, nine months, or 12 months or more. In some embodiments, the one or more additional therapeutic agents is administered over a period of up to 2 weeks, 4 weeks, 1 month, 3 months, 6 months, 9 months, or 12 months.

In some embodiments, the subject is a mammalian subject, e.g., a human or a non-human primate. In some embodiments, the subject is a human. In some embodiments, the subject is a non-human primate. In some embodiments, the non-human primate is a cynomolgus monkey.

### B. Methods of Diagnosis and Assessment of IL-18Rβ Expression

Provided herein are methods of diagnosis of a disease or disorder, e.g., a disease or disorder associated with an increase in levels of IL-18Rβ and/or an increase in activity of the IL-18 signaling pathway, as compared to a subject not having the disease or disorder.

In some embodiments, provided herein is a method of diagnosing a disease or disorder comprising: (i) contacting a biological sample with any anti-IL-18Rβ antibody or antigen-binding fragment thereof described herein, any multi-specific antibody described herein, any conjugate described herein, or any composition described herein, wherein the biological sample is from a subject having or suspected of having a disease or disorder; and (ii) detecting the presence, absence, or level of binding to IL-18Rβ in the biological sample.

In some embodiments, provided herein is a method of diagnosing a disease or disorder comprising: (i) contacting a biological sample with any anti-IL-18Rβ antibody or antigen-binding fragment thereof described herein, wherein the biological sample is from a subject having or suspected of having a disease or disorder; and (ii) detecting the presence, absence, or level of binding between the anti-IL-18Rβ antibody or antigen-binding fragment thereof and the IL-18Rβ in the biological sample.

In some embodiments, the method of diagnosing a disease or condition is a method of diagnosing a disease or condition that is associated with an increase in IL-18Rβ in a biological sample of the subject as compared to a subject not having the disease or disorder.

In some embodiments, the method of diagnosing a disease or condition further comprises determining that the disease or condition is associated with an increase in IL-18Rβ in a biological sample of the subject as compared to a subject not having the disease or disorder.

In some embodiments, provided herein is a method of assessing expression of IL-18Rβ in a biological sample, comprising: (i) contacting a biological sample with any anti-IL-18Rβ antibody or antigen-binding fragment thereof described herein, wherein the biological sample is from a subject having or suspected of having a disease or disorder; and (ii) detecting the presence, absence, or level of binding between the anti-IL-18Rβ antibody or antigen-binding fragment thereof and the IL-18Rβ in the biological sample. In some embodiments, the method of assessing is used to identify subjects for administration of an anti-IL-18Rβ antibody or antigen-binding fragment thereof for treatment of the disease or disorder.

In some embodiments, provided herein is a method of identifying a subject for treatment with an anti-IL-18Rβ antibody or antigen-binding fragment thereof, comprising: (i) contacting a biological sample with any anti-IL-18Rβ antibody or antigen-binding fragment thereof described herein, wherein the biological sample is from a subject having or suspected of having a disease or disorder; (ii) detecting the presence, absence, or level of binding between the anti-IL-18Rβ antibody or antigen-binding fragment thereof and the IL-18Rβ in the biological sample, wherein if the biological sample exhibits the presence of binding between the anti-IL-18Rβ antibody or antigen-binding fragment thereof and the IL-18Rβ in the biological sample, or if the biological sample exhibits a level of binding between the anti-IL-18Rβ antibody or antigen-binding fragment thereof and the IL-18Rβ in the biological sample that is greater than a threshold value, then the subject is identified for treatment with the anti-IL-18Rβ antibody or antigen-binding fragment thereof. In some embodiments, the method further comprises: (iii) determining that the biological sample exhibits the presence of binding between the anti-IL-18Rβ antibody or antigen-binding fragment thereof and the IL-18Rβ in the biological sample, or determining that the biological sample exhibits a level of binding between the anti-IL-18Rβ antibody or antigen-binding fragment thereof and the IL-18Rβ in the biological sample that is greater than a threshold value, or determining that the presence or level of binding to IL-18Rβ in the biological sample is equal to or greater than a threshold value that is associated with a subject having the disease or disorder, thereby identifying the subject for treatment with the anti-IL-18Rβ antibody or antigen-binding fragment thereof.

In some embodiments, also provided herein is a method of treatment that comprises administering any anti-IL-18Rβ antibody or antigen-binding fragment thereof described herein, any multi-specific antibody described herein, any conjugate described herein, or any composition, e.g., pharmaceutical composition, described herein, to a subject identified as having a disease or disorder, wherein the subject is identified for treatment by a method comprising: (i) contacting a biological sample with any anti-IL-18Rβ antibody or antigen-binding fragment thereof described herein, wherein the biological sample is from the subject; and determining that the biological sample exhibits the presence of binding between the anti-IL-18Rβ antibody or antigen-binding fragment thereof and the IL-18Rβ in the biological sample, or determining that the biological sample exhibits a level of binding between the anti-IL-18Rβ antibody or antigen-binding fragment thereof and the IL-18Rβ in the biological sample that is greater than a threshold value, or determining that the presence or level of binding to IL-18Rβ in the biological sample is equal to or greater than a threshold value that is associated with a subject having the disease or disorder.

In some embodiments, the threshold value is a value of IL-18Rβ binding in a biological sample from the subject that is greater than a value of IL-18Rβ binding in a biological sample from a subject not having the disease or disorder or is greater than an average value of IL-18Rβ binding in a plurality of biological samples each from a subject not having the disease or disorder.

In some embodiments, the threshold value is a value of IL-18Rβ binding in a biological sample from the subject that is at least 25%, 50%, 75%, 100%, 200%, 300%, 400%, or 500% greater than a value of IL-18Rβ binding in a biological sample from a subject not having the disease or disorder or is at least 25%, 50%, 75%, 100%, 200%, 300%, 400%, or 500% greater than a value of IL-18Rβ binding in a plurality of biological samples each from a subject not having the disease or disorder.

In some embodiments, the threshold value that is associated with a subject having the disease or disorder is an average or mean value of IL-18Rβ binding in a plurality of biological samples each from a subject having the disease or disorder. In some embodiments, the threshold value that is associated with a subject having the disease or disorder is within 1 standard deviation below an average or mean value of IL-18Rβ binding in a plurality of biological samples each from a subject having the disease or disorder.

In some embodiments, the method is an in vitro method. In some embodiments, the method is an in vivo method. In some embodiments, the method is an ex vivo method.

In some embodiments, the biological sample comprises, consists essentially of, or consists of body fluids, such as blood, plasma, serum, cerebrospinal fluid, synovial fluid, urine or sweat; or tissue or organ samples, including processed samples derived therefrom. In some embodiments, the biological sample is a serum sample. In some embodiments, the biological sample is a tissue sample.

In some embodiments, IL-18Rβ expression or protein levels are detected in a serum sample from a subject. In some embodiments, IL-18Rβ expression or protein levels are detected in a tissue from a subject, e.g., heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons. In some embodiments, the tissue is obtained via a biopsy.

In some embodiments, the subject is a mammalian subject, e.g., a human or a non-human primate. In some embodiments, the subject is a human. In some embodiments, the subject is a non-human primate. In some embodiments, the non-human primate is a cynomolgus monkey.

In some embodiments, the subject has, or is suspected of having, or is at risk for developing, a disease or disorder, such as any disease or disorder described herein. In some embodiments, the disease or disorder is an autoimmune disease, an inflammatory disease, a cancer, or an infectious disease. In some embodiments, the disease or disorder is inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH) (e.g., monogenic HLH), dry eye disease (DED), cytokine release syndrome (CRS), systemic inflammatory response syndrome (SIRS), autoinflammation with infantile enterocolitis (AIFEC), XIAP deficiency. NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), monogenic enterocolitis, asthma, Celiac disease, age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2). In some embodiments, the disease or disorder is inflammatory bowel disease (IBD). In some embodiments, the inflammatory bowel disease is Crohn's disease or ulcerative colitis. In some embodiments, the inflammatory bowel disease is Crohn's disease. In some embodiments, the inflammatory bowel disease is ulcerative colitis.

In some embodiments, the disease or disorder is an infectious disease. The infectious disease can, in some embodiments, be any autoimmune disease, such as any infectious disease associated with an increase in levels of IL-18Rβ and/or an increase in activity of the IL-18 signaling pathway, as compared to a subject not having the infectious disease. In some embodiments, the infectious disease is a disease caused by a viral infection. In some embodiments, the infectious disease is caused by acute respiratory syndrome coronavirus (SARS-CoV) or acute respiratory syndrome coronavirus 2 (SARS-CoV-2). In some embodiments, the infectious disease is caused by SARS-CoV. In some embodiments, the infectious disease is caused by SARS-CoV-2. In some embodiments, the disease or disorder is COVID-19.

Various methods known in the art for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Exemplary labels include radionuclides (e.g, ¹²⁵I, ¹³¹I, ³⁵S, ³H, or ³²P and/or chromium (⁵¹Cr), cobalt (⁵⁷Co), fluorine (¹⁸F), gadolinium (¹⁵³Gd, ¹⁵⁹Gd), germanium (⁶⁸Ge), holmium (¹⁶⁶Ho), indium (¹¹⁵In, ¹¹³In, ¹¹²In, ¹¹¹In), iodine (¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), prascodymium (¹⁴²Pr), promethium (¹⁴⁹Pm), rhenium (¹⁸⁶Re, ¹⁸⁸Re), rhodium (¹⁰⁵Rh), rutheroium (⁹⁷Ru), samarium (¹⁵³Sm), scandium (⁴⁷Sc), selenium (⁷⁵Se), (⁸⁵Sr), sulphur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y),), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Various general techniques to be used in performing the various immunoassays noted above are known.

For purposes of diagnosis, the antibodies can be labeled with a detectable moiety including but not limited to radioisotopes, fluorescent labels, and various enzyme-substrate labels know in the art. Methods of conjugating labels to an antibody are known in the art.

In some embodiments, the anti-IL-18Rβ antibody is conjugated to tracer (e.g. radioisotope, MRI contrast agent, or CT contrast agents). In some embodiments, the anti-IL-18Rβ antibody or antigen-binding fragment thereof is conjugated to a radioisotope, wherein the radio isotope is a metal or non-metal radioisotope. In some embodiments, the anti-IL-18Rβ antibody or antigen-binding fragment thereof is conjugated to a radioisotope, wherein the radioisotope is selected from ¹¹C, ¹⁸F, ⁷⁶Br, ¹²⁴I, ⁶⁸Ga, ⁴⁴Sc, ⁶⁴Cu, ⁸⁶Y, ⁵⁵Co, ⁷²As, ⁸⁹Zr, ¹²³I, ¹³¹I, ^{99m}Tc, ¹¹¹In, ⁶⁷Ga, and ¹⁷⁷Lu. In some embodiments, the anti-IL-18Rβ antibody or antigen-binding fragment thereof is conjugated to an MRI contrast agent. In some embodiments, the MRI contrast agent is selected from paramagnetic metal complexes (e.g. complexes containing gadolinium (III), dysprosium (III), or manganese (II)) or superparamagnetic agents (e.g. iron oxide nanoparticles (SPION) or ultrasmall superparamagnetic iron oxide (USPIO)), In some embodiments, the anti-IL-18Rβ is conjugated to a CT contrast agent (e.g. iodinated compounds).

In some embodiments, the anti-IL-18Rβ antibody is conjugated to a tracer and is administered to a subject before the antibody localization and/or quantity is detected using magnetic resonance imaging (MRI), computed tomography (CT), ultrasound, positron emission tomography (PET), single-photon emission computed tomography (SPECT), optical imaging, scintigraphy, planar gamma imaging or photoacoustic imaging.

In some embodiments, antibodies need not be labeled, and the presence thereof can be detected using a labeled antibody which binds to any of the antibodies.

The antibodies provided herein can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized in vivo following administration to a subject.

The antibody may also be used as staining reagent in pathology, e.g., using known techniques.

In some embodiments, the anti-IL-18Rβ antibody or antigen-binding fragment thereof, or multi-specific antibody, or conjugate, is administered to a subject having or suspected of having a disease or disorder, such as inflammatory bowel disease. In some embodiments, after administering to the subject, the location and/or quantity of the anti-IL-18Rβ antibody or antigen-binding fragment thereof, or multi-specific antibody, or conjugate is detected and/or measured. In some embodiments, the location and/or quantity of the anti-IL-18Rβ antibody or antigen-binding fragment thereof, or multi-specific antibody, or conjugate is used to determine if the subject has a disease or disorder, e.g., a disease or disorder associated an increase in levels of IL-18Rβ and/or an increase in activity of the IL-18 signaling pathway, as compared to a subject not having the autoimmune disease.

In some embodiments, the anti-IL-18Rβ antibody or antigen-binding fragment thereof is conjugated to a heterologous molecule or moiety that is an imaging agent or a detectable moiety. In some embodiments, the imaging agent is radionuclide. In some embodiments, the detectable moiety is selected from a group consisting of radionuclides, enzymes, fluorescent moieties or proteins, and luminescent moieties.

### C. Methods of Monitoring

Provided herein are methods for the monitoring of a disease or disorder in a subject that comprises: (a) administering a conjugate comprising any anti-IL-18Rβ antibody or antigen binding fragment thereof described herein and a heterologous molecule or moiety that is a imaging agent or a detectable moiety, to a subject having a disease or disorder, wherein the subject was previously administered a therapy for treating the disease or disorder; and (b) imaging or detecting the imaging agent or the detectable moiety. In some embodiments, the method is an in vivo method.

Also provided herein are methods of monitoring that include (a) contacting a composition comprising a biological sample from a subject with any anti-IL-18Rβ antibody or antigen-binding fragment thereof described herein, any multi-specific antibody described herein, any conjugate described herein, or any composition described herein, and (b) detecting the anti-IL-18Rβ antibody or antigen-binding fragment thereof bound to IL-18Rβ in the biological sample. In some embodiments, the method is an in vitro method. In some embodiments, the method is an ex vivo method.

Also provided herein are methods of monitoring that include (a) contacting a composition comprising a biological sample from a subject with any composition described herein that comprises an anti-IL-18Rβ antibody or antigen binding fragment thereof and a heterologous molecule or moiety that is a imaging agent or a detectable moiety, and (b) detecting the anti-IL-18Rβ antibody or antigen-binding fragment thereof bound to IL-18Rβ in the biological sample. In some embodiments, the method is an in vitro method. In some embodiments, the method is an ex vivo method.

In some embodiments, the biological sample comprises, consists essentially of, or consists of body fluids, such as blood, plasma, serum, cerebrospinal fluid, synovial fluid, urine or sweat; or tissue or organ samples, including processed samples derived therefrom. In some embodiments, the biological sample is a serum sample. In some embodiments, the biological sample is a tissue sample.

In some embodiments, the methods provided herein include a method of monitoring treatment of a disease or disorder, such as any disease or disorder described herein.

In some embodiments, the imaging or detecting indicates the presence of or expression level, e.g., relative expression level, of IL-18Rβ in the biological sample, and/or the presence of or number of IL-18Rβ-expressing cells in the biological sample. In some embodiments, the expression of IL-18Rβ correlates with severity of the disease, and is useful in monitoring response to therapy, monitoring residual disease, monitoring for the presence of metastases (when monitoring cancer), monitoring total tumor burden (when monitoring cancer), and the like.

In some embodiments, the anti-IL-18Rβ antibodies or antigen-binding fragments thereof (or multi-specific antibodies or conjugates containing the same) described herein can be used after to the administration of a therapy, to monitor the subject and/or to assess treatment outcomes and response to the therapy. For example, in some embodiments, the article of manufacture and/or kits further contain reagents for measuring the level of particular biomarkers that are associated with response to therapy for the disease or disorder, and instructions for measuring. The methods in some embodiments include incubating a biological sample with the antibody or antigen-binding fragment thereof and/or administering the antibody or antigen-binding fragment thereof to a subject.

In some embodiments, the anti-IL-18Rβ antibodies or antigen-binding fragments thereof (or multi-specific antibodies or conjugates containing the same) described herein are used to determine whether a biological sample, e.g., serum sample or tissue sample, from a subject who was previously administered a therapy for a disease or disorder has responded to the therapy. In some embodiments, the therapy comprises, consists essentially of, or consists of an inhibitor of IL-18Rβ and/or the IL-18 signaling pathway.

In some embodiments, the imaging or detecting can be performed using any suitable method available in the art, including any method for imaging or detecting any imaging agent, such as a radionuclide, or any detectable moieties, such as radionuclides, enzymes, fluorescent moieties or proteins, and luminescent moieties.

In some embodiments, measurement of the expression level of IL-18Rβ is used to monitor a disease or disorder. In some embodiments, the measurement is in vitro. Various methods known in the art for detecting specific antibody-antigen binding can be used. Exemplary immunoassays include an immunofluorescence assay, an aptamer-based assay, a histological or cytological assay, or an mRNA expression level assay. In some embodiments, the in vitro assay is selected from among an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay and avidity assay. In some embodiments, the assay is an immunoassay, in situ hybridization, immunohistochemistry, multiplexed immunohistochemistry, or 5-plex immunofluorescent immunohistochemistry. In some embodiments, the antibody specifically binds the biomarker, e.g., immune cell biomarker. General techniques to be used in performing the various immunoassays noted above are known to those of ordinary skill in the art.

Various methods known in the art for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Exemplary labels include radionuclides (e.g, ¹²⁵I, ¹³¹I, ³⁵S, ³H, or ³²P and/or chromium (⁵¹Cr), cobalt (⁵⁷Co), fluorine (¹⁸F), gadolinium (¹⁵³Gd, ¹⁵⁹Gd), germanium (⁶⁸Ge), holmium (¹⁶⁶Ho), indium (¹¹⁵In, ¹¹³In, ¹¹²In, ¹¹¹In), iodine (¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), rutheroium (⁹⁷Ru), samarium (¹⁵³Sm), scandium (⁴⁷Sc), selenium (⁷⁵Se), (⁸⁵Sr), sulphur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y),), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Various general techniques to be used in performing the various immunoassays noted above are known.

In some embodiments, the anti-IL-18Rβ antibody is conjugated to tracer (e.g. radioisotope, MRI contrast agent, or CT contrast agents). In some embodiments, the anti-IL-18Rβ antibody or antigen-binding fragment thereof is conjugated to a radioisotope, wherein the radio isotope is a metal or non-metal radioisotope. In some embodiments, the anti-IL-18Rβ antibody or antigen-binding fragment thereof is conjugated to a radioisotope, wherein the radioisotope is selected from ¹¹C, ¹⁸F, ⁷⁶Br, ¹²⁴I, ⁶⁸Ga, ⁴⁴Sc, ⁶⁴Cu, ⁸⁶Y, ⁵⁵Co, ⁷²As, ⁸⁹Zr, ¹²³I, ¹³¹I, ^{99m}Tc, ¹¹¹In, ⁶⁷Ga, and ¹⁷⁷Lu. In some embodiments, the anti-IL-18Rβ antibody or antigen-binding fragment thereof is conjugated to an MRI contrast agent. In some embodiments, the MRI contrast agent is selected from paramagnetic metal complexes (e.g. complexes containing gadolinium (III), dysprosium (III), or manganese (II)) or superparamagnetic agents (e.g. iron oxide nanoparticles (SPION) or ultrasmall superparamagnetic iron oxide (USPIO)), In some embodiments, the anti-IL-18Rβ is conjugated to a CT contrast agent (e.g. iodinated compounds).

In some embodiments, measurement of the expression level of IL-18Rβ for the purpose of monitoring a disease or disorder is performed in vivo. For purposes of monitoring a disease or disorder, the antibody can be labeled with a detectable moiety including but not limited to radioisotopes, fluorescent labels, and various enzyme-substrate labels know in the art. Methods of conjugating labels to antibodies are known in the art. An indicator moiety, or label group, can be attached to the anti-IL-18Rβ antibody and may be selected so as to meet the needs of various uses of the method which are often dictated by the availability of assay equipment and compatible immunoassay procedures. Exemplary labels include radionuclides (e.g, ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). In some embodiments, the antibody is conjugated to an indicator moiety, or label group, and is administered to a subject prior to antibody detection using magnetic resonance imaging (MRI), computed tomography (CT), ultrasound, positron emission tomography (PET), single-photon emission computed tomography (SPECT), optical imaging, scintigraphy, planar gamma imaging or photoacoustic imaging.

In some embodiments, the monitoring is used to indicate the presence of IL-18Rβ or the presence of or number or density of IL-18Rβ-expressing cells, such as the presence of or the number or density of IL-18Rβ-expressing cells in a tissue affected by the disease or disorder.

In some embodiments, the anti-IL-18Rβ antibody or antigen-binding fragment thereof need not be labeled, and the presence thereof can be detected using a labeled secondary antibody which binds to the primary antibody.

In some embodiments, the provided methods may help monitoring a subject's response to a treatment or therapy, such as any treatment or therapy for the disease or disorder.

In some embodiments, the subject is a mammalian subject, e.g., a human or a non-human primate. In some embodiments, the subject is a human. In some embodiments, the subject is a non-human primate. In some embodiments, the non-human primate is a cynomolgus monkey.

In some embodiments, the subject has or is suspected of having a disease or disorder, such as any disease or disorder described herein. In some embodiments, the disease or disorder is an autoimmune disease, an inflammatory disease, a cancer, or an infectious disease. In some embodiments, the disease or disorder is inflammatory bowel disease (e.g., ulcerative colitis, Crohn's disease), chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome (SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, monogenic enterocolitis, Celiac disease, age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2). In some embodiments, the disease or disorder is inflammatory bowel disease (IBD). In some embodiments, the inflammatory bowel disease is Crohn's disease or ulcerative colitis. In some embodiments, the inflammatory bowel disease is Crohn's disease. In some embodiments, the inflammatory bowel disease is ulcerative colitis.

In some embodiments, the disease or disorder is an infectious disease. The infectious disease can, in some embodiments, be any autoimmune disease, such as any infectious disease associated with an increase in levels of IL-18Rβ and/or an increase in activity of the IL-18 signaling pathway, as compared to a subject not having the infectious disease. In some embodiments, the infectious disease is a disease caused by a viral infection. In some embodiments, the infectious disease is caused by acute respiratory syndrome coronavirus (SARS-CoV) or acute respiratory syndrome coronavirus 2 (SARS-CoV-2). In some embodiments, the infectious disease is caused by SARS-CoV. In some embodiments, the infectious disease is caused by SARS-CoV-2. In some embodiments, the disease or disorder is COVID-19.

In some embodiments, the disease or disorder is a cancer. In some embodiments, the cancer is a bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, gastric cancer, esophageal cancer, ovarian cancer, renal cancer, melanoma, prostate cancer, or thyroid cancer.

In some embodiments, the disease or disorder is asthma, cancer, diabetic nephropathy, Alzheimer's disease, osteoporosis, Parkinson's disease, arthritis, rheumatoid arthritis, multiple sclerosis, psoriasis, psoriatic arthritis, inflammatory bowel disease (e.g., ulcerative colitis, Crohn's disease, indeterminate colitis inflammatory bowel disease), lupus, systemic lupus erythematosus, multiple sclerosis, myasthenia gravis, malignant anemia, dry eye syndrome, type I diabetes, type II diabetes, vasculitis, uveitis, sepsis, atherosclerosis, ankylosing spondylitis, phagocytic lymphatic tissue cell increase, macrophage activation syndrome, sepsis, septic arthritis, epileptic sepsis, atopic dermatitis, idiopathic pulmonary fibrosis, juvenile idiopathic arthritis (e.g., systemic juvenile idiopathic arthritis), adult-onset Still's disease, or chronic obstructive pulmonary disease (COPD) or pulmonary nodules.

### IV. Articles of Manufacture or Kits

Provided herein are articles of manufacture and kits comprising the provided compositions. In some embodiments, the kit comprises any of the provided compositions and instructions for administering the composition to a subject. In some embodiments, the kit comprises any of the provided anti-IL-18Rβ antibodies or antigen-binding fragments thereof, any of the provided multi-specific antibodies, any of the provided conjugates, or any of the provided compositions, and instructions for administering the same to a subject.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromogens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, reagents and/or antibodies and, optionally, other components included in the kit, and methods for using such. In some embodiments, the instructions indicate methods for using the cell compositions and antibodies for administration to a subject for treating a disease or disorder, such as in accord with any of the provided embodiments. In some embodiments, the instructions are provided as a label or a package insert, which is on or associated with the container. In some embodiments, the instructions may indicate directions for reconstitution and/or use of the composition.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

The term “antibody” herein is used in the broadest sense and includes polyclonal and monoclonal antibodies, including intact antibodies and functional (antigen-binding) antibody fragments, including fragment antigen binding (Fab) fragments. F(ab′)₂ fragments. Fab′ fragments. Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody, VHH) fragments. The term “antibody” encompasses genetically engineered and/or otherwise modified forms of immunoglobulins, such as intrabodies, peptibodies, chimeric antibodies, fully human antibodies, humanized antibodies, heteroconjugate antibodies, multi-specific antibodies, e.g., bispecific or trispecific antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region.” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

Unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the numbering schemes described herein, or other known schemes. Possible numbering schemes include, e.g., Kabat numbering, Chothia numbering, AbM numbering, Contact numbering, and IMGT numbering. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L}, or V_{H} domains, respectively. Sec, e.g., Portolano et al., J. Immunol. 150:880-887 (1993); Clarkson et al., Nature 352:624-628 (1991).

The term “constant region” or “constant domain” is a domain in an antibody heavy or light chain that is distinct from the variable region and is associated with certain effector functions.

The term “antibody fragment” or “antigen-binding fragment” as used herein refers to a molecule other than an intact antibody that comprises a portion of an intact antibody that binds the antigen to which the intact antibody binds. Typically, an antigen binding fragment includes all CDRs of a variable heavy chain (V_{H}) and variable light chain (V_{L}) sequence from an anti-IL-18Rβ antibody disclosed herein. In some embodiments, an antigen binding fragment includes all amino acids, or all CDRs, that constitute the paratope of an anti-IL-18Rβ antibody disclosed herein. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multi-specific antibodies formed from antibody fragments. Papain digestion of antibodies produce two identical antigen-binding fragments, called “Fab” fragments, and a residual “Fc” fragment, a designation reflecting the ability to crystallize readily. The Fab fragment is composed of an entire L chain along with the variable region domain of the H chain (V_{H}), and the first constant domain of one heavy chain (CH1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “F(ab)” refers to two of the protein fragments resulting from proteolytic cleavage of immunoglobulin G (IgG) molecules by the enzyme papain. Each F(ab) comprises a covalent heterodimer of the V_{H} chain and V_{L} chain and includes an intact antigen-binding site.

The term “F(ab)₂” refers to a protein fragment of IgG generated by proteolytic cleavage by the enzyme pepsin. Each F(ab′)₂ fragment comprises two F(ab) fragments, thus comprising both antigen-binding sites.

An “Fv fragment” for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule, but lacking the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In general, “human” antibodies are antibodies with an amino acid sequence corresponding to that of an antibody produced by a human or a human cell, or a non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

In general, “chimeric” antibodies are created by fusing the variable regions of an antibody from one species (e.g., mouse) to the constant domain of an antibody from another species (e.g., human). These antibodies may be produced through recombinant molecular biological techniques or may be physically conjugated together. In certain embodiments, the heterologous Fc domain is of human origin. In further embodiments, the heterologous Fc domain may be from a different Ig class from the parent antibody, including IgA (including subclasses IgA1 and IgA2), IgD. IgE, IgG (including subclasses IgG1, IgG2, IgG3, and IgG4), and IgM. In further embodiments, the heterologous Fc domain may be comprised of CH2 and CH3 domains from one or more of the different Ig classes.

In general, a “humanized antibody” is an antibody in which all of substantially all amino acid residues of the CDRs are derived from non-human CDRs and all of substantially all amino acid residues of the framework regions (FRs) are derived from human FRs. A humanized antibody may optionally comprise at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody, such as the antibody from which the CDRs are derived, e.g., to restore or improve the specificity and/or affinity of the antibody.

As used herein, “non-naturally occurring” antibodies can refer to antibodies that comprise one or more amino acid modifications, such that the resultant antibody is substantially non-naturally occurring (e.g., does not exist in nature). These amino acid modifications can include point mutations, wherein a naturally occurring amino acid is substituted for another naturally occurring amino acid. In some embodiments, the amino acid modifications can include point mutations wherein a non-naturally occurring amino acid is substituted for a naturally occurring amino acid. Non-naturally occurring antibodies can also refer to antibodies that are conjugated to a heterologous protein or compound, such as a detectable marker.

The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “isolated protein” referred to herein means that a subject protein (1) is free of at least some other proteins with which it would typically be found in nature, (2) is essentially free of other proteins from the same source, e.g., from the same species, (3) is expressed by a cell from a different species. (4) has been separated from at least about 50 percent of polynucleotides, lipids, carbohydrates, or other materials with which it is associated in nature. (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA. cDNA, mRNA or other RNA, of may be of synthetic origin, or any combination thereof. In certain embodiments, the isolated protein is substantially pure or substantially free from proteins or polypeptides or other contaminants that are found in its natural environment that would interfere with its use (therapeutic, diagnostic, prophylactic, research or otherwise).

The term “purified” as applied to nucleic acids or proteins generally denotes a nucleic acid or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or protein is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule.” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as a immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest.

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, for example, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide, such as a heavy chain and/or a light chain of an antibody, having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. In some embodiments, an amino acid can be substituted with an isosteric non-natural amino acid. For instance, in some embodiments, an isoleucine (Ile) can be substituted with an isosteric non-natural amino acid, such as norleucine. Amino acid substitutions may be introduced into an antibody of interest and the products screened for a desired activity, for example, retained/improved antigen binding, decreased immunogenicity, or improved ADCC or CDC.

TABLE 2

| Original Residue | Exemplary Substitutions |
|---|---|
| Ala (A) | Val; Leu; Ile |
| Arg I | Lys; Gln; Asn |
| Asn (N) | Gln; His; Asp, Lys; Arg |
| Asp (D) | Glu; Asn |
| CI(C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

The term. “corresponding to” with reference to positions of a protein, such as recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence based on structural sequence alignment or using a standard alignment algorithm, such as the GAP algorithm. By aligning the sequences, one skilled in the art can identify corresponding residues, for example, using conserved and identical amino acid residues as guides.

The term “specifically binds” as used herein means the ability of a protein, under specific binding conditions, to bind to a target protein such that its affinity or avidity is at least 10 times as great, but optionally 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to more than one target molecule. In some cases, a specifically binding protein may bind to a protein that has similarity in structural conformation with the target protein (e.g., paralogs or orthologs). Those of skill will recognize that specific binding to a molecule having the same function in a different species of animal (i.e., ortholog) or to a molecule having a substantially similar epitope as the target molecule (e.g., paralog) is possible and does not detract from the specificity of binding which is determined relative to a statistically valid collection of unique non-targets (e.g., random polypeptides). Thus, a protein of the invention may specifically bind to more than one distinct species of target molecule due to cross-reactivity. Solid-phase ELISA immunoassays, ForteBio Octet or Biacore measurements can be used to determine specific binding between two proteins. Generally, interactions between two binding proteins have dissociation constants (Kd) less than about 1×10⁻⁵ M, and sometimes as low as about 1×10⁻¹² M. In certain aspects of the present disclosure, interactions between two binding proteins, e.g., an anti-IL-18Rβ antibody or antigen-binding fragment thereof disclosed herein and 18Rβ) have dissociation constants of less than about 1×10⁻⁶ M, 1×10⁻⁷ M. 1×10⁻⁸ M, or 1×10⁻⁹ M.

The term “affinity” with reference to binding refers to the strength of the sum total of noncovalent interactions between a single binding site of a molecule (for example, an antibody) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry. In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad).

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “expression” refers to the process by which a polynucleotide is transcribed from a DNA template (such as into an mRNA or other RNA transcript) and/or the process by which a transcribed mRNA is subsequently translated into peptide, polypeptides or proteins. Transcripts and encoded polypeptides may be collectively referred to as “gene product.” If the polynucleotide is derived from genomic DNA, expression may include splicing of the mRNA in a eukaryotic cell.

The term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.6 to 6.8, more commonly in the range of 6.0 to 6.8, in the range of 6.2-6.8. On the other hand, a normal physiological pH is in the range of 7.2-7.8, more commonly 7.35 to 7.45, such as about 7.4. The tumor microenvironment has been discussed in Gillies et al., “MRI of the Tumor Microenvironment,” Journal of Magnetic Resonance Imaging, vol. 16, pp. 430-450, 2002, hereby incorporated by reference herein its entirety. The term “non-tumor microenvironment” refers to a microenvironment at a site other than a tumor.

The terms “individual” and “subject” are used interchangeably herein to refer to an animal: for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

The term “composition” refers to any mixture of two or more products, substances, or compounds, including cells or antibodies. It may be a solution, a suspension, liquid, powder, a paste, aqueous, non-aqueous or any combination thereof. The preparation is generally in such form as to permit the biological activity of the active ingredient (e.g. antibody) to be effective.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disorder (e.g., an inflammatory bowel disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum dose required to produce a measurable improvement of a particular disorder. In some embodiments, a therapeutically effective amount is the amount of a composition that reduces the severity, the duration and/or the symptoms associated with the disease or disorder being treated in the subject. A therapeutically effective amount herein may vary according to factors such as the disease state, age, sex, and weight of the patient. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

A “disease.” “condition,” or “disorder” as used herein refers to a condition where treatment is needed and/or desired. Exemplary diseases or disorders include, inter alia, inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome (SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, monogenic enterocolitis, Celiac disease, age-related macular degeneration (AMD), osteoporosis, Parkinson's disease, Grave's disease, Hashimoto's thyroiditis, Addison's disease, dermatomyositis, myasthenia gravis, pernicious anemia. Sjogren syndrome, vasculitis, uveitis, sepsis, atherosclerosis, ankylosing spondylitis, cancers, infectious diseases, acute respiratory syndrome coronavirus (SARS-CoV), or acute respiratory syndrome coronavirus 2 (SARS-CoV-2), as well as specific subtypes of any of the foregoing.

The term “inflammatory bowel disease” refers to a group of inflammatory conditions characterized by chronic and recurrent inflammation of the gastrointestinal tract. Exemplary signs and symptoms of inflammatory bowel disease include, but is not limited to, diarrhea, abdominal pain and cramping, fatigue, blood in the stool, anemia, fever, reduced appetite, and unintended weight loss. Exemplary inflammatory bowel diseases include, for instance, Crohn's disease and ulcerative colitis.

The term “immune-mediated disease” refers to a disease, condition, or disorder that results from an abnormal or dysregulated immune system response. Immune-mediated diseases encompass inflammatory diseases and autoimmune diseases, e.g., inflammatory diseases and autoimmune diseases that involve an abnormal or dysregulated immune system response.

The term “autoimmune disease” refers to a disease characterized by a loss of immune tolerance to a self-antigen. Exemplary autoimmune diseases include, for instance, inflammatory bowel disease, rheumatoid arthritis, systemic lupus erythematosus (SLE), type 1 diabetes, multiple sclerosis, psoriasis. Hastimoto's thyroidosis, myasthenia gravis, Grave's disease, vasculitis, and scleroderma.

The term “inflammatory disease” refers to a disease or disorder characterized by an inflammatory response. Exemplary inflammatory diseases include, for instance, ulcerative colitis, arthritis, uveitis, SLE, ankylosing spondylitis, scleroderma, and Sjorden's syndrome.

The term “tumor cell.” “cancer cell.” “cancer.” “tumor,” and/or “neoplasm,” unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Also, included in this definition are cells having abnormal proliferation that is not impeded (e.g. immune evasion and immune escape mechanisms) by the immune system (e.g. virus infected cells). Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as β-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

As used herein, combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional agents and instructions for use of the combination or elements thereof, for a purpose including, but not limited to, therapeutic uses.

The term “wild-type” or “natural” or “native,” which are used interchangeably, as used herein is used in connection with biological materials such as nucleic acid molecules, proteins, host cells, and the like, that are found in nature and not modified by human intervention.

### VI. Exemplary Embodiments

Among the provided embodiments are:
1. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 3 (CDR-L3) comprising the amino acid sequence of SEQ ID NO: 24.
2. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 3 (CDR-L3) contained within the amino acid sequence of SEQ ID NO: 35.
3. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and the V_{L} region comprises a light chain complementarity determining region 1 (CDR-L1) comprising the amino acid sequence of SEQ ID NO: 22, a light chain complementarity determining region 2 (CDR-L2) comprising the amino acid sequence of SEQ ID NO: 36, and a light chain complementarity determining region 3 (CDR-L3) comprising the amino acid sequence of SEQ ID NO: 24.
4. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 35.
5. The anti-IL-18Rβ antibody or antigen-binding fragment thereof of any one of embodiments 1-4, wherein the V_{H} region is or comprises an amino acid sequence having at least or at least about 85% sequence identity to the amino acid sequence of SEQ ID NO: 34.
6. The anti-IL-18Rβ antibody or antigen-binding fragment thereof of any one of embodiments 1-4, wherein the V_{H} region comprises an amino acid sequence having at least, or at least about 90% sequence identity to the amino acid sequence of SEQ ID NO: 34.
7. The anti-IL-18Rβ antibody or antigen-binding fragment thereof of any one of embodiments 1-4, wherein the V_{H} region comprises an amino acid sequence having at least, or at least about 95% sequence identity to the amino acid sequence of SEQ ID NO: 34.
8. The anti-IL-18Rβ antibody or antigen-binding fragment thereof of any one of embodiments 1-4, wherein the V_{H} region comprises an amino acid sequence having at least, or at least about 97% sequence identity to the amino acid sequence of SEQ ID NO: 34.
9. The anti-IL-18Rβ antibody or antigen-binding fragment thereof of any one of embodiments 1-4, wherein the V_{H} region comprises an amino acid sequence having at least, or at least about 99% sequence identity to the amino acid sequence of SEQ ID NO: 34.
10. The anti-IL-18Rβ antibody or antigen-binding fragment thereof of any one of embodiments 1-9, wherein the V_{L} region comprises an amino acid sequence having at least, or at least about 85% sequence identity to the amino acid sequence of SEQ ID NO: 35.
11. The anti-IL-18Rβ antibody or antigen-binding fragment thereof of any one of embodiments 1-9, wherein the V_{L} region comprises an amino acid sequence having at least, or at least about 90% sequence identity to the amino acid sequence of SEQ ID NO: 35.
12. The anti-IL-18Rβ antibody or antigen-binding fragment thereof of any one of embodiments 1-9, wherein the V_{L} region comprises an amino acid sequence having at least, or at least about 95% sequence identity to the amino acid sequence of SEQ ID NO: 35.
13. The anti-IL-18Rβ antibody or antigen-binding fragment thereof of any one of embodiments 1-9, wherein the V_{L} region comprises an amino acid sequence having at least, or at least about 97% sequence identity to the amino acid sequence of SEQ ID NO: 35.
14. The anti-IL-18Rβ antibody or antigen-binding fragment thereof of any one of embodiments 1-9, wherein the V_{L} region comprises an amino acid sequence having at least, or at least about 99% sequence identity to the amino acid sequence of SEQ ID NO: 35.
15. The anti-IL-18Rβ antibody or antigen-binding fragment thereof of any one of embodiments 1-14, wherein the V_{H} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 34; and the V_{L} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 35.
16. The anti-IL-18Rβ antibody or antigen-binding fragment thereof of any one of embodiments 1-15, wherein the V_{H} region comprises the amino acid sequence of SEQ ID NO: 34.
17. The anti-IL-18Rβ antibody or antigen-binding fragment thereof of any one of embodiments 1-16, wherein the V_{L} region comprises the amino acid sequence of SEQ ID NO: 35.
18. The anti-IL-18Rβ antibody or antigen-binding fragment thereof of any one of embodiments 1-17, wherein the V_{H} region comprises the amino acid sequence of SEQ ID NO: 34; and the V_{L} region comprises the amino acid sequence of SEQ ID NO: 35.
19. The anti-IL-18Rβ antibody or antigen-binding fragment thereof of any of embodiments 1-18, wherein the antibody or antigen-binding fragment thereof is an antibody comprising a constant domain.
20. The anti-IL-18Rβ antibody or antigen-binding fragment thereof of embodiment 19, wherein the constant domain is a constant domain selected from the group consisting of an IgA1 constant domain, an IgA2 constant domain, an IgD constant domain, an IgE constant domain, an IgG1 constant domain, an IgG2 constant domain, an IgG3 constant domain, an IgG4 constant domain, and an IgM constant domain.
21. The anti-IL-18Rβ antibody or antigen-binding fragment thereof of embodiment 19 or embodiment 20, wherein the constant domain is an IgG1 constant domain.
22. The anti-IL-18Rβ antibody or antigen-binding fragment thereof of any one of embodiments 19-21, wherein the antibody comprises: a heavy chain constant domain comprising the amino acid sequence of SEQ ID NO: 30, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and a light chain constant domain comprising the amino acid sequence of SEQ ID NO: 31, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31.
23. The anti-IL-18Rβ antibody or antigen-binding fragment thereof of any one of embodiments 19-22, wherein the antibody comprises a heavy chain constant domain comprising the amino acid sequence of SEQ ID NO: 30, and a light chain constant domain comprising the amino acid sequence of SEQ ID NO: 31.
24. The anti-IL-18Rβ antibody or antigen-binding fragment thereof of any one of embodiments 19-23, wherein the antibody comprises: a heavy chain comprising the amino acid sequence of SEQ ID NO: 32, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 32; and a light chain comprising the amino acid sequence of SEQ ID NO: 33, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 33.
25. The anti-IL-18Rβ antibody or antigen-binding fragment thereof of any one of embodiments 19-24, wherein the antibody comprises a heavy chain comprising the amino acid sequence of SEQ ID NO: 32, and a light chain comprising the amino acid sequence of SEQ ID NO: 33.
26. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1) comprising the amino acid sequence of SEQ ID NO: 52, a heavy chain complementarity determining region 2 (CDR-H2) comprising the amino acid sequence of SEQ ID NO: 53, and a heavy chain complementarity determining region 3 (CDR-H3) comprising the amino acid sequence of SEQ ID NO: 54; and the V_{L} region comprises a light chain complementarity determining region 1 (CDR-L1) comprising the amino acid sequence of SEQ ID NO: 66, a light chain complementarity determining region 2 (CDR-L2) comprising an amino acid sequence selected from the group consisting of SEQ ID NOs: 67, and a light chain complementarity determining region 3 (CDR-L3) comprising the amino acid sequence of SEQ ID NO: 68.
27. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein: the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 75; and the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 76.
28. The anti-IL-18Rβ antibody or antigen-binding fragment thereof of embodiment 26 or embodiment 27, wherein the V_{H} region comprises an amino acid sequence having at least, or at least about 85% sequence identity to the amino acid sequence of SEQ ID NO: 75.
29. The anti-IL-18Rβ antibody or antigen-binding fragment thereof of embodiment 26 or embodiment 27, wherein the V_{H} region comprises an amino acid sequence having at least, or at least about 90% sequence identity to the amino acid sequence of SEQ ID NO: 75.
30. The anti-IL-18Rβ antibody or antigen-binding fragment thereof of embodiment 26 or embodiment 27, wherein the V_{H} region comprises an amino acid sequence having at least, or at least about 95% sequence identity to the amino acid sequence of SEQ ID NO: 75.
31. The anti-IL-18Rβ antibody or antigen-binding fragment thereof of embodiment 26 or embodiment 27, wherein the V_{H} region comprises an amino acid sequence having at least, or at least about 97% sequence identity to the amino acid sequence of SEQ ID NO: 75.
32. The anti-IL-18Rβ antibody or antigen-binding fragment thereof of embodiment 26 or embodiment 27, wherein the V_{H} region comprises an amino acid sequence having at least, or at least about 99% sequence identity to the amino acid sequence of SEQ ID NO: 75.
33. The anti-IL-18Rβ antibody or antigen-binding fragment thereof of any one of embodiments 26-32, wherein the V_{L} region comprises an amino acid sequence having at least, or at least about 85% sequence identity to the amino acid sequence of SEQ ID NO: 76.
34. The anti-IL-18Rβ antibody or antigen-binding fragment thereof of any one of embodiments 26-32, wherein the V_{L} region comprises an amino acid sequence having at least, or at least about 90% sequence identity to the amino acid sequence of SEQ ID NO: 76.
35. The anti-IL-18Rβ antibody or antigen-binding fragment thereof of any one of embodiments 26-32, wherein the V_{L} region comprises an amino acid sequence having at least, or at least about 95% sequence identity to the amino acid sequence of SEQ ID NO: 76.
36. The anti-IL-18Rβ antibody or antigen-binding fragment thereof of any one of embodiments 26-32, wherein the V_{L} region comprises an amino acid sequence having at least, or at least about 97% sequence identity to the amino acid sequence of SEQ ID NO: 76.
37. The anti-IL-18Rβ antibody or antigen-binding fragment thereof of any one of embodiments 26-32, wherein the V_{L} region comprises an amino acid sequence having at least, or at least about 99% sequence identity to the amino acid sequence of SEQ ID NO: 76.
38. The anti-IL-18Rβ antibody or antigen-binding fragment thereof of any one of embodiments 26-37, wherein the V_{H} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 75; and the V_{L} region comprises an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 76.
39. The anti-IL-18Rβ antibody or antigen-binding fragment thereof of any one of embodiments 26-38, wherein the V_{H} region comprises the amino acid sequence of SEQ ID NO: 75.
40. The anti-IL-18Rβ antibody or antigen-binding fragment thereof of any one of embodiments 26-39, wherein the V_{L} region comprises the amino acid sequence of SEQ ID NO: 76.
41. The anti-IL-18Rβ antibody or antigen-binding fragment thereof of any one of embodiments 26-40, wherein the V_{H} region comprises the amino acid sequence of SEQ ID NO: 75; and the V_{L} region comprises the amino acid sequence of SEQ ID NO: 76.
42. The anti-IL-18Rβ antibody or antigen-binding fragment thereof of any of embodiments 26-41, wherein the antibody or antigen-binding fragment thereof is an antibody comprising a constant domain.
43. The anti-IL-18Rβ antibody or antigen-binding fragment thereof of embodiment 42, wherein the constant domain is a constant domain selected from the group consisting of an IgA1 constant domain, an IgA2 constant domain, an IgD constant domain, an IgE constant domain, an IgG1 constant domain, an IgG2 constant domain, an IgG3 constant domain, an IgG4 constant domain, and an IgM constant domain.
44. The anti-IL-18Rβ antibody or antigen-binding fragment thereof of embodiment 42 or embodiment 43, wherein the constant domain is an IgG1 constant domain.
45. The anti-IL-18Rβ antibody or antigen-binding fragment thereof of any one of embodiments 42-44, wherein the antibody comprises: a heavy chain constant domain comprising the amino acid sequence of SEQ ID NO: 30, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 30; and a light chain constant domain comprising the amino acid sequence of SEQ ID NO: 31, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 31.
46. The anti-IL-18Rβ antibody or antigen-binding fragment thereof of any one of embodiments 42-45, wherein the antibody comprises a heavy chain constant domain comprising the amino acid sequence of SEQ ID NO: 30, and a light chain constant domain comprising the amino acid sequence of SEQ ID NO: 31.
47. The anti-IL-18Rβ antibody or antigen-binding fragment thereof of any one of embodiments 42-46, wherein the antibody comprises: a heavy chain comprising the amino acid sequence of SEQ ID NO: 73, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 73; and a light chain comprising the amino acid sequence of SEQ ID NO: 74, or an amino acid sequence having at least, or at least about 85%, 86%, 87%, 88%, 89%, 90%, 91%, 92%, 93%, 94%, 95%, 96%, 97%, 98% or 99% sequence identity to the amino acid sequence of SEQ ID NO: 74.
48. The anti-IL-18Rβ antibody or antigen-binding fragment thereof of any one of embodiments 42-47, wherein the antibody comprises a heavy chain comprising the amino acid sequence of SEQ ID NO: 73, and a light chain comprising the amino acid sequence of SEQ ID NO: 74.
49. The anti-IL-18Rβ antibody or antigen-binding fragment thereof of any of embodiments 1-18 and 26-41, wherein the antibody or antigen-binding fragment thereof is an antigen-binding fragment.
50. The anti-IL-18Rβ antibody or antigen-binding fragment thereof of embodiment 49, wherein the antigen-binding fragment is selected from the group consisting of a single domain antibody, a single chain antibody, an unibody, a single chain variable fragment (scFv), a Fab fragment, and a F(ab′)₂ fragment.
51. The anti-IL-18Rβ antibody or antigen-binding fragment thereof of any one of embodiments 1-50, wherein the antibody or antigen-binding fragment thereof is recombinant.
52. The anti-IL-18Rβ antibody or antigen-binding fragment thereof of any of embodiments 1-51, wherein the antibody or antigen-binding fragment thereof is monoclonal.
53. The anti-IL-18Rβ antibody or antigen-binding fragment thereof of any of embodiments 1-52, wherein the antibody or antigen-binding fragment thereof is human.
54. The anti-IL-18Rβ antibody or antigen-binding fragment thereof of any of embodiments 1-25 and 49-53, wherein the antibody or antigen-binding fragment thereof binds to human IL-18Rβ at pH 7.4 with an equilibrium dissociation constant (K_{D}) that is about 1.5 nM or is less than about 1.5 nM.
55. The anti-IL-18Rβ antibody or antigen-binding fragment thereof of any of embodiments 1-25 and 49-54, wherein the antibody or antigen-binding fragment thereof binds to cynomolgus IL-18Rβ at pH 7.4 with an equilibrium dissociation constant (K_{D}) that is about 2 nM or is less than about 2 nM.
56. The anti-IL-18Rβ antibody or antigen-binding fragment thereof of any of embodiments 26-53, wherein the antibody or antigen-binding fragment thereof binds to human IL-18Rβ at pH 7.4 with an equilibrium dissociation constant (K_{D}) that is, is about, is less than, or is less than about 2 nM.
57. The anti-IL-18Rβ antibody or antigen-binding fragment thereof of any one of embodiments 1-25 and 49-56, wherein the antibody or antigen-binding fragment thereof has a K_{D} ratio of K_{D} at pH 5 to K_{D} at pH 7 of 0.33 to 3.33, or of about 0.33 to about 3.33.
58. The anti-IL-18Rβ antibody or antigen-binding fragment thereof of embodiment 57, wherein the K_{D} ratio is 0.8 to 1.3, or about 0.8 to about 1.3.
59. The anti-IL-18Rβ antibody or antigen-binding fragment thereof of any one of embodiments 26-56, wherein the antibody or antigen-binding fragment thereof has a K_{D} ratio of K_{D} at pH 5 to K_{D} at pH 7 of 0.25 to 1.00, or of about 0.25 to about 1.00.
60. The anti-IL-18Rβ antibody or antigen-binding fragment thereof of embodiment 59, wherein the K_{D} ratio is 0.25 to 0.5, or about 0.25 to about 0.5.
61. The anti-IL-18Rβ antibody or antigen-binding fragment thereof of any one of embodiments 1-25 and 49-58, wherein the anti-IL-18Rβ antibody or antigen-binding fragment thereof binds to an epitope of human IL-18Rβ that comprises, consists essentially of, or consists of residues 242-248, 279, 281, 282, 312, 342, and 343, with residue numbering corresponding to the amino acid sequence of SEQ ID NO: 126.
62. The anti-IL-18Rβ antibody or antigen-binding fragment thereof of any one of embodiments 54-61, wherein the human IL-18Rβ comprises, consists essentially of, or consists of the amino acid sequence of SEQ ID NO: 126.
63. The anti-IL-18Rβ antibody or antigen-binding fragment thereof of any one of embodiments 1-62, wherein the anti-IL-18Rβ antibody or antigen-binding fragment thereof blocks IL-18Rβ binding to an IL-18/IL-18Rα complex.
64. The anti-IL-18Rβ antibody or antigen-binding fragment thereof of any one of embodiments 1-63, comprising at least one post-translational modification of the amino acid sequence, optionally wherein the at least one post-translational modification comprises a post-translational modification of a heavy chain N-terminal glutamine (Q) to a pyroglutamate.
65. A multi-specific antibody, comprising a first antigen-binding domain that binds to IL-18Rβ and a second antigen-binding domain that binds to a second antigen, wherein the first antigen-binding domain comprises the anti-IL-18Rβ antibody or antigen-binding fragment thereof of any one of embodiments 1-64.
66. The multi-specific antibody of embodiment 65, that is a bispecific antibody.
67. A conjugate, comprising the anti-IL-18Rβ antibody or antigen-binding fragment thereof of any of embodiments 1-64 or the multi-specific antibody of embodiment 65 or embodiment 66, and a heterologous molecule or moiety.
68. A polynucleotide comprising a nucleic acid(s) encoding the anti-IL-18Rβ antibody or antigen-binding fragment thereof of any of embodiments 1-64, or a heavy chain or a light chain thereof.
69. A nucleic acid(s) encoding the anti-IL-18Rβ antibody or antigen-binding fragment thereof of any of embodiments 1-64, or a heavy chain or a light chain thereof.
70. The nucleic acid(s) of embodiment 69, wherein the nucleic acid(s) comprises a nucleic acid encoding a V_{H} region comprising the nucleotide sequence of SEQ ID NO: 127 or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 127; and a nucleic acid encoding a V_{L} region comprising the nucleotide sequence of SEQ ID NO: 128, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 128.
71. The nucleic acid(s) of embodiment 69 or embodiment 70, wherein the nucleic acid(s) comprises a nucleic acid encoding a V_{H} region comprising the nucleotide sequence of SEQ ID NO: 127; and a nucleic acid encoding a V_{L} region comprising the nucleotide sequence of SEQ ID NO: 128.
72. The nucleic acid(s) of any one of embodiments 69-71, wherein the nucleic acid(s) comprises a nucleic acid encoding a heavy chain comprising the nucleotide sequence of SEQ ID NO: 129, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 129; and a nucleic acid encoding a light chain comprising the nucleotide sequence of SEQ ID NO: 130, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 130.
73. The nucleic acid(s) of any one of embodiments 69-72, wherein the nucleic acid(s) comprises a nucleic acid encoding a heavy chain comprising the nucleotide sequence of SEQ ID NO: 129; and a nucleic acid encoding a light chain comprising the nucleotide sequence of SEQ ID NO: 130.
74. The nucleic acid(s) of embodiment 69, wherein the nucleic acid(s) comprises a nucleic acid encoding a V_{H} region comprising the nucleotide sequence of SEQ ID NO: 131, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 131; and a nucleic acid encoding a V_{L} region comprising the nucleotide sequence of SEQ ID NO: 132, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 132.
75. The nucleic acid(s) of embodiment 69 or embodiment 74, wherein the nucleic acid(s) comprises a nucleic acid encoding a V_{H} region comprising the nucleotide sequence of SEQ ID NO: 131; and a nucleic acid encoding a V_{L} region comprising the nucleotide sequence of SEQ ID NO: 132.
76. The nucleic acid(s) of any one of embodiments 69, 74, and 75, wherein the nucleic acid(s) comprises a nucleic acid encoding a heavy chain comprising the nucleotide sequence of SEQ ID NO: 133, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 133; and a nucleic acid encoding a light chain comprising the nucleotide sequence of SEQ ID NO: 134, or a nucleotide sequence having at least, or at least about 85%, 86%, 87%, 88%, 89%, 90%, 91%, 92%, 93%, 94%, 95%, 96%, 97%, 98% or 99% sequence identity to the nucleotide sequence of SEQ ID NO: 134.
77. The nucleic acid(s) of any one of embodiments 69 and 74-76, wherein the nucleic acid(s) comprises a nucleic acid encoding a heavy chain comprising the nucleotide sequence of SEQ ID NO: 133; and a nucleic acid encoding a light chain comprising the nucleotide sequence of SEQ ID NO: 134.
78. A vector, comprising the polynucleotide of embodiment 68 or the nucleic acid(s) of any one of embodiments 69-77.
79. The vector of embodiment 78, wherein the vector is a viral vector.
80. The vector of embodiment 79, wherein the viral vector is a retroviral vector or a lentiviral vector.
81. A host cell comprising the anti-IL-18Rβ antibody or antigen-binding fragment thereof of any one of embodiments 1-64, the polynucleotide of embodiment 68, the nucleic acid(s) of any one of embodiments 69-77, or the vector of any one of embodiments 78-80.
82. A method of producing an antibody or antigen-binding fragment thereof comprising culturing the host cell of embodiment 81 under a condition that produces the antibody or antigen-binding fragment thereof.
83. The method of embodiment 82, further comprising recovering the antibody or antigen-binding fragment thereof produced by the host cell.
84. An antibody or antigen-binding fragment thereof produced by the method of embodiment 82 or embodiment 83.
85. A composition comprising the anti-IL-18Rβ antibody or antigen-binding fragment thereof of any one of embodiments 1-64, the multi-specific antibody of embodiment 65 or embodiment 66, or the conjugate of embodiment 67.
86. The composition of embodiment 85, further comprising a pharmaceutically acceptable excipient.
87. A method of treatment, comprising administering the anti-IL-18Rβ antibody or antigen-binding fragment thereof of any one of embodiments 1-64 and 84, the multi-specific antibody of embodiment 65 or embodiment 66, the conjugate of embodiment 67, or the composition of embodiment 85 or embodiment 86, to a subject having a disease or disorder.
88. The method of embodiment 88, further comprising administering one or more additional therapeutic agents.
89. The method of embodiment 88, wherein the one or more additional therapeutic agents comprises, consists essentially of, or consists of an aminosalicylate, a corticosteroid, a thiopurine, a methotrexate, a tumor necrosis factor (TNF) inhibitor, an α4β7 integrin inhibitor, a TNF-α inhibitor, an IL-23 inhibitor, a dual IL-12 and IL-23 inhibitor, a tumor necrosis factor-like cytokine 1A (TL1A) inhibitor, an IL-2-CD25 fusion protein, a bispecific anti-TL1A/anti-TNF-α binding protein, an E-type prostanoid receptor 4 (EP4) agonist, a TYK2 inhibitor, a sphingosine-1-phosphate receptor (S1PR) agonist, a Janus kinase (JAK) inhibitor, an interkeukin-1 receptor associated kinase 4 (IRAK-4) inhibitor, a toll-like receptor 7 and 8 (TLR7/8) inhibitor, or an IL-22 inhibitor.
90. The method of embodiment 88 or embodiment 89, wherein the one or more additional therapeutic agents is administered concurrently with the anti-IL-18Rβ antibody or antigen-binding fragment thereof, the multi-specific antibody, the conjugate, or the composition.
91. The method of embodiment 88 or embodiment 89, wherein the one or more additional therapeutic agents is administered before or after the anti-IL-18Rβ antibody or antigen-binding fragment thereof, the multi-specific antibody, the conjugate, or the composition.
92. The method of any one of embodiments 87-91, wherein the disease or disorder is an immune-mediated disease, an autoimmune disease, an inflammatory disease, a cancer, or an infectious disease.
93. The method of any one of embodiments 87-92, wherein the disease or disorder is associated with an increase in IL-18Rβ in a biological sample of the subject as compared to a subject not having the disease or disorder.
94. The method of embodiment 93, wherein the biological sample is a serum sample.
95. The method of any one of embodiments 87-94, wherein the disease or disorder is an immune-mediated disease.
96. The method of any one of embodiments 87-95, wherein the disease or disorder is an inflammatory disease.
97. The method of any one of embodiments 87-96, wherein the disease or disorder is an inflammatory bowel disease.
98. The method of embodiment 97, wherein the inflammatory bowel disease is Crohn's disease.
99. The method of embodiment 97, wherein the inflammatory bowel disease is ulcerative colitis. 100. The method of any one of embodiments 87-96, wherein the disease or disorder is chronic obstructive pulmonary disease (COPD).
101. The method of any one of embodiments 87-95, wherein the disease or disorder is an autoimmune disease.
102. The method of any one of embodiments 87-95, wherein the disease or disorder is a cancer.
103. The method of any one of embodiments 87-95, wherein the disease or disorder is an infectious disease.
104. The method of any one of embodiments 87-95, wherein the disease or disorder is inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, Celiac disease, or age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).
105. The method of embodiment 104, wherein: the inflammatory bowel disease is Crohn's disease or ulcerative colitis; the arthritis is rheumatoid arthritis, psoriatic arthritis, idiopathic arthritis, or giant cell arthritis; the psoriasis is plaque psoriasis; the organ transplant rejection is a kidney transplant rejection; the heart disease is ischemic heart disease; the MAS is infantile MAS; or the sarcoidosis is pulmonary sarcoidosis.
106. The method of embodiment 105, wherein: the rheumatoid arthritis is juvenile rheumatoid arthritis; or the idiopathic arthritis is juvenile idiopathic arthritis.
107. The method of any one of embodiments 87-106, wherein the subject is a human.
108. Use of a composition of embodiment 85 or embodiment 86 for the manufacture of a medicament for treatment of a disease or disorder.
109. Use of a composition of embodiment 85 or embodiment 86 for treatment of a disease or disorder.
110. A composition of embodiment 85 or embodiment 86 for use in treatment of a disease or disorder.
111. The use of embodiment 108 or embodiment 109 or the composition for use of embodiment 110, wherein the treatment of the disease or disorder further comprises administering one or more additional therapeutic agents.
112. The use of any one of embodiments 108, 109, and 111, or the composition for use of embodiment 110 or embodiment 111, wherein the one or more additional therapeutic agents comprises, consists essentially of, or consists of an aminosalicylate, a corticosteroid, a thiopurine, a methotrexate, a tumor necrosis factor (TNF) inhibitor, an α4β7 integrin inhibitor, a TNF-α inhibitor, an IL-23 inhibitor, a dual IL-12 and IL-23 inhibitor, a tumor necrosis factor-like cytokine 1A (TL1A) inhibitor, an IL-2-CD25 fusion protein, a bispecific anti-TL1A/anti-TNF-α binding protein, an E-type prostanoid receptor 4 (EP4) agonist, a TYK2 inhibitor, a sphingosine-1-phosphate receptor (S1PR) agonist, a Janus kinase (JAK) inhibitor, an interkeukin-1 receptor associated kinase 4 (IRAK-4) inhibitor, a toll-like receptor 7 and 8 (TLR7/8) inhibitor, or an IL-22 inhibitor.
113. The use of any one of embodiments 108, 109, 111, and 112, or the composition for use of any one of embodiments 110-112, wherein the one or more additional therapeutic agents is to be administered concurrently with the anti-IL-18Rβ antibody or antigen-binding fragment thereof, the multi-specific antibody, the conjugate, or the composition.
114. The use of any one of embodiments 108, 109, 111, and 112, or the composition for use of any one of embodiments 110-112, wherein the one or more additional therapeutic agents is to be administered sequentially with the anti-IL-18Rβ antibody or antigen-binding fragment thereof, the multi-specific antibody, the conjugate, or the composition.
115. The use of any one of embodiments 108, 109, and 111-114, or the composition for use of any one of embodiments 110-114, wherein the disease or disorder is an immune-mediated disease, an autoimmune disease, an inflammatory disease, a cancer, or an infectious disease.
116. The use of any one of embodiments 108, 109, and 111-115, or the composition for use of any one of embodiments 110-115, wherein the disease or disorder is an immune-mediated disease.
117. The use of any one of embodiments 108, 109, and 111-116, or the composition for use of any one of embodiments 110-116, wherein the disease or disorder is an inflammatory disease.
118. The use of any one of embodiments 108, 109, and 111-117, or the composition for use of any one of embodiments 110-117, wherein the disease or disorder is an inflammatory bowel disease.
119. The use or composition for use of embodiment 118, wherein the inflammatory bowel disease is Crohn's disease.
120. The use or composition for use of embodiment 118, wherein the inflammatory bowel disease is ulcerative colitis.
121. The use of any one of embodiments 108, 109, and 111-117, or the composition for use of any one of embodiments 110-117, wherein the disease or disorder is chronic obstructive pulmonary disease (COPD).
122. The use of any one of embodiments 108, 109, and 111-116, or the composition for use of any one of embodiments 110-116, wherein the disease or disorder is an autoimmune disease.
123. The use of any one of embodiments 108, 109, and 111-116, or the composition for use of any one of embodiments 110-116, wherein the disease or disorder is a cancer.
124. The use of any one of embodiments 108, 109, and 111-116, or the composition for use of any one of embodiments 110-116, wherein the disease or disorder is an infectious disease.
125. The use of any one of embodiments 108, 109, and 111-116, or the composition for use of any one of embodiments 110-116, wherein the disease or disorder is inflammatory bowel disease, allergy, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, Celiac disease, or age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).
126. The use or composition for use of embodiment 125, wherein: the inflammatory bowel disease is Crohn's disease or ulcerative colitis: the arthritis is rheumatoid arthritis, psoriatic arthritis, idiopathic arthritis, or giant cell arthritis; the psoriasis is plaque psoriasis; the organ transplant rejection is a kidney transplant rejection; the heart disease is ischemic heart disease; the MAS is infantile MAS; or the sarcoidosis is pulmonary sarcoidosis.
127. The use or composition for use of embodiment 126, wherein: the rheumatoid arthritis is juvenile rheumatoid arthritis; or the idiopathic arthritis is juvenile idiopathic arthritis.

### VII. Examples

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1—Antibody Discovery and Initial Screening of IL-18Rβ Antibodies

This example provides an antibody discovery campaign to yield human anti-interleukin-18 receptor-β (IL-18Rβ) monoclonal antibodies that bind to IL-18Rβ, block IL-18 ternary complex formation, and block associated interferon gamma (IFNγ) production at low concentrations, which can then be further characterized and studied. To uncover such antibodies, the campaign consisted of many steps, including the generation of candidate antibody libraries and multiple assays to screen IL-18Rβ binding, blocking of IL-18 ternary complex formation, epitope binning, and ability of candidate antibodies to block IL-18 dependent IFNγ production.

### A. Generating Human Antibody Libraries

Human anti-interleukin-18 receptor-β (IL-18Rβ) monoclonal antibodies (mAbs) were generated by immunizing chimeric mice. Murine subjects were immunized following either a short (3-4 weeks) or a long (6-8 week) protocol using footpad, tail, and/or subcutaneous injections. Four cohorts of mice were immunized with one of two antigens, huIL-18Rβ-mFc (SEQ ID NO: 1) or huIL-18Rβ-HisAvi (SEQ ID NO: 2). Characterization of the immunized mouse cohorts and antigens are disclosed in Table E1.

TABLE E1
Description of recombinant protein antigen
and protocol used during immunization.

| Mouse | Name of | | |
|---|---|---|---|
| Cohort | Immunogen | SEQ ID NO: of Immunogen | Protocol |
| 1 | huIL-18Rβ-mFc | 1 | Short |
| 2 | huIL-18Rβ-mFc | 1 | Short |
| 3 | huIL-18Rβ-HisAvi | 2 | Long |
| 4 | huIL-18Rβ-HisAvi | 2 | Long |

Following immunization, spleen and lymph node cells from the mice were harvested and antibodies specific to IL-18Rβ were identified by immune library selections. Specifically, V_{H} mRNA and V_{L} mRNA were isolated from the antibody-producing B cells of the immunized mice. An immune library (library size 106-107) was constructed in a manner that maintained the original V_{H}-V_{L} pairing that was present in the mouse B cell. This library was transfected into and expressed by yeast surface display and sorted against human IL-18Rβ using fluorescence-activated cell sorting (FACS). Single yeast cells that displayed IL-18Rβ positive antibodies were sorted into plates, and transcriptionally active PCR (TAP) DNA products were generated from the antibody genes. The TAP DNA was transiently transfected into HEK cells for expression as IgGs.

In parallel, deep sequencing of the immune libraries was also carried out using next generation sequencing (NGS). Libraries were sorted for binding to human IL-18Rβ as well as for a loss of binding when IL-18/IL-18Rα was in complex with IL-18Rβ. This latter criterion during sorting was performed because members that do not bind to IL-18Rβ in the presence of IL-18/IL-18Rα are most likely to bind to the epitope involved in active ternary complex formation. By carrying out NGS antibodies with desired binding profile were identified and were gene synthesized in IgG expression vectors and transiently transfected into HEK cells for expression.

In addition to the immune libraries, fully human synthetic antibody libraries composed of fixed antibody framework and defined complementarity-determining regions (CDRs) were also constructed. These libraries (library size >10¹²) were taken through selection using mRNA display followed by yeast surface display to sort for binders to huIL-18Rβ using FACS, followed by deep sequencing and the same single yeast cell sorting as performed for the immune libraries.

The selections using both immune and synthetic libraries, followed by TAP or deep sequencing then transient transfection in HEK cells, produced 1,380 candidate antibodies that were then subjected to screening for binding to IL-18Rβ and for the ability of the antibody to block IL-18 ternary complex formation.

### B. Surface Plasmon Resonance (SPR) to Assess Binding to IL-18Rβ

The kinetics of the candidate antibodies were screened to assess how well each candidate antibody bound to human IL-18Rβ using two different surface plasmon resonance (SPR) instruments: a LSA and a Biacore 8K instrument.

For the first instrument, an HC30M biosensor (Carterra) was docked onto the LSA (Carterra), and the instrument was primed with 50 mM MES pH 5.5, 0.05% Tween-20. Sensor temperature was configured at 25° C. and plate temperature was 20° C. The sensor was conditioned with 2 minute injections of 50 mM NaOH, 1M NaCl, and 10 mM glycine pH 2.0. Antibodies for screening were diluted to 10 μg/ml in 10 mM sodium acetate pH 5.0. The sensor was activated for amine coupling with a 7 minute injection of 33 mM sulfo-NHS, 133 mM EDC diluted into 100 mM MES pH 5.5. Candidate antibodies were printed onto the biosensor array for 15 minutes, followed by a 7 minute 1 M ethanolamine pH 8.5 quenching injection, and lastly two 15 second injections of the MES running buffer.

After the α-huIL-18Rβ antibodies were immobilized onto the biosensor array, the LSA was primed into HBSTE (10 mM HEPES, 150 mM NaCl, 3 mM EDTA, 0.05% Tween 20), 0.5 mg/ml BSA, pH 7.4. The sensor temperature was adjusted to 37° C. Non-regenerative single-cycle kinetics were collected as follows: a 0.27-200 nM titration series of human IL-18Rβ (SEQ ID NO: 2) and mouse IL-18Rβ (SEQ ID NO: 4) proteins were injected for 5 min association with 15 min dissociation. Exemplary antigen sequences used during the single-cycle kinetics titrations are disclosed in Table E2 below.

TABLE E2
Antigens Used During LSA Single-Cycle Kinetics Titrations

| Antigen Name | SEQ ID NO: of Antigen |
|---|---|
| huIL-18Rβ-HisAvi | 2 |
| muIL-18Rβ-HisAvi | 4 |

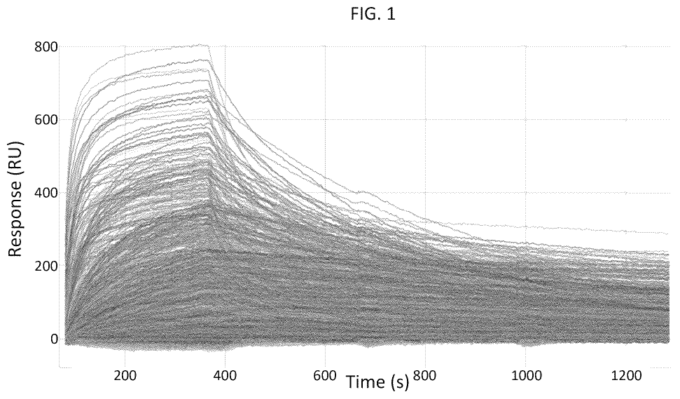

In between titration injections, the biosensor surface was regenerated using two 30 sec injections of 10 mM glycine pH 2.0. Representative SPR binding sensorgrams for a panel (four 96-well plates) of α-human IL-18Rβ antibodies screened using Carterra LSA are shown in FIG. 1. The data shows that a range of kinetic profiles for human IL-18Rβ was observed.

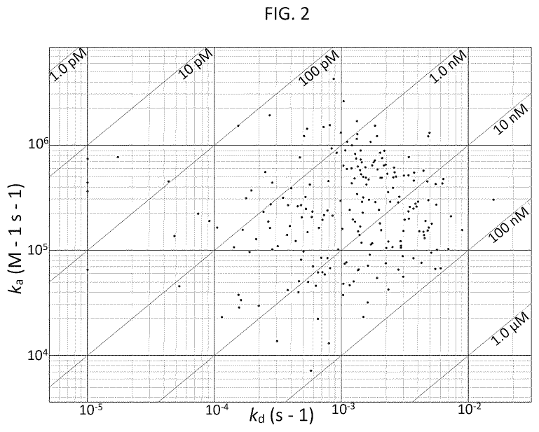

Sensorgrams were then analyzed and fit to a 1:1 binding model using Carterra Kinetics software. FIG. 2 depicts analyzed, fitted kinetic parameters for an exemplary panel (four 96-well plates) of α-human IL-18Rβ antibodies binding to human IL-18Rβ. As shown, data were graphed in an isoaffinity plot, which shows the on-rate (ka) plotted against the off-rate (kd). The diagonal lines represent the dissociation constant (K_{D}). Each node plots the kinetics for an antibody. The α-human IL-18Rβ antibodies from this screening experiment exhibited multiple logs of affinities, ranging from ˜100 nM to ˜10 pM under the assay conditions tested.

Kinetics were also screened using an alternative instrument. Specifically, Biacore CM5 Series S sensors (Cytiva) were docked onto Biacore 8K instruments (Cytiva), primed with HBS-P+ (10 mM HEPES, 150 mM NaCl, 0.05% Tween 20, pH 7.4). An α-human-Fc capture sensor surface (Cytiva #29234600) was prepared using the manufacturer recommended protocol. The sample compartment was adjusted to 15° C., and sensor surface to 37° C. Screening was conducted on α-human IL-18Rβ antibodies in 96-well format using the following conditions. Antibodies were diluted to 25 nM in HBS-P+ and captured for 10 μl/min for 45 seconds. Single-cycle kinetics titrations of 0, 7.4, 22.2, 66.6, and 200 nM human IL-18Rβ and cynomolgus monkey (cyno) IL-18Rβ proteins (SEQ ID NOS: 2 and 3, respectively) were injected across all flow cells at 30 μl/min for 3 minutes, and dissociation was collected for 5 minutes. Exemplary antigen sequences used in the single-cycle kinetics titrations are disclosed in Table E3 below.

TABLE E3
Antigens Used During BiaCore Single-Cycle Kinetics Titrations

| Antigen Name | SEQ ID NO: of Antigen |
|---|---|
| huIL-18Rβ-HisAvi | 2 |
| cyIL-18Rβ-HisAvi | 3 |

While hull-18Rβ-HisAvi was used to assess candidate antibody binding to human IL-18Rβ, cyIL-18Rβ-HisAvi was used to evaluate each candidate antibody's cross-species reactivity.

The α-human Fc biosensor surface was regenerated between cycles using two 30 second injections of 3M magnesium chloride. Sensorgrams were double-reference subtracted and fit to a 1:1 binding model in Insight Evaluation software (Cytiva).

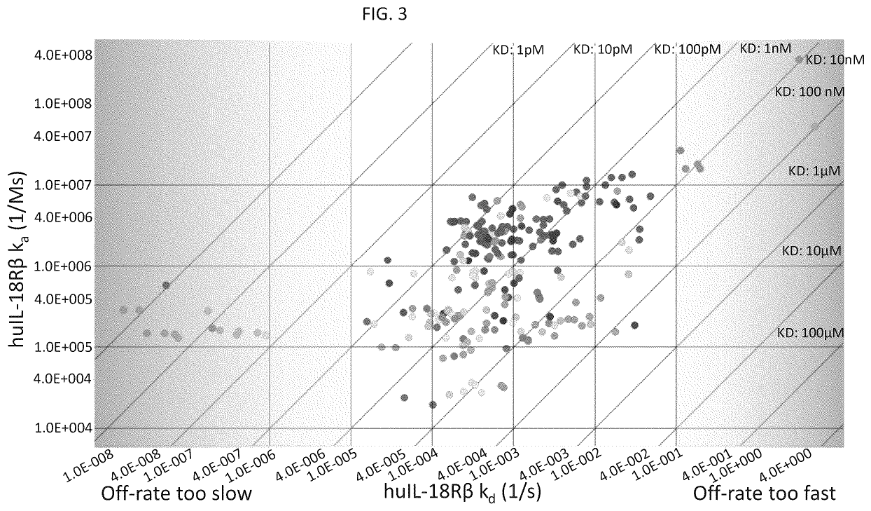

FIG. 3 depicts analyzed, fitted kinetic parameters for an exemplary panel (four 96-well plates) of α-human IL-18Rβ antibodies binding to human IL-18Rβ (huIL-18Rβ-HisAvi; SEQ ID NO: 2). Data were graphed in an isoaffinity plot, which shows the on-rate (ka) plotted against the off-rate (kd). The diagonal lines represent the dissociation constant (KD). Each node plots the kinetics for an antibody. The α-human IL-18Rβ antibodies from this screening experiment exhibited multiple logs of affinities, ranging from ˜100 nM to ˜1 pM under the assay conditions tested, with several antibodies identified with off-rates at the limit of SPR detection (slower than 1×10⁻⁶s⁻¹).

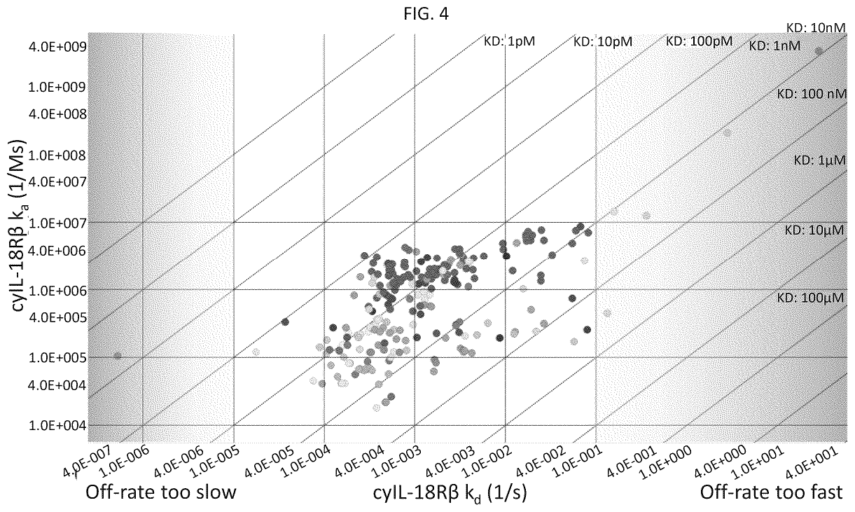

Similarly, FIG. 4 depicts analyzed, fitted kinetic parameters for an exemplary panel (four 96-well plates) of α-human IL-18Rβ antibodies binding to cyno IL-18Rβ (cyIL-18Rβ-HisAvi; SEQ ID NO: 3). Data were graphed in an isoaffinity plot, which shows the on-rate (ka) plotted against the off-rate (kd). The diagonal lines represent the dissociation constant (KD). Each node plots the kinetics for an antibody. The α-human IL-18Rβ antibodies from this screening experiment exhibited multiple logs of affinities, with the majority ranging from ˜ 100 nM to ˜ 100 pM under the assay conditions tested.

Together, these results show that high-throughput SPR techniques were able to screen multiple α-huIL-18Rβ candidate antibodies for binding to human IL-18Rβ and measure kinetics for the receptor, as well as cross-species reactivity with cyno IL-18Rβ. In total, 1380 IL-18Rβ antibodies were evaluated. Out of these, 242 antibodies representing unique sequence families were further characterized for their ability to prevent IL-18 ternary signaling complex formation. For sequence families with multiple family members, the antibody with the best affinity to huIL-18Rβ was advanced.

### C. Assay to Assess Prevention of IL-18 Ternary Signaling Complex Formation

242 candidate IL-18Rβ antibodies were further assessed using competitive Octet bio-layer interferometry (BLI) assays to identify α-IL-18Rβ antibodies that bind human (hu) IL-18Rβ and prevent formation of the IL-18 signaling complex by blocking the interaction with IL-18: IL-18Ra.

Binding assays were performed on an OctetRed384 bio-layer interferometry (BLI) instrument (Sartorius/ForteBio) in 16-channel mode. All assay steps were performed at 30° C. at 1000 rpm shake speed, and the buffer used was PBST (137 mM sodium chloride, 2.7 mM potassium chloride, 10 mM phosphate buffer. 0.05% Tween 20, pH 7.4). Biotinylated human IL-18Rβ-Avi-His (SEQ ID NO: 2) was diluted to 100 nM in PBST and captured on High Precision Streptavidin (SAX) biosensors (Sartorius). The SAX sensors were blocked next with a mixture of 100 μg/ml total human IgG (Jackson #009-000-002), 100 μg/ml total mouse IgG (Jackson #015-000-002), and 10 μg/ml biocytin (ThermoFisher #B1592). The α-huIL-18Rβ antibodies were diluted to 50 nM and binding responses to the captured IL-18Rβ were observed. The IL-18: IL-18Rα complex was created by mixing 1.6 uM human IL-18 (R&D Systems #9124-IL/CF) with 100 nM human IL-18Ra-Fc-His protein (R&D Systems #816-LR), a 1:8 molar excess of IL-18. Binding of complexed IL-18: IL-18Rα was measured to identify antibodies which prevented the IL-18: IL-18Rα complex from interacting with captured IL-18Rβ.

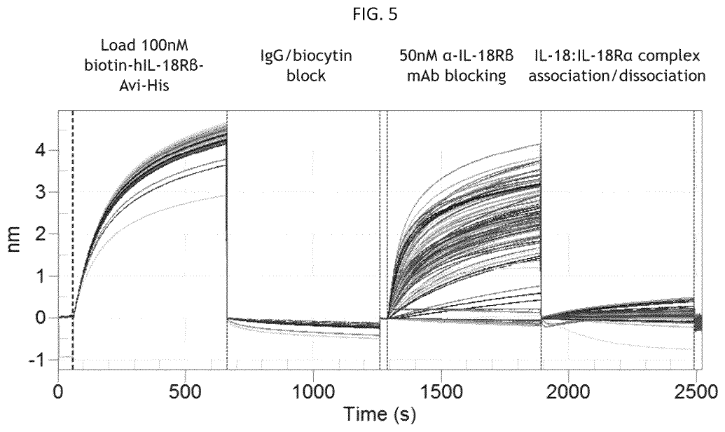

An exemplary sensorgram overlay for one set of 96 α-huIL-18Rβ antibodies screened in the blocking assay is shown in FIG. 5. The beginning of each assay step is zeroed to 0 nm response, to compare responses over the course of each step. Effective loading of biotinylated IL-18Rβ was observed (˜4 nm) and resulted in robust α-IL-18Rβ antibody responses (˜1-4 nm). The IL-18: IL-18Rα binding or blocking response was measured in the final assay step (˜0.5 nm).

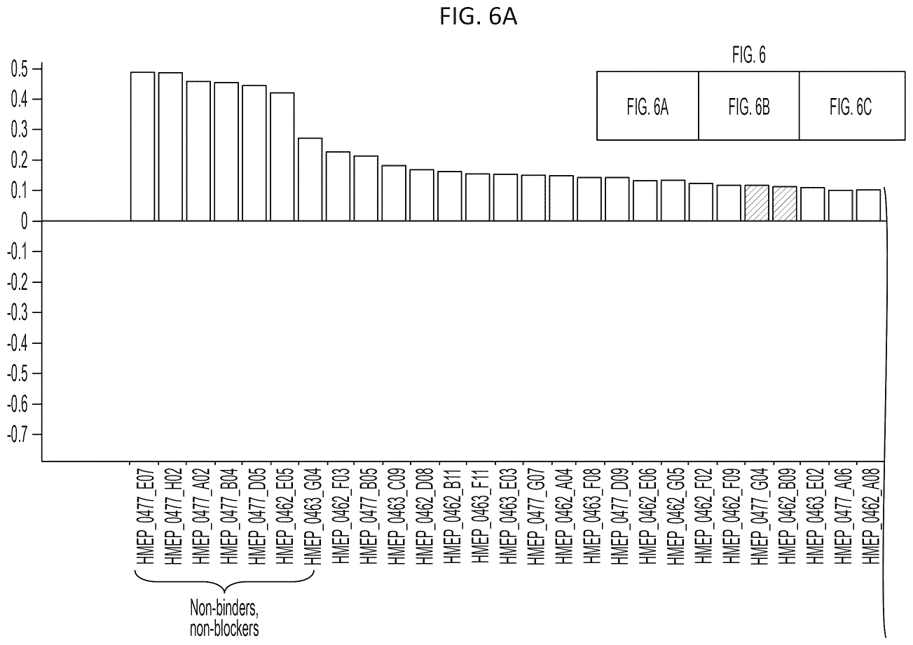

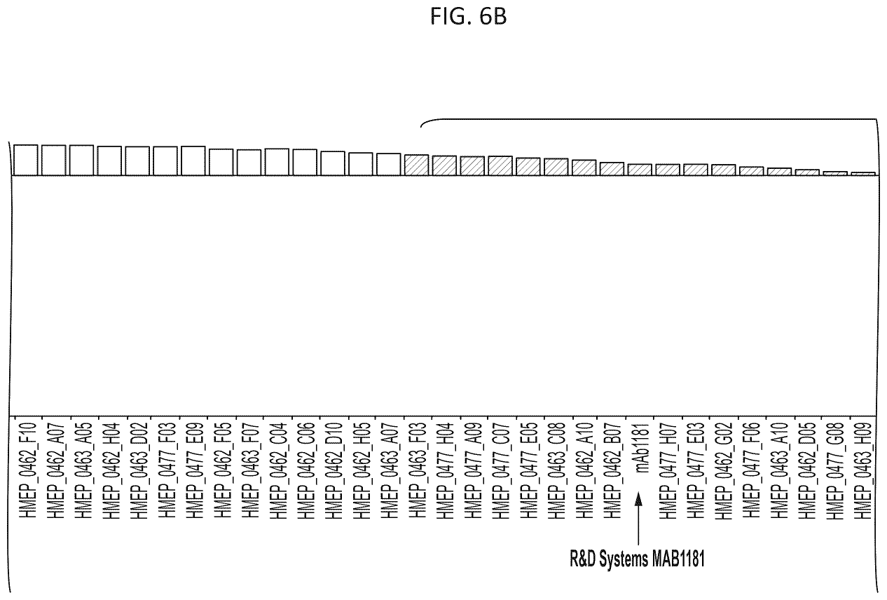

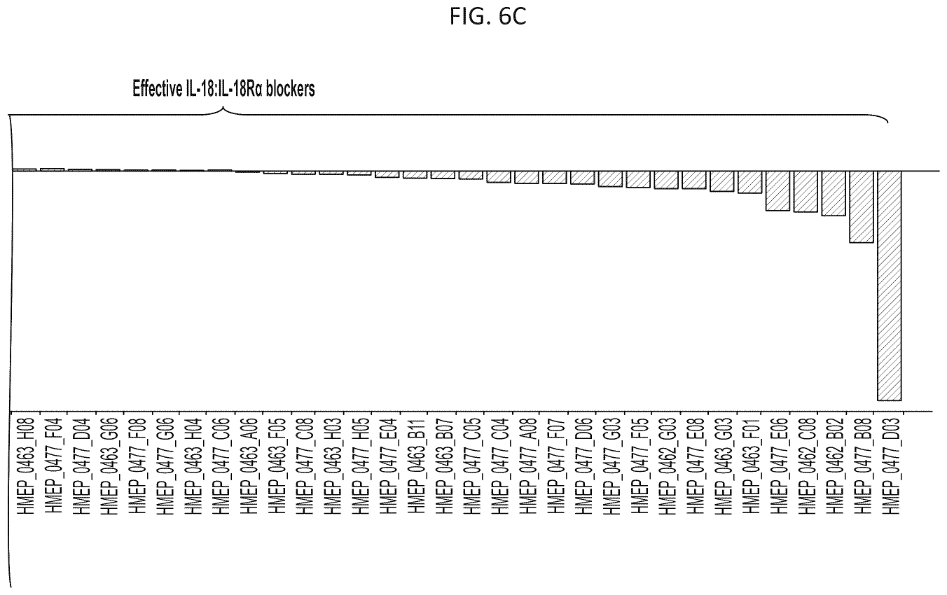

The last 10 second responses for the IL-18: IL-18Rα complex binding step shown in FIG. 5, or maximal binding responses (nm shift) for the IL-18: IL-18Rα binding step, were averaged and rank-ordered to differentiate non-blocking responses (i.e., maximal IL-18: IL-18Rα responses) from the most effective blockers. The results are shown in FIG. 6. Each bar on the X-axis represents a unique α-IL-18Rβ antibody from one representative screen of ninety-six antibodies (HMEP_XXXX_Well-ID), and blocking antibodies were benchmarked against control mouse α-huIL-18Rβ blocking antibody from R&D Systems (#MAB1181). The antibodies highlighted as “Effective IL-18: IL-18Rα blockers” were advanced for further characterization as likely IL-18 ternary signaling complex blockers. Weaker blockers (also in grey) were also advanced for comparison in subsequent experiments.

The results indicate that the blocking assay was able to identify 122 out of the 242 α-huIL-18Rβ candidate antibodies that blocked IL-18 ternary signaling complex formation.

### D. Epitope Binning

To further characterize candidate antibodies into different bins based on similar epitope properties, a LSA competitive epitope binding assay was performed.

Specifically, an HC30M biosensor (Carterra) was docked onto the LSA, and the instrument was primed with 50 mM MES pH 5.5, 0.05% Tween-20. Sensor temperature was configured at 25° C. and plate temperature was 20° C. The sensor was conditioned with 2 minute injections of 50 mM NaOH, 1M NaCl, and 10 mM glycine pH 2.0. Antibodies for binning were diluted to 10 μg/ml in 10 mM sodium acetate pH 5.0. Antibody controls included a mouse α-His tag control (R&D Systems #MAB050) and a mouse α-huIL-18Rβ antibody (R&D Systems #MAB1181). The sensor was activated for amine coupling with a 7 minute injection of 33 mM sulfo-NHS, 133 mM EDC diluted into 100 mM MES pH 5.5. Antibodies were printed onto the biosensor array for 15 minutes, followed by a 7 minute 1 M ethanolamine pH 8.5 quenching injection, and lastly two 15 second injections of the MES running buffer.

After the α-human (hu) IL-18Rβ antibodies were immobilized onto the biosensor array, the LSA was primed into HBSTE (10 mM HEPES, 150 mM NaCl, 3 mM EDTA, 0.05% Tween 20), 0.5 mg/ml BSA. pH 7.4. All samples were diluted into the assay buffer. The classical binning experiment consisted of the following cycles: first a 1 min injection of buffer is injected as a baseline, followed by a 5 min injection of 200 nM huIL-18Rβ protein (SEQ ID NO: 2). Next, the competitor antibody, diluted to 150 nM, is flowed for 5 min across the surface to allow for sandwiching or blocking interactions with the captured IL-18Rβ. Dissociation is measured for 1 min, then the surface is regenerated between cycles with two 2 min injections of 10 mM glycine pH 1.75. The injection cycle is repeated for each antibody included in the binning experiment. Binning data was analyzed using Carterra Epitope software, and the sandwiching/blocking threshold was manually assigned for each antibody assayed.

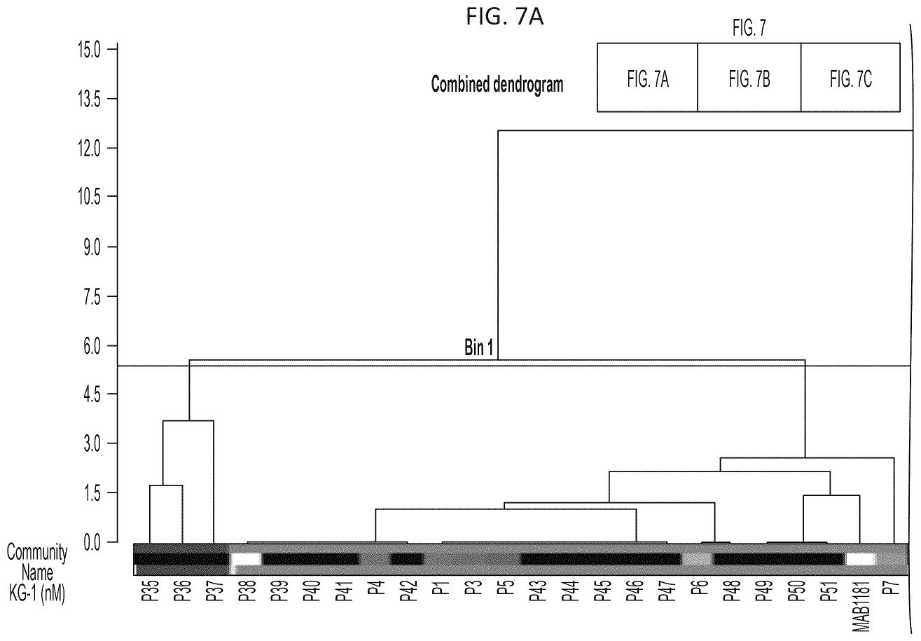

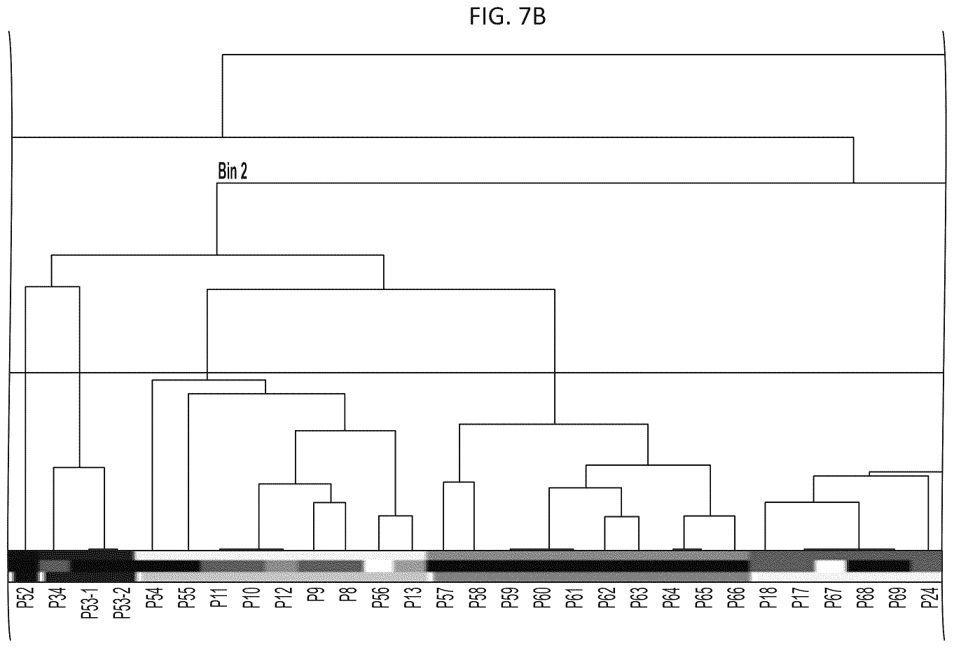

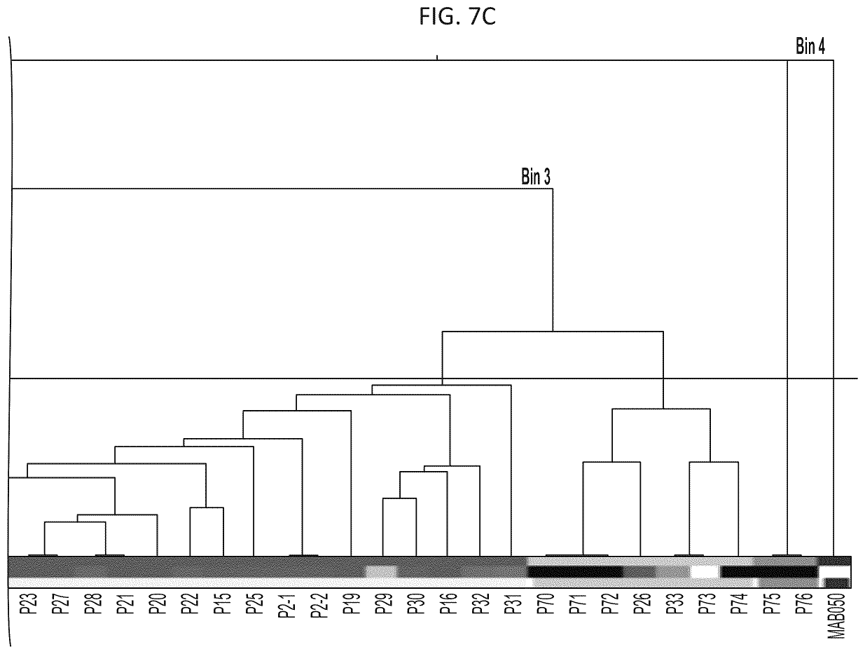

The LSA classical sandwich assay indicated that there was a total of four blocking epitopes, including three primary epitope bins, for this exemplary α-huIL-18Rβ antibody panel. Hierarchical correlational clustering applied to the binarized binding/blocking data in the Carterra Epitope software yielded the combined dendrogram shown in FIG. 7, with the primary epitope bin branches labeled on the dendrogram. Antibodies are listed along the x-axis and grouped according to the dendrogram. The horizontal bar represents the threshold at which the epitope community groupings were defined, then labeled as shown on the dendrogram. The control α-IL-18Rβ antibody MAB1181 is in epitope bin 1. Bins 2, 3 and 4 are distinct epitopes.

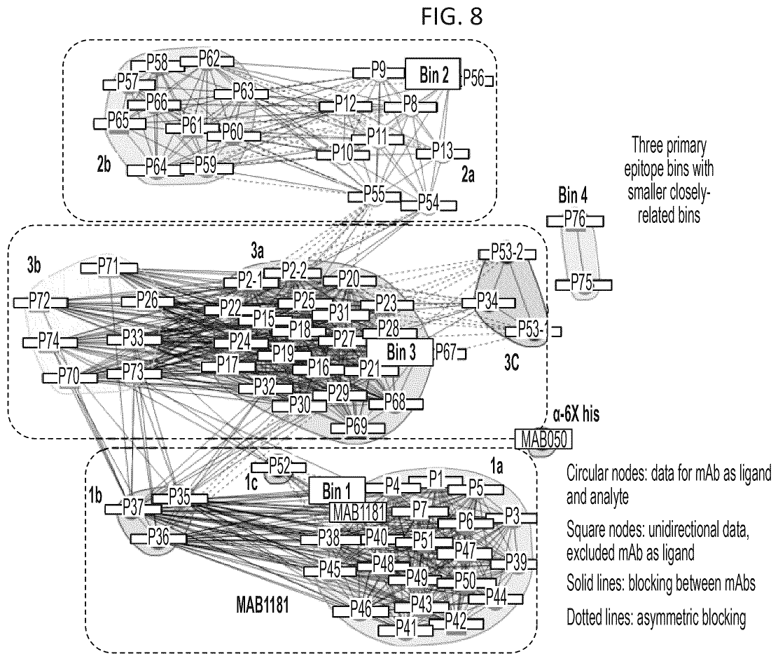

The dendrogram, along with sensorgrams generated from the LSA classical sandwich assay, were used to help refine the bin node plot shown in FIG. 8, which showcases the binding and blocking relationships within and between the defined epitope bins. Three primary epitope bins were identified and labeled. Each antibody is represented by one node, and lines connecting two nodes indicate a blocking interaction between the two antibodies. The control α-IL-18Rβ antibody MAB1181 is in epitope bin 1, and the control α-His antibody MAB050 binds only to the His-tag on the IL-18Rβ protein and is not grouped with any other antibodies. The data shows that blocking relationships within each bin were highly correlated, and for some of the smaller subpopulations (e.g., a and b), there were weak cross-blocking interactions across the three main bins. The few antibodies exhibiting blocking interactions across bins indicate some potential overlap between bins and the potential for blocking epitopes to be in close proximity on IL-18Rβ. Resolution of this binning format was excellent, and the assay indicated that the blocking α-huIL-18Rβ antibodies included in this experiment bound to four distinct epitopes on huIL-18Rβ.

Thus, the classical sandwich competitive epitope binning assay indicated that the blocking IL-18Rβ antibodies identified belong to four epitope bins, and the majority were grouped into one of three primary epitope bins.

### E. KG-1 Assay to Assess Potency of Antibodies to Block IL-18 Dependent IFNγ Production

After identifying 122 candidate antibodies that blocked ternary complex formation, each antibody's potency was assessed by determining the inhibition of IL-18 mediated IFNγ release from KG-1 cells using a broad range of antibody concentration from 0.04 nM to 100 nM.

Specifically, KG-1 cells (ATCC CCL-246) were grown in IMDM (Thermo Fisher, cat #12200) with 20% Heat Inactivated FBS (Gibco, 10082-147) and Penicillin-Streptomycin in a 37C 5% CO2 incubator. 50,000 cells per well were plated in a 96-well plate (Thermo-Fisher, cat #07-200-90) in presence of recombinant human TNF-α at 20 ng/mL final concentration (R&D, cat #210-TA-100/CF). They were then pre-treated for 1 h with anti-human (hu) IL-18Rβ antibody with a final concentration range from 100 nM to 0.04 nM. One hour later, the cells were then stimulated with recombinant human IL-18 (Sino Biologics, cat #10119-HNCE) at a final concentration of 8 ng/ml in the assay well. After a 16-24 hours incubation at 37° C., 5% CO2, the plates were centrifuged for 5 minutes at 2300 rpm. Supernatants were then collected for IFNγ secretion measurement using the MSD platform (IFNγ MSD assay, cat #K151QO). To calculate percent inhibition, the activity (IFNg level) in the presence of inhibitory antibody was compared to the activity in the absence of the inhibitor (such as isotype control). The following general formula for percent inhibition was used:

Percent Inhibition=[(Control Activity−Inhibited Activity)/Control activity]×100.

The KG-1 assay found that 33 of the 122 tested antibodies demonstrated IL-18 mediated IFNγ secretion in a dose-dependent manner. The KG-1 assay results, including IC50 values along with affinity and epitope bins of the exemplary 33 antibodies binding to human and cynomolgus monkey (cyno) IL-18Rβ are disclosed in Table E4 below.

TABLE E4
Affinity and potency of anti-huIL-18Rβ blocking antibodies

| | Antibody | K_{D}, | IC₅₀, | K_{D}, |
|---|---|---|---|---|
| Bin | Name | nM, hu | nM, KG-1 | nM, cy |
| 1a | P1 | 4.5 | 113 | 1.8 |
| 1a | P2 | 6.8 | 34.8 | poor fit/no |
| | | | | binding |
| 1a | P3 | 8.2 | 84 | 19.4 |
| 1a | P4 | 12 | 100 | 68 |
| 1a | P5 | poor fit | 59 | poor fit |
| 1a | P6 | poor fit | 362 | poor fit |
| 1a | P7 | poor fit | >270 | poor fit |
| 2a | P8 | 0.094 | 0.3 | Weak |
| 2a | P9 | 0.22 | 2.7 | no binding |
| 2a | P10 | 0.6 | 4.2 | no binding |
| 2a | P11 | 4.4 | 1.6 | Weak |
| 2a | P12 | 4.0 | 213 | no binding |
| 2a | P13 | 11 | 291 | fast on/fast off |
| 2a | P14 | 0.2 | N.D. | 73 |
| 3a | P15 | sub-nM* | 24.1 | 7.7 |
| 3a | P16 | sub-nM* | 3.4 | sub-nM* |
| 3a | P17 | 0.9 | 4.5 | ND |
| 3a | P18 | 0.9 | 3.1 | 1.7 |
| 3a | P19 | 1.1 | 8.9 | 4 |
| 3a | P20 | 1.4 | 0.2 | 3.8 |
| 3a | P21 | 1.4 | 0.7 | 18 |
| 3a | P22 | 3 | 49.4 | 3.7 |
| 3a | P23 | 3.1 | 7.3 | 82 |
| 3a | P24 | 4 | 14.7 | 43.8 |
| 3a | P25 | 4.4 | 27.8 | poor fit |
| 3b | P26 | 6.5 | 1.4 | 4.9 |
| 3a | P27 | 15 | 1.2 | 36 |
| 3a | P28 | 18.7 | 67 | 5.7 |
| 3a | P29 | 21 | >460 | |
| 3a | P30 | 24.4 | 38 | 18 |
| 3a | P31 | 37 | 103 | 78.7 |
| 3a | P32 | 39 | 89 | 26.5 |
| 3b | P33 | 19.6 | 257 | 10.1 |
| 3c | P34 | 1.9 | 7.1 | 12.8 |

The 33 antibodies listed in Table E4 blocked ternary complex formation and IL-18Rβ dependent IFNγ production in KG-1 cells, spanned 3 epitope bins, and exhibited diverse affinity binding to human and cyno IL-18Rβ. As antibodies belonging to Bin 1 demonstrated an affinity-potency disconnect, they were not further assessed. Instead, 8 of the 33 antibodies that demonstrated a high affinity and high potency as well as having an IC50 value in the single-digit nM range or better were chosen for further characterization.

### Example 2—Further Characterization of Selected IL-18Rβ Antibodies

This example provides additional characterization of 8 selected antibodies, including assessments of developability, binding by surface plasmon resonance (SPR), potency in human whole blood, and immunogenicity. The 8 human anti-interleukin-18 receptor-β (huIL-18Rβ) antibodies chosen for further study were: P8, P9, P11, P14, P18, P19, P20, and P21.

A. Assessing Effect of pH on binding to hulL-18Rβ

One desired indication for an exemplary anti-human IL-18 receptor-β (anti-bulL-18Rβ) antibody is to treat inflammatory bowel disease (IBD). As a result, several parameters were considered in proposing optimal kinetic parameters of the optimal anti-hulL-18Rβ antibody. The first parameter was risk of target mediated drug disposition (TMDD). While TMDD is a risk for antibodies targeting receptors, the expression of IL-18Rb is quite low in the IBD colon (below the lower limit of quantification via targeted mass spectrometry), thus making TMDD an unlikely risk. The second parameter was gut pH. Luminal colon pH is acidic in healthy gut (see, e.g., Newton and Kumar, Interdiscip J Microinflammation 2014 1:2), and pH of the inflamed tissue is likely lower. The final parameter is that IBD is a disease with extraintestinal manifestations (Rogler et al., Gastroenterology, 2021) and is therefore a systemic disease. Taken together, these parameters suggest that a pH-independent anti-huIL-18Rβ antibody that can bind to IL-18Rb at both acidic and neutral pH is desired in the treatment of IBD. Thus, the antibodies were tested for binding at pHs ranging from 5 to 7 using surface plasmon resonance (SPR).

Specifically, Biacore CM5 Series S sensors (Cytiva) were docked onto Biacore T200 instruments (Cytiva), primed with PBST (10 mM Na2HPO4, 137 mM NaCl, 2.7 mM KCl, and 1.8 mM KH2PO4, 0.05% Tween 20, pH 7.4). An α-human-Fc capture sensor surface (Cytiva #29234600) was prepared using the manufacturer recommended protocol. The sample compartment was adjusted to 10° C., and sensor surface to 37° C. Kinetics were measured for α-huIL-18Rβ antibodies using the following conditions: the entire assay was run once using PBST pH 7.4 running buffer, then the cycles were repeated using PBST (pH 6.0, 5.5, or 5.0) running buffer across the same biosensor. Antibodies were diluted to 15 nM in PBST (pH 7.4) and captured for 10 μl/min for 25 seconds. Multi-cycle kinetics titrations of 0, 0.78, 1.56, 3.12, 6.25, 12.5, 25, 50, 100 and 200 nM human IL-18Rβ protein (SEQ ID NO: 2) and cynomolgus monkey (cyno) IL-18Rβ protein (SEQ ID NO: 3), diluted in PBST buffer at the appropriate pH, were injected across all flow cells at 30 μl/min for 3 minutes, then dissociation was collected for 5 minutes. The α-human Fc biosensor surface was regenerated between cycles using two 30 second injections of 3 M magnesium chloride. Sensorgrams were double-reference subtracted and fit to a 1:1 binding model in T200 Evaluation software or Insight Evaluation software (Cytiva).

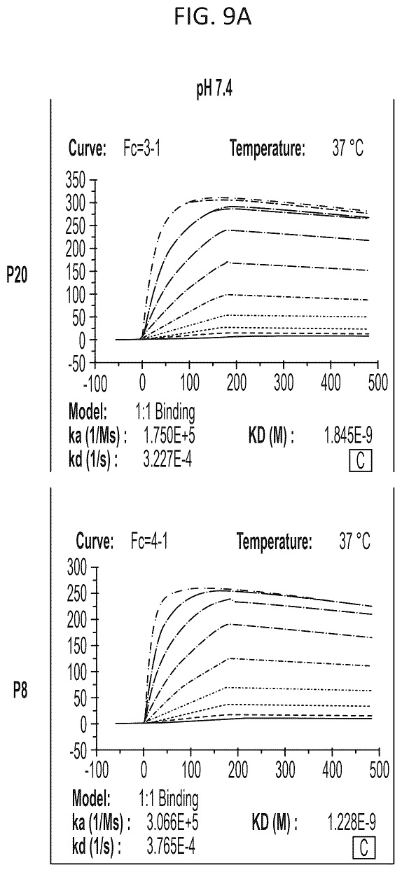

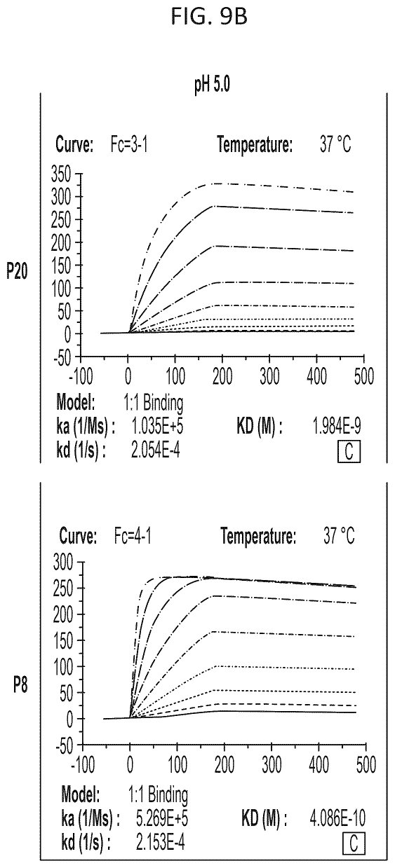

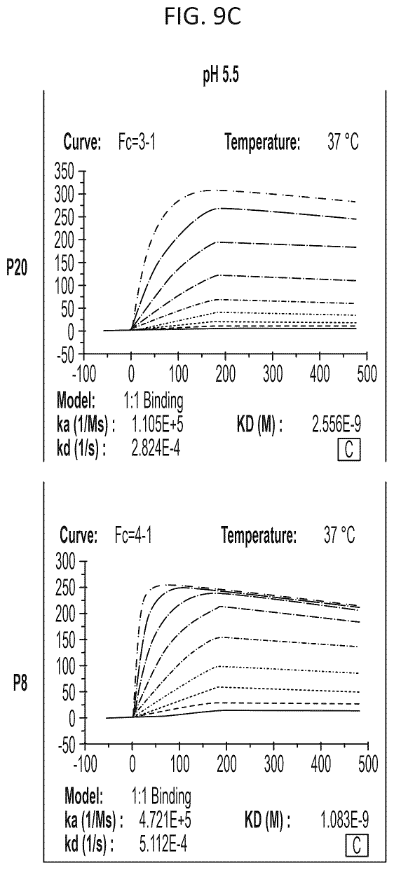

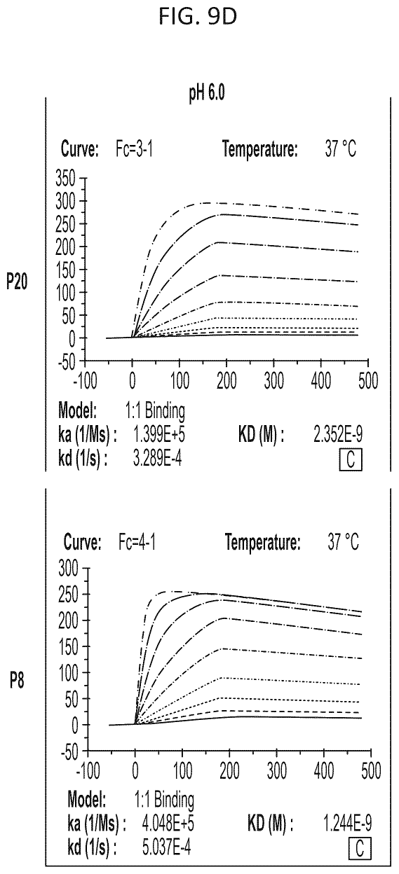

FIGS. 9A-9D showcase the sensorgrams for two exemplary anti-IL-18Rβ antibodies, P20 and P8. FIG. 9A showcases the sensorgrams at a pH of 7.4; FIG. 9B showcases the sensorgrams at a pH of 5.0; FIG. 9C showcases the sensorgrams at a pH of 5.5; and FIG. 9D showcases the sensorgrams at a pH of 6.0. As shown, these exemplary antibodies appear to exhibit pH-independent binding to human IL-18Rβ.

Kinetic parameters for the antibody panel assessed are shown at the two extreme pHs tested (pH 5.0 and pH 7.4) in Table E5 below. Epitope bins were previously generated in Example 1.D.

TABLE E5
Multi-pH human IL-18Rβ kinetics for the panel of antibodies

| Antibody | Epitope | | ka | kd | KD | kd ratio (kd pH | KD ratio (KD pH |
|---|---|---|---|---|---|---|---|
| Name | Bin | pH | (1/Ms) | (1/s) | (M) | 5/kd pH 7) | 5/KD pH 7) |
| P19 | 3a | 5 | 1.9 × 10⁵ | 5.2 × 10⁻⁴ | 2.7 × 10⁻⁹ | 0.91 | 0.65 |
| P20 | Ba | | 1.0 × 10⁵ | 2.1 × 10⁻⁴ | 2.0 × 10⁻⁹ | 0.63 | 1.08 |
| P8 | 2a | | 5.3 × 10⁵ | 2.2 × 10⁻⁴ | 4.1 × 10⁻¹⁰ | 0.57 | 0.33 |
| P9 | 2a | | 1.2 × 10⁵ | 2.6 × 10⁻⁴ | 2.1 × 10⁻⁹ | 0.50 | 0.59 |
| P11 | 2a | | 2.9 × 10⁵ | 1.1 × 10⁻³ | 3.8 × 10⁻⁹ | 1.08 | 0.51 |
| P21 | 3a | | 1.6 × 10⁵ | 4.0 × 10⁻⁴ | 2.5 × 10⁻⁹ | 0.79 | 1.09 |
| P19 | 3a | 7.4 | 1.4 × 10⁵ | 5.8 × 10⁻⁴ | 4.2 × 10⁻⁹ | | |
| P20 | 3a | | 1.8 × 10⁵ | 3.2 × 10⁻⁴ | 1.8 × 10⁻⁹ | | |
| P8 | 2a | | 3.1 × 10⁵ | 3.8 × 10⁻⁴ | 1.2 × 10⁻⁹ | | |
| P9 | 2a | | 1.5 × 10⁵ | 5.2 × 10⁻⁴ | 3.6 × 10⁻⁹ | | |
| P11 | 2a | | 1.4 × 10⁵ | 1.0 × 10⁻³ | 7.4 × 10⁻⁹ | | |
| P21 | 3a | | 2.2 × 10⁵ | 5.0 × 10⁻⁴ | 2.3 × 10⁻⁹ | | |

As shown in Table E5, the off-rates (kd) and dissociation constants (KD) measured at pH 5 are within 3-fold of the neutral pH measurements, indicating parity in kinetics across the pH range tested, and pH-independent binding for IL-18Rb.

In conclusion, the antibodies exhibited pH-independent binding to human IL-18Rβ, with both off-rate (kd) and dissociation constant (K_{D}) within three-fold comparing the most acidic pH tested (pH 5) to neutral pH (i.e., kd and K_{D} ratios between 0.33 and 3.33), suggesting that they could successfully treat IBD.

### B. Biophysical Assessments to Determine Antibody Developability

The selected antibodies were also biophysically assessed by three different assays to determine antibody developability. An extracellular matrix (ECM) ELISA assay was performed to assess if antibodies exhibited polyreactivity, i.e., might bind non-specifically to extracellular matrix proteins. Additionally, hydrophobic interaction chromatography (HIC) and clone self-interaction by bio-layer interferometry (CSI-BLI) were used to measure surface hydrophobicity and self-interaction propensity of the antibodies, respectively. Surface hydrophobicity and self-interaction propensity were measured because they may cause low solubility and aggregation, thus impeding the developability of the antibody.

The ECM ELISA assay to measure polyreactivity was performed by first incubating 96-well Corning Thin-Layer Matrigel Matrix pre-coated extracellular matrix (ECM) plates (Corning catalog #354607) for one hour at room temperature with 300 μL of blocking buffer (10% FCS in TBS, Alfa Aesar catalog #J61327). After incubation, the blocking solution was removed, and antibodies at 1 and 0.1 μM in 100 μL of fresh blocking buffer were added to the wells. Five wells had no sample addition for background and ECM score calculations. After one hour of sample incubation, the samples were removed and plates were washed with PBS-T wash buffer 3 times. 10 ng/ml of goat anti-human IgG-HRP conjugated detection antibody (Jackson ImmunoResearch catalog #109-035-008) was added at 100 μl to each well. After another hour incubation at room temperature, the wells were washed 3 times with PBS-T wash buffer. After washing, 100 μl of TMB substrate (Surmodics catalog #TMBW-1000-01) was added to each well and allowed to react for 15 minutes, followed by addition of 100 μl of BioFX 450 nm stop reagent for TMB microwell substrates (Surmodics catalog #STPR-1000-01). Absorbance was then read on a microplate reader at 450 nm and referenced at 620 nm. ECM scores, which are shown in Table E6 below, were then calculated by dividing the absorbance values of the sample wells by the absorbance of the wells with no sample addition.

TABLE E6
ECM scores at 1 and 0.1 μM for the anti-huIL-18Rβ antibodies.

| Antibody Name | 1 μM | 0.1 μM |
|---|---|---|
| P20 | 3.4 | 2.1 |
| P8 | 1.1 | 1 |
| P9 | 1.2 | 1 |
| P18 | 24.9 | 24.1 |
| P11 | 1.1 | 1 |
| P21 | 1.2 | 1.2 |
| P19 | 1.6 | 1.1 |
| P14 | 24.0* | N.D. |
| low control mAb | 1.1 | |
| high control mAb | 24.2 | |

*Duplicate measurements were performed at 1 μM.
Average value at 1 μM is listed, standard deviation is 3.6.

A lower ECM score is generally desirable because it indicates less binding to extracellular matrix proteins. An ECM score of 6 at 1 μM antibody was set as the cutoff for “high” since all of the 48 clinically approved monoclonal antibodies that were tested using the same protocol showed ECM scores less than 6. All antibodies except for P18 and P14 showed low ECM scores, which indicates low polyreactivity.

Additionally, surface hydrophobicity of each humanized antibody was assessed by hydrophobic interaction chromatography (HIC). Ten μg of each antibody sample was loaded on a TSKgel Butyl-NPR column (4.6 mm×3.5 cm, 2.5 μm particle size, Tosoh P/N 14947) on an Agilent 1260 Infinity II HPLC system. A linear gradient of mobile phase A (0.1 M sodium phosphate pH 7.0, 2 M ammonium sulfate) and mobile phase B (solution 0.1 M sodium phosphate pH 7.0) was used for 20 min at a flow rate of 1.0 ml/min at 25° C. column temperature.

HIC retention times (RT) of the anti-huIL-18Rβ antibodies are listed in Table E7 below.

TABLE E7
HIC retention time for the anti-huIL-18Rβ antibodies

| Antibody Name | RT (min) |
|---|---|
| P20 | 10.8 |
| P8 | 9.6 |
| P9 | 10.0 |
| P18 | Did not elute |
| P11 | 9.8 |
| P21 | 11.6 |
| P19 | 11.6 |
| P14 | 11.1 |

A RT of 11 min was set as the cutoff, because ˜75% of 48 clinically approved monoclonal antibodies that were tested using the same experimental protocol showed RT lower than 11 minutes. Antibodies P20, P8, P9, and P11 had RT values lower than 11 min, which was considered more desirable because it indicates that the antibodies are less hydrophobic and therefore less likely to aggregate and/or exhibit solubility issues.

Finally, the self-interaction propensity of the antibodies was measured using an Octet RED384 (Sartorius). The protocol was as described in Sun et al. (2013) MAbs 2 (6): 838-41, with the following modifications. Briefly, 20 μg/mL of each antibody in the 1×PBS-T with 1 mg/mL BSA assay buffer was loaded on AHQ biosensors and allowed to incubate for 10 minutes. After blocking the biosensors with an internal human Fc (IgG1) molecule at 100 μg/mL, antibody self-interaction was measured using 1 μM of antibody solution.

The CSI-BLI ΔResponse values listed in Table E8 below were determined by subtracting the binding response from association step of a reference antibody from the binding response of the antibodies that were being evaluated.

TABLE E8
Average CSI-BLI values and standard deviations
for 1 μM antibody from 2 separate measurements.

| | Average CSI − BLI | Standard |
|---|---|---|
| Antibody Name | ΔResponse (nm) | deviation |
| P20 | 0.02 | 0.00 |
| P8 | −0.01 | 0.00 |
| P9 | −0.01 | 0.01 |
| P18 | 0.43 | 0.19 |
| P11 | −0.03 | 0.00 |
| P21 | −0.03 | 0.00 |
| P19 | 0.30 | 0.08 |
| P14 | 1.26* | |
| low control mAb | 0.00 | 0.00 |
| high control mAb | 0.25 | 0.02 |

*One measurement was carried out for PPB-6936.

Lower CSI-BLI values are generally desirable because they indicate that the antibody has less propensity to self-interact. All antibodies except for 3, specifically P14, P19, and P18, showed low CSI-BLI values.

The early developability assessment for selected anti-huIL-18Rb mAbs is summarized in Table E9 below.

TABLE E9
Summary of early developability assessment
for selected anti-huIL-18Rb mAbs

| Antibody Name | HIC | ECM | CSI-BLI |
|---|---|---|---|
| P20 | Acceptable | Acceptable | Acceptable |
| P8 | Acceptable | Acceptable | Acceptable |
| P9 | Acceptable | Acceptable | Acceptable |
| P18 | Did not elute | High | High |
| P11 | Acceptable | Acceptable | Acceptable |
| P21 | Hydrophobic | Acceptable | Acceptable |
| P19 | Hydrophobic | Acceptable | High |
| P14 | Hydrophobic | High | High |

Of the eight antibodies characterized, five of the antibodies, specifically P20, P8, P9, P11, and P21, performed favorably on at least two out of three biophysical assessments.

### C. Human Whole Blood Assay to Assess Antibody Potency

The potency of the antibodies was also assessed in LPS+IL-12 dependent stimulation of interferon gamma (IFNg) production in human whole blood.

Specifically, human whole blood was collected from healthy donors (BMS internal Blood Donor program) in EDTA vacutainer tubes (BD, ref 366643), diluted 1:1 with RPMI1640 (Gibco/Thermo-Fisher, cat #11875093) without FBS. 200 μL per well were dispensed in 96-well plates (Thermo-Fisher, cat #07-200-90). Cells were then pre-treated for 1 h with one of the 8 anti huIL-18Rb antibodies listed in Table E10 or isotype control monoclonal antibody huIgG1.3f with a final concentration range from 100 nM to 0.04 nM. One hour later, the cells were then stimulated LPS (Sigma-Aldrich, cat #L4391-1 MG)+human IL-12p70 (PeproTech, cat #200-12-50 μg) at final concentrations of 5 μg/mL and 5 ng/mL respectively in the assay well. After a 16-24 hours incubation at 37° C., 5% CO2, the plates were centrifuged for 5 minutes at 4000 rpm. Supernatants were then collected for IFNg secretion measurement using the MSD platform (IFNg MSD assay, cat #K151QO). To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

The results of this assay disclosed in Table E10 below.

TABLE E10
Antibody potency in inhibiting LPS + IL-12
dependent IFNg production in human whole blood

| Antibody Name | IC50, nM (y-max) |
|---|---|
| P20 | 0.7 ± 0.8 (98%) |
| P8 | 0.3 ± 0.1 (98%) |
| P9 | 6.7 ± 5.0 (94%) |
| P18 | N.D. |
| P11 | 7.5 ± 7.7 (91%) |
| P21 | 0.3 ± 0.2 (98%) |
| P19 | 5.7 |
| P14 | 0.4 ± 0.1 (95%) |

As shown, three of the antibodies tested in human whole blood assay maintained high potency (IC50 of <1 nM) and demonstrated high y-max (>95%) in inhibition of IFNg production, specifically P20, P8, and P21. A higher y-max indicates a more complete inhibition of the IL-18 dependent IFNg production.

### D. Immunogenicity Assessment

After an HLA class II molecule binds a peptide antigen, the next critical step in developing an anti-drug immune response is the activation of cognate T cells. This step occurs when the peptide-MHC complex is presented by an antigen presenting cell (APC) to a matching T cell receptor (TCR) on a cluster of differentiation 4 (CD4) positive T cell. In vitro assays, using diverse human donors, are often used to determine whether a candidate therapeutic contains functional T cell epitopes based on its ability to stimulate antigen specific CD4+ T cells in vitro. Thus, the three antibodies P20, P8, and P21, which exhibited the best potency and demonstrated low developability risks, were evaluated in an in vitro DC-T cell proliferation assay to determine if the—antibodies are capable of activating naïve CD4 T cells and leading to T cell proliferation.

Briefly, peripheral blood mononuclear cells (PBMCs) from healthy volunteers were isolated by negative bead-based selection (Miltenyi Biotec Inc, Bergisch Gladbach, Germany) and HLA typed using polymerase chain reaction (PCR) amplification and hybridization with oligonucleotide probes (ProImmune, Sarasota, FL). A panel of 40 PBMC donors composed of HLA-DR class II alleles closely matching the world population frequencies was used for an assay run.

Monocytes were isolated from PBMCs using a negative selection bead-based method (Miltenyi Biotec Inc, Germany) and cultured for 5 days in DC media (Lonza, Basel, Switzerland) containing Interleukin 4 (IL-4) and granulocyte-macrophage colony stimulating factor (GM-CSF) to generate immature DCs. These cells were pulsed with one of the 3 anti huIL-18Rb antibodies listed in Table E11 or with quality control proteins overnight followed by a thorough washing, and then matured by incubating the cells overnight in media containing TNF-α, IL-1b, IL-6, and PGE2.

2,000 pulsed mature DCs were co-cultured with 200,000 autologous PBMCs, labeled with carboxyfluorescein succinimidyl ester (CFSE) (Invitrogen, Carlsbad, CA) to monitor proliferation. Six replicates of each condition were plated in 96-well plates in DC media containing pen-strep (Gibco, Waltham, MA). After seven days, media was washed away and cells were stained with a fluorescently labeled (APC) anti-human CD4 (BD Biosciences, San Jose, CA) monoclonal antibody. Unbound anti-CD4 monoclonal antibody was removed with a wash step, cells were fixed with 3.7% formalin (Sigma, St. Louis, MO) in phosphate-buffered saline (PBS) and analyzed by flow cytometry to determine the percentage of proliferating antigen-specific CD4+ T cells (Table E11).

TABLE E11
Antibody-induced CD4+ T cell proliferative response
in a cohort of 40 donors for selected antibodies

| Antibody Name | Result |
|---|---|
| Avastin (low immunogenicity control) | 5% |
| P20 | 17.5% |
| P8 | 12.5% |
| P21 | 22.5% |
| VL6 (high immunogenicity control) | 20% |

As disclosed in Table E11, P20, P8, and P21 generated a CD4+ T cell proliferative response in 17.5%, 12.5%, and 22.5% of healthy donors, respectively. The low control monoclonal antibody (Avastin or bevacizumab, Genentech, Inc.), which has been demonstrated to have low immunogenicity in the clinic (Saffari et al., Int J Cancer Manag., 2018) was positive in 5% of the donors. The high control IL-21R monoclonal antibody (internally made ATR-107, Pfizer, Inc.) induced a positive response in 20% of donors and has been shown to have a high ADA rate of 76% in clinical studies (Hua et al., J Clin Pharm, 2014).

Typically, proteins that induce less than 20% response in donors are considered to have low immunogenicity risk. Thus, P20 and P8 appeared to have a low overall risk for potential clinical immunogenicity.

In summary, based on the initial characterization of the 8 antibodies that were further profiled, three pH-independent antibodies, P20, P8, and P21 demonstrated acceptable biophysical properties and high potency. In view of these results, these three antibodies were chosen for further studies to further assess their developability.

The heavy chain (HC), light chain (LC), variable heavy chain (VH) variable light chain (VL), and heavy and light chain CDR amino acid sequences contained within P20, P8, and P21 are disclosed in Table E12A below. The nucleotide sequences encoding the VH, VL, HC, and LC amino acid sequences contained within P20, P8, and P21 are disclosed in Table E12B below.

TABLE E12A
SEQ ID NOs for amino acid sequences contained in P20, P8, and P21.
SEQ ID NOs of Select Antibodies

| | | | | | CDR-H1, -H2, -H3 | CDR-L1, -L2, -L3 | HC | LC |
|---|---|---|---|---|---|---|---|---|
| | | | | | (numbering | (numbering | Constant | Constant |
| Antibody | HC | LC | VH | VL | scheme) | scheme) | Domain | Domain |
| P20 | 5 | 6 | 7 | 21 | 8, 9, 10 (Kabat) | 22, 23, 24 (Kabat) | 30 | 31 |
| | | | | | 11, 12, 10 (Chothia) | 22, 23, 24 (Chothia) | | |
| | | | | | 13, 14, 10 (AbM) | 22, 23, 24 (AbM) | | |
| | | | | | 15, 16, 17 (Contact) | 25, 26, 27 (Contact) | | |
| | | | | | 18, 19, 20 (IMGT) | 28, 29, 24 (IMGT) | | |
| P8 | 49 | 50 | 51 | 65 | 52, 53, 54 (Kabat) | 66, 67, 68 (Kabat) | 30 | 31 |
| | | | | | 55, 56, 54 (Chothia) | 66, 67, 68 (Chothia) | | |
| | | | | | 57, 58, 54 (AbM) | 66, 67, 68 (AbM) | | |
| | | | | | 59, 60, 61 (Contact) | 69, 70, 71 (Contact) | | |
| | | | | | 62, 63, 64 (IMGT) | 72, 29, 68 (IMGT) | | |
| P21 | 77 | 78 | 79 | 93 | 80, 81, 82 (Kabat) | 22, 94, 95 (Kabat) | 30 | 31 |
| | | | | | 83, 84, 82 (Chothia) | 22, 94, 95 (Chothia) | | |
| | | | | | 85, 86, 82 (AbM) | 22, 94, 95 (AbM) | | |
| | | | | | 87, 88, 89 (Contact) | 25, 96, 97 (Contact) | | |
| | | | | | 90, 91, 92 (IMGT) | 28, 29, 95 (IMGT) | | |

TABLE E12B
SEQ ID NOs for nucleotide sequences encoding amino
acid sequences contained in P20, P8, and P21.
SEQ ID NOs of nucleotide sequences encoding Select Antibodies

| Antibody | HC | LC | VH | VL |
|---|---|---|---|---|
| P20 | 137 | 138 | 135 | 136 |
| P8 | 149 | 150 | 147 | 148 |
| P21 | 153 | 154 | 151 | 152 |

### Example 3—Stability Assessment of Three Selected IL-18Rβ Antibodies

This example provides a preliminary assessment carried out to investigate the developability risks of 3 selected human anti-interleukin-18 receptor-β (IL-18Rβ) antibodies, namely P20, P8, and P21, that exhibited acceptable biophysical properties and high potency in other characterization studies. The preliminary stability assessments included measurements of monomericity, charge heterogeneity, chemical stability, and binding to human IL-18Rβ (huIL-18Rβ) of the 3 antibodies following freeze/thaw and/or different temperatures, pH, and time points.

### A. Stability Assessment Study Design

The stability of the 3 human anti-interleukin-18 receptor-β (IL-18Rβ) antibodies P20, P8, and P21 were assessed in formulation, concentration, timepoints, and storage temperatures as summarized in Table E13 below.

TABLE E13
Stability assessment study design

| Antibody | | [Conc.] | Time | | | |
|---|---|---|---|---|---|---|
| Name | Formulation | (mg/mL) | point | Storage temperature | | |
| P20 | 20 mM Histidine, 260 mM Sucrose, | 1.0 | T0 | 4° C. | 25° C. | 40° C. |
| P21 | 50 μM DTPA, 0.05% PS80, pH 6.0 | | 2 wk | | | |
| P8 | | | 4 wk | | | |
| | | | 5 × F/T | | | |
| | | | Low pH hold | | | |
| P20 | 20 mM Tris, 260 mM Sucrose, 50 μM | 1.0 | T0 | 4° C. | 25° C. | 40° C. |
| P21 | DTPA, 0.05% PS80, pH 8.0 | | 2 wk | | | |
| P8 | | | 4 wk | | | |
| | | | 5 × F/T | | | |

Specifically, the freeze-thaw stability of the three antibodies were determined by performing 5 freeze/thaw cycles. A concentration of 1 mg/mL 200 μL/vial of each antibody was added to a 2 mL screw cap clear vial (Agilent). The vials were placed in a Biocision CoolCell and stored at −80° C. for 2 hours. The CoolCell was removed and placed at room temperature (˜23° C.) for 2 hours. This was repeated a total of 5 times and the samples were analyzed.

Low pH hold was also used to evaluate stability through viral inactivation protocol. 300 μL of material was equilibrated to 25° C. using VWR Thermal Shake Touch 120V (Cat #89232-908). HCl was added to the antibody formulation (20 mM Histidine, 260 mM Sucrose, 50 μM DTPA, 0.05% PS80, pH 6.0) to decrease the pH to 3. The resulting low pH antibody solution was incubated for 3 hours and the pH was increased to 6 using NaOH, and was then analyzed by SEC.

Additionally, thermal incubation of the three antibodies was performed at 4° C., 25° C., and at 40° C. in a Binder incubator. A volume of 1.5 mL with a concentration of 1 mg/mL in a formulation of 20 mM Histidine, 260 mM Sucrose, 50 μM DTPA. 0.05% PS80 for pH 6.0 or 20 mM Tris, 260 mM Sucrose, 50 μM DTPA. 0.05% PS80, pH 8.0 was prepared for the stability assessment. 1.0 mL/vial of each antibody in pH 6 or pH 8 formulation was added to a 2 mL screw cap clear vial (Agilent) and parafilm tape was secured around the top to ensure a tight seal for the stability study and stored in the appropriate incubator. 500 μL of each formulation was analyzed at t0. The same volume was withdrawn from the vials stored in the incubator and analyzed at each of the 2 week and at 4 week timepoints. Chemical liabilities were analyzed by size exclusion chromatography, capillary isoelectric focusing (cIEF), peptide mapping, and surface plasmon resonance (SPR).

B. Monomericity Assessment after Varying Time, Temperature, and pH

To assess stability, including aggregation and fragmentation, size exclusion chromatography was used to determine the size homogeneity of each of the 3 antibodies following stress by freeze/thaw (F/T), low pH hold, and thermal stability (storage at 25 degrees C. and 40 degrees C.) at pH 6.0 or pH 8.0.

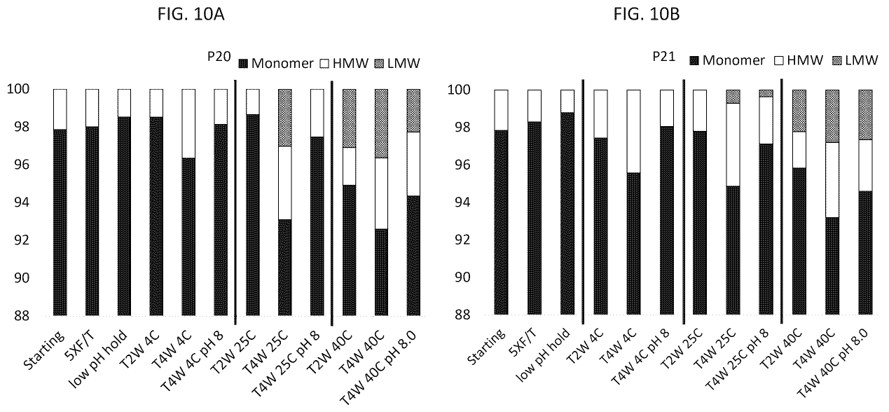

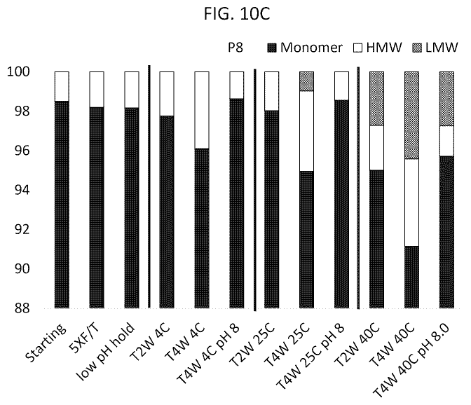

Specifically, an Agilent 1260 Infinity system was used with an Advancebio SEC 300 Å, 4.6 ×300 mm, 2.7 μm column. The running buffer was 100 mM potassium phosphate, 250 mM sodium chloride, pH 6.8. An amount of 25 μg of the sample was injected onto the column and ran at a rate of 0.2 mL/minute for 25 minutes. The chromatograms were analyzed at 280 nm and the area of the peaks were used to determine the percentage of monomer, high molecular weight (HMW) species, and low molecular weight (LMW) species for each tested antibody (FIGS. 10A-C). A gel filtration standard (Bio-Rad) was used before and after runs to ensure column integrity.

As shown in FIGS. 10A-C, each sample was analyzed initially (T0), at 2 weeks (2wk), at 4 weeks (4wk), and following 5 freeze-thaws (5×FT). The monomeric, HMW, and LMW were calculated to be within acceptable ranges, less than 5% loss of monomeric peak over the course of 4 weeks.

Overall, the three antibodies demonstrated an increase in aggregation and fragmentation that was more pronounced at higher temperatures and longer time points. The antibodies appeared more stable in storage at pH 8.0, resulting in lower aggregation and fragmentation compared to the pH 6.0 samples.

C. Assessing Charge Heterogeneity Using Capillary Isoelectric Focusing (cIEF)

Capillary isoelectric focusing (cIEF) was used to assess how formulation and temperature affected charge heterogeneity for each of the three anti-IL-18Rβ antibodies.

Specifically, cIEF was performed using a Maurice (ProteinSimple). The cIEF master mix was prepared using 46.25% diH20, 43.75% Methyl Cellulose (1%), 5% Pharmalyte 3-10, 2.5% arginine (500 mM), 1.25% pI marker 9.99, and 1.25% pI marker 4.05, to a 1.6 mL microcentrifuge tube, 40 μL of each antibody (2 mg/mL) was added to 160 μL of the master mix and vortex until completely mixed. The samples were then centrifuged at 13,000×g for 5 minutes. 160 μL of each sample was aspirate from the top of the solution and transferred to a 96-well plate. The 96-well plate was centrifuged at 1000×g for 5 minutes. The 96-well sample plate was placed in the Maurice along with the cIEF cartridge, and running reagents of 0.5% methyl cellulose, fluorescence calibration standard, and diH20. The samples were run in replicates with the method parameters of focusing period 1 (Time: 1 minute; Voltage: 3000V), focusing period 2 (Time: 8 minutes; Voltage: 3000V), detection (Absorbance 0.005 s: Florescence: 3, 5, 10, and 20 s), and sample loading duration (55 s).

The cIEF results assessing change in main peak after storage at pH 6 and 8 are shown in Table E14.

TABLE E14
cIEF results for P20, P21, and P8

| | P20 | | | P8 | | | P21 | | |
|---|---|---|---|---|---|---|---|---|---|
| | T0% | | Δ Main | T0% | | Δ Main | T0% | | Δ Main |
| | Main | Δ Main | T4W | Main | Δ Main | T4W | Main | Δ Main | T4W |
| pH | Peak | T4W 4° C. | 40° C. | Peak | T4W 4° C. | 40° C. | Peak | T4W 4° C. | 40° C. |
| 6.0 | 44.7 | 2.7 | 18.5 | 55.1 | −0.6 | 24.1 | 56.2 | 0.2 | 14.6 |
| 8.0 | 43.7 | −1.3 | 30.4 | 55 | −0.2 | 26.4 | 56.4 | 0.6 | 33.4 |

Comparing readout at baseline (T0% Main Peak) to that at 4 weeks (T4W) at 4° C., whose change is shown in A Main T4W 4° C., minimal change is observed in all three constructs at both pHs. A greater loss of main peak is observed after 4 weeks of storage at 40° C., which is shown in A Main T4W 40° C., resulting in more acidic and basic variant species. Further, this change is greater after 4 weeks of storage at pH 8 than at pH 6.

D. Chemical Stability Assessment after Varying Time, Temperature, and pH

Chemical stability and its effect on IL-18Rβ binding of the three anti-IL-18Rβ antibodies were further characterized using peptide mapping and surface plasmon resonance (SPR).

Specifically, proteins were denatured in the presence of 0.2% Rapigest surfactant (Waters Corp.) and reduced by dithiothreitol (DTT) at 80° C. for 30 minutes, alkylated by iodoacetamide (IAM) at room temp for 30 minutes in the dark, and digested by trypsin at 37° C. for 4 hours in Tris-HCL pH 7.4, followed by acidic quench.

Peptides were analyzed on an ACQUITY UPLC system (Waters™, Manchester, U.K.) coupled to a Q-Exactive™ Plus mass spectrometer (Thermo Scientific™, San Jose, CA). Peptides were eluted from a Waters BEH C18 column (130 Å 1.7 μm 2.1×150 mm, product #186002353), heated at 50° C., using a 60-minute LC gradient. The gradient was set from 0.2% to 30% solvent B in 46 minutes with a flow rate of 0.2 mL/min. Solvent A was 0.1% formic acid in water and solvent B was 0.1% formic acid in acetonitrile. Mass spectrometer was operated under positive ion mode with ESI voltage at 3.5 kV, a capillary temperature of 250° C., a scan range of 320-1800 m/z, and a sheath gas flow rate of 45.

Peptide mapping data was analyzed by Biologic™ software (Protein Metrics, Cupertino, CA) with precursor mass tolerance of 6 ppm, and fragment mass tolerance of 20 ppm. Carbamidomethylation was set as the fixed modification, and variable modifications were searched against oxidation and deamidation. The MS/MS spectra and the levels of modification were manually verified and calculated. The oxidation, deamidation, and isomerization levels of peptides were monitored. Percent Relative Modification was calculated based on the area under the curve (AUC) of modified peptide/(AUC of modified peptide+AUC of native peptide)*100.

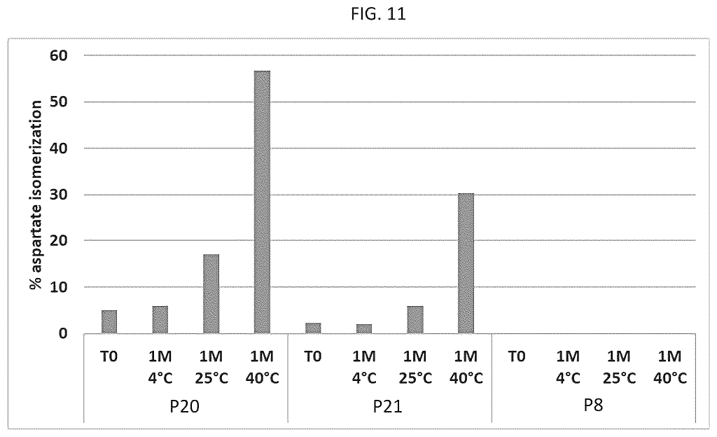

By using peptide mapping and monitoring changes over 1 month storage at 4° C., 25° C., and 40° C. pH 6 buffer, chemical stability of each of the three antibodies was determined. FIG. 11 depicts aspartate isomerization in the light chain complementarity-determining region 2 (LCDR2) of each of the three tested antibodies that was observed at baseline (T0) and after storage for one month (1M) at the indicated temperature. The data shows that, except for P8, which exhibited no chemical liability risk after 1 month of storage at all three temperatures, both P20 and P21 exhibited an aspartate isomerization liability in LCDR2.

Given that both P20 and P21 demonstrated chemical liability in LCDR2, a SPR experiment was then carried out to determine whether the aspartate isomerization in LCDR2 impacted binding of the antibodies to human IL-18Rβ (huIL-18Rβ).

The SPR kinetics were performed by docking Biacore CM5 Series S sensors (Cytiva) onto Biacore T200 instruments (Cytiva), primed with PBST (10 mM Na2HPO4, 137 mM NaCl, 2.7 mM KCl, and 1.8 mM KH2PO4, 0.05% Tween 20, pH 7.4). An α-human-Fc capture sensor surface (Cytiva #29234600) was prepared using the manufacturer recommended protocol. The sample compartment was adjusted to 10° C., and sensor surface to 37° C. Kinetics were measured for α-huIL-18Rβ antibodies using the following conditions. The entire assay was run once using PBST pH 7.4 running buffer across the same biosensor. Antibodies were diluted to 15 nM in PBST (pH 7.4) and captured for 10 μl/min for 25 seconds. Multi-cycle kinetics titrations of 0, 0.78, 1.56, 3.12, 6.25, 12.5, 25, 50, 100 and 200 nM human IL-18Rβ protein (SEQ ID NO: 2), diluted in PBST buffer, were injected across all flow cells at 30 μl/min for 3 minutes, then dissociation was collected for 5 minutes. The α-human Fc biosensor surface was regenerated between cycles using two 30 second injections of 3 M magnesium chloride. Sensorgrams were double-reference subtracted and fit to a 1:1 binding model in T200 Evaluation software or Insight Evaluation software (Cytiva).

SPR kinetics were measured at baseline (T0), 4 weeks at 4° C. (4w 4° C.), and 4 weeks at 40° C. (4w 40° C.). The results are disclosed in Table E15 below.

TABLE E15
SPR of anti-IL-18Rβ antibodies binding to human IL-18Rβ

| Antibody | | | | | | |
|---|---|---|---|---|---|---|
| Name | pH | Timepoint | ka (1/Ms) | Kd (1/s) | KD (M) | % Rmax |
| P8 | 6 | T0 | 5.7 × 10⁵ | 4.7 × 10⁻⁴ | 8.3 × 10⁻¹⁰ | 97.8 |
| | | 4 w 4° C. | 5.7 × 10⁵ | 4.5 × 10⁻⁴ | 8.0 × 10⁻¹⁰ | 90.7 |
| | | 4 w 40° C. | 7.3 × 10⁵ | 4.4 × 10⁻⁴ | 6.0 × 10⁻¹⁰ | 100.9 |
| | 8 | T0 | 5.4 × 10⁵ | 4.6 × 10⁻⁴ | 8.5 × 10⁻¹⁰ | 103.3 |
| | | 4 w 4° C. | 5.2 × 10⁵ | 4.3 × 10⁻⁴ | 8.1 × 10⁻¹⁰ | 94.1 |
| | | 4 w 40° C. | 5.9 × 10⁵ | 4.6 × 10⁻⁴ | 7.8 × 10⁻¹⁰ | 103.5 |
| P21 | 6 | T0 | 2.4 × 10⁵ | 3.8 × 10⁻⁴ | 1.6 × 10⁻⁹ | 102.9 |
| | | 4 w 4° C. | 2.6 × 10⁵ | 3.8 × 10⁻⁴ | 1.4 × 10⁻⁹ | 95.1 |
| | | 4 w 40° C. | 2.3 × 10⁵ | 1.3 × 10⁻³ | 5.6 × 10⁻⁹ | 104.1 |
| | 8 | T0 | 2.5 × 10⁵ | 3.7 × 10⁻⁴ | 1.5 × 10⁻⁹ | 101.7 |
| | | 4 w 4° C. | 2.7 × 10⁵ | 3.4 × 10⁻⁴ | 1.3 × 10⁻⁹ | 93.8 |
| | | 4 w 40° C. | 2.6 × 10⁵ | 5.0 × 10⁻⁴ | 1.9 × 10⁻⁹ | 107.7 |
| P20 | 6 | T0 | 2.3 × 10⁵ | 3.3 × 10⁻⁴ | 1.4 × 10⁻⁹ | 95.1 |
| | | 4 w 4° C. | 2.4 × 10⁵ | 3.6 × 10⁻⁴ | 1.5 × 10⁻⁹ | 89.5 |
| | | 4 w 40° C. | 3.0 × 10⁵ | 3.9 × 10⁻⁴ | 1.3 × 10⁻⁹ | 99.0 |
| | 8 | T0 | 2.2 × 10⁵ | 3.5 × 10⁻⁴ | 1.6 × 10⁻⁹ | 97.2 |
| | | 4 w 4° C. | 2.3 × 10⁵ | 3.5 × 10⁻⁴ | 1.5 × 10⁻⁹ | 88.0 |
| | | 4 w 40° C. | 2.4 × 10⁵ | 3.8 × 10⁻⁴ | 1.6 × 10⁻⁹ | 102.4 |

Table E15 shows that the stressed samples of P20 and P8 exhibited comparable binding as the TO (baseline measurement). In contrast, the 1-month 40° C. sample for P21 exhibited faster off-rate (kd) and weaker binding (values shown in bold in Table E15), suggesting that isomerization impacted binding.

In summary, the three antibodies were assessed for stability after 1 month of storage at pH 6 and at pH 8. While storage at pH 8 results in greater thermal stability for all three antibodies, the higher pH led to a higher level of change in main peak in capillary isoelectric focusing (cIEF), suggesting chemical modification. Storage at pH 6 results in a greater loss of thermal stability, and, for two antibodies P20 and P21, an increase in aspartate isomerization in LCDR2. Binding of stressed samples to human IL-18Rβ was not impacted for P20. In contrast, P21 exhibited a loss of binding in stressed sample.

### Example 4—Development and Further Characterization of Framework Reverted IL-18Rβ Antibodies

Given the findings from the stability assessment on 3 IL-18Rβ antibodies, this example describes further experiments that were performed to investigate: (a) mutations in each antibody that might eliminate the chemical liability described in Example 3.D without compromising binding of the antibodies and (b) framework reversion that might improve developability and further lower immunogenicity risk of the antibodies. Following the development of variants based on these experiments, further characterization to validate the improved function of these variant antibodies was then performed.

### A. Deep Mutational Scan

Since antibodies P20 and P21 both exhibited an aspartate isomerization liability in LCDR2 (see Example 3.D), deep mutational scan was carried out for both P20 and P21 to assess whether position D50 in LCDR2 can be engineered to a different amino acid to remove the chemical liability without impacting activity.

A mutational scan library was designed such that each member of the library contained only a single mutation, relative to its parent antibody, within a complementarity-determining region (CDR) of the antibody. Each position within the CDR was mutated to each of the 20 possible amino acid residues, and the library was designed to scan all 6 CDRs of the antibody in this manner. Accordingly, a total of 1280 and 1360 single mutation variants were generated from parental antibodies P20 and P21, respectively. The mutational scan library was constructed as scFvs using overlapping PCR. To ensure there was only one mutation per library member, two mutational scan libraries were generated; one each for heavy chain and light chain (accordingly, if the mutational scan was in the heavy chain CDRs, then the light chain sequence was held constant and vice versa).

In vitro selection of the mutational scan libraries was carried out using mRNA display, with the individual member made into mRNA-protein fusion molecules using methods previously described in Example 1.A. Prior to the selection, an aliquot of the fusion molecules for each library was set aside for downstream analysis (“input”). The libraries were then selected for binding against 1 nM biotinylated human IL-18Rβ at pH 7.4 and at pH 6. cDNA portion of the binding population at both pH values, as well as the input, were amplified for analysis by next generation sequencing (NGS). The frequency of each library member in the eluted fraction was divided by the frequency of each library member in the input fraction to derive an enrichment ratio (ER). This value was further normalized against that of the wildtype sequence to allow for comparison of each variant against the parental sequence to assess for tolerance of a particular substitution at that position. Variants with an ER value of close to or equal to 1 (i.e., 0.5<ER<1.5) have a neutral mutation, in which the amino acid substitution is tolerated for binding, whereas those with an ER value of less than 0.5 have a mutation that negatively impacts binding. The calculated ER ratios for individual members of the library were represented as heat maps to readily identify variants that have comparable binding to the wildtype sequence at both pH values.

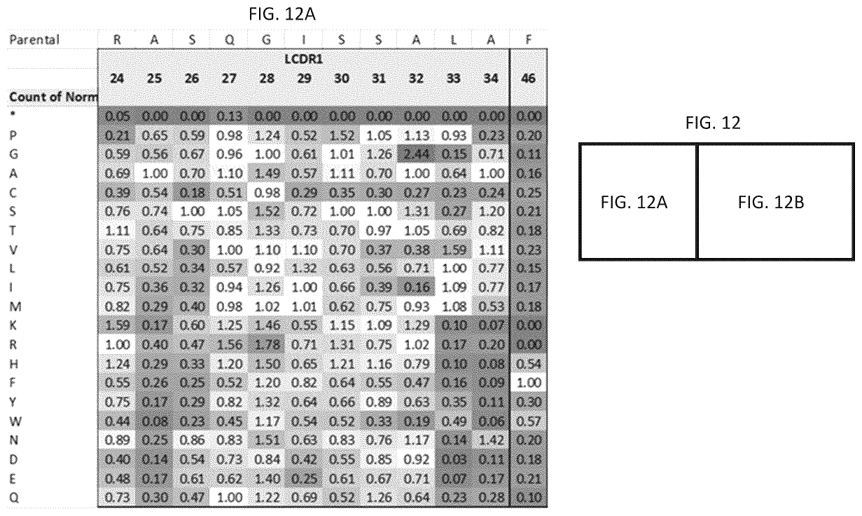

The resulting heat maps for light chain of P20 are shown below in FIG. 12. Heat maps were also generated for the heavy chain of P20 well as both chains of P21.

Based on the heat maps from deep mutational scan, the following mutations were proposed to LCDR2 of P20 to remove the chemical liability as well as to maintain low in silico immunogenicity risk: D50E, D50G, and D50G_A51E. For P21, only D50E variant was made to remove chemical liability in LCDR2.

### B. Development of Framework Reverted and Engineered Framework Reverted Versions of Selected IL-18Rβ Antibodies

Framework reversion to germline was carried out for the three IL-18Rβ antibodies P20. P21, and P8 to investigate whether additional mutations would further lower the immunogenicity risk of these antibodies.

Framework reversion was carried out by aligning and comparing the antibody heavy and light chain sequences against that of the closest human germline, identifying positions that differed from those of human germline, and changing the amino acid at these positions to that of human germline. The framework reverted antibodies were gene synthesized in IgG expression vectors and transiently transfected into HEK cells for expression.

Additionally, the proposed mutations described in Example 4.A (D50E, D50G, or D50G_A51E to P20 and D50E to P21) were grafted onto the framework reverted versions of P20 and P21 to remove chemical liability.

These resulting variant antibodies were tested for activity, stability, and immunogenicity. Amino acid sequences for a subset of the resulting variant antibodies are disclosed in Table E16A below. Nucleotide sequences encoding amino acid sequences for a subset of the resulting variant antibodies are disclosed in Table E16B below. Variant antibody P20-V1 includes a Q6E substitution in the heavy chain and a D50E substitution in the light chain of P20. Variant antibody P20-V2 includes a Q6E substitution in the heavy chain and D50E and A51G substitutions in the light chain of P20. Variant antibody P20-V3 includes a Q6E substitution in the heavy chain and a D50G substitution in the light chain of P20. Variant antibody P8-V1 includes a R19K substitution in the heavy chain and R3Q and M75I substitutions in the light chain of P8. Variant antibody P21-V1 includes a Q6E substitution in the heavy chain and R39K and D50E substitutions in the light chain of P21.

TABLE E16A
SEQ ID NOs of amino acid sequences for select variant antibodies.

| | | Mutations | | | | | CDR-H1, | CDR-L1, | | |
|---|---|---|---|---|---|---|---|---|---|---|
| | | Relative to | | | | | -H2, -H3 | -L2, -L3 | HC | LC |
| | Parent | Parent | | | | | (numbering | (numbering | Constant | Constant |
| Antibody | Antibody | Antibody* | HC | LC | VH | VL | scheme) | scheme) | Domain | Domain |
| P20-V1 | P20 | HC_Q6E; | 32 | 33 | 34 | 35 | 8, 9, 10 (Kabat) | 22, 36, 24 | 30 | 31 |
| | | LC_D50E | | | | | 11, 12, 10 | (Kabat) | | |
| | | | | | | | (Chothia) | 22, 36, 24 | | |
| | | | | | | | 13, 14, 10 | (Chothia) | | |
| | | | | | | | (AbM) | 22, 36, 24 | | |
| | | | | | | | 15, 16, 17 | (AbM) | | |
| | | | | | | | (Contact) | 25, 37, 27 | | |
| | | | | | | | 18, 19, 20 | (Contact) | | |
| | | | | | | | (IMGT) | 28, 38, 24 | | |
| | | | | | | | | (IMGT) | | |
| P20-V2 | P20 | HC_Q6E; | 32 | 39 | 34 | 40 | 8, 9, 10 (Kabat) | 22, 41, 24 | 30 | 31 |
| | | LC_D50E | | | | | 11, 12,10 | (Kabat) | | |
| | | A51G | | | | | (Chothia) | 22, 41, 24 | | |
| | | | | | | | 13, 14, 10 | (Chothia) | | |
| | | | | | | | (AbM) | 22, 41, 24 | | |
| | | | | | | | 15, 16, 17 | (AbM) | | |
| | | | | | | | (Contact) | 25, 42, 27 | | |
| | | | | | | | 18, 19, 20 | (Contact) | | |
| | | | | | | | (IMGT) | 28, 43, 24 | | |
| | | | | | | | | (IMGT) | | |
| P20-V3 | P20 | HC_Q6E; | 32 | 44 | 34 | 45 | 8, 9, 10 (Kabat) | 22, 46, 24 | 30 | 31 |
| | | LC_D50G | | | | | 11, 12,10 | (Kabat) | | |
| | | | | | | | (Chothia) | 22, 46, 24 | | |
| | | | | | | | 13, 14, 10 | (Chothia) | | |
| | | | | | | | (AbM) | 22, 46, 24 | | |
| | | | | | | | 15, 16, 17 | (AbM) | | |
| | | | | | | | (Contact) | 25, 47, 27 | | |
| | | | | | | | 18, 19, 20 | (Contact) | | |
| | | | | | | | (IMGT) | 28, 48, 24 | | |
| | | | | | | | | (IMGT) | | |
| P8-V1 | P8 | HC R19K; | 73 | 74 | 75 | 76 | 52, 53, 54 | 66, 67, 68 | 30 | 31 |
| | | LC_R3Q_M75I | | | | | (Kabat) | (Kabat) | | |
| | | | | | | | 55, 56, 54 | 66, 67, 68 | | |
| | | | | | | | (Chothia) | (Chothia) | | |
| | | | | | | | 57, 58, 54 | 66, 67, 68 | | |
| | | | | | | | (AbM) | (AbM) | | |
| | | | | | | | 59, 60, 61 | 69, 70, 71 | | |
| | | | | | | | (Contact) | (Contact) | | |
| | | | | | | | 62, 63, 64 | 72, 29, 68 | | |
| | | | | | | | (IMGT) | (IMGT) | | |
| P21-V1 | P21 | HC_Q6E; | 98 | 99 | 100 | 101 | 80, 81, 82 | 22, 102, 95 | 30 | 31 |
| | | LC_R39K | | | | | (Kabat) | (Kabat) | | |
| | | D50E | | | | | 83, 84, 82 | 22, 102, 95 | | |
| | | | | | | | (Chothia) | (Chothia) | | |
| | | | | | | | 85, 86, 82 | 22, 102, 95 | | |
| | | | | | | | (AbM) | (AbM) | | |
| | | | | | | | 87, 88, 89 | 25, 103, 97 | | |
| | | | | | | | (Contact) | (Contact) | | |
| | | | | | | | 90, 91, 92 | 28, 38, 95 | | |
| | | | | | | | (IMGT) | (IMGT) | | |

*mutations designated using Kabat numbering

TABLE E16B
SEQ ID NOs for nucleotide sequences
encoding select variant antibodies.

| | | Mutations | | | | |
|---|---|---|---|---|---|---|
| | | Relative to | | | | |
| | Parent | Parent | | | | |
| Antibody | Antibody | Antibody* | HC | LC | VH | VL |
| P20-V1 | P20 | HC_Q6E; | 129 | 130 | 127 | 128 |
| | | LC_D50E | | | | |
| P20-V2 | P20 | HC_Q6E; | 141 | 142 | 139 | 140 |
| | | LC_D50E | | | | |
| | | A51G | | | | |
| P20-V3 | P20 | HC_Q6E; | 145 | 146 | 143 | 144 |
| | | LC_D50G | | | | |
| P8-V1 | P8 | HC R19K; | 133 | 134 | 131 | 132 |
| | | LC_R3Q_M75I | | | | |
| P21-V1 | P21 | HC_Q6E; | 157 | 158 | 155 | 156 |
| | | LC_R39K | | | | |
| | | D50E | | | | |

### C. Surface Plasmon Resonance (SPR) to Assess Framework Reverted Binding to IL-18Rβ

The kinetics of framework reverted antibodies were assessed to determine how well each antibody bound to human (hu) and cynomolgus monkey (cyno) IL-18Rβ using surface plasmon resonance (SPR).

Specifically, Biacore CM5 Series S sensors (Cytiva) were docked onto Biacore T200 instruments (Cytiva), primed with PBST (10 mM Na2HPO4, 137 mM NaCl, 2.7 mM KCl, and 1.8 mM KH2PO4, 0.05% Tween 20, pH 7.4). An α-human-Fc capture sensor surface (Cytiva #29234600) was prepared using the manufacturer recommended protocol. The sample compartment was adjusted to 10° C., and sensor surface to 37° C. Kinetics were measured for α-huIL-18Rβ antibodies using the following conditions. The entire assay was run once using PBST pH 7.4 running buffer, then the cycles were repeated using PBST at pH 6.0 running buffer across the same biosensor. Antibodies were diluted to 15 nM in PBST (pH 7.4) and captured for 10 μl/min for 25 seconds. Multi-cycle kinetics titrations of 0, 0.78, 1.56, 3.12, 6.25, 12.5, 25, 50, 100 and 200 nM human IL-18Rβ (SEQ ID NO: 2) and cyno IL-18Rβ proteins (SEQ ID NO: 3), diluted in PBST buffer at the appropriate pH, were injected across all flow cells at 30 μl/min for 3 minutes, then dissociation was collected for 5 minutes. The α-human Fc biosensor surface was regenerated between cycles using two 30 second injections of 3 M magnesium chloride. Sensorgrams were double-reference subtracted and fit to a 1:1 binding model in T200 Evaluation software or Insight Evaluation software (Cytiva).

While huIL-18Rβ-HisAvi was used to assess candidate antibody binding to human IL-18Rβ, cyno IL-18Rβ-HisAvi was used to evaluate each candidate antibody's cross-species reactivity.

Table E17 summarizes the analyzed binding kinetics for the panel of anti-IL-18Rβ antibodies that included parental and framework reverted variants, assessed at pH 6 and pH 7.4.

TABLE E17
Framework reverted variants dual-pH SPR kinetics for human and cyno IL-18Rβ

| | Anti- | Parent | Mutations Relative | pH 7.4 | pH 7.4 | pH 7.4 | pH 6 | pH 6 | pH 6 | |
|---|---|---|---|---|---|---|---|---|---|---|
| | body | Anti- | to Parent | ka | kd | KD | ka | kd | KD | |
| Antigen | Name | body | Antibody* | (1/Ms) | (1/s) | (M) | (1/Ms) | (1/s) | (M) | Comment |
| huIL- | P20 | P20 | None | 3.3 × 10⁵ | 1.7 × 10⁻⁴ | 5.1 × 10⁻¹⁰ | 3.8 × 10⁵ | 1.6 × 10⁻⁴ | 4.3 × 10⁻¹⁰ | |
| 18Rβ- | P20-V4 | P20 | HC_Q6E::LC_F46L | 4.4 × 10⁴ | 1.8 × 10⁻³ | 4.2 × 10⁻⁸ | 2.7 × 10⁵ | 3.7 × 10⁻³ | 1.4 × 10⁻⁸ | Weaker |
| HisAvi | | | | | | | | | | binding |
| (SEQ ID | P20-V5 | P20 | LC_F46L | 5.6 × 10⁴ | 1.1 × 10⁻³ | 1.9 × 10⁻⁸ | 3.4 × 10⁵ | 5.9 × 10⁻⁴ | 1.8 × 10⁻⁹ | Weaker |
| NO: 2) | | | | | | | | | | binding |
| | P20-V6 | P20 | HC_Q6E | 2.1 × 10⁵ | 2.2 × 10⁻⁴ | 1.0 × 10⁻⁹ | 5.2 × 10⁵ | 6.8 × 10⁻⁵ | 1.3 × 10⁻¹⁰ | |
| | P8 | P8 | None | 5.2 × 10⁵ | 5.0 × 10⁻⁴ | 9.6 × 10⁻¹⁰ | 1.4 × 10⁶ | 2.4 × 10⁻⁴ | 1.8 × 10⁻¹⁰ | |
| | P8-V1 | P8 | HC_R19K::LC_R3Q_M75I | 5.8 × 10⁵ | 4.7 × 10⁻⁴ | 8.0 × 10⁻¹⁰ | 1.3 × 10⁶ | 2.8 × 10⁻⁴ | 2.2 × 10⁻¹⁰ | |
| | P8-V2 | P8 | HC_R19K::LC_R3Q | 6.8 × 10⁵ | 4.9 × 10⁻⁴ | 7.3 × 10⁻¹⁰ | 1.6 × 10⁶ | 2.8 × 10⁻⁴ | 1.8 × 10⁻¹⁰ | |
| | P21 | P21 | None | 2.4 × 10⁵ | 4.3 × 10⁻⁴ | 1.8 × 10⁻⁹ | 4.8 × 10⁵ | 3.1 × 10⁻⁴ | 6.5 × 10⁻¹⁰ | |
| | P21-V2 | P21 | LC_R39K | 2.3 × 10⁵ | 5.3 × 10⁻⁴ | 2.3 × 10⁻⁹ | 6.1 × 10⁵ | 4.3 × 10⁻⁴ | 7.0 × 10⁻¹⁰ | |
| | P21-V3 | P21 | HC_Q6E::LC_R39K | 2.4 × 10⁵ | 5.4 × 10⁻⁴ | 2.3 × 10⁻⁹ | 5.9 × 10⁵ | 3.9 × 10⁻⁴ | 6.6 × 10⁻¹⁰ | |
| cyIL- | P20 | P20 | None | 1.8 × 10⁵ | 2.4 × 10⁻⁴ | 1.4 × 10⁻⁹ | 1.8 × 10⁵ | 2.8 × 10⁻⁴ | 1.6 × 10⁻⁹ | |
| 18Rβ- | P20-V4 | P20 | HC_Q6E::LC_F46L | 2.3 × 10⁴ | 2.0 × 10⁻³ | 8.8 × 10⁻⁸ | 4.8 × 10⁴ | 2.0 × 10⁻³ | 4.2 × 10⁻⁸ | Weaker |
| HisAvi | | | | | | | | | | binding |
| (SEQ ID | P20-V5 | P20 | LC_F46L | 3.4 × 10⁴ | 1.3 × 10⁻³ | 4.0 × 10⁻⁸ | 1.8 × 10⁵ | 9.7 × 10⁻⁴ | 5.5 × 10⁻⁹ | Weaker |
| NO: 3) | | | | | | | | | | binding |
| | P20-V6 | P20 | HC_Q6E | 1.1 × 10⁵ | 3.2 × 10⁻⁴ | 2.9 × 10⁻⁹ | 2.5 × 10⁵ | 1.5 × 10⁻⁴ | 6.1 × 10⁻¹⁰ | |
| | P8 | P8 | None | Weak, heterogeneous binding | | | | | | |
| | P8-V1 | P8 | HC_R19K::LC_R3Q_M75I | | | | | | | |
| | P8-V2 | P8 | HC_R19K::LC_R3Q | | | | | | | |
| | P21 | P21 | None | 1.5 × 10⁵ | 2.9 × 10⁻³ | 1.9 × 10⁻⁸ | 1.8 × 10⁵ | 2.0 × 10⁻³ | 1.1 × 10⁻⁸ | |
| | P21-V2 | P21 | LC_R39K | 1.5 × 10⁵ | 3.2 × 10⁻³ | 2.2 × 10⁻⁸ | 1.6 × 10⁵ | 2.2 × 10⁻³ | 1.4 × 10⁻⁸ | |
| | P21-V3 | P21 | HC_Q6E::LC_R39K | 1.6 × 10⁵ | 2.7 × 10⁻³ | 1.7 × 10⁻⁸ | 1.9 × 10⁵ | 1.6 × 10⁻³ | 8.4 × 10⁻⁹ | |

*mutations designated using Kabat numbering

As shown in Table E17, most of the framework revertants had off-rates (kd) and dissociation constants (K_{D}) measured at pH 6 and pH 7.4 that were comparable to that of the parental antibodies. Furthermore, pH-independent binding was observed for the framework revertants, consistent with the profile of the parent antibodies characterized within the experiment (and earlier in Table E5). These results suggested that reversion of the mutations in these framework positions back to germline is tolerated. In contrast, framework revertant P20-V4, which is a revertant of P20 with two positions in the framework reverted to germline (HC_Q6E, LC_F46L) demonstrated 100-fold weaker binding at both pHs tested. This is further supported by the kinetic parameters for the light chain only framework revertant P20-V5 (P20_LC_F46L) in which the off-rates and dissociation constants at both pHs were 10-fold weaker than that of the parent antibody P20. In contrast, the off-rates (kd) and dissociation constants (KD) for framework revertant P20-V6 (P20_HC_Q6E) were comparable to that of the parent antibody. Taken together, these results indicated that F46L reversion to germline in the light chain is not tolerated.

In addition, pH-independent binding to cyno IL-18Rβ was observed for P20, P21, and associated framework revertants. In contrast, very weak binding to cyno IL-18 Rβ was observed for P8 and associated framework revertants.

### D. Human Whole Blood Assay to Assess Framework Reverted Antibody Potency

Framework revertant antibodies were also progressed into potency assessment in LPS+IL-12 dependent stimulation of IFNg production in human whole blood.

Specifically, human whole blood was collected from healthy donors (BMS internal Blood Donor program) in EDTA vacutainer tubes (BD, ref 366643), diluted 1:1 with RPMI1640 (Gibco/Thermo-Fisher, cat #11875093) without FBS. 200 μL per well were dispensed in 96-well plates (Thermo-Fisher, cat #07-200-90). Cells were then pre-treated for 1 h with anti-huIL-18Rβ antibodies listed in Table E18 or isotype control monoclonal antibody huIgG1.3f with a final concentration range from 100 nM to 0.04 nM. One hour later, the cells were then stimulated LPS (Sigma-Aldrich, cat #L4391-1 MG)+human IL-12p70 (PeproTech, cat #200-12-50 μg) at final concentrations of 5 μg/mL and 5 ng/mL respectively in the assay well. After a 16-24 hours incubation at 37° C., 5% CO2, the plates were centrifuged for 5 minutes at 4000 rpm. Supernatants were then collected for IFNg secretion measurement using the MSD platform (IFNg MSD assay, cat #K151QO). To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

Results of the human whole blood assay on variant antibodies are disclosed in Table E18 below.

TABLE E18
Antibody potency in inhibiting LPS + IL-12
dependent IFNg production in human whole blood

| | Parent | Mutations Relative to | |
|---|---|---|---|
| Antibody | Antibody | Parent Antibody* | IC50 (nM) |
| P20 | P20 | None | 1.66 ± 0.54 |
| P20-V6 | P20 | HC_Q6E | 0.85 ± 0.38 |
| P20-V5 | P20 | LC_F46L | 21.19 ± 3.74 |
| P20-V4 | P20 | HC_Q6E::LC_F46L | 37.64 ± 6.14 |
| P21 | P21 | None | 1.34 ± 0.14 |
| P21-V2 | P21 | LC_R39K | 1.65 ± 0.91 |
| P21-V3 | P21 | HC_Q6E::LC_R39K | 1.63 ± 0.89 |
| P8 | P8 | None | 1.12 ± 0.66 |
| P8-V1 | P8 | HC_R19K::LC_R3Q_M75I | 1.09 ± 0.45 |
| P8-V2 | P8 | HC_R19K::LC_R3Q | 0.87 ± 0.71 |

*mutations designated using Kabat numbering

Results from Table E18 supported the observations from the surface plasmon resonance (SPR) characterization, in which most of the framework revertants demonstrated comparable potency as the parent antibodies, indicating that reversion of these mutations in framework positions back to germline is tolerated and does not lead to a loss of activity. The results further showed that, for P20, reversion to germline at position 46 of the light chain (by inclusion of F46L) led to a loss of activity.

### E. Characterization of Further Engineered Framework Reverted Antibodies Using Surface Plasmon Resonance and Human Whole Blood Assay

Given the surface plasmon resonance (SPR) and potency assessment findings of framework reverted antibodies, framework reverted versions of parental antibodies P20 and P21 that were further engineered to include both framework reversion and mutation in LCDR2 to remove chemical liability (as described in Example 4.A and 4.B), were also characterized for binding to human and cynomolgus monkey (cyno) IL-18Rβ at both pH 6 and at pH 7.4, and for potency in the human whole blood assay. Surface plasmon resonance (SPR) kinetics were measured as previously described in Example 4.C, and the human whole blood assay to assess potency was performed as previously described in Example 4.D. The SPR kinetics results are disclosed in Table E19 and the antibody potency results using the human whole blood assay are disclosed in Table E20 below.

TABLE E19
SPR kinetics of the engineered framework reverted anti-IL-18Rβ antibodies

| | | | Mutations Relative | pH 7.4 | pH 7.4 | pH 7.4 | pH 6 | pH 6 | pH 6 |
|---|---|---|---|---|---|---|---|---|---|
| | Antibody | Parent | to Parent | ka | kd | KD | ka | kd | KD |
| Antigen | Name | Antibody | Antibody* | (1/Ms) | (1/s) | (M) | (1/Ms) | (1/s) | (M) |
| huIL-18Rβ- | P20 | P20 | None | 3.3E+05 | 1.7E−04 | 5.1E−10 | 3.8E+05 | 1.6E−04 | 4.3E−10 |
| HisAvi | P20-V6 | P20 | HC_Q6E | 2.1E+05 | 2.2E−04 | 1.0E−09 | 5.2E+05 | 6.8E−05 | 1.3E−10 |
| (SEQ ID | P20-V1 | P20 | HC_Q6E::LC_D50E | 3.4E+05 | 1.5E−04 | 4.3E−10 | 3.4E+05 | 1.5E−04 | 4.5E−10 |
| NO: 2) | P20-V2 | P20 | HC_Q6E::LC_ D50E A51G | 3.6E+05 | 8.9E−05 | 2.4E−10 | 4.4E+05 | 1.3E−04 | 2.9E−10 |
| | P20-V3 | P20 | HC_Q6E::LC_D50G | 2.7E+05 | 1.9E−04 | 7.2E−10 | 3.9E+05 | 1.4E−04 | 3.6E−10 |
| | P21 | P21 | None | 2.4E+05 | 4.3E−04 | 1.8E−09 | 4.8E+05 | 3.1E−04 | 6.5E−10 |
| | P21-V3 | P21 | HC_Q6E::LC_R39K | 2.4E+05 | 5.4E−04 | 2.3E−09 | 5.9E+05 | 3.9E−04 | 6.6E−10 |
| | P21-V1 | P21 | HC_Q6E::LC_R39K_D50E | 3.6E+05 | 2.2E−04 | 6.3E−10 | 4.6E+05 | 1.7E−04 | 3.7E−10 |
| cynoIL-18Rβ- | P20 | P20 | None | 1.8E+05 | 2.4E−04 | 1.4E−09 | 1.8E+05 | 2.8E−04 | 1.6E−09 |
| HisAvi | P20-V6 | P20 | HC_Q6E | 1.1E+05 | 3.2E−04 | 2.9E−09 | 2.5E+05 | 1.5E−04 | 6.1E−10 |
| (SEQ ID | P20-V1 | P20 | HC_Q6E::LC_D50E | 2.0E+05 | 2.8E−04 | 1.4E−09 | 1.6E+05 | 2.8E−04 | 1.7E−09 |
| NO: 3) | P20-V2 | P20 | HC_Q6E::LC_ D50E A51G | 1.8E+05 | 1.6E−04 | 9.2E−10 | 1.6E+05 | 2.3E−04 | 1.4E−09 |
| | P20-V3 | P20 | HC_Q6E::LC_D50G | 2.0E+05 | 3.0E−04 | 1.5E−09 | 1.8E+05 | 4.2E−04 | 2.4E−09 |
| | P21 | P21 | None | 1.5E+05 | 2.9E−03 | 1.9E−08 | 1.8E+05 | 2.0E−03 | 1.1E−08 |
| | P21-V3 | P21 | HC_Q6E::LC_R39K | 1.6E+05 | 2.7E−03 | 1.7E−08 | 1.9E+05 | 1.6E−03 | 8.4E−09 |
| | P21-V1 | P21 | HC_Q6E::LC_R39K_D50E | 5.4E+05 | 4.7E−03 | 8.7E−09 | 2.5E+05 | 3.1E−03 | 1.3E−08 |

*mutations designated using Kabat numbering

TABLE E20
Engineered framework reverted anti-IL-18Rβ antibody
potency in inhibiting LPS + IL-12 dependent IFNg
production in human whole blood

| Antibody Name | Average IC50 (nM) |
|---|---|
| P20-V1 | 1.04 ± 0.24 |
| P21-V1 | 1.23 ± 0.66 |
| P20-V2 | 0.37 ± 0.03 |
| P20-V3 | 1.52 ± 0.33 |

Based on the results, the engineered variants of P20 and P21 retained comparable binding to both human and cyno IL-18Rβ at both pH and exhibited comparable potency as the parent antibodies (Table E18).

### F. Stability Assessment of Further Engineered Framework Reverted Antibodies

A 1 month-long stability assessment, including measurements of size homogeneity, chemical stability, and binding, was carried out to determine whether the engineered framework reverted anti-IL-18Rβ antibodies had an improved stability profile compared to their parental antibodies P20 and P21. The study design of the stability assessment is disclosed in Table E21 below.

TABLE E21
Stability assessment study design for
engineered framework reverted variants

| Antibody | | [Conc] | Time | Storage | |
|---|---|---|---|---|---|
| Names | Formulation | (mg/mL) | point | temperature | |
| P20-V1 | 20 mM Histidine, | 1.0 | T0 | 4° C. | 40° C. |
| P21-V1 | 260 mM Sucrose, | | 2 wk | | |
| | 50 μM DTPA, | | 4 wk | | |
| | 0.05% PS80, pH 6.0 | | 5X F/T | | |

Specifically, the freeze-thaw stability of the three antibodies were determined by performing 5 freeze/thaw cycles. A concentration of 1 mg/mL 200 μL/vial of each antibody was added to a 2 mL screw cap clear vial (Agilent). The vials were placed in a Biocision CoolCell and stored at −80° C. for 2 hours. The CoolCell was removed and placed at room temperature (˜23° C.) for 2 hours. This was repeated a total of 5 times and the samples were analyzed.

Additionally, thermal incubation of the two antibodies was performed at 4° C., 25° C., and at 40° C. in a Binder incubator. A volume of 1.5 mL with a concentration of 1 mg/mL in a formulation of 20 mM Histidine, 260 mM Sucrose, 50 M DTPA, 0.05% PS80 for pH 6.0 or 20 mM Tris. 260 mM Sucrose, 50 μM DTPA. 0.05% PS80. pH 8.0 was prepared for the stability assessment. 1.0 mL/vial of each antibody in pH 6 or pH 8 formulation was added to a 2 mL screw cap clear vial (Agilent) and parafilm tape was secured around the top to ensure a tight seal for the stability study and stored in the appropriate incubator. 500 μL of each formulation was analyzed at t0. The same volume was withdrawn from the vials stored in the incubator and analyzed at each of the 2 week and at 4 week timepoints. Chemical liabilities were analyzed by size exclusion chromatography, peptide mapping, and surface plasmon resonance (SPR).

Size exclusion chromatography (SEC) was used to determine the size homogeneity of the antibodies. An Agilent 1260 Infinity system was used with an Advancebio SEC 300 Å, 4.6×300 mm, 2.7 μm column. The running buffer was 100 mM potassium phosphate, 250 mM sodium chloride, pH 6.8. An amount of 25 μg of the sample was injected onto the column and ran at a rate of 0.2 mL/minute for 25 minutes. The chromatograms were analyzed at 280 nm and the area of the peaks were used to determine the percentage of monomer, high molecular weight (HMW) species, and low molecular weight (LMW) species. A gel filtration standard (Bio-Rad) was used before and after runs to ensure column integrity.

As shown in FIG. 13, each sample was analyzed initially (T0), at 2 weeks (2wk), at 4 weeks (4wk), and following 5 freeze-thaws (5×FT) timepoints. The monomeric, HMW, and LMW were calculated to be within acceptable ranges, less than 5% loss of monomeric peak over the course of 4 weeks.

The data suggests that P20-V1 and P21-V1 showed little change in profile over 1 month storage at 4° C. and at 40° C.

In addition, chemical stability and its effect on IL-18Rβ binding of the engineered framework reverted anti-IL-18Rβ antibodies were further characterized using peptide mapping. Specifically, proteins were denatured in the presence of 0.2% Rapigest surfactant (Waters Corp.) and reduced by dithiothreitol (DTT) at 80° C. for 30 minutes, alkylated by iodoacetamide (IAM) at room temp for 30 minutes in the dark, and digested by trypsin at 37° C. for 4 hours in Tris-HCL pH 7.4, followed by acidic quench.

Peptides were analyzed on an ACQUITY UPLC system (Waters™, Manchester, U.K.) coupled to a Q-Exactive™ Plus mass spectrometer (Thermo Scientific™, San Jose, CA). Peptides were eluted from a Waters BEH C18 column (130 Å 1.7 μm 2.1×150 mm, product #186002353), heated at 50° C., using a 60-minute LC gradient. The gradient was set from 0.2% to 30% solvent B in 46 minutes with a flow rate of 0.2 mL/min. Solvent A was 0.1% formic acid in water and solvent B was 0.1% formic acid in acetonitrile. Mass spectrometer was operated under positive ion mode with ESI voltage at 3.5 kV, a capillary temperature of 250° C., a scan range of 320-1800 m/z, and a sheath gas flow rate of 45.

Peptide mapping data was analyzed by Biologic™ software (Protein Metrics, Cupertino, CA) with precursor mass tolerance of 6 ppm, and fragment mass tolerance of 20 ppm. Carbamidomethylation was set as the fixed modification, and variable modifications were searched against oxidation and deamidation. The MS/MS spectra and the levels of modification were manually verified and calculated. The oxidation, deamidation, and isomerization levels of peptides were monitored. % Relative Modification was calculated based on the area under the curve (AUC) of modified peptide/(AUC of modified peptide+AUC of native peptide)*100. The results of the peptide mapping experiment on the variants showed no liability in stressed samples, confirming that aspartate isomerization observed in the parent antibodies (see Example 3.D) were eliminated (results not shown).

The stressed samples were also characterized by surface plasmon resonance (SPR), whose method is described in Example 4.C, to determine whether there is an impact on binding to human IL-18Rβ. The stressed samples were measured after 2 weeks at 40° C. (2w 40° C.), 4 weeks at 4° C. (4w 4° C.), and 4 weeks at 40° C. (4w 40° C.). Additionally, these stressed samples were also compared for binding to respective parent antibodies, as well as to the baseline samples (T0). The SPR kinetics results from this experiment are disclosed in Table E22 below.

TABLE E22
SPR characterization of stressed engineered
framework reverted antibodies

| | | pH 7 | pH 7 | pH 7 | pH 7 |
|---|---|---|---|---|---|
| Antibody | | ka | kd | KD | Rmax |
| Name | Timepoint | (1/Ms) | (1/s) | (M) | (RU) |
| P20 | Control | 2.8E+05 | 5.2E−05 | 1.8E−10 | 104.9 |
| P20-V1 | T0 | 2.7E+05 | 7.2E−05 | 2.7E−10 | 107.2 |
| | 2 w 40° C. | 2.9E+05 | 3.7E−05 | 1.3E−10 | 120.9 |
| | 4 w 4° C. | 2.8E+05 | 5.7E−05 | 2.0E−10 | 102.5 |
| | 4 w 40° C. | 2.9E+05 | 1.4E−04 | 5.0E−10 | 77.8 |
| P21 | Control | 3.5E+05 | 7.0E−05 | 2.0E−10 | 49.3 |
| P21-V1 | T0 | 2.7E+05 | 7.2E−05 | 2.6E−10 | 93.3 |
| | 2 w 40° C. | 2.7E+05 | 4.9E−05 | 1.8E−10 | 115.5 |
| | 4 w 4° C. | 2.7E+05 | 6.5E−05 | 2.4E−10 | 93.8 |
| | 4 w 40° C. | 2.8E+05 | 1.4E−04 | 5.1E−10 | 70.1 |

As shown in Table E22, the stressed samples of P20-V1 and P21-V1 exhibited comparable binding as the TO (baseline measurement) as well as to the respective parent antibodies.

### G. Immunogenicity Assessment of Further Engineered Framework Reverted Antibodies

After an HLA class II molecule binds a peptide antigen, the next critical step in developing an anti-drug immune response is the activation of cognate T cells. This step occurs when the peptide-MHC complex is presented by an antigen presenting cell (APC) to a matching T cell receptor (TCR) on a cluster of differentiation 4 (CD4) positive T cell. In vitro assays, using diverse human donors, are often used to determine whether a candidate therapeutic contains functional T cell epitopes based on its ability to stimulate antigen specific CD4+ T cells in vitro. Thus, the engineered framework reverted anti-IL-18Rβ antibodies were evaluated in an in vitro DC-T cell proliferation assay to determine whether any of these candidate therapeutics contained functional T cell epitopes based on its ability to stimulate antigen specific CD4+ cells in vitro.

Briefly, peripheral blood mononuclear cells (PBMCs) from healthy volunteers were isolated by negative bead-based selection (Miltenyi Biotec Inc, Bergisch Gladbach, Germany) and HLA typed using polymerase chain reaction (PCR) amplification and hybridization with oligonucleotide probes (ProImmune, Sarasota, FL). A panel of 40 PBMC donors composed of HLA-DR class II alleles closely matching the world population frequencies was used for an assay run.

Monocytes were isolated from PBMCs using a negative selection bead-based method (Miltenyi Biotec Inc, Germany) and cultured for 5 days in DC media (Lonza, Basel, Switzerland) containing Interleukin 4 (IL-4) and granulocyte-macrophage colony stimulating factor (GM-CSF) to generate immature DCs. These cells were pulsed with one of the 6 anti-IL-18Rβ antibodies or quality control proteins overnight followed by a thorough washing, and then matured by incubating the cells overnight in media containing TNF-α, IL-1b, IL-6, and PGE2.

2,000 pulsed mature DCs were co-cultured with 200,000 autologous PBMCs, labeled with carboxyfluorescein succinimidyl ester (CFSE) (Invitrogen, Carlsbad, CA) to monitor proliferation. Six replicates of each condition were plated in 96-well plates in DC media containing pen-strep (Gibco, Waltham, MA). After seven days, media was washed away and cells were stained with a fluorescently labeled (APC) anti-human CD4 (BD Biosciences, San Jose, CA) monoclonal antibody. Unbound anti-CD4 monoclonal antibody was removed with a wash step, cells were fixed with 3.7% formalin (Sigma, St. Louis, MO) in phosphate-buffered saline (PBS) and analyzed by flow cytometry to determine the percentage of proliferating antigen-specific CD4+ T cells (Table E23).

TABLE E23
Antibody-induced CD4+ T cell proliferative response in
a cohort of 40 donors for engineered framework reverted variants

| | Parent | Mutations Relative to | |
|---|---|---|---|
| Antibody Name | Antibody | Parent Antibody* | Result |
| Avastin (low | | | 10% |
| immunogenicity | | | |
| control) | | | |
| P8-V1 | P8 | HC_R19K::LC_R3Q_M75I | 9.1% |
| P20 | P20 | None | 7.7% |
| P20-V1 | P20 | HC_Q6E::LC_D50E | 12.8% |
| P20-V2 | P20 | HC_Q6E::LC_D50E A51G | 17.9% |
| P20-V3 | P20 | HC_Q6E::LC_D50G | 17.9% |
| P21-V1 | P21 | HC_Q6E::LC R39K D50E | 28% |
| VL6 (high | | | 40% |
| immunogenicity | | | |
| control) | | | |

*mutations designated using Kabat numbering

As disclosed in Table E23, the low control monoclonal antibody (Avastin, [bevacizumab], Genentech, Inc.), which has been demonstrated to have low immunogenicity in the clinic (Saffari et al., Int J Cancer Manag., 2018) was positive in 10% of the donors. The high control IL-21R monoclonal antibody (internally made ATR-107, Pfizer, Inc.) induced a positive response in 40% of donors and has been shown to have a high ADA rate of 76% in clinical studies (Hua et al., J Clin Pharm, 2014).

Typically, proteins that induce less than 20% response in donors are considered to have low immunogenicity risk. Thus, many of the engineered variants appeared to have a low overall risk for potential clinical immunogenicity. In contrast, P21-V1, a variant of P21 with both framework reversions and chemical liability removed in LCDR2, generated a proliferative response in 28% of healthy donors, indicating potential for immunogenicity.

In conclusion, engineered variants of P20 retained comparable affinity and potency of the parent antibody while maintaining low immunogenicity risk. Furthermore, P20-V1 exhibited favorable stability profile after 1 month storage at 40° C. P20-V1 was found to have favorable developability properties and exhibited the least immunogenicity risk of the engineered P20 variants, and therefore it was selected as the development candidate.

### Example 5—Crystal Structure of the IL-18Rβ+Selected IL-18Rβ Antibody Fab Complex

### A. Crystal Structure of the IL-18Rβ+P20 Antibody Fab Complex

This example relates to using X-ray crystallography to determine the structure of the interleukin 18 receptor B (IL-18Rβ)+P20 antibody fragment antibody binding (Fab) complex at atomic resolution. The structure of this Fab complex structure was desired in order to elucidate the molecular basis for IL-18Rβ: P20-V1 epitope: paratope interactions, given that P20-V1 is an engineered framework reverted variant of the parental P20 antibody.

To determine the structure, IL-18Rβ (D3 domain residues 242-356) was recombinantly expressed in the Spodoptera frugiperda SF9 insect cell line and purified using standard chromatographic methods. P20 (Fab fragment) was recombinantly expressed in the human embryonic kidney (HEK) Expi293 cell line and purified using standard chromatographic methods. The IL-18Rβ+P20 Fab complex was formed by combining an equimolar ratio of each component and purified from uncomplexed protein using gel filtration chromatography. The purified IL-18Rβ+P20 Fab complex was crystallized by combining 0.2 μL IL18Rβ+P20 Fab complex (at 20 mg/mL) with an equal volume of precipitant (0.2 M potassium thiocyanate, 20% (w/v) polyethylene glycol 3,350) over a reservoir of 75 μL precipitant in an MRC UVXPO sitting drop vapor diffusion crystallization tray (Swissci) and the crystallization tray then sealed and incubated at 20° C. Crystals formed within 24 hours and grew to maximum size within one week. Crystals were cryoprotected by washing briefly through precipitant supplemented with 10% (v/v) each glycerol and ethylene glycol after which they were immediately flash frozen in liquid nitrogen for X-ray diffraction screening. X-ray diffraction data was collected at a wavelength of 1.0 Å and a temperature of 100 K at the Advanced Photon Source (IMCA-CAT beamline 17-ID). Data reduction was performed using autoPROC (Global Phasing Ltd.). The structure was phased by molecular replacement using Phaser (Adams, P. D. et al. (2010) Acta Crystallogr. D v66: pp213-221), the IL18Rβ D3 coordinates from RCSB PDB structure 3WO4, and Fab coordinates from an internally determined crystal structure of an apo Fab fragment. The structure was completed through iterative cycles of model building using Coot (Emsley, P. et al. (2010) Acta Crystallogr. D v66: pp486-501) and restrained refinement using autoBUSTER (Global Phasing Ltd.) to a final crystallographic R factor of 23.9% and free R factor of 27.7%.

The crystal structure of the IL-18Rβ+P20 Fab complex was determined at atomic resolution (Table E24).

TABLE E24
X-Ray Crystallographic Data Collection and Refinement Statistics
Crystal Parameters

| Space group | P4₂ |
|---|---|
| Cell dimensions | a = b = 92.1 Å, c = 139.7 Å |
| IL-18Rβ + P20 Fab Complexes per AU^{a} | 2 |

Data Collection

| Beamline | APS, IMCA-CAT 17-ID | |
|---|---|---|
| Wavelength (Å) | 1.0 | |
| Resolution Range (Å)^{b} | 92.10-2.90 | (3.10-2.90) |
| Observed/unique^{c} reflections | 112149/19940 | (6520/994) |
| Completeness (%)^{b} | 94.1 | (58.3) |
| I/σ(I)^{b} | 8.5 | (1.3) |

Refinement

| Resolution (Å) | 39.51-2.90 |
|---|---|
| R_{work}/R_{free}^{d} | 23.9/27.7 |
| No. atoms | |
| Protein | 7945 |
| Sugar | 56 |
| B-factors | |
| IL-18Rβ Molecule 1 | 88.21 |
| IL-18Rβ Molecule 2 | 111.60 |
| P20 Light Chain Molecule 1 | 103.42 |
| P20 Light Chain Molecule 2 | 91.18 |
| P20 Heavy Chain Molecule 1 | 75.27 |
| P20 Heavy Chain Molecule 2 | 91.99 |
| r.m.s.d.^{e} | |

| Bond lengths (Å) | 0.005 |
|---|---|
| Bond angles (°) | 0.81 |
| Ramachandran (%)^{g} | 91.63/98.10/1.90 |

^{a}Asymmetric unit.
^{b}Values in parentheses for resolution range, completeness, and I/σ(I) correspond to the last resolution shell.
^{c}Friedel pairs were treated as identical reflections.
^{d}R = Σₕₖₗ||F_{obs}| − |F_{calc}||/Σₕₖₗ|F_{obs}|, where R_{free} is calculated without a σ cutoff for a randomly chosen 5% of reflections, which were not used for structure refinement, and R_{work} is calculated for the remaining reflections.
^{e}Root mean square deviations from ideal bond lengths/angles.
^{g}Number of residues in favored region/allowed region/outlier region.

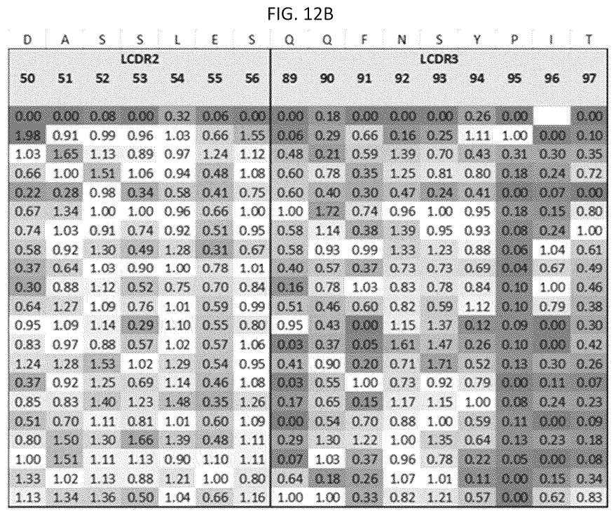

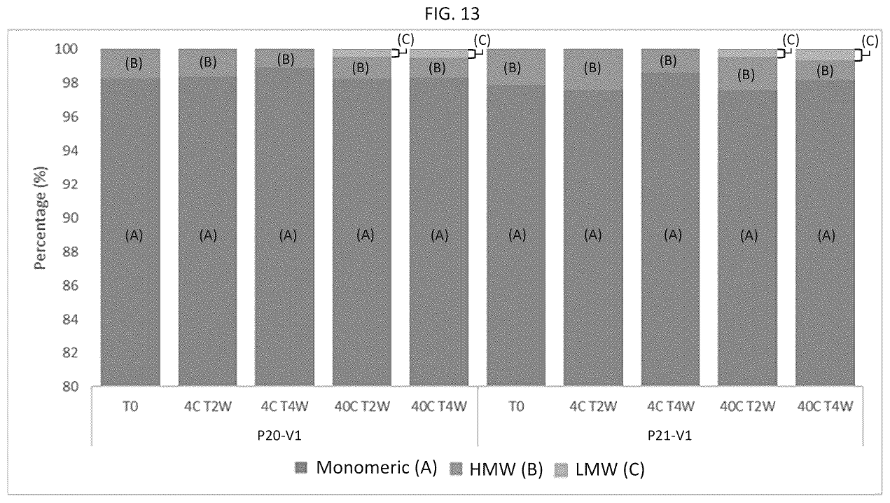

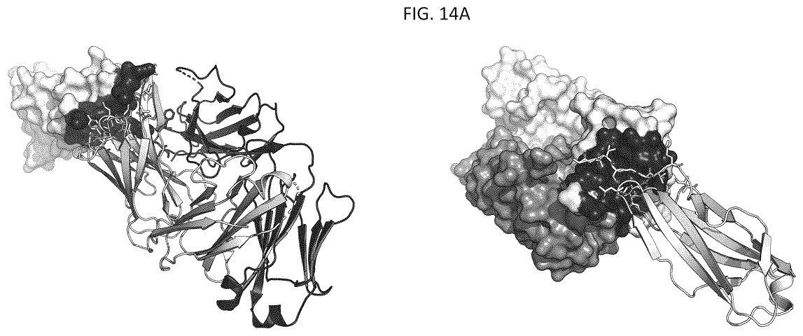

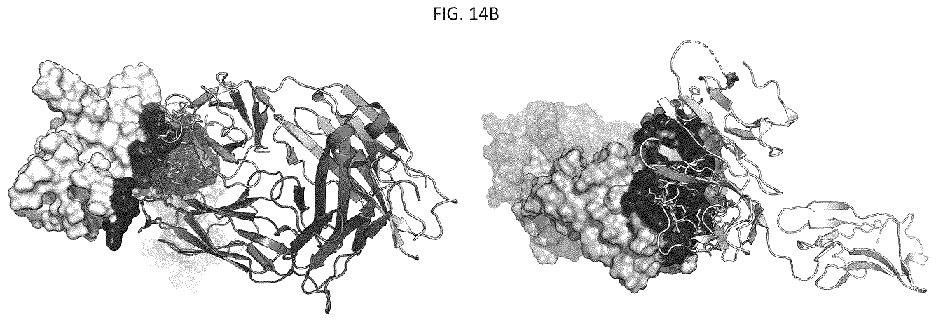

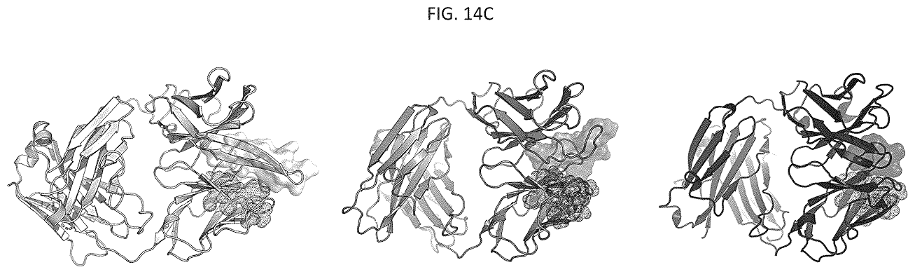

FIG. 14A shows the crystal structure of the IL-18Rβ+P20 complex, where the left image is a representation of the IL-18Rβ (molecular surface) epitope (dark grey) with the P20 Fab (cartoon; heavy chain, light grey; light chain, dark grey) paratope (sticks). Likewise, the right image is a representation of the IL-18Rβ (cartoon) epitope (sticks) with the P20 Fab (molecular surface; heavy chain, light grey; light chain, medium grey) paratope (dark grey).

The crystal structure revealed that IL-18Rβ binds to P20 via an array of hydrogen bonding, salt bridge, hydrophobic, and aromatic-aromatic interactions. The contacts made by IL-18Rβ which comprise the P20-binding epitope include residues 242-248, Phe 279, Arg 281, Val 282, Leu 312, Ser 342, and Ile343 (Table E25, Columns 1 & 3). The contacts made by P20 which comprise the IL-18Rβ-binding paratope include heavy chain CDR1 residues Asn 31, Tyr 32, and Tyr 33; heavy chain CDR2 residues Tyr 50, Phe 52, Tyr 53, Ser 54, Thr 56, and Asn 58; heavy chain CDR3 residues Asp 98, Val 99, Gly 100, Ile 101, Ala 102, Ala 103, Asn 105, Tyr 107, and Tyr 109; and light chain CDR3 residues Phe 91, Tyr 94, and Ile 96 (Table E25, Columns 2 & 3).

TABLE E25
Summary of IL-18Rβ:P20 Fab Intermolecular Interactions

| IL-18Rβ Residue | P20 Fab Residue | Distance (Å) |
|---|---|---|
| Ile 343 | HCDR2•Thr 56 | 3.2 |
| Ile 343 | HCDR2• Ser 54 | 3.7 |
| Ser 342 | HCDR2•Thr 56 | 3.2 |
| Leu 312 | HCDR3•Tyr 107 | 2.4 |
| Leu 312 | HCDR3•Ala 102 | 2.5 |
| Leu 312 | HCDR3•Asn 105 | 2.4 |
| Val 282 | HCDR2•Tyr 50 | 2.6 |
| Val 282 | LCDR3•Tyr 94 | 2.7 |
| Val 282 | HCDR2•Asn 58 | 2.8 |
| Arg 281 | HCDR1•Tyr 33 | 2.4 |
| Arg 281 | LCDR3•Tyr 94 | 2.1* |
| Arg 281 | HCDR2•Tyr 50 | 2.5 |
| Arg 281 | HCDR3•Tyr 109 | 2.5 |
| Arg 281 | LCDR3•Ile 96 | 2.4 |
| Arg 281 | HCDR3•Asp 98 | 2.3* |
| Arg 281 | LCDR3•Phe 91 | 2.7 |
| Phe 279 | HCDR3•Tyr 107 | 2.8 |
| Phe 279 | HCDR1•Tyr 33 | 2.7 |
| Phe 279 | HCDR3•Tyr 109 | 2.3 |
| Lys 248 | HCDR2•Ser 54 | 2.7 |
| Lys 248 | HCDR2•Tyr 53 | 2.3 |
| Lys 248 | HCDR2•Phe 52 | 3.9 |
| Thr 247 | HCDR2•Ser 54 | 4.0 |
| Asp 246 | HCDR2•Phe 52 | 2.7 |
| Asp 246 | HCDR1•Tyr 33 | 2.0* |
| Asp 246 | HCDR2•Thr 56 | 2.9 |
| Asp 246 | HCDR2•Ser 54 | 3.4 |
| Gly 245 | HCDR1•Tyr 33 | 2.6 |
| Gly 245 | HCDR2•Phe 52 | 2.4 |
| Val 244 | HCDR3•Gly 100 | 2.0* |
| Val 244 | HCDR3•Ile 101 | 3.1 |
| Val 244 | HCDR1•Tyr 33 | 2.8 |
| Val 244 | HCDR3•Tyr 107 | 2.3 |
| Val 244 | HCDR3•Ala 102 | 3.0 |
| Val 244 | HCDR3•Tyr 109 | 2.9 |
| Ile 243 | HCDR3•Ile 101 | 2.6 |
| Ile 243 | HCDR3•Gly 100 | 2.7 |
| Ile 243 | HCDR3•Val 99 | 2.8 |
| Ile 243 | HCDR1•Asn 31 | 2.5 |
| Ile 243 | HCDR1•Tyr 32 | 2.8 |
| Ile 243 | HCDR3•Ala 102 | 3.8 |
| Thr 242 | HCDR3•Ile 101 | 2.5 |
| Thr 242 | HCDR3•Ala 102 | 1.9* |

*Denotes hydrogen bonding and/or salt bridge interactions.

As the primary sequence of P20 differs from that of P20-V1 only outside of the heavy chain CDRs and LCDR3, the crystal structure of the IL-18Rβ+P20 Fab complex provides a comprehensive description of the specific IL-18Rβ: P20-V1 epitope: paratope interactions on a molecular level. When the structure was aligned to that of the active ternary complex (PDB accession ID 3WO4), the result suggests that binding of the antibody likely leads to a conformational change of D2 of IL-18Rβ, thereby disrupting the formation of active ternary complex. Thus, the antibody achieves its potent inhibitory activity as an allosteric blocker.

### B. Crystal Structure of the IL-18Rβ+P8 Antibody Fab Complex

This example relates to using X-ray crystallography to determine the structure of the interleukin 18 receptor B (IL-18Rβ)+P8 antibody fragment antibody binding (Fab) complex at atomic resolution. The structure of this Fab complex structure was desired in order to elucidate the molecular basis for IL-18Rβ: P8 epitope: paratope interactions.

To determine the structure, the IL-18Rβ full ectodomain (residues 20-357) and P8 (Fab fragment) were individually recombinantly expressed in the human embryonic kidney (HEK) Expi293 mammalian cell line and individually purified using standard chromatographic methods. The IL-18Rβ+P8 Fab complex was formed by combining an equimolar ratio of each component and purified from uncomplexed protein using gel filtration chromatography. The purified IL-18Rβ+P8 Fab complex was crystallized by combining 0.2 μL IL18Rβ+P8 Fab complex (at 24 mg/mL) with an equal volume of precipitant (0.2 M calcium chloride, 20% (w/v) polyethylene glycol 3,350) over a reservoir of 75 μL precipitant in an MRC UVXPO sitting drop vapor diffusion crystallization tray (Swissci) and the crystallization tray then sealed and incubated at 20° C. Crystals formed within 24 hours and grew to maximum size within one week. Crystals were cryoprotected by washing briefly through precipitant supplemented with 10% (v/v) each glycerol and ethylene glycol after which they were immediately flash frozen in liquid nitrogen for X-ray diffraction screening. X-ray diffraction data was collected at a wavelength of 1.0 Å and a temperature of 100 K at the Advanced Photon Source (IMCA-CAT beamline 17-ID). Data reduction was performed using autoPROC (Global Phasing Ltd.). The structure was phased by molecular replacement using Phaser (Adams, P. D. et al. (2010) Acta Crystallogr. D v66: pp213-221), the IL18Rβ coordinates from RCSB PDB structure 3WO4, and Fab coordinates from an internally determined crystal structure of an apo Fab fragment. The structure was completed through iterative cycles of model building using Coot (Emsley, P. et al. (2010) Acta Crystallogr. D v66: pp486-501) and restrained refinement using autoBUSTER (Global Phasing Ltd.) to a final crystallographic R factor of 23.6% and free R factor of 28.9%.

The crystal structure of the IL-18Rβ+P8 Fab complex was determined at atomic resolution (Table E26).

TABLE E26
X-Ray Crystallographic Data Collection and Refinement Statistics
Crystal Parameters

| Space group | P2₁2₁2₁ |
|---|---|
| Cell dimensions | a = 49.5 Å, b = 75.9 Å, c = 296.9 Å |
| IL-18Rβ + P8 Fab Complexes | 1 |
| per AU^{a} | |

Data Collection

| Beamline | APS, IMCA-CAT 17-ID | |
|---|---|---|
| Wavelength (Å) | 1.0 | |
| Resolution Range (Å)^{b} | 148.44-2.99 | (3.28-2.99) |
| Observed/unique^{c} reflections | 223118/16771 | (11674/840) |
| Completeness (%)^{b} | 91.1 | (57.7) |
| I/σ(I)^{b} | 10.0 | (1.3) |

Refinement

| Resolution (Å) | 38.23-2.99 |
|---|---|
| R_{work}/R_{free}^{d} | 23.6/28.9 |
| No. atoms | |
| Protein | 5120 |
| Sugar | 28 |
| B-factors | |
| IL-18Rβ | 159.58 |
| P8 Light Chain | 86.89 |
| P8 Heavy Chain | 77.16 |
| r.m.s.d.^{e} | |

| Bond lengths (Å) | 0.009 |
|---|---|
| Bond angles (°) | 1.08 |
| Ramachandran (%)^{g} | 85.69/95.62/4.38 |

^{a}Asymmetric unit.
^{b}Values in parentheses for resolution range, completeness, and I/σ(I) correspond to the last resolution shell.
^{c}Friedel pairs were treated as identical reflections.
^{d}R = Σₕₖₗ||F_{obs}| − |F_{calc}||/Σₕₖₗ|F_{obs}|, where R_{free} is calculated without a σ cutoff for a randomly chosen 5% of reflections, which were not used for structure refinement, and R_{work} is calculated for the remaining reflections.
^{e}Root mean square deviations from ideal bond lengths/angles.
^{g}Number of residues in favored region/allowed region/outlier region.

FIG. 14B shows the crystal structure of the IL-18Rβ+P8 complex, where the left image is a representation of the IL-18Rβ (molecular surface) epitope (dark grey) with the P8 Fab (cartoon; heavy chain, light grey; light chain, dark grey) paratope (sticks). Likewise, the right image is a representation of the IL-18Rβ (cartoon) epitope (sticks) with the P8 Fab (molecular surface; heavy chain, light grey; light chain, medium grey) paratope (dark grey).

The crystal structure revealed that IL-18Rβ binds to P8 via an array of hydrogen bonding, salt bridge, hydrophobic, and aromatic-aromatic interactions. The contacts made by IL-18Rβ which comprise the P8-binding epitope include residues Glu 50, Pro 51, Ser 54, His 55, Ile 173, Lys 193, 196-203, 206-210, Tyr 212, Tyr 214, His 215, and Tyr 219 (Table E27, Columns 1 & 3). The contacts made by P8 which comprise the IL-18Rβ-binding paratope include heavy chain CDR1 residues Thr 30, Ser 31, and Tyr 33; heavy chain CDR2 residues 50-52, Arg 54, and 57-59; heavy chain CDR3 residues 101-106 and Tyr 108; and light chain Asp 1; light chain CDR1 residues Gln 27, Ser 28, and Tyr 32; light chain CDR3 residues 92-94 (Table E27, Columns 2 & 3).

TABLE E27
Summary of IL-18Rβ:P8 Fab Intermolecular Interactions

| IL-18Rβ Residue | P20 Fab Residue | Distance (Å) |
|---|---|---|
| Tyr 219 | HCDR3•Pro 104 | 2.2 |
| His 215 | HCDR3•Pro 104 | 2.2 |
| His 215 | HCDR3•Ser 103 | 2.5 |
| His 215 | HCDR3•Val 102 | 1.9* |
| His 215 | HCDR3•Thr 105 | 3.9 |
| Tyr 214 | HCDR3•Arg 101 | 2.6 |
| Tyr 214 | HCDR3•Val 102 | 2.3* |
| Tyr 214 | HCDR3•Thr 105 | 4.0 |
| Tyr 212 | HCDR3•Val 102 | 2.4 |
| Glu 210 | HCDR3•Val 102 | 2.5 |
| Glu 210 | HCDR3•Ser 103 | 3.5 |
| Asp 209 | HCDR3•Ser 103 | 2.3 |
| Asp 209 | HCDR2•Arg 54 | 2.3* |
| Asp 209 | HCDR1•Ser 31 | 2.1* |
| Asp 209 | HCDR1•Thr 30 | 2.1* |
| Val 208 | HCDR3•Ser 103 | 2.2 |
| Val 208 | HCDR3•Pro 104 | 2.7 |
| Val 208 | HCDR3•Val 102 | 3.2 |
| Val 207 | HCDR3•Ser 103 | 3.0 |
| Val 207 | HCDR2•Arg 54 | 2.6 |
| Val 207 | HCDR3•Pro 104 | 3.6 |
| Ile 206 | HCDR3•Pro 104 | 2.2 |
| Ile 206 | HCDR3•Ser 103 | 4.0 |
| Ile 206 | HCDR2•Arg 54 | 4.0 |
| Ser 203 | HCDR2•Tyr 57 | 2.4 |
| Arg 202 | HCDR2•Tyr 57 | 2.8 |
| Arg 202 | HCDR2•Thr 58 | 2.3* |
| Arg 202 | HCDR2•Thr 59 | 2.9 |
| Arg 202 | HCDR2•Asn 52 | 3.9 |
| Glu 201 | HCDR1•Tyr 33 | 2.1* |
| Glu 201 | HCDR2•Asn 52 | 2.6 |
| Glu 201 | HCDR3•Pro 104 | 2.8 |
| Glu 201 | HCDR2•Arg 54 | 3.1 |
| Glu 201 | HCDR2•Tyr 57 | 3.5 |
| Val 200 | HCDR2•Thr 59 | 2.6 |
| Val 200 | HCDR1•Tyr 33 | 2.2 |
| Val 200 | HCDR2•Tyr 57 | 2.5 |
| Val 200 | HCDR2•Asn 52 | 2.1* |
| Val 200 | HCDR2•Ile 50 | 2.3 |
| Val 200 | HCDR2•Ile 51 | 2.8 |
| Val 200 | HCDR2•Thr 58 | 2.6 |
| Val 200 | HCDR3•Tyr 108 | 3.3 |
| Ser 199 | HCDR3•Tyr 108 | 2.4 |
| Ser 199 | HCDR2•Thr 59 | 2.1* |
| Leu 198 | HCDR3•Tyr 108 | 3.1 |
| Leu 198 | HCDR3•Pro 104 | 2.3 |
| Leu 198 | HCDR3•Asp 106 | 2.8 |
| Leu 198 | HCDR3•Thr 105 | 2.7 |
| Leu 197 | HCDR3•Tyr 108 | 2.7 |
| Lys 196 | HCDR3•Asp 106 | 2.7 |
| Lys 196 | LCDR1•Tyr 32 | 2.9 |
| Lys 196 | LCDR3•Tyr 92 | 2.4 |
| Lys 193 | HCDR3•Asp 106 | 2.5 |
| Lys 193 | HCDR3•Pro 104 | 3.9 |
| Ile 173 | HCDR3•Pro 104 | 2.6 |
| His 55 | LCDR1•Ser 28 | 3.5 |
| Ser 54 | LCDR3•Tyr 92 | 2.1* |
| Ser 54 | LCDR1•Ser 28 | 2.8 |
| Ser 54 | LCDR1•Gln 27 | 3.0 |
| Pro 51 | LCDR1•Gln 27 | 2.8 |
| Glu 50 | LCDR1•Gln 27 | 3.3 |
| Glu 50 | LC•Asp 1 | 4.0 |
| Glu 50 | LCDR3•Ser 93 | 3.9 |

*Denotes hydrogen bonding and/or salt bridge interactions.

The crystal structure of the IL-18Rβ+P8 Fab complex provides a comprehensive description of the specific IL-18Rβ: P8 epitope: paratope interactions on a molecular level. When the structure was aligned to that of the active ternary complex (PDB accession ID 3WO4), the result suggests that binding of the antibody is directly competitive with binding of IL-18Ra/IL-18. Thus, the P8 antibody achieves its potent inhibitory activity as a steric blocker. This contrasts with P20, which was observed to be an allosteric blocker.

### Example 6—Structural Basis of Isomerization Liability in LC-CDR2

Given the findings from the stability assessment, this example describes further experiments to investigate A) if Molecular Dynamic (MD) simulation predicted isomerization liability; and B) the structural basis of isomerization.

### A. Unpredictability of the Observed Aspartate Isomerization

Algorithms predicting aspartic acid isomerization in monoclonal antibodies (mAbs) leverage structural and sequence-based data to identify potential degradation sites. These in silico approaches analyze the local structural environment of aspartic acid residues, considering factors such as solvent accessibility, neighboring amino acids, and conformational flexibility. Current predictive algorithms and models, while advanced, still rely on experimental data that may not cover all possible scenarios. The high dimensionality and non-linear interactions within protein structures make it uniquely challenging to predict isomerization with high accuracy. Asp residues in motifs such as Asp-Gly (DG), Asp-Ser (DS), and Asp-Thr (DT) within the CDRs of mAbs are often identified as “hot spots” for isomerization and can be successfully predicted computationally. However, the Asp-Ala (DA) motif present in LCDR2 of antibodies disclosed herein (including P20, P21, and P8) is generally considered silent, with a very low propensity for isomerization. Such a DA motif is present in multiple published monoclonal antibody (mAb) sequences (see e.g., Raybould M. et al., Nucleic Acids Research (2020)). Based on a literature search, no published data on DA isomerization liability currently exists.

In an attempt to predict aspartate isomerization liabilities, a molecular dynamics (MD) simulation-based prediction pipeline (see, e.g., F. J. Irudayanathan et al., mAbs (2022) doi=10.1080/19420862.2022.2143006) was applied. Structures of the variable fragment (Fv) of the antibodies were predicted by AlphaFold-Multimer (version 2.3) (see, e.g., Evans R. et al., bioRxiv (2022)) using the ColabFold (version 1.5.5) Python package (Mirdita M. et al., Nature Methods (2022)). The crystal structure of the antibody was given as additional input for the structure prediction. To account for the disordered regions around LCDR2 of P20, which imply structural heterogeneity, two additional structures were predicted using a protocol described in Alamo D. et al., eLife (2022). The MD simulation pipeline was implemented using OpenMM (version 8.1.2) Python package (Eastman P. et al., Plos Comput. Biol. (2017)) with the AMBER 14SB force field (Maier J. et al., JCTC (2015)). The protonation states at pH 6.0 were assigned using predicted pKa values by PROPKA3. (Olsson M. et al., JCTC (2011)). For each variable fragment, three independent equilibrium MD simulations were carried for 500 ns. Structural features for aspartate isomerization prediction as described in F. J. Irudayanathan et al., mAbs (2022) were calculated for selected antibodies using MDTraj (version 1.9.8) Python library. (McGibbon R. et al. Biophys. J. (2015)); the results are shown in Table E28. The first 100 ns of each simulation trajectory was considered as the equilibration process and excluded from the analysis.

The prediction pipeline was unable to predict the aspartate isomerization of P20 light chain D50 that was observed experimentally (see Example 3.D and FIG. 11). For the prediction pipeline to predict an aspartate isomerization, an aspartate residue must satisfy three structural features. Feature (i) pertains to the backbone dihedral angles of the next residue and evaluates the likelihood of deprotonation of the backbone nitrogen atom of that residue. It must be in the alpha-helix region. Especially if the next residue is non-polar such as Ala, it must be in the left-handed alpha-helix region. Feature (ii) concerns the dihedral angles of χ1 and ψ of the aspartate residue. This ensures that the Cγ atom of the aspartate residue and the backbone nitrogen atom of the next residue are in a reactive orientation to form an intermediate chemical species, succinimide. Feature (iii) relates to the solvent exposure of the aspartate residue, requiring that the solvent accessible surface area of the aspartate residue be greater than 25 Å². An aspartate residue is predicted to isomerize if these structural features are satisfied for at least 75 percent of simulation time. In the case of predicted structure (1) of P20 (see Table E28 below), the backbone conformation of the next residue was in a favored conformation, specifically in the left-handed alpha-helix region. However, the Cγ atom of the aspartate residue and the backbone nitrogen atom of the next residue were not in a reactive orientation to form an intermediate chemical species, succinimide. The aspartate residue was not fully solvated. In the other predicted structures (2) and (3) of P20, the backbone conformations of the next residue were not in a favored conformation but in the region s of right-handed alpha-helix and beta-strand, respectively. Therefore, none of the predicted structures for P20 satisfied at least two structural features necessary for aspartate isomerization, and P20 was not predicted to isomerize. Similarly, P21 light chain D50 could not satisfy the structural features of the reactive orientation, and the solvent exposure of the residue as observed for predicted structure (1) of P20, and it was predicted to not isomerize.

Conversely, even though P8 light chain D50 did not actually isomerize as determined experimentally from the stress test results reported in Example 3.D and FIG. 11, the results from the prediction pipeline indicated that it was predicted to isomerize. The backbone structure of the next residue was in the left-handed alpha-helix region. The aspartate residue sidechain and the backbone nitrogen atom had reactive near-attack conformations, and the aspartate residue was solvated throughout the simulations. Based on these structural features, it was predicted to isomerize. Burosumab, which has the same LCDR2 motif was predicted not to isomerize. The backbone structure of the next residue was in the left-handed alpha-helix region, and the aspartate residue was solvated throughout the simulations. However, the Cγ atom of the aspartate residue and the backbone nitrogen atom of the next residue were not in a reactive orientation as was observed for P20 predicted structure (1). Since Burosumab did not satisfy one structural feature necessary for aspartate isomerization, it was not predicted to isomerize.

The MD simulation-based aspartate isomerization prediction pipeline exhibited a tendency to predict that an aspartate will isomerize rather than not isomerize. According to its benchmark test results (as reported in Irudayanathan et al.), when it predicts that an aspartate will isomerize, approximately 20 percent (19 out of 96 test cases) of the predictions were correct. Conversely, when it predicts that an aspartate will not isomerize, approximately 99 percent (397 out of 402 test cases) of the predictions were correct. In light of these benchmark test results, the failure of the prediction pipeline to predict the aspartate isomerization that was observed for P20 is not a common occurrence.

TABLE E28
Summary of MD simulation-based aspartate in LCDR2 isomerization prediction results

| | | Feature (i) | Feature (i) | | | |
|---|---|---|---|---|---|---|
| Antibody | Target residue | (left- | (right- | Feature | Feature | |
| Name | (Kabat numbering) | handed)¹ | handed)² | (ii)³ | (iii)⁴ | Prediction |
| P20-rep1⁵ | LC D50/A51 | 100% | 0% | 29.0% | 74.8% | X (No isomerization) |
| P20-rep2⁵ | LC D50/A51 | 0.1% | 98.7% | 7.2% | 71.5% | X (No isomerization) |
| P20-rep3⁵ | LC D50/A51 | 0.1% | 24.5% | 32.2% | 17.6% | X (No isomerization) |
| P21 | LC D50/A51 | 100% | 0% | 0.3% | 37.0% | X (No isomerization) |
| P8 | LC D50/A51 | 100% | 0% | 89.8% | 98.2% | ◯ (Isomerization) |
| Burosumab | LC D50/A51 | 100% | 0% | 45.5% | 100% | X (No isomerization) |

¹Time fraction of backbone conformation of the next residue in the left-handed alpha-helix region must be greater than 75% for aspartate isomerization if the next residue is non-polar.
²Time fraction of backbone conformation of the next residue in the right-handed alpha-helix region. The backbone conformation of the next residue must be in the alpha-helix region for more than 75% of the time for aspartate isomerization.
³Time fraction of backbone and sidechain dihedral conformation of aspartate in a reactive near-attack orientation must be greater than 75% for aspartate isomerization.
⁴Time fraction of the aspartate residue solvent accessible (solvent accessible surface area >25 Å²) must be greater than 75% for aspartate isomerization.
⁵To account for the disordered region around LCDR2 in the crystal structure of P20, which implies structural heterogeneity, three predicted structures (referred to herein as P20-rep1, P20-rep2, and P20-rep3) with distinctive conformations for the disordered region were subjected to the prediction method.

### B. Structural Basis of Isomerization

To understand the structural basis of isomerization liability, the crystal structure of P20 was compared to that of the Fab fragment of P8 as well as Burosumab. Burosumab and P20 share the same linear sequence of LC-CDR2, whereas P8 shares six out of the seven amino acids in the LC-CDR2 sequence, namely DASSLQS in Burosumab and P20 vs. DASSLES in P8. In the P8 and Burosumab structures, all residues of the heavy and light chain variable regions were fully-ordered and visible in the electron density maps, indicating that the Fv regions in these molecules stably adopt the experimentally observed structures. In contrast, in the structure of the P20 Fab fragment bound to the IL18Rβ D3 domain, while every residue of IL18Rβ (except the C-terminal arginine) and the P20 heavy chain variable region was fully-ordered and visible in the electron density maps, notably light chain CDR2 residues Ser52/Ser53/Leu54 were disordered in crystallographic asymmetric unit light chain copy 1 and light chain CDR2 residues Asp50/Ala51/Ser52/Ser53/Leu54/Glu55/Ser56/Gly57/Val58/Pro59 are disordered in crystallographic asymmetric unit light chain copy 2. Together, these data strongly suggest that P20 light chain CDR2 is conformationally flexible and more prone to structural disorder than the P8 and Burosumab light chain CDR2s, despite P20, Burosumab, and P8 having the same DA motif at the start of light chain CDR2. Because P8 and Burosumab were not prone to Asp50-Ala51 isomerization at light chain CDR2, while P20 is prone to Asp50-Ala51 isomerization at light chain CDR2, and because of the nearly identical light chain CDR2 primary sequences of P8, Burosumab, and P20, whether susceptibility of light chain CDR2 Asp-Ala isomerization is attributable to differences in primary sequence and/or tertiary structure within the Fv region adjacent to light chain CDR2 was interrogated. Most notably, both the primary sequences and experimentally observed tertiary structures of the P20, P8, and Burosumab heavy chain CDR3s were significantly different from one another. The P8 heavy chain CDR3 primary sequence was EGRVSPTDYYGMDV (SEQ ID NO: 54) and formed an elongated loop structure that projected away from the light chain and inserted into a cleft on the surface of the IL-18Rβ D2 domain (see FIG. 14C; middle, medium gray; HCDR3 shown as transparent surface and LCDR2 shown as dots). The Burosumab heavy chain CDR3 primary sequence was much shorter, comprising DIVDAF (SEQ ID NO: 161), and formed a short-hairpin structure that lied adjacent to that of light chain CDR2 (see FIG. 14C; right, dark gray; HCDR3 shown as transparent surface and LCDR2 shown as dots). However, in contrast, the P20 heavy chain CDR3 was significantly longer with many more bulky amino acid sidechains, comprising the primary sequence DVGIAARNSYYYVMDV (SEQ ID NO: 10) and forming an elongated two-stranded β-sheet that extended into the three dimensional space into which light chain CDR2 would be predicted to occupy if it adopted the tertiary structure observed in P8 and Burosumab (see FIG. 14C; left, light gray; HCDR3 shown as transparent surface and LCDR2 shown as dots). These observations indicate that P20's bulkier heavy chain CDR3 β-sheet structure may induce disorder in the light chain CDR2, which may be associated with its susceptibility to Asp-Ala isomerization.

These results show that, even using a state-of-the art prediction method, the isomerization in LCDR2 of P20 was not predictable.

### Example 7—Characterizing Potency of Antibody P20-V1

This example provides an assessment of the potency of antibody P20-V1, an engineered framework reverted variant of antibody P20, to inhibit the IL-18 mediated production of interferon gamma (IFNg) using different species models and/or cell types.

### A. Potency Assessment in Human Myelomonocytic KG-1 Cell Line

Human myelomonocytic KG-1 cells produce interferon gamma (IFNg) in response to human IL-18 and can therefore be used to assess functional response to IL-18 in presence of inhibitors, as described by Konishi et al. (1997). Thus, the potency of P20-V1 was profiled in this cellular functional assay, using huIgG1.3f as an isotype negative control.

Specifically, KG-1 cells (ATCC CCL-246) were grown in IMDM (Thermo Fisher, cat #12200) with 20% Heat Inactivated FBS (Gibco, 10082-147) and Penicillin-Streptomycin in a 37C 5% CO2 incubator. 50,000 cells per well were plated in a 96-well plate (Thermo-Fisher, cat #07-200-90) in presence of recombinant human TNF-α at 20 ng/ml final concentration (R&D, cat #210-TA-100/CF). They were then pre-treated for 1 hour with anti-huIL-18Rβ antibody-P20-V1 or isotype control monoclonal antibody with a final concentration range from 100 nM to 0.04 nM. One hour later, the cells were then stimulated with recombinant human IL-18 (Sino Biologics, cat #10119-HNCE) at a final concentration of 8 ng/ml in the assay well. After a 16-24 hours incubation at 37° C., 5% CO2, the plates were centrifuged for 5 minutes at 2300 rpm. Supernatants were then collected for IFNg secretion measurement using the MSD platform (IFNg MSD assay, cat #K151QO). To calculate percent inhibition, the activity (IFNg level) in the presence of inhibitory antibody was compared to the activity in the absence of the inhibitor (such as isotype control). The following general formula for percent inhibition was used:

Percent Inhibition=[(Control Activity−Inhibited Activity)/Control activity]×100.

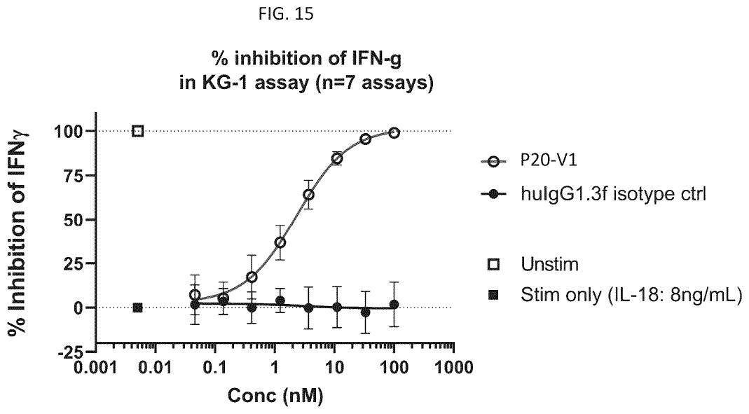

The potency of P20-V1 was assessed by determining the inhibition of IL-18-mediated IFNg release from KG-1 cells using a broad range of concentration from 0.04 nM to 100 nM. As shown in FIG. 15, P20-V1 treatment decreased IL-18-mediated IFNg secretion in a dose-dependent manner, while the isotype control antibody did not show any activity. The IC50 values and maximum IFNg inhibition level in 7 independent experiments are shown in Table E29.

TABLE E29
Summary of potencies (IC50, nM) and maximum interferon gamma inhibition
levels (Ymax, %) of P20-V1 (7 independent KG-1 experiments)

| | | | | | | | | | Standard |
|---|---|---|---|---|---|---|---|---|---|
| | Exp. | Exp. | Exp. | Exp. | Exp. | Exp. | Exp. | | Deviation |
| | #1 | #2 | #3 | #4 | #5 | #6 | #7 | Average | (S.D.) |
| P20-V1 IC50 | 0.96 | 2.1 | 1.46 | 1.2 | 2.94 | 2.8 | 2.6 | 2.01 | 0.81 |
| (nM) | | | | | | | | | |
| P20-V1 | 98.9 | 98 | 99.1 | 98.9 | 99.3 | 99.6 | 99.3 | 99.01 | 0.51 |
| Ymax (%) | | | | | | | | | |

Based on the results, P20-V1 potency was able to be evaluated in KG-1 cells using IL-18-mediated IFNg secretion as functional readout. An average potency of 2 nM with 99% maximal IFNg inhibition was observed with this monoclonal antibody, indicating that P20-V1 achieves strong potency at nanomolar dosing in KG-1 cells.

### B. Potency Assessment in Human Whole Blood

Human whole blood collected from heathy donors display secretion of interferon gamma (IFNg) when stimulated with LPS+IL-12 or IL-18+IL-12. LPS+IL-12 is a physiologically relevant stimulation in the context of inflamed gastrointestinal tract, where the local epithelial damage leads to microbiome/LPS exposure and increased IL-12 contributes to inflammatory bowel disease (IBD) pathogenesis. LPS-induced IL-18 levels in whole blood are similar to circulating IL-18 levels in IBD patient serum (500 to 1500 μg/mL. Leach et al. 2008). IL-12 synergizes with IL-18 for IFNg production from T cells and increases IL-18R expression (Tominaga et al. 2000). Given one desired indication of an exemplary anti-human IL-18Rβ (huIL-18Rβ) antibody is to treat IBD, the ability of P20-V1 to inhibit IFNg release under these conditions was assessed in vitro in freshly collected human whole blood.

Human whole blood was collected from healthy donors (BMS internal Blood Donor program) in EDTA vacutainer tubes (BD, ref 366643), diluted 1:1 with RPMI1640 (Gibco/Thermo-Fisher, cat #11875093) without FBS. 200 μL per well were dispensed in 96-well plates (Thermo-Fisher, cat #07-200-90). Cells were then pre-treated for 1 hour with anti huIL-18Rβ antibody-P20-V1 or isotype control monoclonal antibody (hulgG1.3f)—with a final concentration range from 100 nM to 0.04 nM (LPS+IL-12 stimulation) or 300 nM to 0.14 nM (IL-18+IL-12 stimulation). One hour later, the cells were then stimulated with (i) recombinant human IL-18 (Sino Biologics, cat #10119-HNCE)+human IL-12p70 (PeproTech, cat #200-12-50 μg) at a final concentration of 10 ng/mL each in the assay well, or (ii) LPS (Sigma-Aldrich, cat #L4391-1 MG)+human IL-12p70 (PeproTech, cat #200-12-50 μg) at final concentrations of 5 μg/mL and 5 ng/ml respectively in the assay well. After a 16-24 hours incubation at 37° C., 5% CO2, the plates were centrifuged for 5 minutes at 4000 rpm. Supernatants were then collected for IFNg secretion measurement using the MSD platform (IFNg MSD assay, cat #K151QO). To calculate percent inhibition, the activity (IFNg level) in the presence of inhibitory antibody was compared to the activity in the absence of the inhibitor (such as isotype control). The following general formula for percent inhibition was used:

Percent Inhibition=[(Control Activity−Inhibited Activity)/Control activity]×100.

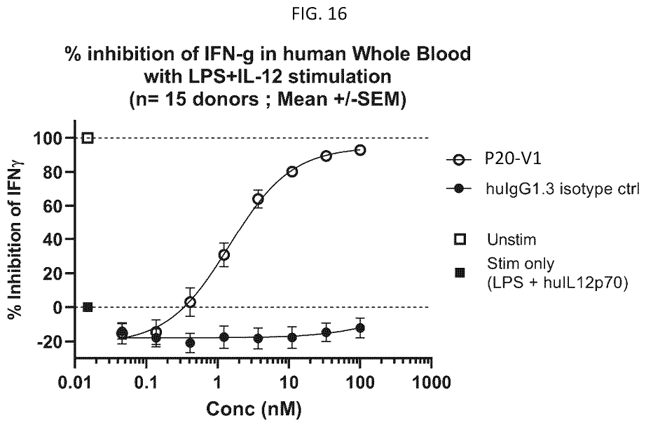

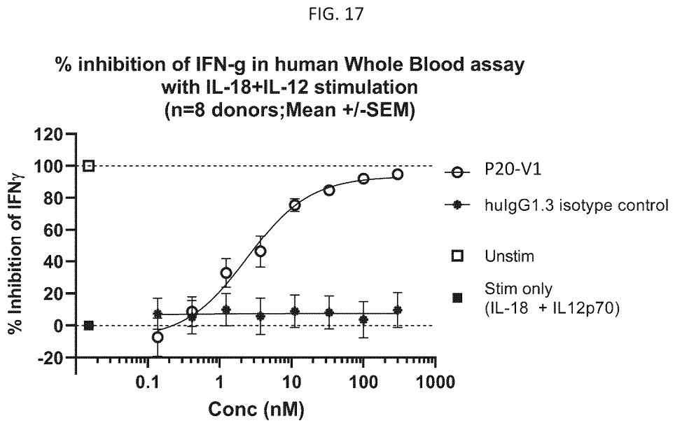

IFNg levels were measured in human whole blood after stimulation in vitro with LPS+IL-12 (FIG. 16) or IL-18+IL-12 (FIG. 17), and in the presence of increasing concentrations of P20-V1. As shown in FIGS. 16-17, IFNg release was inhibited by P20-V1 in a dose-dependent manner in whole blood, while the isotype control antibody did not show any activity. The IC50 values and maximum IFNg inhibition levels for LPS+IL-12 stimulation is shown in Table E30 while the IC50 values and maximum IFNg inhibition levels for IL-18+IL-12 stimulation is shown in Table E31 below.

TABLE E30
Summary of potencies (IC50, nM) and maximum IFNg levels
(Ymax, %) of P20-V1 (15 independent human whole blood
with LPS + IL-12 stimulation experiments)

| Donor # | P20-V1 IC50 (nM) | P20-V1 Ymax (%) |
|---|---|---|
| C223 | 0.83 | 98.3 |
| C245 | 1.22 | 97.4 |
| C229 | 1.3 | 91.4 |
| C177 | 0.47 | 99.8 |
| C198 | 4.9 | 97.8 |
| C241 | 1.2 | 95.5 |
| C251 | 1.7 | 95.5 |
| C163 | 1.1 | 86.2 |
| C215 | 1.54 | 95.7 |
| C238 | 0.57 | 95.9 |
| C257 | 2.22 | 91.3 |
| C200 | 2.55 | 86.2 |
| C205 | 1.56 | 92.96 |
| C256 | 1.38 | 95.8 |
| C175 | 1.48 | 93.6 |
| Average | 1.60 | 94.22 |
| Standard Deviation (S.D.) | 1.06 | 4.04 |

TABLE E31
Summary of potencies (IC50, nM) and maximum IFNg levels
(Ymax, %) of P20-V1 (8 independent human whole blood
with IL-18 + IL-12 stimulation experiments)

| Donor # | P20-V1 IC50 (nM) | P20-V1 Ymax (%) |
|---|---|---|
| C121 | 3.60 | 93.30 |
| C175 | 0.50 | 96.00 |
| C237 | 2.74 | 94.40 |
| C253 | 1.24 | 90.80 |
| C121 | 1.30 | 97.82 |
| C238 | 1.35 | 95.35 |
| C249 | 4.29 | 94.65 |
| C252 | 3.23 | 94.40 |
| Average | 2.28 | 94.59 |
| Standard Deviation (S.D.) | 1.36 | 2.04 |

Based on the data, the mean IC50 value for the inhibition of the IFNg response was 1.6 nM in LPS+IL-12 condition (n=15 donors) and 2.3 nM in IL-18+IL-12 condition (n=8 donors).

In conclusion, P20-V1 potency was evaluated in human whole blood cells using LPS+IL-12- or IL-18+IL-12-mediated IFNg secretion as a functional readout. In both cases, P20-V1 achieved IC50s at nanomolar dosing, indicating that P20-V1 has strong potency in human whole blood.

### C. Potency Assessment in Cynomolgus Monkey (Cyno) Whole Blood

Whole blood collected from cynomolgus monkey (cyno) display secretion of interferon gamma (IFNg) when stimulated with IL-18+IL-12. The ability of P20-V1 to inhibit IFNg release under these conditions was assessed in vitro in freshly collected cyno whole blood.

Specifically, cyno whole blood was collected from healthy animals in Na Heparin vacutainer tubes (BD367671), diluted 1:1 with RPMI1640 (Gibco/Thermo-Fisher, cat #11875093) without FBS. 200 μL per well were dispensed in U-bottom 96-well plates (Falcon, cat #353077). Cells were then pre-treated for 1 hour with anti-human IL-18Rβ antibody-P20-V1 or isotype control monoclonal antibody (huIgG1.3f)—with a final concentration range from 300 nM to 0.14 nM. One hour later, the cells were then stimulated with recombinant human IL-18 (Sino Biologics; cat #10119-HNCE)+human IL-12p70 (PeproTech, cat #200-12H) at a final concentration of 30 ng/ml and 10 ng/mL respectively in the assay well. After a 16-24 hours incubation at 37° C., 5% CO2, the plates were centrifuged for 5 minutes at 4000 rpm. Supernatants were then collected for IFNg secretion measurement using the MSD platform using V-plex NHP IFNg detection kit (cat #K156QOD-4) modified with primate IFNg standard (R&D systems, cat #DY961-840876) and primate IFNg detection biotinylated antibody (R&D systems, cat #DY961-840875), as detection antibody for the assay. To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

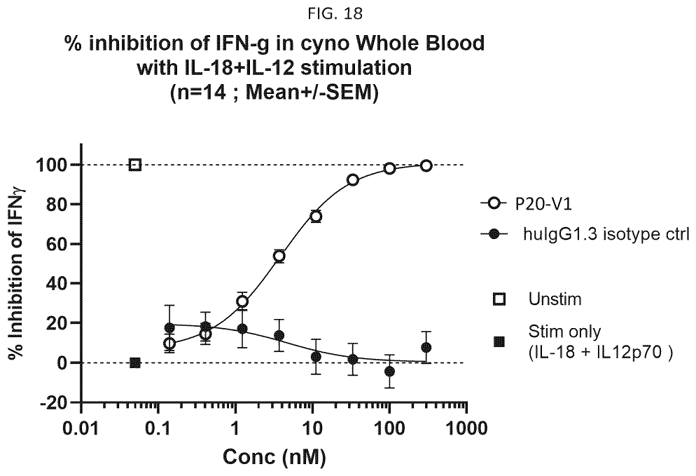

IFNg levels were measured in cyno whole blood after stimulation in vitro with IL-18+IL-12, and in the presence of increasing concentrations of P20-V1 (concentration range 0.14 nM to 300 nM), as shown in FIG. 18. The data shows that IFNg release was inhibited by P20-V1 in a dose-dependent manner in whole blood, while the isotype control antibody did not show any activity.

The IC50 values and maximum IFNg inhibition levels for each individual blood donor are shown in Table E32 below.

TABLE E32
Summary of potencies (IC50, nM) and maximum IFNg levels
(Ymax, %) of P20-V1 (14 independent cyno whole blood
with IL-18 + IL-12 stimulation experiments)

| Donor # | P20-V1 IC50 (nM) | P20-V1 Ymax (%) |
|---|---|---|
| B21438 (f) | 2.2 | 99 |
| B21333 (f) | 1.8 | 100 |
| B21374 (f) | 2.5 | 99 |
| B21394 (f) | 6.0 | 100 |
| B21411 (f) | 5.1 | 100 |
| B21435 (f) | 10.5 | 98 |
| B22017 (f) | 5.7 | 100 |
| B22187 (m) | 3.9 | 100 |
| B7619 (f) | 5.1 | 100 |
| B8476 (m) | 3.3 | 99 |
| B9301 (f) | 4.7 | 99 |
| B9313 (m) | 2.4 | 100 |
| B9447 (m) | 5.1 | 100 |
| B9496 (m) | 1.9 | 99 |
| Average | 4.30 | 99.5 |
| Standard Deviation (S.D.) | 2.32 | 0.65 |

Based on the data, the mean IC50 value for the inhibition of the IFNg response was 4.3 nM (n=14 donors) with >99% maximal IFNg inhibition.

In conclusion, P20-V1 potency was able to be evaluated in cyno whole blood cells using IL-18+IL-12-mediated IFNg secretion as a functional readout. P20-V1 achieved a single digit nanomolar IC50, indicating P20-V1 has strong potency in cyno whole blood.

### D. Cynomolgus Monkey (Cyno) In Vivo Pharmacokinetics and Potency Assessment

Given P20-V1's strong human/cynomolgus monkey (cyno) cross reactivity, the cyno was determined to be an ideal model for assessing both in vivo pharmacokinetics (PK) and potency to inhibit interferon gamma (IFNg) for P20-V1. While PK was determined by analyzing blood samples taken from live cyno following P20-V1 administration, a concurrent ex vivo cyno whole blood assay was also developed to measure potency. Specifically, human IL-18 and IL-12 were used to stimulate the production of interferon gamma (IFNg) in sampled cyno whole blood to assess if P20-V1 inhibited the crucial biomarker IFNg.

To determine in vivo PK and ex vivo IFNg stimulation, protein naïve female and male cynomolgus (Buckshire) aged 3-6 years were used. All protocols and housing were approved by the BMS IACUC. Vehicle, 0.2 mg/kg or 2 mg/kg P20-V1 were dosed subcutaneously at 0 hours (Day 1). Blood for PK and whole blood (WB) assay were collected at times after 0 hrs. The PK collection timepoints were at 4, 24, 48, 72, 96, 168, 240, 336, 432, and 504 hours. The WB assay time points were at 24, 72, 168, 336, and 504 hours. Blood was collected via saphenous, femoral, or cephalic vein from chaired, conscious animals.

For serum preparation, whole blood was collected in serum separator tubes, allowed to clot (15 mins) and tubes spun at 2500 rpm for 8 minutes, with serum being collected for analysis.

Serum samples were analyzed using the Gyros platform. Biotinylated human IL-18Rβ was used as a capture reagent at 25 μg/mL and mouse α-human IgGFc clone 10C7-A647 was used for detection at 0.5 μg/mL. Standards and quality controls were prepared in 100% cyno serum.

Following either vehicle, 0.2 mg/kg of P20-V1, or 2 mg/kg P20-V1 subcutaneous administration, WB was taken at the appropriate time points for the in vivo PK or ex vivo assay.

For the in vivo PK assay, serum was separated and analyzed for P20-V1 concentration using an ELISA based assay. Pharmacokinetic parameters were calculated using non-compartmental analysis and reported in Table E33.

TABLE E33
PK parameters after subcutaneous administration
of P20-V1 in cynomolgus monkeys
Pharmacokinetic (PK) parameters after subcutaneous administration

Dose (mg/kg)

| | 0.2 mg/kg | 2 mg/kg |
|---|---|---|
| | Mean ± SD | Mean ± SD |
| Cmax (nM) | 13 ± 2 | 124 ± 23 |
| Tmax (h) | 1.8 ± 0.5 | 1.5 ± 1.7 |
| AUClast (uM*h) | 2.6 ± 0.3 | 32.6 ± 8.2 |
| Vss/F (mL/kg) | 90 ± 9 | 89 ± 19 |
| CL/F (mL/h/kg) | 0.33 ± 0.09 | 0.31 ± 0.09 |
| Half-life (days) | 8.7 ± 4.0 | 8.9 ± 4.0 |
| MRTINF (days) | 13 ± 5.2 | 14 ± 4.2 |

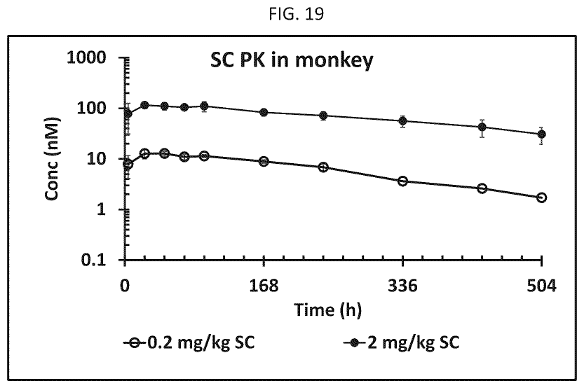

FIG. 19 shows the dose-proportional exposure of P20-V1 in monkey serum over time.

Additionally. P20-V1 demonstrates linear PK between 0.2 and 2 mg/kg and no evidence of target mediated drug disposition (TMDD) as seen by comparable half-life and mean residence time between the two dose levels.

For the ex vivo assay, cyno whole blood was collected in sodium heparin tubes and diluted 1:1 with RPMI 1640 (without FBS). 200 μl was added/well in 96 well U-bottom plate for triplicates. Samples were then stimulated with human IL-18 (Sino Biological #10119-HNCE; 30 ng/ml)+human IL-12p70 (Peprotech Ref #200-12H; 10 ng/ml) for 24 hr at 37° C.+5% CO2. Samples were then centrifuged at 4000 rpm/5 min and plasma was collected. Samples were stored at −80° C.

To measure IFNg, plasma samples were diluted 20× and the NHP IFNg MSD protocol (K156QOD-4) was modified to use Primate IFNg standard and Primate IFNg biotinylated monoclonal antibody (mAb) from R&D duoset ELISA kit DY961 (Primate IFNγ Standard: 840876 and Primate IFNγ Detection Biotinylated Antibody: 840875).

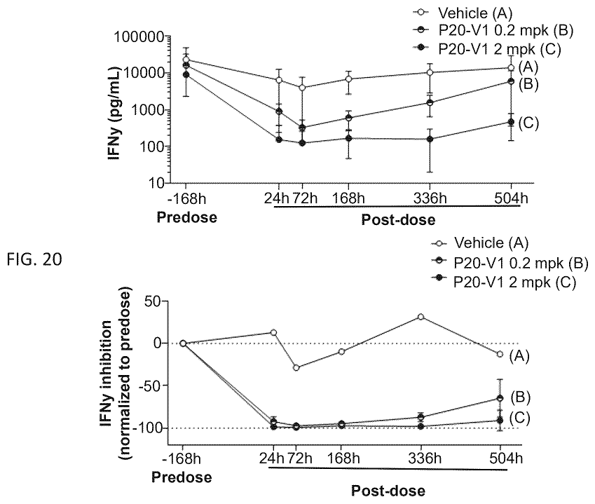

FIG. 20 shows the sustained inhibition of IFNg in the ex vivo cyno WB assay following P20-V1 administration in vivo. Based on the data, the 0.2 mg/kg and 2 mg/kg P20-V1 doses dependently reduced IFNγ levels over the time course. In fact, 2 mg/kg of P20-V1 inhibited IFNγ greater than 90% over the entire time course, indicating P20-V1 achieved high potency.

In conclusion, 2 mg/kg subcutaneous dose of P20-V1 inhibited IFNγ at greater than 90% for 3 weeks in ex vivo stimulated whole blood. P20-V1 also showed dose-dependent exposure in the serum, indicating that P20-V1 potently inhibited IL-18 mediated production of IFNγ at nanomolar doses.

### E. Potency Assessment in Mouse Whole Blood and Splenocytes

Following ex vivo stimulation of murine whole blood, interferon gamma (IFNg) can be produced in response to recombinant murine (rm) IL-12/-18 stimulation. Thus, the functional impact of P20-V1 on rmIL-12/-18- or rmIL-12/LPS-induced IFNg production from murine whole blood and splenocytes was investigated. For the generation of human IL-18Rβ knock-in (huIL-18Rβ-KI) mice, the IL-18Rβ extracellular domain (ECD) was humanized at the endogenous muIl18rap locus. The signal peptide, transmembrane domain (TM) and intracellular domain TIR all remained murine.

HuIL-18Rβ-KI and wild type (WT) C57BL/6 mice were allowed to acclimate for at least 72 hours before the study. Identical enrichment was provided for each cage: InnoWheel and Bed'r nest, along with standard corn cob bedding and rodent chow. Female mice were used at greater than 10 weeks of age. HuIL-18Rβ-KI mice were ear notched using Stoelting™ Ear Punch (Fisher Scientific) and genotyping was verified.

To collect whole blood and spleen, mice were culled using C02 and cervical dislocation. Upon confirmation of death, cardiac puncture was carried out to collect whole blood and placed in lithium heparin coated tubes. Spleens were collected 70 μm filters in RPMI on ice in 6-well plates for functional assays.

To perform the whole blood assay, blood was diluted 2× with plain RPMI and aliquoted at 180 μL per well in a 96-well round bottom plate. Blood was pre-treated with either human isotype control (huIgG1.3f) or P20-V1 for 1 hour at 37° C. After 1 hour, blood was stimulated with rmIL-18 (8 ng/ml; Sino Biologic 50073-MNCE) and rmIL-12 (10 ng/ml; Peprotech 210-12) for 18 hours. After 18 hours, samples were centrifuged 1000×g for 10 min at 4° C. and transferred to a 96-well v-bottom plate. Samples were diluted 25× for MSD, where samples were analyzed with kits for IFNg (K152QOD-2), 19-plex (K15255D-4; Meso Scale Discovery) or Th1/Th2 12-plex Luminex kit (EXP110-20820-901; ThermoFisher). To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

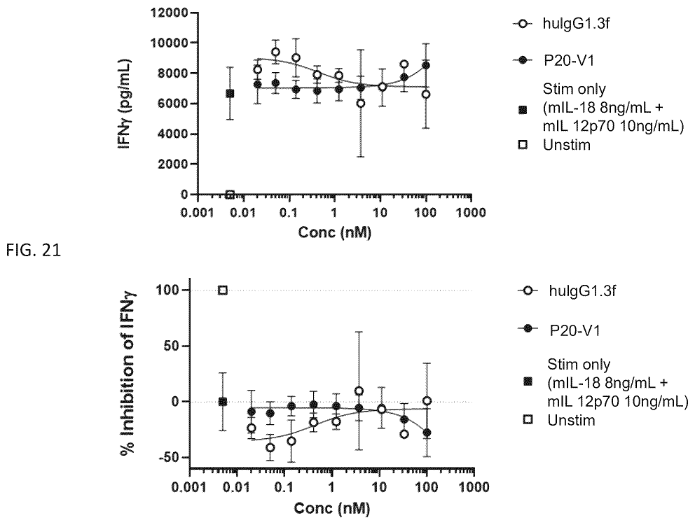

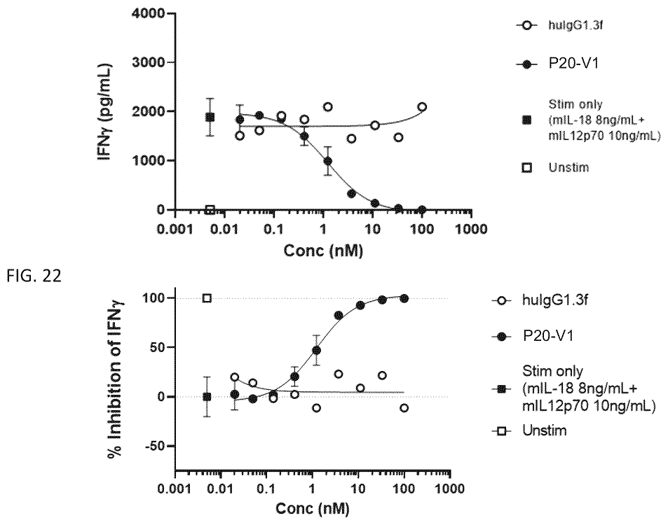

When whole blood taken from WT mice was stimulated with rmIL-12 and rmIL-18 to induce IFNg production, P20-V1 did not inhibit IFNg (FIG. 21). In contrast, when whole blood taken from huIL-18Rβ-KI mice was stimulated with rmIL-12 and rmIL-18 to induce IFNg production, P20-V1 inhibited IFNg production dose-dependently and an IC50 value of 1.228 nM was determined (FIG. 22).

Likewise, a similar experiment was also performed using splenocytes from the collected spleens of the mice. Specifically, splenocytes were isolated using the non-enzymatic spleen dissociation kit protocol (Miltenyl; order no. 130-095-926). Cells were then lysed and seeded at 400,000 cells/well in 180 μl of complete media (RPMI, 10% HiFBS, Pen/Strep). Blood was pre-treated with either isotype control (huIgG1.3f) or P20-V1 for 1 hour at 37° C. After 1 hr, splenocytes were stimulated with LPS (100 ng/ml; Sigma L4391) and rmIL-12 (10 ng/ml; Peprotech 210-12) for 18 hours. After 18 hours, samples were centrifuged 1000×g for 10 min at 4° C. and transferred to a 96-well v-bottom plate. Samples diluted 25× for MSD, where samples were analyzed with kits for IFNg (K152QOD-2), 19-plex (K15255D-4; Meso Scale Discovery) or Th1/Th2 12-plex Luminex kit (EXP110-20820-901; ThermoFisher). To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

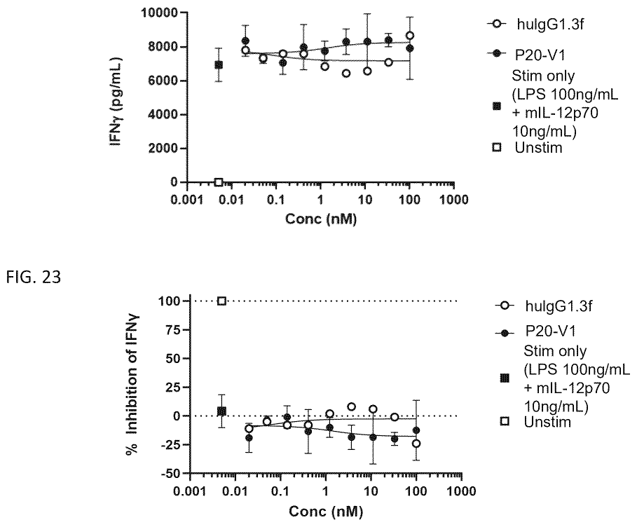

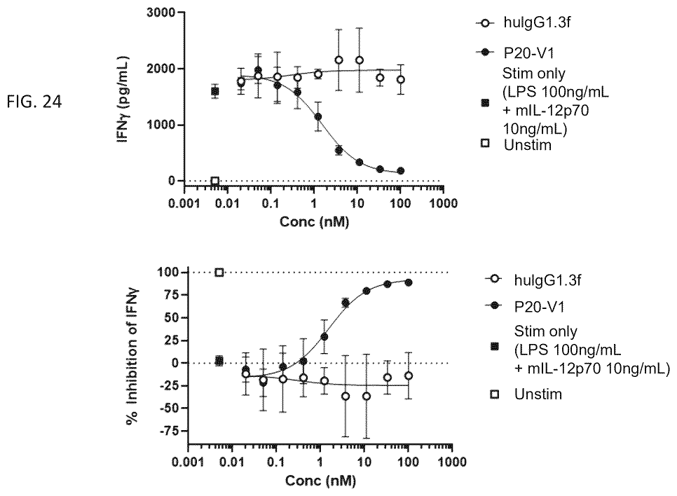

When splenocytes isolated from WT mice were stimulated with rmIL-12 and LPS to induce IFNg production, P20-V1 did not inhibit IFNg (FIG. 23). In contrast, when splenocytes isolated from huIL-18Rβ-KI mice were stimulated with rmIL-12 and LPS to induce IFNg production, P20-V1 inhibited IFNg production dose-dependently and an IC50 value of 1.29 nM was determined (FIG. 24).

In summary, P20-V1 inhibited IFNg production from stimulated whole blood and splenocytes from huIL-18Rβ-KI mice, with IC₅₀s of 1.2 nM and 1.29 nM, respectively. No functional activity (IFNγ inhibition) was observed in whole blood and splenocytes from WT mice with P20-V1. Taken together, these results suggest that P20-V1 specifically binds to huIL-18Rβ to inhibit IFNg production at potent nanomolar doses in mice whole blood and splenocytes.

### F. Mouse In Vivo Pharmacokinetics and Potency Assessment

Following ex vivo stimulation of murine whole blood, interferon gamma (IFNg) can be produced in response to recombinant murine (rm) IL-12/-18 stimulation. Further, human IL-18Rβ knock-in (huIL-18Rβ-KI) mice allowed for the pharmacokinetics (PK) of P20-V1 to be assessed.

HuIL-18Rβ-KI mice were allowed to acclimate for at least 72 hours before the study. Identical enrichment was provided for each cage: InnoWheel and Bed'r nest, along with standard corn cob bedding and rodent chow. Male mice were used at greater than 12 weeks of age. HuIL-18Rβ-KI were car notched using Stoelting™ Ear Punch (Fisher Scientific), and genotyping was verified.

Isotype control (huIgG1.3f) was dosed at 1 mg/kg or P20-V1 was dosed subcutaneously at 1, 0.3, 0.1, 0.03, 0.01 and 0.003 mg/kg on Day 0 using a 1cc syringe and 27G needle. 24 hours later on Day 1, whole blood and serum were collected. P20-V1−. The vehicle for the isotype control and P20-V1 formulation was 20 mM histidine, 260 mM sucrose, 50 μM DTPA, and 0.05% PS80 pH 6.0.

To collect whole blood, mice were culled using C02 and cervical dislocation. Upon confirmation of death, cardiac puncture was carried out to collect whole blood and placed in lithium heparin coated tubes or serum separator tubes.

For the ex vivo whole blood assay, blood was diluted 2× with plain RPMI and aliquoted in 180 μL diluted whole blood per well of 96-well round bottom plate. Blood was stimulated with rmIL-18 (8 ng/ml; Sino Biologic 50073-MNCE) and rmIL-12 (10 ng/ml; Peprotech 210-12) for 18 hours. After 18 hours, samples were centrifuged 1000×g for 10 min at 4° C. and transferred to a 96-well v-bottom plate. Samples were diluted 25× for MSD. To calculate percent inhibition, the activity (IFNg level) in the presence of inhibitory antibody was compared to the activity in the absence of the inhibitor (such as isotype control). The following general formula for percent inhibition was used:

Percent Inhibition=[(Control Activity−Inhibited Activity)/Control activity]×100.

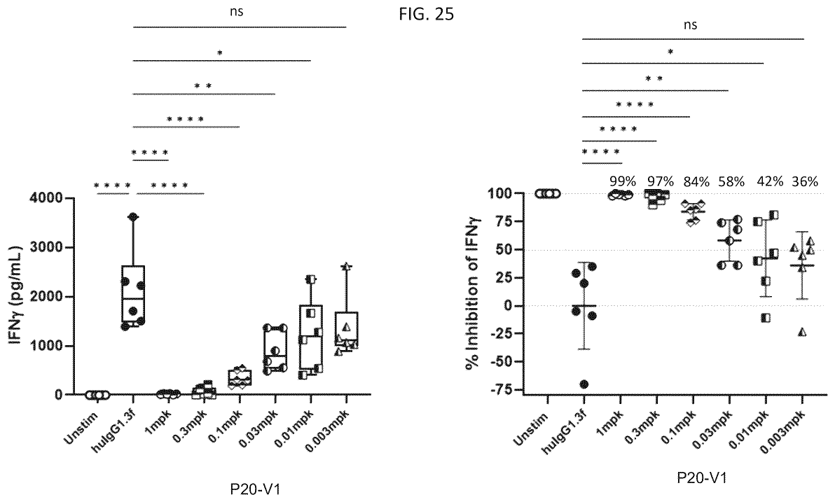

FIG. 25 shows the results of the ex vivo blood assay, where following in vivo administration of P20-V1 to huIL-18Rβ-KI mice, whole blood was stimulated with IL-12 and IL-18 to induce IFNg production. The results show that P20-V1 dose-dependently reduced IFNg production.

In addition to the ex vivo whole blood assay, PK was measured using the serum from the collected whole blood. Specifically, serum was isolated using serum separator tubes. Collected blood was stored on ice for a maximum of 30 min prior to centrifugation (8000 rpm for 8 minutes) and separation. The serum was then quantified for P20-V1 using the Gyros platform (3-step-2-Wash Wizard method with Gyrolab Bioaffy 200 CD 5% PMT Wash buffer 1: PBS+0.05% Tween 20 (PBST); Wash Buffer 2:0.5% SDS in 50 mM Glycine, pH 9.5; Column wash: PBST). Prior to processing for analysis, standards, quality controls (QCs), and study samples were plated and centrifuged (set to 3000 rpm) for 5 minutes. Biotinylated huIL-18Rβ (concentration: 1.2 mg/mL) was used as a capture reagent at 25 μg/mL and mu α-huIgGFc clone 10C7-A647 (concentration: 0.5 mg/mL) was used for detection at 0.5 μg/mL. Standards and QCs were prepared in 100% murine serum and all standards, QCs, and samples were diluted 10-fold (MRD) in PTB 1% BSA in PBS with 0.05% Tween 20.

For mediator quantification in the serum, V-plex Mouse IFNg (K152QOD-2-Lot #K0082090) and mu 19-plex MSD kit (K15255D-4) were utilized (Meso Scale Discovery). Samples were run fresh and with 1 in 25 dilution.

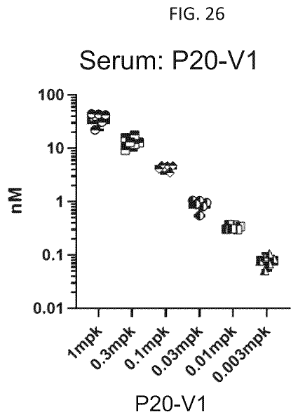

FIG. 26 shows the serum exposure of P20-V1 in huIL-18Rβ-KI mice at 24 hours after subcutaneous dosing. The data shows that dose-dependent exposure of P20-V1 was observed.

In summary, in vivo administration of P20-V1 dose-dependently inhibited IFNg from ex vivo stimulated whole blood. This was accompanied by dose-dependent exposure in the serum of P20-V1. Further, the 0.3 mpk dose of P20-V1 achieved an exposure level of 10 nM (IC90) at 24 hr and inhibited IFNg by 97%. The 0.03 mpk dose of P20-V1 achieved an exposure level of 1 nM (IC50) at 24 hr and inhibited IFNg by 58%. This was consistent with the results of the previous mouse whole blood assay where an IC50 value around 1 nM and an IC90 value around 10 nM were determined previously (see Example 7.E).

### G. Potency Assessment in Healthy and IBD Lamina Propria Lymphocytes

Interleukin 18 (IL-18) is a pro-inflammatory cytokine that is produced by intestinal epithelial cells, macrophages, and dendritic cells. IL-18 is stored at rest and cleavage is activated by the inflammasome pathway. IL-18 has been shown to be increased in inflammatory bowel disease (IBD) patient biopsies (Pizzaro et al., 1999). Signaling through IL-18Rαβ, which is expressed on T cells and NK cells, drives IFNg secretion. Thus, the effect of P20-V1 on IFNg secretion and selected genes was assessed in healthy and IBD lamina propria lymphocytes (LPLs).

To study the effect of P20-V1 on IFNg secretion, healthy volunteer and Ulcerative Colitis (UC) LPLs were obtained. LPLs were pre-treated for 1 hour with 1200 nM-0.006 nM of P20-V1 anti-IL-18Rb antibody or recombinant human IL-18 binding protein (rhIL-18BP). After 1 hour, LPLs were stimulated with 5 ng/ml rhIL-12p70 plus 6 ng/ml rhIL-18 and incubated for 24 hours. Recombinant proteins were purchased from R&D Systems (Minneapolis, MN). After 24 hours, supernatant was harvested and tested for IFNg secretion and analyzed using electrochemiluminescence technology (Mesoscale Discovery). To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

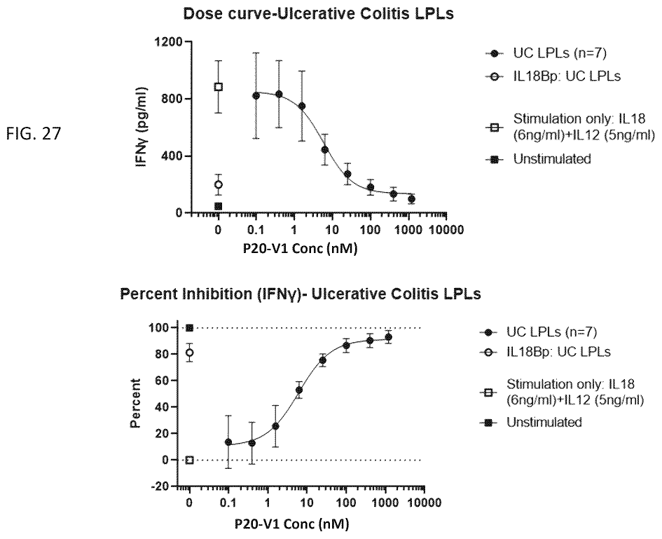

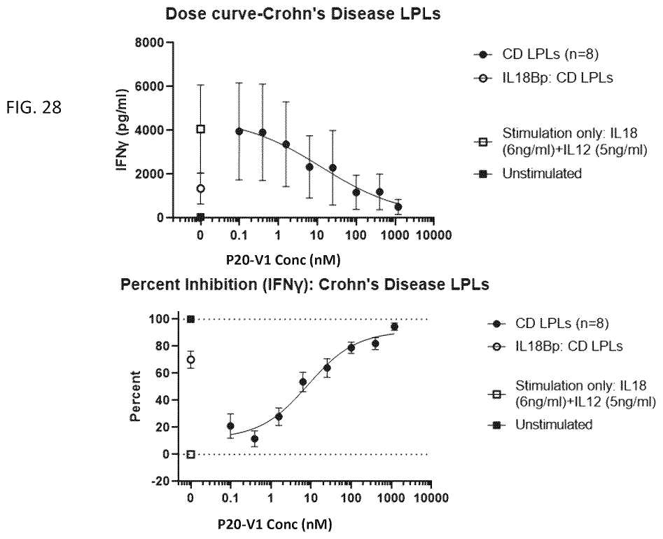

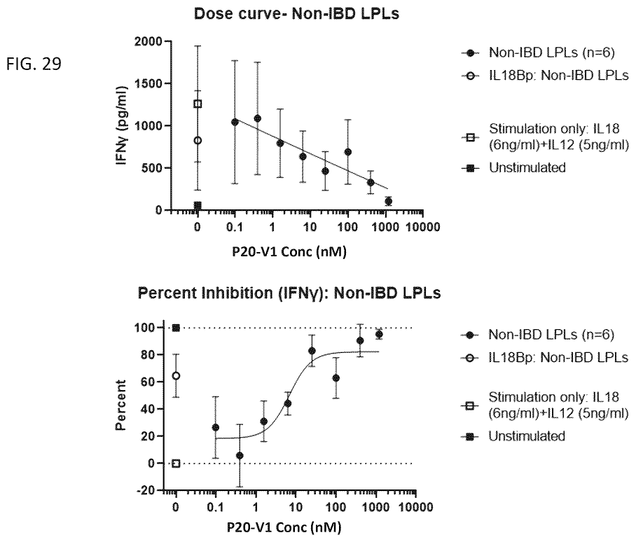

FIGS. 27-29 depict dose curves for UC LPLs (FIG. 27), CD LPLs (FIG. 28), and non-IBD (healthy) LPLs (FIG. 29) pre-treated with P20-V1 or rhIL-18BP at various concentrations for 1 hour and stimulated with rhIL-12p70 (5 ng/ml) and rhIL-18 (6 ng/ml) for 24 hours. For each cell type, IFNg levels dropped at higher concentrations of P20-V1, indicating that P20-V1 dose-dependently reduced IFNg production. P20-V1 inhibition on IFNg cytokine induced by IL-18 and IL-12 stimulation in each cell type is further summarized as IC50 values in Table E34 below.

TABLE E34
IC50 (nM) of P20-V1 inhibition of IFNg cytokine induced
by IL-18 and IL-12 stimulation in UC and non-IBD LPLs

| Disease | Replicate | IC50 (nM) | Average IC50 (nM) |
|---|---|---|---|
| Ulcerative colitis | 1 | 34.44 | 16.5 |
| | 2 | 10.85 | |
| | 3 | 10.33 | |
| | 4 | 2.635 | |
| | 5 | 0.0024 | |
| | 6 | 0.4015 | |
| | 7 | 56.7 | |
| Crohn's Disease | 1 | 3.818 | 18.3 |
| | 2 | 15.36 | |
| | 3 | 89.9 | |
| | 4 | 9.475 | |
| | 5 | 19.6 | |
| | 6 | 5.68 | |
| | 7 | 0.9 | |
| | 8 | 1.326 | |
| Healthy (non-IBD) | 1 | 12.75 | 39.8 |
| | 2 | 9.57 | |
| | 3 | 44.76 | |
| | 4 | 132 | |
| | 5 | 0.0116 | |
| | 6 | Unstable | |

Additionally, mRNA gene expression of 3 donors of UC LPLs pre-treated with P20-V1 at indicated concentrations for 1 hour and stimulated with rhIL-12p70 (5 ng/ml) and rhIL-18 (6 ng/ml) for 24 hours was evaluated. LPLs were lysed with MagMAX lysis/binding solution. Total RNA was isolated using the QIACube automation system. Complimentary DNA was prepared, and gene expression was analyzed using TaqMan Low-Density Gene Array (TLDA). The gene data was normalized to the average of GAPDH, ACTB, HPRT, RPLP0 and 18S house-keeping genes.

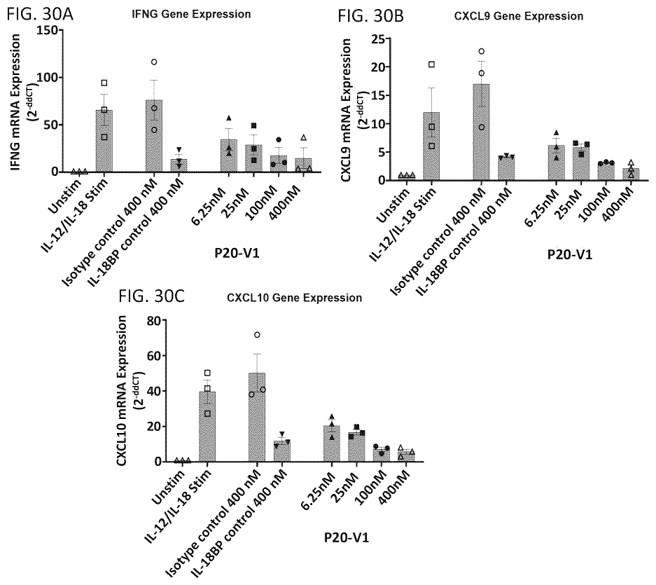

FIGS. 30A-C depict gene expression for IFNG (FIG. 30A), CXCL9 (FIG. 30B), and CXCL10 (FIG. 30C), respectively. For each target, gene expression levels raised in response to IL-12+IL-18 stimulation and treatment with P20-V1 led to decreased expression of each gene. These results shows that P20-V1 dose-dependently inhibited IFNg secretion and reduced expression of IFNG, CXCL9, and CXCL10 genes in healthy, UC, and CD patient LPL samples.

### Example 8—Characterizing Impact of Antibody P20-V1 on IL-12/-18-Induced Colitis in Mice

This example provides an in vivo assessment of the efficacy of antibody P20-V1 to reduce symptoms associated with IL-12/-18-induced colitis. IL-12/-18-induced colitis was modeled using repeated daily intraperitoneal dosing of recombinant murine IL-12 and IL-18 in mice, as repeated daily intraperitoneal dosing has been shown to induce prominent intestinal mucosal inflammation, fatty liver, piloerection, bloody diarrhea, and weight loss. These symptoms are also accompanied by elevation in serum levels of interferon gamma (IFNg).

The generation of human IL-18Rβ knock-in (huIL-18Rβ-KI) mice was substantially the same as previously described in Example 7.E. Briefly, the IL-18Rβ extracellular domain (ECD) in huIL-18Rβ-KI mice was humanized at the endogenous mull 18rap locus while the signal peptide, transmembrane domain (TM) and intracellular domain TIR all remained murine.

HuIL-18Rβ-KI mice were allowed to acclimate for at least 72 hours before the study. Identical enrichment was provided for each cage: InnoWheel and Bed'r nest, along with standard corn cob bedding and rodent chow. Female mice were used at greater than 14 weeks of age. HuIL-18Rβ-KI mice were ear notched using Stoelting™ Ear Punch (Fisher Scientific) or tail tipped and genotyping was verified.

To generate a 4-day IL-12/-18-induced colitis model, huIL-18Rβ-KI mice (n=5, 10, or 15 per group) were injected intraperitoneally (ip) each day for 4 days (Days 0-3) with 20 ng/g/mouse of recombinant murine (rm) IL-12 and 200 ng/g/mouse of rmIL-18 in 100 μl of phosphate buffered saline (PBS). Control mice were injected ip each day with 100 μl of PBS alone. rmIL-12 (#419-ML/CF) and rmIL-18 (#9139-ML/CF) were purchased from R&D Systems.

To test the efficacy of P20-V1 in treating 4-day IL-12/-18 colitis, either P20-V1 or a human isotype control (huIgG1.3f) were dosed subcutaneously using a 1cc syringe and 27G needle at either 10, 3, 1, or 0.3 mg/kg 24 hours prior to the first ip injection (Day-1). The vehicle for the P20-V1 and isotype control (huIgG1.3f) formulations was 20 mM histidine, 260 mM sucrose, 50 μM DTPA, and 0.05% PS80, pH 6.0.

Mice were harvested 24 hours after the 4th ip injection of either PBS or rmIL-12/-18 on Day 4. Mice were weighed daily and monitored for appearance and signs of illness, such as loose stools, diarrhea, and physical activity.

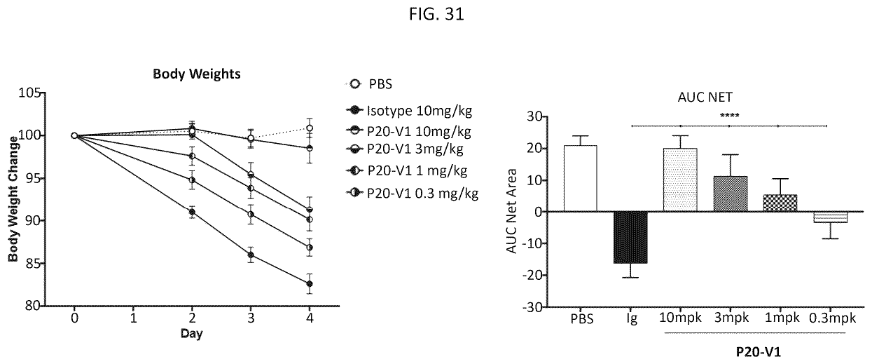

FIG. 31 shows the body weights of mice in each of the groups as change in body weight over each day (left panel) or the net area under the curve (AUC) of body weight over the 4 days (right panel) of IL-12/-18 injections or PBS control injections. The results indicate that all doses of P20-V1 administered at Day-1 subcutaneously attenuated IL-12/-18-induced body weight loss compared to the isotype control group.

To collect additional measurements, mice were culled using CO2 and cervical dislocation as per IACUC guidelines. Upon confirmation of death, the ileum and colon were also excised and flushed with PBS. Two pieces of the ileum were placed in formalin cassettes for histology analysis and three pieces of the colon were placed in formalin cassettes for histology analysis. All histology was analyzed and scored blinded to treatment groups. The summed score is a composite measure of inflammation, gland loss, erosion and hyperplasia. The summed damage score is a composite measure of gland loss, erosion and hyperplasia.

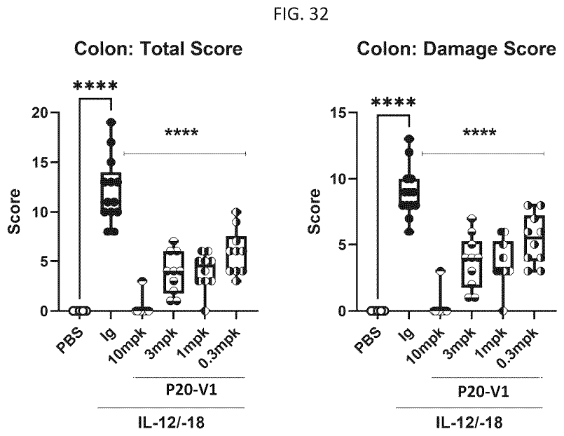

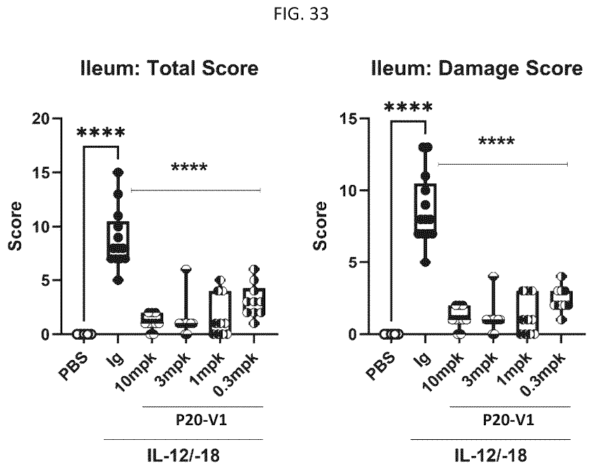

FIG. 32 and FIG. 33 show the colon and ileum histology scores, respectively, as total scores (left) and damage scores (right). The results indicate that in both the colon and ileum, each of the P20-V1 doses provided significant reductions in histology total and damage scores in comparison to the human isotype control.

For pharmacokinetic (PK) analysis, 20 mg of colon from the mid-section of the tissue and 20 mg from the terminal ileum were weighed and collected in Precellys tubes. In addition to measurements on the colon and ileum, cardiac puncture was carried out to collect whole blood. PK was then measured using the serum from the collected whole blood. Specifically, collected blood was stored on ice for a maximum of 30 minutes prior to centrifugation (8000 rpm for 8 minutes) and separation using serum separator tubes. The serum was quantified for levels of P20-V1 using the Gyros platform serum (3-step-2-Wash Wizard) method with Gyrolab Bioaffy 200 CD 5% PMT Wash buffer 1: PBS+0.05% Tween 20 (PBST); Wash Buffer 2:0. 5% SDS in 50 mM Glycine, pH 9.5; Column wash: PBST). Prior to processing for analysis, standards, quality controls (QCs), and study samples were plated and centrifuged (set to 3000 rpm) for 5 minutes. Biotinylated huIL-18Rβ (concentration: 1.2 mg/mL) was used as a capture reagent at 25 μg/mL and mu α-huIgGFc clone 10C7-A647 (concentration: 0.5 mg/mL) was used for detection at 0.5 μg/mL. Standards and QCs were prepared in 100% murine serum and all STD, QCs and samples are diluted 10-fold (MRD) in PTB 1% BSA in PBS with 0.05% Tween 20.

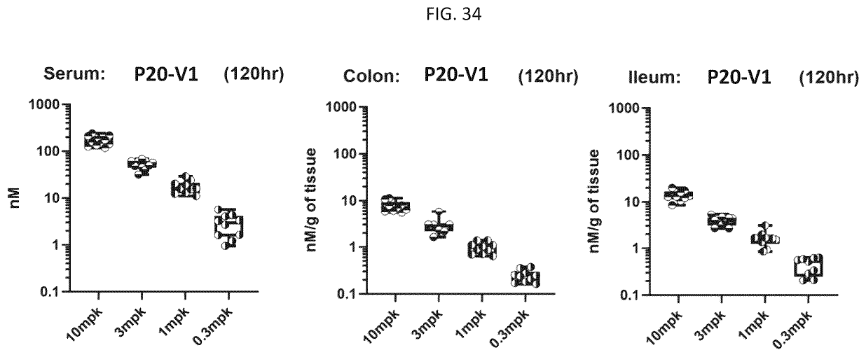

FIG. 34 shows the levels of P20-V1 in the serum (left), colon (middle), and ileum (right) 120 hours after P20-V1 administration. The results indicate that the highest levels of P20-V1 were found after the 10 mg/kg dose and that exposure was dose proportional.

To quantify IFNg inhibition, V-plex Mouse IFNg (K152QOD-2-Lot #K0082090) and mu 19-plex MSD kit (K15255D-4) were utilized (Meso Scale Discovery). Samples were run fresh and with 1 in 25 dilution. To calculate percent inhibition, the activity (IFNg level) in the presence of inhibitory antibody was compared to the activity in the absence of the inhibitor (such as isotype control). The following general formula for percent inhibition was used: Percent Inhibition=[(Control Activity−Inhibited Activity)/Control activity]×100.

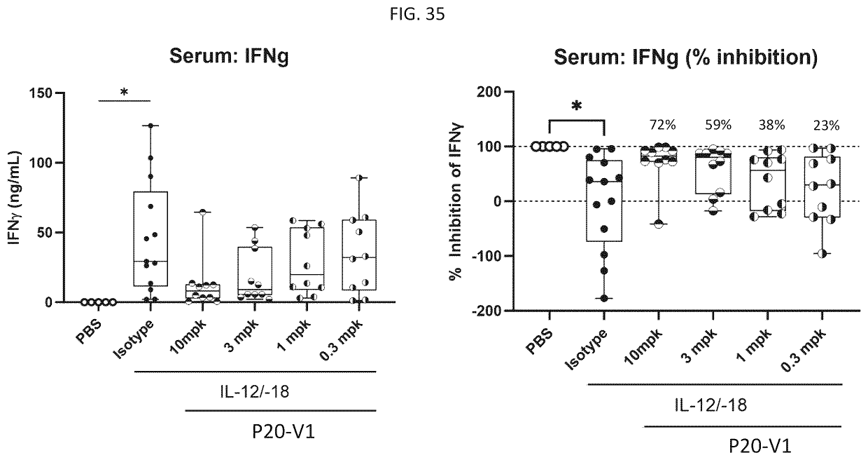

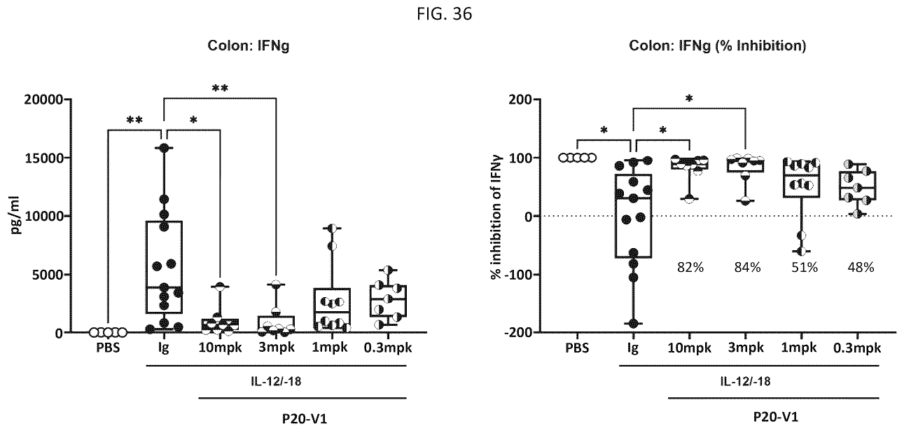

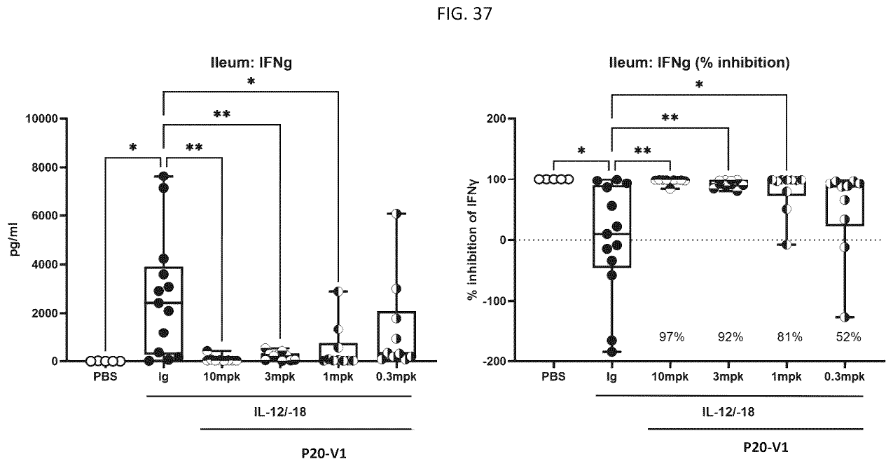

FIGS. 35-37 show levels of IFNg inhibition in serum, colon, and ileum respectively. IFNg concentrations are shown on the left, while percent inhibition of IFNg is shown on the right. Across serum, colon, and ileum, elevated levels of IFNg were shown in mice treated with IL-12/-18. In serum (FIG. 35), P20-V1 attenuated these high IFNg levels compared to the control human isotype. In the colon (FIG. 36), P20-V1 also attenuated high IFNg levels compared to the control human isotype, and the attenuation was statistically significant at doses of 3 mg/kg and 10 mg/kg. In the ileum (FIG. 37), P20-V1 also attenuated high IFNg levels compared to the control human isotype, and the attenuation was statistically significant at doses of 1 mg/kg, 3 mg/kg, and 10 mg/kg.

In summary, a single dose of P20-V1 delivered subcutaneously on Day-1 showed dose-proportional exposure in the serum, colon, and ileum at 120 hours post-administration, and this exposure was accompanied by attenuated features of IL-12/-18-induced colitis in huIL-18Rβ-KI mice. At all dose levels, P20-V1 significantly reduced body weight loss and colon and ileum histology scores, as compared to the isotype control. Further, P20-V1 significantly reduced IFNg levels in both the colon and ileum, at 10 mg/kg and 3 mg/kg in the colon and 10 mg/kg, 3 mg/kg, and 1 mg/kg in the ileum.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO | Annotation | Sequence |
| 1 | huIL-18Rβ-mFc | FNISGCSTKKLLWTYSTRSEEEFVLFCDLPEPQKSHFCHRNRLSPKQVPEHLP |
| | | FMGSNDLSDVQWYQQPSNGDPLEDIRKSYPHIIQDKCTLHFLTPGVNNSGSY |
| | | ICRPKMIKSPYDVACCVKMILEVKPQTNASCEYSASHKQDLLLGSTGSISCPS |
| | | LSCQSDAQSPAVTWYKNGKLLSVERSNRIVVDEVYDYHQGTYVCDYTQSD |
| | | TVSSWTVRAVVQVRTIVGDTKLKPDILDPVEDTLEVELGKPLTISCKARFGF |
| | | ERVFNPVIKWYIKDSDLEWEVSVPEAKSIKSTLKDEIIERNIILEKVTQRDLRR |
| | | KFVCFVQNSIGNTTQSVQLKEKRIEGRMDVPRDCGCKPCICTVPEVSSVFIFP |
| | | PKPKDVLTITLTPKVTCVVVAISKDDPEVQFSWFVDDVEVHTAQTQPREEQF |
| | | NSTFRSVSELPIMHQDWLNGKEFKCRVNSAAFPAPIEKTISKTKGRPKAPQV |
| | | YTIPPPKEQMAKDKVSLTCMITDFFPEDITVEWQWNGQPAENYKNTQPIMD |
| | | TDGSYFVYSKLNVQKSNWEAGNTFTCSVLHEGLHNHHTEKSLSHSPG |
| 2 | huIL-18Rβ- | FNISGCSTKKLLWTYSTRSEEEFVLFCDLPEPQKSHFCHRNRLSPKQVPEHLP |
| | HisAvi | FMGSNDLSDVQWYQQPSNGDPLEDIRKSYPHIIQDKCTLHELTPGVNNSGSY |
| | | ICRPKMIKSPYDVACCVKMILEVKPQTNASCEYSASHKQDLLLGSTGSISCPS |
| | | LSCQSDAQSPAVTWYKNGKLLSVERSNRIVVDEVYDYHQGTYVCDYTQSD |
| | | TVSSWTVRAVVQVRTIVGDTKLKPDILDPVEDTLEVELGKPLTISCKARFGF |
| | | ERVFNPVIKWYIKDSDLEWEVSVPEAKSIKSTLKDEIIERNIILEKVTQRDLRR |
| | | KFVCFVQNSIGNTTQSVQLKEKRGSGGLNDIFEAQKIEWHETVRFQSHHHH |
| | | HH |
| 3 | cyIL-18Rβ- | FNISGCSTKKLLWAYSTRSEEEFILFCDLPEPQKSHFCHRSQLSPTQVPDHLPF |
| | HisAvi | TDSKDLPDVQWYRQPSNGDPLEDIRKSYPHIIQDKCTLRFLTPGVNNTGSYIC |
| | | RPKMIKSPYDVACCVKMILEVKPQSNASCENSASHKQDLLLGSTGSISCPSLS |
| | | CQSDAQSPEVTWYKNGKLLSMTRSNRIVVDEVYIYHQGTYVCDYTQSDTA |
| | | SSWTVRAVVQVRTIVGDTKLKPDILDPVESTLEVELGKPLTISCKARFGFERV |
| | | FNPVMKWYIKDADLEWEVSVPEAKSIKSTLKDEIIERNIILEKVTQRDLRRTF |
| | | VCFVQNSIGNTTQSVQLKEKRGSGGLNDIFEAQKIEWHETVRFQSHHHHHH |
| 4 | muIL-18Rβ- | FNHSACATKKLLWTYSARGAENFVLFCDLQELQEQKFSHASQLSPTQSPAH |
| | HisAvi | KPCSGSQKDLSDVQWYMQPRSGSPLEEISRNSPHMQSEGMLHILAPQTNSIW |
| | | SYICRPRIRSPQDMACCIKTVLEVKPQRNVSCGNTAQDEQVLLLGSTGSIHCP |
| | | SLSCQSDVQSPEMTWYKDGRLLPEHKKNPIEMADIYVFNQGLYVCDYTQSD |
| | | NVSSWTVRAVVKVRTIGKDINVKPEILDPITDTLDVELGKPLTLPCRVQFGF |
| | | QRLSKPVIKWYVKESTQEWEMSVFEEKRIQSTFKNEVIERTIFLREVTQRDLS |
| | | RKFVCFAQNSIGNTTRTIRLRKKEEDGSGGLNDIFEAQKIEWHETVRFQSHH |
| | | HHHH |
| 5 | heavy chain (aa) | QVQLQQSGPGLVKPSETLSLTCTVSGGSINNYYWSWIRQPPGKGLEWIGYIF |
| | (P20) | YSGTTNYNPSLKSRVTISVDTSKNQFSLKLSSVTAADTAVYYCARDVGIAAR |
| | | NSYYYVMDVWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTYICN |
| | | VNHKPSNTKVDKRVEPKSCDKTHTCPPCPAPEAEGAPSVFLFPPKPKDTLMI |
| | | SRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFL |
| | | YSKLTVDKSRWQQGNVFSCSVMHEALHNHYTQKSLSLSPG |
| 6 | light chain (aa) | AIQLTQSPSSLSASVGDRVTITCRASQGISSALAWYQQKPGKAPKFLIYDASS |
| | (P20) | LESGVPSRFSGSGSGTDFTLTISSLQPEDFATYYCQQFNSYPITFGQGTRLEIK |
| | | RTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSEN |
| | | RGEC |
| 7 | VH region (aa) | QVQLQQSGPGLVKPSETLSLTCTVSGGSINNYYWSWIRQPPGKGLEWIGYIF |
| | (P20) | YSGTTNYNPSLKSRVTISVDTSKNQFSLKLSSVTAADTAVYYCARDVGIAAR |
| | | NSYYYVMDVWGQGTTVTVSS |
| 8 | CDR-H1 (Kabat) | NYYWS |
| | (P20, P20-V1, | |
| | P20-V2, P20-V3) | |
| 9 | CDR-H2 (Kabat) | YIFYSGTTNYNPSLKS |
| | (P20, P20-V1, | |
| | P20-V2, P20-V3) | |
| 10 | CDR-H3 (Kabat, | DVGIAARNSYYYVMDV |
| | Chothia, AbM) | |
| | (P20, P20-V1, | |
| | P20-V2, P20-V3) | |
| 11 | CDR-H1 | GGSINNY |
| | (Chothia) (P20, | |
| | P20-V1, P20-V2, | |
| | P20-V3) | |
| 12 | CDR-H2 | FYSGT |
| | (Chothia) (P20, | |
| | P20-V1, P20-V2, | |
| | P20-V3) | |
| 13 | CDR-H1 (AbM) | GGSINNYYWS |
| | (P20, P20-V1, | |
| | P20-V2, P20-V3) | |
| 14 | CDR-H2 (AbM) | YIFYSGTTN |
| | (P20, P20-V1, | |
| | P20-V2, P20-V3) | |
| 15 | CDR-H1 | NNYYWS |
| | (Contact) (P20, | |
| | P20-V1, P20-V2, | |
| | P20-V3) | |
| 16 | CDR-H2 | WIGYIFYSGTTN |
| | (Contact) (P20, | |
| | P20-V1, P20-V2, | |
| | P20-V3) | |
| 17 | CDR-H3 | ARDVGIAARNSYYYVMD |
| | (Contact) (P20, | |
| | P20-V1, P20-V2, | |
| | P20-V3) | |
| 18 | CDR-H1 | GGSINNYY |
| | (IMGT) (P20, | |
| | P20-V1, P20-V2, | |
| | P20-V3) | |
| 19 | CDR-H2 | IFYSGTT |
| | (IMGT) (P20, | |
| | P20-V1, P20-V2, | |
| | P20-V3) | |
| 20 | CDR-H3 | ARDVGIAARNSYYYVMDV |
| | (IMGT) (P20, | |
| | P20-V1, P20-V2, | |
| | P20-V3) | |
| 21 | VL region (aa) | AIQLTQSPSSLSASVGDRVTITCRASQGISSALAWYQQKPGKAPKFLIYDASS |
| | (P20) | LESGVPSRFSGSGSGTDFTLTISSLQPEDFATYYCQQFNSYPITFGQGTRLEIK |
| 22 | CDR-L1 (Kabat, | RASQGISSALA |
| | Chothia, AbM) | |
| | (P20, P20-V1, | |
| | P20-V2, P20-V3, | |
| | P21, P21-V1) | |
| 23 | CDR-L2 (Kabat, | DASSLES |
| | Chothia, AbM) | |
| | (P20) | |
| 24 | CDR-L3 (Kabat, | QQFNSYPIT |
| | Chothia, AbM, | |
| | IMGT) (P20, | |
| | P20-V1, P20-V2, | |
| | P20-V3) | |
| 25 | CDR-L1 | SSALAWY |
| | (Contact) (P20, | |
| | P20-V1, P20-V2, | |
| | P20-V3, P21, | |
| | P21-V1) | |
| 26 | CDR-L2 | FLIYDASSLE |
| | (Contact) (P20) | |
| 27 | CDR-L3 | QQFNSYPI |
| | (Contact) (P20, | |
| | P20-V1, P20-V2, | |
| | P20-V3) | |
| 28 | CDR-L1 (IMGT) | QGISSA |
| | (P20, P20-V1, | |
| | P20-V2, P20-V3, | |
| | P21, P21-V1) | |
| 29 | CDR-L2 (IMGT) | DA |
| | (P20) | |
| 30 | heavy chain | ASTKGPSVFPLAPSSKSTSGGTAALGCLVKDYFPEPVTVSWNSGALTSGVHT |
| | constant domain | FPAVLQSSGLYSLSSVVTVPSSSLGTQTYICNVNHKPSNTKVDKRVEPKSCD |
| | (aa) | KTHTCPPCPAPEAEGAPSVFLFPPKPKDTLMISRTPEVTCVVVDVSHEDPEVK |
| | | FNWYVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSV |
| | | MHEALHNHYTQKSLSLSPG |
| 31 | light chain | RTVAAPSVFIFPPSDEQLKSGTASVVCLLNNFYPREAKVQWKVDNALQSGN |
| | constant domain | SQESVTEQDSKDSTYSLSSTLTLSKADYEKHKVYACEVTHQGLSSPVTKSEN |
| | (aa) | RGEC |
| 32 | heavy chain (aa) | QVQLQESGPGLVKPSETLSLTCTVSGGSINNYYWSWIRQPPGKGLEWIGYI |
| | (P20-V1, P20- | FYSGTTNYNPSLKSRVTISVDTSKNQFSLKLSSVTAADTAVYYCARDVGIA |
| | V2, P20-V3) | ARNSYYYVMDVWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGTQT |
| | | YICNVNHKPSNTKVDKRVEPKSCDKTHTCPPCPAPEAEGAPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG |
| 33 | light chain (aa) | AIQLTQSPSSLSASVGDRVTITCRASQGISSALAWYQQKPGKAPKFLIYEASS |
| | (P20-V1) | LESGVPSRFSGSGSGTDFTLTISSLQPEDFATYYCQQFNSYPITFGQGTRLEIK |
| | | RTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFN |
| | | RGEC |
| 34 | VH region (aa) | QVQLQESGPGLVKPSETLSLTCTVSGGSINNYYWSWIRQPPGKGLEWIGYI |
| | (P20-V1, P20- | FYSGTTNYNPSLKSRVTISVDTSKNQFSLKLSSVTAADTAVYYCARDVGIA |
| | V2, P20-V3) | ARNSYYYVMDVWGQGTTVTVSS |
| 35 | VL region (aa) | AIQLTQSPSSLSASVGDRVTITCRASQGISSALAWYQQKPGKAPKFLIYEASS |
| | (P20-V1) | LESGVPSRFSGSGSGTDFTLTISSLQPEDFATYYCQQFNSYPITFGQGTRLEIK |
| 36 | CDR-L2 (Kabat, | EASSLES |
| | Chothia, AbM) | |
| | (P20-V1) | |
| 37 | CDR-L2 (Contact) | FLIYEASSLE |
| | (P20-V1) | |
| 38 | CDR-L2 (IMGT) | EA |
| | (P20-V1) | |
| 39 | light chain (aa) | AIQLTQSPSSLSASVGDRVTITCRASQGISSALAWYQQKPGKAPKFLIYEGSS |
| | (P20-V2) | LESGVPSRFSGSGSGTDFTLTISSLQPEDFATYYCQQFNSYPITFGQGTRLEIK |
| | | RTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSEN |
| | | RGEC |
| 40 | VL region (P20- | AIQLTQSPSSLSASVGDRVTITCRASQGISSALAWYQQKPGKAPKFLIYEGSS |
| | V2) | LESGVPSRFSGSGSGTDFTLTISSLQPEDFATYYCQQFNSYPITFGQGTRLEIK |
| 41 | CDR-L2 (Kabat, | EGSSLES |
| | Chothia) (P20- | |
| | V2) | |
| 42 | CDR-L2 | FLIYEGSSLE |
| | (Contact) (P20- | |
| | V2) | |
| 43 | CDR-L2 (IMGT) | EG |
| | (P20-V2) | |
| 44 | light chain (aa) | AIQLTQSPSSLSASVGDRVTITCRASQGISSALAWYQQKPGKAPKFLIYGASS |
| | (P20-V3) | LESGVPSRFSGSGSGTDFTLTISSLQPEDFATYYCQQFNSYPITFGQGTRLEIK |
| | | RTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFN |
| | | RGEC |
| 45 | VL region (aa) | AIQLTQSPSSLSASVGDRVTITCRASQGISSALAWYQQKPGKAPKFLIYGASS |
| | (P20-V3) | LESGVPSRFSGSGSGTDFTLTISSLQPEDFATYYCQQFNSYPITFGQGTRLEIK |
| 46 | CDR-L2 (Kabat, | GASSLES |
| | Chothia) (P20-V3) | |
| 47 | CDR-L2 | FLIYGASSLE |
| | (Contact) (P20- | |
| | V3) | |
| 48 | CDR-L2 (IMGT) | GA |
| | (P20-V3) | |
| 49 | heavy chain (aa) | QVQLVQSGAEVKKPGASVRVSCKASGYTFTSYYMHWVRQAPGQGLEWMG |
| | (P8) | IINPRGGYTTYAQKFQGRVTMTRDTSTSTVYMELSSLRSEDTAVYYCAKEG |
| | | RVSPTDYYGMDVWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGTQT |
| | | YICNVNHKPSNTKVDKRVEPKSCDKTHTCPPCPAPEAEGAPSVFLFPPKPKD |
| | | TLMISRTPEVTCVVVDVSHEDPEVKFNWYVDGVEVHNAKTKPREEQYNST |
| | | YRVVSVLTVLHQDWL |
| | | NGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRW |
| | | QQGNVFSCSVMHEALHNHYTQKSLSLSPG |
| 50 | light chain (aa) | DIRMTQSPSSLSASVGDRVTITCRASQSISSYLNWYQQKPGKAPKLLIYDAS |
| | (P8) | SLQSGVPSRFSGSGSGTDFTLTMSSLQPEDFATYYCQQSYSTPLTFGGGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 51 | VH region (P8) | QVQLVQSGAEVKKPGASVRVSCKASGYTFTSYYMHWVRQAPGQGLEWMG |
| | | IINPRGGYTTYAQKFQGRVTMTRDTSTSTVYMELSSLRSEDTAVYYCAKEG |
| | | RVSPTDYYGMDVWGQGTTVTVSS |
| 52 | CDR-H1 (Kabat) | SYYMH |
| | (P8, P8-V1) | |
| 53 | CDR-H2 (Kabat) | IINPRGGYTTYAQKFQG |
| | (P8, P8-V1) | |
| 54 | CDR-H3 (Kabat, | EGRVSPTDYYGMDV |
| | Chothia, AbM) | |
| | (P8, P8-V1) | |
| 55 | CDR-H1 | GYTFTSY |
| | (Chothia) (P8, | |
| | P8-V1) | |
| 56 | CDR-H2 | NPRGGY |
| | (Chothia) (P8, | |
| | P8-V1) | |
| 57 | CDR-H1 (AbM) | GYTFTSYYMH |
| | (P8, P8-V1) | |
| 58 | CDR-H2 (AbM) | IINPRGGYTT |
| | (P8, P8-V1) | |
| 59 | CDR-H1 | TSYYMH |
| | (Contact) (P8, | |
| | P8-V1) | |
| 60 | CDR-H2 | WMGIINPRGGYTT |
| | (Contact) (P8, | |
| | P8-V1) | |
| 61 | CDR-H3 | AKEGRVSPTDYYGMD |
| | (Contact) (P8, | |
| | P8-V1) | |
| 62 | CDR-H1 | GYTFTSYY |
| | (IMGT) (P8, P8- | |
| | V1) | |
| 63 | CDR-H2 | INPRGGYT |
| | (IMGT) (P8, P8- | |
| | V1) | |
| 64 | CDR-H3 | AKEGRVSPTDYYGMDV |
| | (IMGT) (P8, P8- | |
| | V1) | |
| 65 | VL region (P8) | DIRMTQSPSSLSASVGDRVTITCRASQSISSYLNWYQQKPGKAPKLLIYDAS |
| | | SLQSGVPSRFSGSGSGTDFTLTMSSLQPEDFATYYCQQSYSTPLTFGGGTKV |
| | | EIK |
| 66 | CDR-L1 (Kabat, | RASQSISSYLN |
| | Chothia, AbM) | |
| | (P8, P8-V1) | |
| 67 | CDR-L2 (Kabat, | DASSLQS |
| | Chothia,AbM) | |
| | (P8, P8-V1) | |
| 68 | CDR-L3 (Kabat, | QQSYSTPLT |
| | Chothia, AbM, | |
| | IMGT) (P8, P8-V1) | |
| 69 | CDR-L1 | SSYLNWY |
| | (Contact) (P8, | |
| | P8-V1) | |
| 70 | CDR-L2 | LLIYDASSLQ |
| | (Contact) (P8, | |
| | P8-V1) | |
| 71 | CDR-L3 | QQSYSTPL |
| | (Contact) (P8, | |
| | P8-V1) | |
| 72 | CDR-L1 (IMGT) | QSISSY |
| | (P8, P8-V1) | |
| 73 | heavy chain (aa) | QVQLVQSGAEVKKPGASVKVSCKASGYTFTSYYMHWVRQAPGQGLEWM |
| | (P8-V1) | GIINPRGGYTTYAQKFQGRVTMTRDTSTSTVYMELSSLRSEDTAVYYCAKE |
| | | GRVSPTDYYGMDVWGQGTTVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAEGAPSVFLFPPKP |
| | | KDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPG |
| 74 | light chain (aa) | DIQMTQSPSSLSASVGDRVTITCRASQSISSYLNWYQQKPGKAPKLLIYDAS |
| | (P8-V1) | SLQSGVPSRFSGSGSGTDFTLTISSLQPEDFATYYCQQSYSTPLTFGGGTKV |
| | | EIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKS |
| | | FNRGEC |
| 75 | VH region (aa) | QVQLVQSGAEVKKPGASVKVSCKASGYTFTSYYMHWVRQAPGQGLEWM |
| | (P8-V1) | GIINPRGGYTTYAQKFQGRVTMTRDTSTSTVYMELSSLRSEDTAVYYCAKE |
| | | GRVSPTDYYGMDVWGQGTTVTVSS |
| 76 | VL region (aa) | DIQMTQSPSSLSASVGDRVTITCRASQSISSYLNWYQQKPGKAPKLLIYDAS |
| | (P8-V1) | SLQSGVPSRFSGSGSGTDFTLTISSLQPEDFATYYCQQSYSTPLTFGGGTKV |
| | | EIK |
| 77 | heavy chain | QVQLQQSGPGLVKPSETLSLTCTVSGGSISNYYWTWIRQPPGKGLEWIGYI |
| | (P21) | SDSGSTNYNPSLKSRVTISVDTSKNQFSLKLSSVTAADTAVYYCARETIVVR |
| | | GFYFYVMDVWGQGTTVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAEGAPSVFLFPPKPKDT |
| | | LMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPG |
| 78 | light chain (P21) | AIQLTQSPSSLSASVGDRVTITCRASQGISSALAWYQQRPGKAPKLLIYDAS |
| | | NWESGVPSRFSGSGSGTDFTLTISSLQPEDFATYYCQQFNSYPWTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 79 | VH region (P21) | QVQLQQSGPGLVKPSETLSLTCTVSGGSISNYYWTWIRQPPGKGLEWIGYI |
| | | SDSGSTNYNPSLKSRVTISVDTSKNQFSLKLSSVTAADTAVYYCARETIVVR |
| | | GFYFYVMDVWGQGTTVTVSS |
| 80 | CDR-H1 (Kabat) | NYYWT |
| | (P21, P21-V1) | |
| 81 | CDR-H2 (Kabat) | YISDSGSTNYNPSLKS |
| | (P21, P21-V1) | |
| 82 | CDR-H3 (Kabat, | ETIVVRGFYFYVMDV |
| | Chothia, AbM) | |
| | (P21, P21-V1) | |
| 83 | CDR-H1 | GGSISNY |
| | (Chothia) (P21, | |
| | P21-V1) | |
| 84 | CDR-H2 | SDSGS |
| | (Chothia) (P21, | |
| | P21-V1) | |
| 85 | CDR-H1 (AbM) | GGSISNYYWT |
| | (P21, P21-V1) | |
| 86 | CDR-H2 (AbM) | YISDSGSTN |
| | (P21, P21-V1) | |
| 87 | CDR-H1 | SNYYWT |
| | (Contact) (P21, | |
| | P21-V1) | |
| 88 | CDR-H2 | WIGYISDSGSTN |
| | (Contact) (P21, | |
| | P21-V1) | |
| 89 | CDR-H3 | ARETIVVRGFYFYVMD |
| | (Contact) (P21, | |
| | P21-V1) | |
| 90 | CDR-H1 | GGSISNYY |
| | (IMGT) (P21, | |
| | P21-V1) | |
| 91 | CDR-H2 | ISDSGST |
| | (IMGT) (P21, | |
| | P21-V1) | |
| 92 | CDR-H3 | ARETIVVRGFYFYVMDV |
| | (IMGT) (P21, | |
| | P21-V1) | |
| 93 | VL region (P21) | AIQLTQSPSSLSASVGDRVTITCRASQGISSALAWYQQRPGKAPKLLIYDAS |
| | | NWESGVPSRFSGSGSGTDFTLTISSLQPEDFATYYCQQFNSYPWTFGQGTK |
| | | VEIK |
| 94 | CDR-L2 (Kabat, | DASNWES |
| | Chothia, AbM) | |
| | (P21) | |
| 95 | CDR-L3 (Kabat, | QQFNSYPWT |
| | Chothia, AbM, | |
| | IMGT) (P21, | |
| | P21-V1) | |
| 96 | CDR-L2 | LLIYDASNWE |
| | (Contact) (P21) | |
| 97 | CDR-L3 | QQFNSYPW |
| | (Contact) (P21, | |
| | P21-V1) | |
| 98 | heavy chain (aa) | QVQLQESGPGLVKPSETLSLTCTVSGGSISNYYWTWIRQPPGKGLEWIGYIS |
| | (P21-V1) | DSGSTNYNPSLKSRVTISVDTSKNQFSLKLSSVTAADTAVYYCARETIVVR |
| | | GFYFYVMDVWGQGTTVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAEGAPSVFLFPPKPKDT |
| | | LMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPG |
| 99 | light chain (aa) | AIQLTQSPSSLSASVGDRVTITCRASQGISSALAWYQQKPGKAPKLLIYEAS |
| | (P21-V1) | NWESGVPSRFSGSGSGTDFTLTISSLQPEDFATYYCQQFNSYPWTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| 100 | VH region (aa) | QVQLQESGPGLVKPSETLSLTCTVSGGSISNYYWTWIRQPPGKGLEWIGYIS |
| | (P21-V1) | DSGSTNYNPSLKSRVTISVDTSKNQFSLKLSSVTAADTAVYYCARETIVVR |
| | | GFYFYVMDVWGQGTTVTVSS |
| 101 | VL region (aa) | AIQLTQSPSSLSASVGDRVTITCRASQGISSALAWYQQKPGKAPKLLIYEAS |
| | (P21-V1) | NWESGVPSRFSGSGSGTDFTLTISSLQPEDFATYYCQQFNSYPWTFGQGTK |
| | | VEIK |
| 102 | CDR-L2 (Kabat, | EASNWES |
| | Chothia, AbM) | |
| | (P21-V1) | |
| 103 | CDR-L2 | LLIYEASNWE |
| | (Contact) (P21- | |
| | V1) | |
| 104 | hIgG1 | ASTKGPSVFPLAPSSKSTSGGTAALGCLVKDYFPEPVTVSWNSGALTSGVHT |
| | HC constant | FPAVLQSSGLYSLSSVVTVPSSSLGTQTYICNVNHKPSNTKVDKRVEPKSCD |
| | region | KTHTCPPCPAPELLGGPSVFLFPPKPKDTLMISRTPEVTCVVVDVSHEDPEVK |
| | | FNWYVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSV |
| | | MHEALHNHYTQKSLSLSPGK |
| 105 | hIgG1 | GCGTCGACCAAGGGCCCATCCGTCTTCCCCCTGGCACCCTCCTCCAAGAG |
| | HC constant | CACCTCTGGGGGCACAGCGGCCCTGGGCTGCCTGGTCAAGGACTACTTC |
| | region DNA | CCCGAACCGGTGACGGTGTCGTGGAACTCAGGCGCCCTGACCAGCGGCG |
| | | TGCACACCTTCCCGGCTGTCCTACAGTCCTCAGGACTCTACTCCCTCAGC |
| | | AGCGTGGTGACCGTGCCCTCCAGCAGCTTGGGCACCCAGACCTACATCT |
| | | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGA |
| | | CGTGAGCCACGAAGACCCTGAGGTCAAGTTCAACTGGTACGTGGACGGC |
| | | GTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGCT |
| | | GAATGGCAAGGAGTACAAGTGCAAGGTCTCCAACAAAGCCCTCCCAGCC |
| | | CCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGT |
| | | CAGCCTGACCTGCCTGGTCAAAGGCTTCTATCCCAGCGACATCGCCGTGG |
| | | AGTGGGAGAGCAATGGGCAGCCGGAGAACAACTACAAGACCACGCCTC |
| | | CCGTGCTGGACTCCGACGGCTCCTTCTTCCTCTATAGCAAGCTCACCGTG |
| | | GACAAGAGCAGGTGGCAGCAGGGGAACGTCTTCTCATGCTCCGTGATGC |
| | | ATGAGGCTCTGCACAACCACTACACGCAGAAGAGCCTCTCCCTGTCCCC |
| | | GGGTAAG |
| 106 | hIgG1 | ASTKGPSVFPLAPSSKSTSGGTAALGCLVKDYFPEPVTVSWNSGALTSGVHT |
| | HC constant | FPAVLQSSGLYSLSSVVTVPSSSLGTQTYICNVNHKPSNTKVDKRVEPKSCD |
| | region without | KTHTCPPCPAPELLGGPSVFLFPPKPKDTLMISRTPEVTCVVVDVSHEDPEVK |
| | C-terminal lysine | FNWYVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSV |
| | | MHEALHNHYTQKSLSLSPG |
| 107 | hIgG1 | GCGTCGACCAAGGGCCCATCCGTCTTCCCCCTGGCACCCTCCTCCAAGAG |
| | HC constant | CACCTCTGGGGGCACAGCGGCCCTGGGCTGCCTGGTCAAGGACTACTTC |
| | region DNA | CCCGAACCGGTGACGGTGTCGTGGAACTCAGGCGCCCTGACCAGCGGCG |
| | without C- | TGCACACCTTCCCGGCTGTCCTACAGTCCTCAGGACTCTACTCCCTCAGC |
| | terminal lysine | AGCGTGGTGACCGTGCCCTCCAGCAGCTTGGGCACCCAGACCTACATCT |
| | | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGA |
| | | CGTGAGCCACGAAGACCCTGAGGTCAAGTTCAACTGGTACGTGGACGGC |
| | | GTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGCT |
| | | GAATGGCAAGGAGTACAAGTGCAAGGTCTCCAACAAAGCCCTCCCAGCC |
| | | CCCATCGAGAAAACCATCTCCAAAGCCAAAGGGCAGCCCCGAGAACCAC |
| | | AGGTGTACACCCTGCCCCCATCCCGGGAGGAGATGACCAAGAACCAGGT |
| | | CAGCCTGACCTGCCTGGTCAAAGGCTTCTATCCCAGCGACATCGCCGTGG |
| | | AGTGGGAGAGCAATGGGCAGCCGGAGAACAACTACAAGACCACGCCTC |
| | | CCGTGCTGGACTCCGACGGCTCCTTCTTCCTCTATAGCAAGCTCACCGTG |
| | | GACAAGAGCAGGTGGCAGCAGGGGAACGTCTTCTCATGCTCCGTGATGC |
| | | ATGAGGCTCTGCACAACCACTACACGCAGAAGAGCCTCTCCCTGTCCCC |
| | | GGGT |
| 108 | hIgG1.3f | ASTKGPSVFPLAPSSKSTSGGTAALGCLVKDYFPEPVTVSWNSGALTSGVHT |
| | HC constant | FPAVLQSSGLYSLSSVVTVPSSSLGTQTYICNVNHKPSNTKVDKRVEPKSCD |
| | region; IgG 1.3 | KTHTCPPCPAPEAEGAPSVFLFPPKPKDTLMISRTPEVTCVVVDVSHEDPEVK |
| | comprises | FNWYVDGVEVHNAKTKPREEQYNSTYRVVSVLTVLHQDWLNGKEYKCKV |
| | mutations | SNKALPAPIEKTISKAKGQPREPQVYTLPPSREEMTKNQVSLTCLVKGFYPSD |
| | L234A, L235E, | IAVEWESNGQPENNYKTTPPVLDSDGSFFLYSKLTVDKSRWQQGNVFSCSV |
| | and G237A | MHEALHNHYTQKSLSLSPGK |
| 109 | hIgG1.3f | GCGTCGACCAAGGGCCCATCGGTCTTCCCCCTGGCACCCTCCTCCAAGAG |
| | HC constant | CACCTCTGGGGGCACAGCGGCCCTGGGCTGCCTGGTCAAGGACTACTTC |
| | region DNA | CCCGAACCGGTGACGGTGTCGTGGAACTCAGGCGCCCTGACCAGCGGCG |
| | sequence | TGCACACCTTCCCGGCTGTCCTACAGTCCTCAGGACTCTACTCCCTCAGC |
| | | AGCGTGGTGACCGTGCCCTCCAGCAGCTTGGGCACCCAGACCTACATCT |
| | | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAAGCCGAAGGGGCCCCGTCAGTCTTCCTCTTCCCCCCAAAACCCAAG |
| | | GACACCCTCATGATCTCCCGGACCCCTGAGGTCACATGCGTGGTGGTGG |
| | | ACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCC |
| | | CCGGGTAAG |
| 110 | hIgG1.3f | ASTKGPSVFPLAPSSKSTSGGTAALGCLVKDYFPEPVTVSWNSGALTSGVHT |
| | HC constant | FPAVLQSSGLYSLSSVVTVPSSSLGTQTYICNVNHKPSNTKVDKRVEPKSCD |
| | region without | KTHTCPPCPAPEAEGAPSVELFPPKPKDTLMISRTPEVTCVVVDVSHEDPEVK |
| | the C-terminal | FNWYVDGVEVHNAKTKPREEQYNSTYRVVSVLTVLHQDWLNGKEYKCKV |
| | lysine; IgG 1.3 | SNKALPAPIEKTISKAKGQPREPQVYTLPPSREEMTKNQVSLTCLVKGFYPSD |
| | mutations | IAVEWESNGQPENNYKTTPPVLDSDGSFFLYSKLTVDKSRWQQGNVFSCSV |
| | L234A, L235E, | MHEALHNHYTQKSLSLSPG |
| | and G237A | |
| 111 | hIgG1.3f | GCGTCGACCAAGGGCCCATCGGTCTTCCCCCTGGCACCCTCCTCCAAGAG |
| | HC constant | CACCTCTGGGGGCACAGCGGCCCTGGGCTGCCTGGTCAAGGACTACTTC |
| | region without | CCCGAACCGGTGACGGTGTCGTGGAACTCAGGCGCCCTGACCAGCGGCG |
| | the C-terminal | TGCACACCTTCCCGGCTGTCCTACAGTCCTCAGGACTCTACTCCCTCAGC |
| | lysine; DNA | AGCGTGGTGACCGTGCCCTCCAGCAGCTTGGGCACCCAGACCTACATCT |
| | sequence | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAAGCCGAAGGGGCCCCGTCAGTCTTCCTCTTCCCCCCAAAACCCAAG |
| | | GACACCCTCATGATCTCCCGGACCCCTGAGGTCACATGCGTGGTGGTGG |
| | | ACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCC |
| | | CCGGGT |
| 112 | hIgG1fa L235E, | ASTKGPSVFPLAPSSKSTSGGTAALGCLVKDYFPEPVTVSWNSGALTSGVHT |
| | P238K (LEPK) | FPAVLQSSGLYSLSSVVTVPSSSLGTQTYICNVNHKPSNTKVDKRVEPKSCD |
| | | KTHTCPPCPAPELEGGKSVFLFPPKPKDTLMISRTPEVTCVVVDVSHEDPEV |
| | | KFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVFSCS |
| | | VMHEALHNHYTQKSLSLSPGK |
| 113 | hIgG1fa LEPK | GCGTCGACCAAGGGCCCATCCGTCTTCCCCCTGGCACCCTCCTCCAAGAG |
| | HC constant | CACCTCTGGGGGCACAGCGGCCCTGGGCTGCCTGGTCAAGGACTACTTC |
| | region DNA | CCCGAACCGGTGACGGTGTCGTGGAACTCAGGCGCCCTGACCAGCGGCG |
| | sequence | TGCACACCTTCCCGGCTGTCCTACAGTCCTCAGGACTCTACTCCCTCAGC |
| | | AGCGTGGTGACCGTGCCCTCCAGCAGCTTGGGCACCCAGACCTACATCT |
| | | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAACTCGAGGGGGGAAAGTCAGTCTTCCTCTTCCCCCCAAAACCCAAG |
| | | GACACCCTCATGATCTCCCGGACCCCTGAGGTCACATGCGTGGTGGTGG |
| | | ACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGATGAGCTGACCAAGAACCAG |
| | | GTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCC |
| | | CCGGGTAAA |
| 114 | hIgG1fa LEPK | ASTKGPSVFPLAPSSKSTSGGTAALGCLVKDYFPEPVTVSWNSGALTSGVHT |
| | HC constant | FPAVLQSSGLYSLSSVVTVPSSSLGTQTYICNVNHKPSNTKVDKRVEPKSCD |
| | region without | KTHTCPPCPAPELEGGKSVFLFPPKPKDTLMISRTPEVTCVVVDVSHEDPEV |
| | the C-terminal | KFNWYVDGVEVHNAKTKPREEQYNSTYRVVSVLTVLHQDWLNGKEYKCK |
| | lysine | VSNKALPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVFSCS |
| | | VMHEALHNHYTQKSLSLSPG |
| 115 | hIgG1fa LEPK | GCGTCGACCAAGGGCCCATCCGTCTTCCCCCTGGCACCCTCCTCCAAGAG |
| | HC constant | CACCTCTGGGGGCACAGCGGCCCTGGGCTGCCTGGTCAAGGACTACTTC |
| | region without | CCCGAACCGGTGACGGTGTCGTGGAACTCAGGCGCCCTGACCAGCGGCG |
| | the C-terminal | TGCACACCTTCCCGGCTGTCCTACAGTCCTCAGGACTCTACTCCCTCAGC |
| | lysine DNA | AGCGTGGTGACCGTGCCCTCCAGCAGCTTGGGCACCCAGACCTACATCT |
| | sequence | GCAACGTGAATCACAAGCCCAGCAACACCAAGGTGGACAAGAGAGTTG |
| | | AGCCCAAATCTTGTGACAAAACTCACACATGCCCACCGTGCCCAGCACC |
| | | TGAACTCGAGGGGGGAAAGTCAGTCTTCCTCTTCCCCCCAAAACCCAAG |
| | | GACACCCTCATGATCTCCCGGACCCCTGAGGTCACATGCGTGGTGGTGG |
| | | ACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGATGAGCTGACCAAGAACCAG |
| | | GTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCC |
| | | CCGGGT |
| 116 | hIgG2.3 | ASTKGPSVFPLAPCSRSTSESTAALGCLVKDYFPEPVTVSWNSGALTSGVHT |
| | HC constant | FPAVLQSSGLYSLSSVVTVPSSNFGTQTYTCNVDHKPSNTKVDKTVERKSCV |
| | region | ECPPCPAPPVAGPSVFLFPPKPKDTLMISRTPEVTCVVVDVSHEDPEVQFNW |
| | | YVDGVEVHNAKTKPREEQFNSTFRVVSVLTVVHQDWLNGKEYKCKVSNK |
| | | GLPAPIEKTISKTKGQPREPQVYTLPPSREEMTKNQVSLTCLVKGFYPSDIAV |
| | | EWESNGQPENNYKTTPPMLDSDGSFFLYSKLTVDKSRWQQGNVFSCSVMH |
| | | EALHNHYTQKSLSLSPGK |
| 117 | hIgG2.3 | GCGTCGACCAAGGGCCCCTCTGTGTTTCCTCTGGCCCCTTGCTCCCGGTC |
| | HC constant | CACCTCTGAGTCTACCGCTGCTCTGGGCTGCCTGGTCAAGGACTACTTCC |
| | region DNA | CCGAGCCTGTGACCGTGTCCTGGAACTCTGGCGCTCTGACCTCCGGCGTG |
| | | CACACCTTTCCAGCCGTGCTGCAGTCCTCCGGCCTGTACTCTCTGTCCTCC |
| | | GTCGTGACCGTGCCCTCCTCCAACTTCGGCACCCAGACCTACACCTGTAA |
| | | CGTGGACCACAAGCCCTCCAACACCAAGGTGGACAAGACCGTGGAACG |
| | | GAAGTCCTGCGTGGAATGCCCTCCTTGCCCTGCACCTCCTGTGGCTGGCA |
| | | AATCCGTGTTCCTGTTCCCCCCAAAGCCCAAGGACACCCTGATGATCTCC |
| | | CGGACCCCCGAAGTGACCTGCGTGGTGGTGGATGTGTCCCACGAGGACC |
| | | CCGAGGTGCAGTTCAATTGGTACGTGGACGGCGTGGAAGTGCACAACGC |
| | | CAAGACCAAGCCCAGAGAGGAACAGTTCAACTCCACCTTCCGGGTGGTG |
| | | TCCGTGCTGACCGTGGTGCACCAGGACTGGCTGAACGGCAAAGAGTACA |
| | | AGTGCAAGGTGTCCAACAAGGGCCTGCCTGCCCCCATCGAAAAGACCAT |
| | | CTCCAAGACAAAGGGCCAGCCCCGCGAGCCTCAGGTGTACACACTGCCT |
| | | CCCAGCCGGGAAGAGATGACCAAGAACCAGGTGTCCCTGACCTGTCTGG |
| | | TCAAGGGCTTCTACCCCTCCGATATCGCCGTGGAATGGGAGTCCAACGG |
| | | CCAGCCCGAGAACAACTACAAGACCACCCCCCCCATGCTGGACTCCGAC |
| | | GGCTCATTCTTCCTGTACTCCAAGCTGACAGTGGACAAGTCCCGGTGGCA |
| | | GCAGGGCAACGTGTTCTCCTGCTCTGTGATGCACGAGGCCCTGCACAAC |
| | | CACTACACCCAGAAGTCCCTGTCCCTGAGCCCCGGCAAA |
| 118 | hIgG2.3 | ASTKGPSVFPLAPCSRSTSESTAALGCLVKDYFPEPVTVSWNSGALTSGVHT |
| | HC constant | FPAVLQSSGLYSLSSVVTVPSSNFGTQTYTCNVDHKPSNTKVDKTVERKSCV |
| | region, without | ECPPCPAPPVAGPSVFLFPPKPKDTLMISRTPEVTCVVVDVSHEDPEVQFNW |
| | C-terminal lysine | YVDGVEVHNAKTKPREEQFNSTFRVVSVLTVVHQDWLNGKEYKCKVSNK |
| | | GLPAPIEKTISKTKGQPREPQVYTLPPSREEMTKNQVSLTCLVKGFYPSDIAV |
| | | EWESNGQPENNYKTTPPMLDSDGSFFLYSKLTVDKSRWQQGNVFSCSVMH |
| | | EALHNHYTQKSLSLSPG |
| 119 | hIgG2.3 | GCGTCGACCAAGGGCCCCTCTGTGTTTCCTCTGGCCCCTTGCTCCCGGTC |
| | HC constant | CACCTCTGAGTCTACCGCTGCTCTGGGCTGCCTGGTCAAGGACTACTTCC |
| | region, without | CCGAGCCTGTGACCGTGTCCTGGAACTCTGGCGCTCTGACCTCCGGCGTG |
| | C-terminal lysine | CACACCTTTCCAGCCGTGCTGCAGTCCTCCGGCCTGTACTCTCTGTCCTCC |
| | DNA | GTCGTGACCGTGCCCTCCTCCAACTTCGGCACCCAGACCTACACCTGTAA |
| | | CGTGGACCACAAGCCCTCCAACACCAAGGTGGACAAGACCGTGGAACG |
| | | GAAGTCCTGCGTGGAATGCCCTCCTTGCCCTGCACCTCCTGTGGCTGGCA |
| | | AATCCGTGTTCCTGTTCCCCCCAAAGCCCAAGGACACCCTGATGATCTCC |
| | | CGGACCCCCGAAGTGACCTGCGTGGTGGTGGATGTGTCCCACGAGGACC |
| | | CCGAGGTGCAGTTCAATTGGTACGTGGACGGCGTGGAAGTGCACAACGC |
| | | CAAGACCAAGCCCAGAGAGGAACAGTTCAACTCCACCTTCCGGGTGGTG |
| | | TCCGTGCTGACCGTGGTGCACCAGGACTGGCTGAACGGCAAAGAGTACA |
| | | AGTGCAAGGTGTCCAACAAGGGCCTGCCTGCCCCCATCGAAAAGACCAT |
| | | CTCCAAGACAAAGGGCCAGCCCCGCGAGCCTCAGGTGTACACACTGCCT |
| | | CCCAGCCGGGAAGAGATGACCAAGAACCAGGTGTCCCTGACCTGTCTGG |
| | | TCAAGGGCTTCTACCCCTCCGATATCGCCGTGGAATGGGAGTCCAACGG |
| | | CCAGCCCGAGAACAACTACAAGACCACCCCCCCCATGCTGGACTCCGAC |
| | | GGCTCATTCTTCCTGTACTCCAAGCTGACAGTGGACAAGTCCCGGTGGCA |
| | | GCAGGGCAACGTGTTCTCCTGCTCTGTGATGCACGAGGCCCTGCACAAC |
| | | CACTACACCCAGAAGTCCCTGTCCCTGAGCCCCGGC |
| 120 | hIgG2.3 P238K | ASTKGPSVFPLAPCSRSTSESTAALGCLVKDYFPEPVTVSWNSGALTSGVHT |
| | HC constant | FPAVLQSSGLYSLSSVVTVPSSNFGTQTYTCNVDHKPSNTKVDKTVERKSCV |
| | region | ECPPCPAPPVAGKSVFLFPPKPKDTLMISRTPEVTCVVVDVSHEDPEVQFNW |
| | | YVDGVEVHNAKTKPREEQFNSTFRVVSVLTVVHQDWLNGKEYKCKVSNK |
| | | GLPAPIEKTISKTKGQPREPQVYTLPPSREEMTKNQVSLTCLVKGFYPSDIAV |
| | | EWESNGQPENNYKTTPPMLDSDGSFFLYSKLTVDKSRWQQGNVFSCSVMH |
| | | EALHNHYTQKSLSLSPGK |
| 121 | hIgG2.3 P238K | GCGTCGACCAAGGGCCCCTCTGTGTTTCCTCTGGCCCCTTGCTCCCGGTC |
| | HC constant | CACCTCTGAGTCTACCGCTGCTCTGGGCTGCCTGGTCAAGGACTACTTCC |
| | region DNA | CCGAGCCTGTGACCGTGTCCTGGAACTCTGGCGCTCTGACCTCCGGCGTG |
| | sequence | CACACCTTTCCAGCCGTGCTGCAGTCCTCCGGCCTGTACTCTCTGTCCTCC |
| | | GTCGTGACCGTGCCCTCCTCCAACTTCGGCACCCAGACCTACACCTGTAA |
| | | CGTGGACCACAAGCCCTCCAACACCAAGGTGGACAAGACCGTGGAACG |
| | | GAAGTCCTGCGTGGAATGCCCTCCTTGCCCTGCACCTCCTGTGGCTGGCA |
| | | AATCCGTGTTCCTGTTCCCCCCAAAGCCCAAGGACACCCTGATGATCTCC |
| | | CGGACCCCCGAAGTGACCTGCGTGGTGGTGGATGTGTCCCACGAGGACC |
| | | CCGAGGTGCAGTTCAATTGGTACGTGGACGGCGTGGAAGTGCACAACGC |
| | | CAAGACCAAGCCCAGAGAGGAACAGTTCAACTCCACCTTCCGGGTGGTG |
| | | TCCGTGCTGACCGTGGTGCACCAGGACTGGCTGAACGGCAAAGAGTACA |
| | | AGTGCAAGGTGTCCAACAAGGGCCTGCCTGCCCCCATCGAAAAGACCAT |
| | | CTCCAAGACAAAGGGCCAGCCCCGCGAGCCTCAGGTGTACACACTGCCT |
| | | CCCAGCCGGGAAGAGATGACCAAGAACCAGGTGTCCCTGACCTGTCTGG |
| | | TCAAGGGCTTCTACCCCTCCGATATCGCCGTGGAATGGGAGTCCAACGG |
| | | CCAGCCCGAGAACAACTACAAGACCACCCCCCCCATGCTGGACTCCGAC |
| | | GGCTCATTCTTCCTGTACTCCAAGCTGACAGTGGACAAGTCCCGGTGGCA |
| | | GCAGGGCAACGTGTTCTCCTGCTCTGTGATGCACGAGGCCCTGCACAAC |
| | | CACTACACCCAGAAGTCCCTGTCCCTGAGCCCCGGCAAA |
| 122 | hIgG2.3 P238K | ASTKGPSVFPLAPCSRSTSESTAALGCLVKDYFPEPVTVSWNSGALTSGVHT |
| | HC constant | FPAVLQSSGLYSLSSVVTVPSSNFGTQTYTCNVDHKPSNTKVDKTVERKSCV |
| | region without | ECPPCPAPPVAGKSVFLFPPKPKDTLMISRTPEVTCVVVDVSHEDPEVQFNW |
| | the C-terminal | YVDGVEVHNAKTKPREEQFNSTFRVVSVLTVVHQDWLNGKEYKCKVSNK |
| | lysine | GLPAPIEKTISKTKGQPREPQVYTLPPSREEMTKNQVSLTCLVKGFYPSDIAV |
| | | EWESNGQPENNYKTTPPMLDSDGSFFLYSKLTVDKSRWQQGNVFSCSVMH |
| | | EALHNHYTQKSLSLSPG |
| 123 | hIgG2.3 P238K | GCGTCGACCAAGGGCCCCTCTGTGTTTCCTCTGGCCCCTTGCTCCCGGTC |
| | HC constant | CACCTCTGAGTCTACCGCTGCTCTGGGCTGCCTGGTCAAGGACTACTTCC |
| | region without | CCGAGCCTGTGACCGTGTCCTGGAACTCTGGCGCTCTGACCTCCGGCGTG |
| | the C-terminal | CACACCTTTCCAGCCGTGCTGCAGTCCTCCGGCCTGTACTCTCTGTCCTCC |
| | lysine DNA | GTCGTGACCGTGCCCTCCTCCAACTTCGGCACCCAGACCTACACCTGTAA |
| | sequence | CGTGGACCACAAGCCCTCCAACACCAAGGTGGACAAGACCGTGGAACG |
| | | GAAGTCCTGCGTGGAATGCCCTCCTTGCCCTGCACCTCCTGTGGCTGGCA |
| | | AATCCGTGTTCCTGTTCCCCCCAAAGCCCAAGGACACCCTGATGATCTCC |
| | | CGGACCCCCGAAGTGACCTGCGTGGTGGTGGATGTGTCCCACGAGGACC |
| | | CCGAGGTGCAGTTCAATTGGTACGTGGACGGCGTGGAAGTGCACAACGC |
| | | CAAGACCAAGCCCAGAGAGGAACAGTTCAACTCCACCTTCCGGGTGGTG |
| | | TCCGTGCTGACCGTGGTGCACCAGGACTGGCTGAACGGCAAAGAGTACA |
| | | AGTGCAAGGTGTCCAACAAGGGCCTGCCTGCCCCCATCGAAAAGACCAT |
| | | CTCCAAGACAAAGGGCCAGCCCCGCGAGCCTCAGGTGTACACACTGCCT |
| | | CCCAGCCGGGAAGAGATGACCAAGAACCAGGTGTCCCTGACCTGTCTGG |
| | | TCAAGGGCTTCTACCCCTCCGATATCGCCGTGGAATGGGAGTCCAACGG |
| | | CCAGCCCGAGAACAACTACAAGACCACCCCCCCCATGCTGGACTCCGAC |
| | | GGCTCATTCTTCCTGTACTCCAAGCTGACAGTGGACAAGTCCCGGTGGCA |
| | | GCAGGGCAACGTGTTCTCCTGCTCTGTGATGCACGAGGCCCTGCACAAC |
| | | CACTACACCCAGAAGTCCCTGTCCCTGAGCCCCGGC |
| 124 | Exemplary LC | RTVAAPSVFIFPPSDEQLKSGTASVVCLLNNFYPREAKVQWKVDNALQSGN |
| | constant region | SQESVTEQDSKDSTYSLSSTLTLSKADYEKHKVYACEVTHQGLSSPVTKSFN |
| | | RGEC |
| 125 | Exemplary LC | CGTACGGTGGCTGCACCATCTGTCTTCATCTTCCCGCCATCTGATGAGCA |
| | constant region | GTTGAAATCTGGAACTGCCTCTGTTGTGTGCCTGCTGAATAACTTCTATC |
| | DNA sequence | CCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCT |
| | | ACAGCCTCAGCAGCACCCTGACGCTGAGCAAAGCAGACTACGAGAAAC |
| | | ACAAAGTCTACGCCTGCGAAGTCACCCATCAGGGCCTGAGCTCGCCCGT |
| | | CACAAAGAGCTTCAACAGGGGAGAGTGT |
| 126 | huIL-18Rβ | MLCLGWIFLWLVAGERIKGFNISGCSTKKLLWTYSTRSEEEFVLFCDLPEPQ |
| | | KSHFCHRNRLSPKQVPEHLPFMGSNDLSDVQWYQQPSNGDPLEDIRKSYPHI |
| | | IQDKCTLHELTPGVNNSGSYICRPKMIKSPYDVACCVKMILEVKPQTNASCE |
| | | YSASHKQDLLLGSTGSISCPSLSCQSDAQSPAVTWYKNGKLLSVERSNRIVV |
| | | DEVYDYHQGTYVCDYTQSDTVSSWTVRAVVQVRTIVGDTKLKPDILDPVE |
| | | DTLEVELGKPLTISCKARFGFERVFNPVIKWYIKDSDLEWEVSVPEAKSIKST |
| | | LKDEIIERNIILEKVTQRDLRRKFVCFVQNSIGNTTQSVQLKEKRGVVLLYILL |
| | | GTIGTLVAVLAASALLYRHWIEIVLLYRTYQSKDQTLGDKKDFDAFVSYAK |
| | | WSSFPSEATSSLSEEHLALSLFPDVLENKYGYSLCLLERDVAPGGVYAEDIVS |
| | | IIKRSRRGIFILSPNYVNGPSIFELQAAVNLALDDQTLKLILIKFCYFQEPESLP |
| | | HLVKKALRVLPTVTWRGLKSVPPNSRFWAKMRYHMPVKNSQGFTWNQLR |
| | | ITSRIFQWKGLSRTETTGRSSQPKEW |
| 127 | VH region (nt) | CAAGTGCAACTACAAGAAAGTGGTCCAGGTCTGGTGAAACCATCCGAGA |
| | (P20-V1) | CACTCAGTCTGACCTGCACCGTGTCCGGCGGCTCCATCAACAACTATTAC |
| | | TGGTCCTGGATCAGACAGCCTCCTGGCAAGGGACTGGAATGGATCGGCT |
| | | ACATTTTCTACTCTGGCACCACCAACTACAACCCCTCCCTGAAGAGCAGA |
| | | GTGACAATCTCTGTGGATACCTCCAAGAACCAGTTCTCCCTGAAACTGTC |
| | | CAGCGTGACAGCCGCTGATACAGCCGTCTACTACTGCGCCAGAGATGTG |
| | | GGCATCGCCGCCAGGAACTCCTACTACTACGTGATGGACGTGTGGGGCC |
| | | AGGGCACCACCGTCACCGTGAGCTCT |
| 128 | VL region (nt) | GCAATCCAACTAACTCAAAGTCCAAGTAGTCTGTCCGCTTCCGTGGGCG |
| | (P20-V1) | ACAGAGTGACTATCACCTGCAGAGCCTCTCAGGGCATCTCTTCTGCTCTG |
| | | GCCTGGTACCAGCAGAAGCCTGGCAAGGCTCCTAAGTTCCTGATCTACG |
| | | AAGCCAGCTCTCTGGAATCTGGCGTGCCTTCTCGGTTCTCCGGCTCCGGA |
| | | TCTGGCACCGACTTCACCCTGACCATCTCCTCTCTGCAACCTGAGGACTT |
| | | TGCCACCTACTACTGCCAGCAGTTCAACTCCTACCCCATCACCTTCGGCC |
| | | AGGGCACCAGACTGGAAATCAAG |
| 129 | Heavy chain (nt) | CAAGTGCAACTACAAGAAAGTGGTCCAGGTCTGGTGAAACCATCCGAGA |
| | (P20-V1) | CACTCAGTCTGACCTGCACCGTGTCCGGCGGCTCCATCAACAACTATTAC |
| | | TGGTCCTGGATCAGACAGCCTCCTGGCAAGGGACTGGAATGGATCGGCT |
| | | ACATTTTCTACTCTGGCACCACCAACTACAACCCCTCCCTGAAGAGCAGA |
| | | GTGACAATCTCTGTGGATACCTCCAAGAACCAGTTCTCCCTGAAACTGTC |
| | | CAGCGTGACAGCCGCTGATACAGCCGTCTACTACTGCGCCAGAGATGTG |
| | | GGCATCGCCGCCAGGAACTCCTACTACTACGTGATGGACGTGTGGGGCC |
| | | AGGGCACCACCGTCACCGTGAGCTCTGCTTCTACCAAGGGCCCTAGCGT |
| | | ATTTCCTCTGGCCCCTAGCTCCAAGAGCACCTCCGGCGGAACCGCTGCTC |
| | | TGGGCTGTCTGGTGAAGGACTACTTCCCTGAGCCCGTGACCGTGTCTTGG |
| | | AACTCCGGTGCCCTCACCTCTGGCGTGCATACCTTCCCTGCTGTGCTGCA |
| | | GTCCAGCGGACTGTACTCCCTGTCTTCCGTGGTGACCGTGCCTTCCTCCT |
| | | CTCTGGGCACTCAAACATACATCTGCAATGTGAACCACAAGCCCAGCAA |
| | | CACCAAAGTGGACAAGCGGGTGGAACCCAAGTCCTGCGACAAGACCCA |
| | | CACCTGTCCTCCTTGCCCAGCTCCCGAAGCCGAGGGCGCTCCTTCTGTCT |
| | | TTCTGTTCCCTCCTAAACCTAAGGACACCCTGATGATCTCTAGAACCCCT |
| | | GAAGTGACCTGCGTCGTCGTGGACGTGTCCCACGAGGACCCTGAGGTGA |
| | | AGTTCAACTGGTACGTTGACGGCGTGGAAGTGCATAACGCCAAGACAAA |
| | | GCCTAGAGAAGAGCAGTACAACTCTACCTACCGGGTGGTGTCTGTGCTG |
| | | ACCGTGCTGCACCAGGACTGGCTGAACGGCAAAGAGTACAAGTGCAAG |
| | | GTGTCCAACAAGGCTCTGCCCGCACCCATCGAGAAGACCATCTCTAAGG |
| | | CCAAAGGCCAGCCAAGAGAGCCTCAGGTGTACACCCTGCCTCCATCCAG |
| | | AGAAGAGATGACCAAGAACCAAGTGTCTCTGACCTGTCTGGTGAAGGGC |
| | | TTCTATCCTTCTGACATCGCCGTGGAGTGGGAATCTAATGGCCAGCCTGA |
| | | GAACAACTATAAGACCACACCTCCTGTGCTGGACTCCGACGGCTCTTTCT |
| | | TCCTGTACAGCAAGCTCACCGTGGATAAGTCCCGGTGGCAGCAGGGCAA |
| | | CGTGTTCTCTTGCTCCGTGATGCACGAGGCCCTGCACAATCACTACACCC |
| | | AGAAGTCCCTGTCTCTGTCTCCTGGA |
| 130 | Light chain (nt) | GCAATCCAACTAACTCAAAGTCCAAGTAGTCTGTCCGCTTCCGTGGGCG |
| | (P20-V1) | ACAGAGTGACTATCACCTGCAGAGCCTCTCAGGGCATCTCTTCTGCTCTG |
| | | GCCTGGTACCAGCAGAAGCCTGGCAAGGCTCCTAAGTTCCTGATCTACG |
| | | AAGCCAGCTCTCTGGAATCTGGCGTGCCTTCTCGGTTCTCCGGCTCCGGA |
| | | TCTGGCACCGACTTCACCCTGACCATCTCCTCTCTGCAACCTGAGGACTT |
| | | TGCCACCTACTACTGCCAGCAGTTCAACTCCTACCCCATCACCTTCGGCC |
| | | AGGGCACCAGACTGGAAATCAAGCGGACCGTGGCCGCTCCATCTGTGTT |
| | | CATCTTTCCTCCTTCCGATGAACAGCTGAAGTCTGGAACAGCTTCCGTGG |
| | | TGTGCCTGCTGAACAACTTCTATCCTAGAGAGGCTAAAGTGCAGTGGAA |
| | | GGTGGACAACGCCCTGCAGTCCGGCAATAGCCAAGAGTCCGTCACCGAG |
| | | CAGGATTCCAAGGACTCCACCTACTCCCTGTCCAGCACACTCACACTGAG |
| | | CAAAGCCGACTACGAGAAGCACAAGGTGTACGCCTGCGAGGTGACCCAC |
| | | CAGGGCCTGTCTAGCCCCGTCACCAAGTCCTTCAACCGGGGCGAGTGT |
| 131 | VH region (nt) | CAAGTGCAACTAGTGCAAAGTGGTGCAGAAGTCAAGAAGCCTGGCGCTT |
| | (P8-V1) | CCGTGAAGGTGTCCTGCAAGGCCTCTGGCTATACCTTCACCTCCTACTAC |
| | | ATGCACTGGGTGCGGCAGGCTCCTGGACAAGGCCTGGAGTGGATGGGCA |
| | | TCATCAACCCCAGAGGCGGCTACACCACCTACGCCCAGAAGTTCCAGGG |
| | | CAGAGTGACCATGACCAGAGATACCAGCACCTCTACAGTGTACATGGAA |
| | | CTGTCCTCTCTGCGCTCCGAGGACACCGCCGTGTACTACTGTGCCAAAGA |
| | | GGGACGGGTGTCTCCTACCGACTACTACGGCATGGACGTGTGGGGCCAG |
| | | GGCACCACAGTTACAGTGTCCAGC |
| 132 | VL region (nt) | GATATCCAAATGACTCAAAGTCCAAGTAGTCTCTCTGCTTCCGTGGGCGA |
| | (P8-V1) | CAGAGTGACCATCACCTGTAGAGCCTCTCAGTCCATCAGCAGCTATCTGA |
| | | ACTGGTACCAGCAGAAGCCTGGCAAAGCTCCAAAGCTGCTGATCTACGA |
| | | CGCCTCCAGCCTGCAGTCTGGCGTCCCCTCTCGGTTCTCCGGATCTGGCT |
| | | CCGGCACAGATTTTACCCTGACCATCTCCTCTCTGCAGCCTGAGGACTTC |
| | | GCCACCTACTACTGCCAGCAATCCTACTCCACACCTCTGACCTTCGGCGG |
| | | AGGCACCAAGGTGGAAATCAAG |
| 133 | Heavy chain (nt) | CAAGTGCAACTAGTGCAAAGTGGTGCAGAAGTCAAGAAGCCTGGCGCTT |
| | (P8-V1) | CCGTGAAGGTGTCCTGCAAGGCCTCTGGCTATACCTTCACCTCCTACTAC |
| | | ATGCACTGGGTGCGGCAGGCTCCTGGACAAGGCCTGGAGTGGATGGGCA |
| | | TCATCAACCCCAGAGGCGGCTACACCACCTACGCCCAGAAGTTCCAGGG |
| | | CAGAGTGACCATGACCAGAGATACCAGCACCTCTACAGTGTACATGGAA |
| | | CTGTCCTCTCTGCGCTCCGAGGACACCGCCGTGTACTACTGTGCCAAAGA |
| | | GGGACGGGTGTCTCCTACCGACTACTACGGCATGGACGTGTGGGGCCAG |
| | | GGCACCACAGTTACAGTGTCCAGCGCTAGCACCAAGGGCCCATCGGTCT |
| | | TCCCCCTGGCACCCTCCTCCAAGAGCACCTCTGGGGGCACAGCGGCCCT |
| | | GGGCTGCCTGGTCAAGGACTACTTCCCCGAACCGGTGACGGTGTCGTGG |
| | | AACTCAGGCGCCCTGACCAGCGGCGTGCACACCTTCCCGGCTGTCCTAC |
| | | AGTCCTCAGGACTCTACTCCCTCAGCAGCGTGGTGACCGTGCCCTCCAGC |
| | | AGCTTGGGCACCCAGACCTACATCTGCAACGTGAATCACAAGCCCAGCA |
| | | ACACCAAGGTGGACAAGAGAGTTGAGCCCAAATCTTGTGACAAAACTCA |
| | | CACATGCCCACCGTGCCCAGCACCTGAAGCCGAAGGGGCCCCGTCAGTC |
| | | TTCCTCTTCCCCCCAAAACCCAAGGACACCCTCATGATCTCCCGGACCCC |
| | | TGAGGTCACATGCGTGGTGGTGGACGTGAGCCACGAAGACCCTGAGGTC |
| | | AAGTTCAACTGGTACGTGGACGGCGTGGAGGTGCATAATGCCAAGACAA |
| | | AGCCGCGGGAGGAGCAGTACAACAGCACGTACCGTGTGGTCAGCGTCCT |
| | | CACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAG |
| | | CCAAAGGGCAGCCCCGAGAACCACAGGTGTACACCCTGCCCCCATCCCG |
| | | GGAGGAGATGACCAAGAACCAGGTCAGCCTGACCTGCCTGGTCAAAGGC |
| | | TTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTT |
| | | CTTCCTCTATAGCAAGCTCACCGTGGACAAGAGCAGGTGGCAGCAGGGG |
| | | AACGTCTTCTCATGCTCCGTGATGCATGAGGCTCTGCACAACCACTACAC |
| | | GCAGAAGAGCCTCTCCCTGTCCCCGGGT |
| 134 | Light chain (nt) | GATATCCAAATGACTCAAAGTCCAAGTAGTCTCTCTGCTTCCGTGGGCGA |
| | (P8-V1) | CAGAGTGACCATCACCTGTAGAGCCTCTCAGTCCATCAGCAGCTATCTGA |
| | | ACTGGTACCAGCAGAAGCCTGGCAAAGCTCCAAAGCTGCTGATCTACGA |
| | | CGCCTCCAGCCTGCAGTCTGGCGTCCCCTCTCGGTTCTCCGGATCTGGCT |
| | | CCGGCACAGATTTTACCCTGACCATCTCCTCTCTGCAGCCTGAGGACTTC |
| | | GCCACCTACTACTGCCAGCAATCCTACTCCACACCTCTGACCTTCGGCGG |
| | | AGGCACCAAGGTGGAAATCAAGCGTACGGTGGCTGCACCATCTGTCTTC |
| | | ATCTTCCCGCCATCTGATGAGCAGTTGAAATCTGGAACTGCCTCTGTTGT |
| | | GTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAG |
| | | CAAAGCAGACTACGAGAAACACAAAGTCTACGCCTGCGAAGTCACCCAT |
| | | CAGGGCCTGAGCTCGCCCGTCACAAAGAGCTTCAACAGGGGAGAGTGT |
| 135 | VH region (nt) | CAAGTGCAACTAGTGCAAAGTGGTGCAGAAGTCAAGAAGCCTGGCGCTT |
| | (P20) | CCGTGAAGGTGTCCTGCAAGGCCTCTGGCTATACCTTCACCTCCTACTAC |
| | | ATGCACTGGGTGCGGCAGGCTCCTGGACAAGGCCTGGAGTGGATGGGCA |
| | | TCATCAACCCCAGAGGCGGCTACACCACCTACGCCCAGAAGTTCCAGGG |
| | | CAGAGTGACCATGACCAGAGATACCAGCACCTCTACAGTGTACATGGAA |
| | | CTGTCCTCTCTGCGCTCCGAGGACACCGCCGTGTACTACTGTGCCAAAGA |
| | | GGGACGGGTGTCTCCTACCGACTACTACGGCATGGACGTGTGGGGCCAG |
| | | GGCACCACAGTTACAGTGTCCAGC |
| 136 | VL region (nt) | GCTATTCAACTTACCCAGTCCCCGTCCTCCCTGAGCGCCTCAGTGGGGGA |
| | (P20) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGA |
| | | CGCGAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAG |
| 137 | Heavy chain (nt) | CAAGTCCAACTTCAGCAGAGCGGACCGGGACTGGTCAAGCCCTCCGAGA |
| | (P20) | CTCTGTCCCTCACTTGCACCGTGTCCGGAGGTTCTATCAACAACTACTAC |
| | | TGGTCCTGGATTCGGCAGCCTCCTGGGAAGGGCCTCGAGTGGATCGGCT |
| | | ACATCTTCTACTCCGGCACTACCAATTACAACCCGTCGCTGAAGTCCCGC |
| | | GTGACCATTTCCGTGGACACTTCGAAGAACCAGTTCAGCCTGAAGCTCTC |
| | | CAGCGTGACCGCTGCCGATACCGCCGTCTACTACTGCGCTCGCGATGTCG |
| | | GAATCGCGGCACGGAACTCATATTACTACGTGATGGACGTCTGGGGACA |
| | | GGGAACCACCGTGACCGTCAGCAGCGCAAGCACCAAGGGTCCGAGCGT |
| | | GTTCCCTCTGGCCCCATCCTCGAAGTCAACTTCCGGTGGAACTGCCGCCC |
| | | TGGGATGCTTGGTCAAGGACTACTTCCCGGAACCCGTGACCGTGTCATG |
| | | GAATTCGGGGGCCCTTACCTCGGGCGTGCATACCTTCCCCGCGGTGTTGC |
| | | AGTCCAGCGGCCTCTATTCCCTCTCATCCGTCGTGACTGTGCCGAGTTCC |
| | | TCGCTGGGCACCCAGACCTACATCTGCAACGTGAACCATAAGCCCTCCA |
| | | ACACCAAGGTCGACAAACGGGTGGAGCCTAAGTCCTGCGACAAGACGC |
| | | ACACTTGCCCACCTTGTCCCGCTCCTGAAGCCGAGGGAGCGCCTTCAGTG |
| | | TTCCTGTTTCCCCCGAAACCCAAGGATACCCTGATGATCTCCCGGACCCC |
| | | CGAAGTGACCTGTGTGGTGGTGGATGTCAGCCACGAAGATCCGGAAGTC |
| | | AAGTTCAATTGGTACGTGGACGGCGTGGAAGTGCACAACGCTAAGACTA |
| | | AGCCGCGCGAGGAACAGTACAACTCGACTTACCGCGTCGTGTCCGTGCT |
| | | GACTGTGCTGCATCAGGACTGGCTGAACGGAAAAGAGTACAAGTGCAAA |
| | | GTGTCCAACAAGGCACTGCCTGCGCCAATTGAAAAGACCATCAGCAAGG |
| | | CCAAGGGACAGCCCAGGGAACCTCAAGTGTACACGCTCCCGCCGAGCCG |
| | | GGAAGAAATGACCAAGAACCAAGTGTCACTGACATGCCTCGTGAAGGG |
| | | ATTCTACCCCTCGGACATTGCCGTGGAGTGGGAGTCCAACGGGCAGCCG |
| | | GAGAACAACTATAAGACCACCCCACCGGTATTGGACTCGGACGGCTCCT |
| | | TCTTCCTGTACTCGAAGCTGACTGTGGACAAGTCCAGATGGCAACAGGG |
| | | GAACGTTTTCAGCTGCTCCGTGATGCACGAAGCGCTGCACAACCACTAC |
| | | ACCCAGAAGTCCCTGTCTCTGTCCCCCGGC |
| 138 | Light chain (nt) | GCTATTCAACTTACCCAGTCCCCGTCCTCCCTGAGCGCCTCAGTGGGGGA |
| | (P20) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGA |
| | | CGCGAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAGCGGACTGTGGCGGCACCTAGCGTGTT |
| | | CATCTTCCCTCCATCGGATGAACAGCTGAAGTCCGGAACTGCCAGCGTC |
| | | GTGTGCCTGCTGAACAATTTCTACCCCCGCGAGGCTAAGGTCCAGTGGA |
| | | AGGTCGACAACGCGCTGCAGTCCGGAAACTCCCAGGAATCCGTGACCGA |
| | | ACAGGACTCGAAGGACTCCACTTACTCGCTGAGCTCCACCCTGACGCTTT |
| | | CGAAGGCAGACTACGAGAAGCATAAAGTGTACGCCTGCGAAGTGACCC |
| | | ACCAGGGCCTGAGCTCCCCTGTGACCAAGTCCTTCAACCGCGGGGAGTG |
| | | C |
| 139 | VH region (nt) | CAGGTGCAGCTGCAAGAAAGCGGACCGGGACTGGTCAAGCCCTCCGAG |
| | (P20-V2) | ACTCTGTCCCTCACTTGCACCGTGTCCGGAGGTTCTATCAACAACTACTA |
| | | CTGGTCCTGGATTCGGCAGCCTCCTGGGAAGGGCCTCGAGTGGATCGGC |
| | | TACATCTTCTACTCCGGCACTACCAATTACAACCCGTCGCTGAAGTCCCG |
| | | CGTGACCATTTCCGTGGACACTTCGAAGAACCAGTTCAGCCTGAAGCTCT |
| | | CCAGCGTGACCGCTGCCGATACCGCCGTCTACTACTGCGCTCGCGATGTC |
| | | GGAATCGCGGCACGGAACTCATATTACTACGTGATGGACGTCTGGGGAC |
| | | AGGGAACCACCGTGACCGTCAGCAGC |
| 140 | VL region (nt) | GCTATTCAACTTACCCAGTCCCCGTCCTCCCTGAGCGCCTCAGTGGGGGA |
| | (P20-V2) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGA |
| | | AGGCAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAG |
| 141 | Heavy chain (nt) | CAGGTGCAGCTGCAAGAAAGCGGACCGGGACTGGTCAAGCCCTCCGAG |
| | (P20-V2) | ACTCTGTCCCTCACTTGCACCGTGTCCGGAGGTTCTATCAACAACTACTA |
| | | CTGGTCCTGGATTCGGCAGCCTCCTGGGAAGGGCCTCGAGTGGATCGGC |
| | | TACATCTTCTACTCCGGCACTACCAATTACAACCCGTCGCTGAAGTCCCG |
| | | CGTGACCATTTCCGTGGACACTTCGAAGAACCAGTTCAGCCTGAAGCTCT |
| | | CCAGCGTGACCGCTGCCGATACCGCCGTCTACTACTGCGCTCGCGATGTC |
| | | GGAATCGCGGCACGGAACTCATATTACTACGTGATGGACGTCTGGGGAC |
| | | AGGGAACCACCGTGACCGTCAGCAGCGCAAGCACCAAGGGTCCGAGCG |
| | | TGTTCCCTCTGGCCCCATCCTCGAAGTCAACTTCCGGTGGAACTGCCGCC |
| | | CTGGGATGCTTGGTCAAGGACTACTTCCCGGAACCCGTGACCGTGTCATG |
| | | GAATTCGGGGGCCCTTACCTCGGGCGTGCATACCTTCCCCGCGGTGTTGC |
| | | AGTCCAGCGGCCTCTATTCCCTCTCATCCGTCGTGACTGTGCCGAGTTCC |
| | | TCGCTGGGCACCCAGACCTACATCTGCAACGTGAACCATAAGCCCTCCA |
| | | ACACCAAGGTCGACAAACGGGTGGAGCCTAAGTCCTGCGACAAGACGC |
| | | ACACTTGCCCACCTTGTCCCGCTCCTGAAGCCGAGGGAGCGCCTTCAGTG |
| | | TTCCTGTTTCCCCCGAAACCCAAGGATACCCTGATGATCTCCCGGACCCC |
| | | CGAAGTGACCTGTGTGGTGGTGGATGTCAGCCACGAAGATCCGGAAGTC |
| | | AAGTTCAATTGGTACGTGGACGGCGTGGAAGTGCACAACGCTAAGACTA |
| | | AGCCGCGCGAGGAACAGTACAACTCGACTTACCGCGTCGTGTCCGTGCT |
| | | GACTGTGCTGCATCAGGACTGGCTGAACGGAAAAGAGTACAAGTGCAAA |
| | | GTGTCCAACAAGGCACTGCCTGCGCCAATTGAAAAGACCATCAGCAAGG |
| | | CCAAGGGACAGCCCAGGGAACCTCAAGTGTACACGCTCCCGCCGAGCCG |
| | | GGAAGAAATGACCAAGAACCAAGTGTCACTGACATGCCTCGTGAAGGG |
| | | ATTCTACCCCTCGGACATTGCCGTGGAGTGGGAGTCCAACGGGCAGCCG |
| | | GAGAACAACTATAAGACCACCCCACCGGTATTGGACTCGGACGGCTCCT |
| | | TCTTCCTGTACTCGAAGCTGACTGTGGACAAGTCCAGATGGCAACAGGG |
| | | GAACGTTTTCAGCTGCTCCGTGATGCACGAAGCGCTGCACAACCACTAC |
| | | ACCCAGAAGTCCCTGTCTCTGTCCCCCGGC |
| 142 | Light chain (nt) | GCTATTCAACTTACCCAGTCCCCGTCCTCCCTGAGCGCCTCAGTGGGGGA |
| | (P20-V2) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGA |
| | | AGGCAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAGCGTACGGTGGCTGCACCATCTGTCTT |
| | | CATCTTCCCGCCATCTGATGAGCAGTTGAAATCTGGAACTGCCTCTGTTG |
| | | TGTGCCTGCTGAATAACTTCTATCCCAGAGAGGCCAAAGTACAGTGGAA |
| | | GGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGA |
| | | GCAAAGCAGACTACGAGAAACACAAAGTCTACGCCTGCGAAGTCACCCA |
| | | TCAGGGCCTGAGCTCGCCCGTCACAAAGAGCTTCAACAGGGGAGAGTGT |
| 143 | VH region (nt) | CAGGTGCAGCTGCAAGAAAGCGGACCGGGACTGGTCAAGCCCTCCGAG |
| | (P20-V3) | ACTCTGTCCCTCACTTGCACCGTGTCCGGAGGTTCTATCAACAACTACTA |
| | | CTGGTCCTGGATTCGGCAGCCTCCTGGGAAGGGCCTCGAGTGGATCGGC |
| | | TACATCTTCTACTCCGGCACTACCAATTACAACCCGTCGCTGAAGTCCCG |
| | | CGTGACCATTTCCGTGGACACTTCGAAGAACCAGTTCAGCCTGAAGCTCT |
| | | CCAGCGTGACCGCTGCCGATACCGCCGTCTACTACTGCGCTCGCGATGTC |
| | | GGAATCGCGGCACGGAACTCATATTACTACGTGATGGACGTCTGGGGAC |
| | | AGGGAACCACCGTGACCGTCAGCAGC |
| 144 | VL region (nt) | GCTATTCAACTTACCCAGTCCCCGTCCTCCCTGAGCGCCTCAGTGGGGGA |
| | (P20-V3) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGG |
| | | CGCGAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAG |
| 145 | Heavy chain (nt) | CAGGTGCAGCTGCAAGAAAGCGGACCGGGACTGGTCAAGCCCTCCGAG |
| | (P20-V3) | ACTCTGTCCCTCACTTGCACCGTGTCCGGAGGTTCTATCAACAACTACTA |
| | | CTGGTCCTGGATTCGGCAGCCTCCTGGGAAGGGCCTCGAGTGGATCGGC |
| | | TACATCTTCTACTCCGGCACTACCAATTACAACCCGTCGCTGAAGTCCCG |
| | | CGTGACCATTTCCGTGGACACTTCGAAGAACCAGTTCAGCCTGAAGCTCT |
| | | CCAGCGTGACCGCTGCCGATACCGCCGTCTACTACTGCGCTCGCGATGTC |
| | | GGAATCGCGGCACGGAACTCATATTACTACGTGATGGACGTCTGGGGAC |
| | | AGGGAACCACCGTGACCGTCAGCAGCGCAAGCACCAAGGGTCCGAGCG |
| | | TGTTCCCTCTGGCCCCATCCTCGAAGTCAACTTCCGGTGGAACTGCCGCC |
| | | CTGGGATGCTTGGTCAAGGACTACTTCCCGGAACCCGTGACCGTGTCATG |
| | | GAATTCGGGGGCCCTTACCTCGGGCGTGCATACCTTCCCCGCGGTGTTGC |
| | | AGTCCAGCGGCCTCTATTCCCTCTCATCCGTCGTGACTGTGCCGAGTTCC |
| | | TCGCTGGGCACCCAGACCTACATCTGCAACGTGAACCATAAGCCCTCCA |
| | | ACACCAAGGTCGACAAACGGGTGGAGCCTAAGTCCTGCGACAAGACGC |
| | | ACACTTGCCCACCTTGTCCCGCTCCTGAAGCCGAGGGAGCGCCTTCAGTG |
| | | TTCCTGTTTCCCCCGAAACCCAAGGATACCCTGATGATCTCCCGGACCCC |
| | | CGAAGTGACCTGTGTGGTGGTGGATGTCAGCCACGAAGATCCGGAAGTC |
| | | AAGTTCAATTGGTACGTGGACGGCGTGGAAGTGCACAACGCTAAGACTA |
| | | AGCCGCGCGAGGAACAGTACAACTCGACTTACCGCGTCGTGTCCGTGCT |
| | | GACTGTGCTGCATCAGGACTGGCTGAACGGAAAAGAGTACAAGTGCAAA |
| | | GTGTCCAACAAGGCACTGCCTGCGCCAATTGAAAAGACCATCAGCAAGG |
| | | CCAAGGGACAGCCCAGGGAACCTCAAGTGTACACGCTCCCGCCGAGCCG |
| | | GGAAGAAATGACCAAGAACCAAGTGTCACTGACATGCCTCGTGAAGGG |
| | | ATTCTACCCCTCGGACATTGCCGTGGAGTGGGAGTCCAACGGGCAGCCG |
| | | GAGAACAACTATAAGACCACCCCACCGGTATTGGACTCGGACGGCTCCT |
| | | TCTTCCTGTACTCGAAGCTGACTGTGGACAAGTCCAGATGGCAACAGGG |
| | | GAACGTTTTCAGCTGCTCCGTGATGCACGAAGCGCTGCACAACCACTAC |
| | | ACCCAGAAGTCCCTGTCTCTGTCCCCCGGC |
| 146 | Light chain (nt) | GCTATTCAACTTACCCAGTCCCCGTCCTCCCTGAGCGCCTCAGTGGGGGA |
| | (P20-V3) | TCGGGTCACCATCACTTGTCGGGCCTCACAAGGCATCTCCTCGGCCCTCG |
| | | CTTGGTATCAGCAGAAACCCGGAAAGGCCCCCAAGTTCCTGATCTACGG |
| | | CGCGAGCAGCCTGGAGTCGGGAGTGCCGTCAAGGTTCAGCGGATCCGGC |
| | | TCTGGAACCGACTTCACCCTGACTATTTCCTCACTGCAACCGGAAGATTT |
| | | CGCCACCTACTACTGCCAGCAATTCAACTCCTACCCGATTACCTTTGGCC |
| | | AGGGTACACGCCTCGAGATCAAGCGTACGGTGGCTGCACCATCTGTCTT |
| | | CATCTTCCCGCCATCTGATGAGCAGTTGAAATCTGGAACTGCCTCTGTTG |
| | | TGTGCCTGCTGAATAACTTCTATCCCAGAGAGGCCAAAGTACAGTGGAA |
| | | GGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGA |
| | | GCAAAGCAGACTACGAGAAACACAAAGTCTACGCCTGCGAAGTCACCCA |
| | | TCAGGGCCTGAGCTCGCCCGTCACAAAGAGCTTCAACAGGGGAGAGTGT |
| 147 | VH region (nt) | CAAGTACAACTCGTGCAGTCCGGAGCGGAAGTGAAAAAGCCTGGAGCGT |
| | (P8) | CAGTGCGCGTCAGTTGCAAAGCTAGCGGCTACACTTTCACCTCCTACTAC |
| | | ATGCACTGGGTCCGCCAAGCACCGGGACAGGGCTTGGAGTGGATGGGGA |
| | | TCATTAACCCACGGGGCGGATACACCACTTATGCTCAGAAGTTCCAGGG |
| | | CCGCGTGACTATGACTAGGGACACCAGCACTTCCACCGTGTACATGGAA |
| | | CTGTCGTCGCTGCGGTCGGAAGATACTGCCGTCTACTACTGCGCCAAGG |
| | | AAGGCAGAGTGTCCCCCACCGACTACTACGGAATGGACGTCTGGGGACA |
| | | GGGCACCACCGTCACCGTGTCCTCG |
| 148 | VL region (nt) | GATATTAGAATGACCCAATCCCCCTCCTCCCTGTCGGCGTCAGTGGGGGA |
| | (P8) | TCGCGTGACCATCACTTGCCGGGCGTCACAAAGCATTTCCTCCTATCTCA |
| | | ACTGGTACCAGCAGAAGCCCGGAAAGGCCCCTAAGCTCCTGATCTACGA |
| | | CGCCTCAAGCTTGCAATCTGGAGTGCCGAGCCGCTTCTCCGGATCCGGCT |
| | | CTGGCACTGACTTCACCCTGACCATGAGCAGCCTGCAGCCCGAGGACTT |
| | | CGCCACTTACTACTGTCAGCAGTCGTACTCAACGCCACTCACTTTCGGCG |
| | | GCGGAACCAAGGTCGAGATCAAG |
| 149 | Heavy chain (nt) | CAAGTACAACTCGTGCAGTCCGGAGCGGAAGTGAAAAAGCCTGGAGCGT |
| | (P8) | CAGTGCGCGTCAGTTGCAAAGCTAGCGGCTACACTTTCACCTCCTACTAC |
| | | ATGCACTGGGTCCGCCAAGCACCGGGACAGGGCTTGGAGTGGATGGGGA |
| | | TCATTAACCCACGGGGCGGATACACCACTTATGCTCAGAAGTTCCAGGG |
| | | CCGCGTGACTATGACTAGGGACACCAGCACTTCCACCGTGTACATGGAA |
| | | CTGTCGTCGCTGCGGTCGGAAGATACTGCCGTCTACTACTGCGCCAAGG |
| | | AAGGCAGAGTGTCCCCCACCGACTACTACGGAATGGACGTCTGGGGACA |
| | | GGGCACCACCGTCACCGTGTCCTCGGCGTCCACCAAGGGCCCCAGCGTG |
| | | TTTCCTCTGGCCCCGTCATCCAAGTCCACCTCGGGCGGAACTGCTGCCCT |
| | | GGGATGCCTGGTCAAGGACTACTTCCCGGAACCCGTGACTGTGTCCTGG |
| | | AACTCGGGAGCGCTCACCAGCGGTGTTCACACCTTCCCCGCGGTGCTTCA |
| | | GAGCTCTGGGCTCTATAGCCTGAGCAGCGTCGTGACCGTGCCGAGCTCC |
| | | AGCCTGGGCACCCAAACCTACATCTGCAACGTGAACCATAAGCCCTCGA |
| | | ACACCAAGGTCGATAAGCGCGTGGAGCCCAAGTCCTGTGACAAGACCCA |
| | | CACGTGTCCTCCTTGCCCTGCACCGGAAGCCGAGGGTGCTCCATCCGTGT |
| | | TCCTCTTTCCACCGAAGCCCAAAGATACTCTCATGATCTCCCGGACCCCC |
| | | GAAGTCACTTGCGTGGTGGTGGATGTGTCCCACGAGGATCCCGAAGTCA |
| | | AGTTCAATTGGTACGTGGACGGTGTCGAGGTGCACAATGCCAAGACTAA |
| | | GCCCAGGGAAGAACAGTACAACAGCACCTACCGGGTGGTGTCCGTGCTG |
| | | ACGGTGCTGCATCAGGACTGGCTGAACGGGAAGGAGTACAAGTGCAAA |
| | | GTGTCCAACAAGGCCCTGCCTGCACCTATCGAGAAAACAATCTCCAAGG |
| | | CCAAGGGACAGCCGCGGGAACCTCAGGTCTACACCCTGCCACCATCCCG |
| | | GGAAGAGATGACTAAGAACCAAGTGTCCCTGACTTGTCTGGTCAAGGGA |
| | | TTCTACCCGTCCGACATCGCCGTGGAGTGGGAGTCCAATGGGCAGCCGG |
| | | AGAACAACTACAAGACCACACCTCCTGTGCTCGACTCCGACGGCTCGTT |
| | | CTTCCTGTACTCGAAGCTCACTGTGGACAAGTCACGATGGCAGCAGGGA |
| | | AACGTGTTCTCCTGCTCGGTCATGCACGAAGCCCTGCACAACCACTATAC |
| | | CCAGAAGTCACTGTCACTGTCGCCGGGA |
| 150 | Light chain (nt) | GATATTAGAATGACCCAATCCCCCTCCTCCCTGTCGGCGTCAGTGGGGGA |
| | (P8) | TCGCGTGACCATCACTTGCCGGGCGTCACAAAGCATTTCCTCCTATCTCA |
| | | ACTGGTACCAGCAGAAGCCCGGAAAGGCCCCTAAGCTCCTGATCTACGA |
| | | CGCCTCAAGCTTGCAATCTGGAGTGCCGAGCCGCTTCTCCGGATCCGGCT |
| | | CTGGCACTGACTTCACCCTGACCATGAGCAGCCTGCAGCCCGAGGACTT |
| | | CGCCACTTACTACTGTCAGCAGTCGTACTCAACGCCACTCACTTTCGGCG |
| | | GCGGAACCAAGGTCGAGATCAAGCGGACCGTGGCAGCTCCGTCCGTGTT |
| | | TATCTTCCCTCCGTCGGACGAGCAATTGAAGTCCGGTACCGCGTCCGTCG |
| | | TGTGCCTGCTGAACAACTTCTACCCGAGGGAAGCCAAAGTGCAGTGGAA |
| | | AGTCGACAATGCACTGCAGTCCGGGAACAGCCAGGAATCCGTGACCGAA |
| | | CAGGACTCGAAGGATAGCACCTACTCCCTGTCCTCCACTCTGACCCTGTC |
| | | GAAGGCCGACTACGAGAAGCATAAGGTCTACGCTTGCGAAGTGACACAC |
| | | CAGGGACTGAGCTCCCCTGTGACCAAGTCGTTCAACCGGGGCGAATGT |
| 151 | VH region (nt) | CAAGTGCAACTGCAGCAGTCAGGCCCTGGCCTCGTAAAGCCTTCGGAGA |
| | (P21) | CTCTGTCCCTGACGTGTACCGTCAGCGGGGGATCCATCTCCAACTACTAC |
| | | TGGACCTGGATCAGGCAGCCGCCTGGAAAGGGTCTGGAGTGGATCGGAT |
| | | ACATCAGCGACTCCGGGTCCACCAATTACAACCCCTCGCTGAAGTCCCG |
| | | GGTCACCATTTCCGTGGACACCTCAAAGAACCAGTTCAGCCTGAAGCTG |
| | | AGCTCCGTTACCGCGGCTGACACAGCCGTGTACTACTGCGCCCGGGAAA |
| | | CCATCGTCGTGCGGGGATTCTACTTCTACGTGATGGACGTCTGGGGCCAG |
| | | GGAACCACTGTGACTGTGTCCTCC |
| 152 | VL region (nt) | GCCATTCAACTCACACAGTCCCCGTCCTCTCTGAGCGCGTCCGTGGGTGA |
| | (P21) | TCGCGTGACCATTACTTGCCGCGCCTCACAAGGCATCAGCTCGGCGCTCG |
| | | CTTGGTACCAGCAGCGGCCTGGAAAGGCCCCTAAGCTGCTGATCTATGA |
| | | CGCCTCCAACTGGGAGTCAGGGGTGCCTTCGAGGTTCTCCGGAAGCGGA |
| | | TCCGGAACTGACTTCACGCTCACCATCTCCTCGCTGCAACCCGAAGATTT |
| | | CGCCACCTACTACTGCCAACAGTTCAACTCCTACCCCTGGACTTTCGGAC |
| | | AAGGCACCAAGGTCGAGATTAAG |
| 153 | Heavy chain (nt) | CAAGTGCAACTGCAGCAGTCAGGCCCTGGCCTCGTAAAGCCTTCGGAGA |
| | (P21) | CTCTGTCCCTGACGTGTACCGTCAGCGGGGGATCCATCTCCAACTACTAC |
| | | TGGACCTGGATCAGGCAGCCGCCTGGAAAGGGTCTGGAGTGGATCGGAT |
| | | ACATCAGCGACTCCGGGTCCACCAATTACAACCCCTCGCTGAAGTCCCG |
| | | GGTCACCATTTCCGTGGACACCTCAAAGAACCAGTTCAGCCTGAAGCTG |
| | | AGCTCCGTTACCGCGGCTGACACAGCCGTGTACTACTGCGCCCGGGAAA |
| | | CCATCGTCGTGCGGGGATTCTACTTCTACGTGATGGACGTCTGGGGCCAG |
| | | GGAACCACTGTGACTGTGTCCTCCGCATCAACTAAGGGGCCGTCAGTGTT |
| | | TCCTCTTGCCCCGTCCTCCAAGTCGACTTCTGGAGGAACCGCTGCCCTTG |
| | | GCTGCCTGGTCAAAGACTATTTCCCGGAACCCGTCACGGTGTCCTGGAAC |
| | | AGTGGAGCGCTGACCAGCGGTGTCCACACCTTCCCGGCTGTGCTCCAGTC |
| | | CTCCGGACTGTACAGCCTGAGCTCGGTCGTGACTGTGCCGTCCTCGTCCC |
| | | TGGGCACCCAGACATACATCTGCAACGTGAACCATAAGCCCTCGAACAC |
| | | CAAGGTCGATAAGAGAGTGGAGCCGAAGTCGTGCGATAAGACTCACACT |
| | | TGCCCACCGTGTCCAGCGCCAGAAGCGGAAGGGGCACCTAGCGTGTTCC |
| | | TGTTCCCGCCCAAGCCGAAGGACACCCTCATGATTAGCCGGACCCCCGA |
| | | AGTCACCTGTGTGGTGGTCGACGTGTCCCACGAAGATCCCGAAGTCAAG |
| | | TTCAATTGGTACGTGGACGGCGTGGAAGTGCACAACGCCAAGACCAAGC |
| | | CGCGCGAGGAACAGTACAACTCCACCTACCGCGTGGTGTCCGTGCTCAC |
| | | CGTGTTGCATCAGGATTGGCTGAACGGGAAGGAGTACAAGTGCAAAGTG |
| | | TCCAACAAAGCACTGCCTGCCCCCATTGAGAAAACCATTAGCAAGGCCA |
| | | AGGGACAGCCTCGGGAGCCACAAGTGTACACTCTGCCCCCATCGAGAGA |
| | | AGAAATGACCAAGAATCAAGTGTCACTGACGTGCCTCGTGAAGGGATTT |
| | | TACCCCTCGGACATCGCCGTGGAGTGGGAGTCGAACGGCCAGCCCGAGA |
| | | ACAACTACAAGACTACCCCGCCTGTGTTGGATAGCGACGGCTCGTTCTTC |
| | | CTGTATTCCAAGCTCACTGTGGACAAGTCACGCTGGCAACAGGGCAACG |
| | | TGTTCTCCTGCTCCGTGATGCACGAAGCCCTGCATAACCACTACACCCAG |
| | | AAGTCCCTGTCTCTGAGCCCTGGT |
| 154 | Light chain (nt) | GCCATTCAACTCACACAGTCCCCGTCCTCTCTGAGCGCGTCCGTGGGTGA |
| | (P21) | TCGCGTGACCATTACTTGCCGCGCCTCACAAGGCATCAGCTCGGCGCTCG |
| | | CTTGGTACCAGCAGCGGCCTGGAAAGGCCCCTAAGCTGCTGATCTATGA |
| | | CGCCTCCAACTGGGAGTCAGGGGTGCCTTCGAGGTTCTCCGGAAGCGGA |
| | | TCCGGAACTGACTTCACGCTCACCATCTCCTCGCTGCAACCCGAAGATTT |
| | | CGCCACCTACTACTGCCAACAGTTCAACTCCTACCCCTGGACTTTCGGAC |
| | | AAGGCACCAAGGTCGAGATTAAGCGGACCGTGGCTGCACCGTCCGTGTT |
| | | CATCTTCCCACCCTCCGACGAGCAGCTCAAGAGCGGCACCGCAAGCGTG |
| | | GTCTGCCTGCTGAACAATTTCTACCCGCGCGAAGCGAAAGTGCAGTGGA |
| | | AGGTCGATAACGCGCTGCAGTCAGGAAACTCACAGGAATCCGTGACTGA |
| | | ACAGGACTCCAAGGACAGCACCTACTCGCTGTCCTCCACCTTGACTCTGT |
| | | CGAAAGCCGACTACGAGAAGCACAAGGTCTACGCCTGTGAAGTGACCCA |
| | | TCAGGGTCTGTCGAGCCCAGTGACCAAGTCCTTCAACCGGGGCGAATGC |
| 155 | VH region (nt) | CAGGTGCAGCTGCAGGAGTCAGGCCCTGGCCTCGTAAAGCCTTCGGAGA |
| | (P21-V1) | CTCTGTCCCTGACGTGTACCGTCAGCGGGGGATCCATCTCCAACTACTAC |
| | | TGGACCTGGATCAGGCAGCCGCCTGGAAAGGGTCTGGAGTGGATCGGAT |
| | | ACATCAGCGACTCCGGGTCCACCAATTACAACCCCTCGCTGAAGTCCCG |
| | | GGTCACCATTTCCGTGGACACCTCAAAGAACCAGTTCAGCCTGAAGCTG |
| | | AGCTCCGTTACCGCGGCTGACACAGCCGTGTACTACTGCGCCCGGGAAA |
| | | CCATCGTCGTGCGGGGATTCTACTTCTACGTGATGGACGTCTGGGGCCAG |
| | | GGAACCACTGTGACTGTGTCCTCC |
| 156 | VL region (nt) | GCCATTCAACTCACACAGTCCCCGTCCTCTCTGAGCGCGTCCGTGGGTGA |
| | (P21-V1) | TCGCGTGACCATTACTTGCCGCGCCTCACAAGGCATCAGCTCGGCGCTCG |
| | | CTTGGTACCAGCAGAAGCCTGGAAAGGCCCCTAAGCTGCTGATCTATGA |
| | | AGCCTCCAACTGGGAGTCAGGGGTGCCTTCGAGGTTCTCCGGAAGCGGA |
| | | TCCGGAACTGACTTCACGCTCACCATCTCCTCGCTGCAACCCGAAGATTT |
| | | CGCCACCTACTACTGCCAACAGTTCAACTCCTACCCCTGGACTTTCGGAC |
| | | AAGGCACCAAGGTCGAGATTAAG |
| 157 | Heavy chain (nt) | CAGGTGCAGCTGCAGGAGTCAGGCCCTGGCCTCGTAAAGCCTTCGGAGA |
| | (P21-V1) | CTCTGTCCCTGACGTGTACCGTCAGCGGGGGATCCATCTCCAACTACTAC |
| | | TGGACCTGGATCAGGCAGCCGCCTGGAAAGGGTCTGGAGTGGATCGGAT |
| | | ACATCAGCGACTCCGGGTCCACCAATTACAACCCCTCGCTGAAGTCCCG |
| | | GGTCACCATTTCCGTGGACACCTCAAAGAACCAGTTCAGCCTGAAGCTG |
| | | AGCTCCGTTACCGCGGCTGACACAGCCGTGTACTACTGCGCCCGGGAAA |
| | | CCATCGTCGTGCGGGGATTCTACTTCTACGTGATGGACGTCTGGGGCCAG |
| | | GGAACCACTGTGACTGTGTCCTCCGCATCAACTAAGGGGCCGTCAGTGTT |
| | | TCCTCTTGCCCCGTCCTCCAAGTCGACTTCTGGAGGAACCGCTGCCCTTG |
| | | GCTGCCTGGTCAAAGACTATTTCCCGGAACCCGTCACGGTGTCCTGGAAC |
| | | AGTGGAGCGCTGACCAGCGGTGTCCACACCTTCCCGGCTGTGCTCCAGTC |
| | | CTCCGGACTGTACAGCCTGAGCTCGGTCGTGACTGTGCCGTCCTCGTCCC |
| | | TGGGCACCCAGACATACATCTGCAACGTGAACCATAAGCCCTCGAACAC |
| | | CAAGGTCGATAAGAGAGTGGAGCCGAAGTCGTGCGATAAGACTCACACT |
| | | TGCCCACCGTGTCCAGCGCCAGAAGCGGAAGGGGCACCTAGCGTGTTCC |
| | | TGTTCCCGCCCAAGCCGAAGGACACCCTCATGATTAGCCGGACCCCCGA |
| | | AGTCACCTGTGTGGTGGTCGACGTGTCCCACGAAGATCCCGAAGTCAAG |
| | | TTCAATTGGTACGTGGACGGCGTGGAAGTGCACAACGCCAAGACCAAGC |
| | | CGCGCGAGGAACAGTACAACTCCACCTACCGCGTGGTGTCCGTGCTCAC |
| | | CGTGTTGCATCAGGATTGGCTGAACGGGAAGGAGTACAAGTGCAAAGTG |
| | | TCCAACAAAGCACTGCCTGCCCCCATTGAGAAAACCATTAGCAAGGCCA |
| | | AGGGACAGCCTCGGGAGCCACAAGTGTACACTCTGCCCCCATCGAGAGA |
| | | AGAAATGACCAAGAATCAAGTGTCACTGACGTGCCTCGTGAAGGGATTT |
| | | TACCCCTCGGACATCGCCGTGGAGTGGGAGTCGAACGGCCAGCCCGAGA |
| | | ACAACTACAAGACTACCCCGCCTGTGTTGGATAGCGACGGCTCGTTCTTC |
| | | CTGTATTCCAAGCTCACTGTGGACAAGTCACGCTGGCAACAGGGCAACG |
| | | TGTTCTCCTGCTCCGTGATGCACGAAGCCCTGCATAACCACTACACCCAG |
| | | AAGTCCCTGTCTCTGAGCCCTGGT |
| 158 | Light chain (nt) | GCCATTCAACTCACACAGTCCCCGTCCTCTCTGAGCGCGTCCGTGGGTGA |
| | (P21-V1) | TCGCGTGACCATTACTTGCCGCGCCTCACAAGGCATCAGCTCGGCGCTCG |
| | | CTTGGTACCAGCAGAAGCCTGGAAAGGCCCCTAAGCTGCTGATCTATGA |
| | | AGCCTCCAACTGGGAGTCAGGGGTGCCTTCGAGGTTCTCCGGAAGCGGA |
| | | TCCGGAACTGACTTCACGCTCACCATCTCCTCGCTGCAACCCGAAGATTT |
| | | CGCCACCTACTACTGCCAACAGTTCAACTCCTACCCCTGGACTTTCGGAC |
| | | AAGGCACCAAGGTCGAGATTAAGCGCACCGTGGCCGCCCCTAGCGTGTT |
| | | TATCTTCCCTCCCTCGGATGAGCAGCTTAAGTCAGGCACCGCATCCGTGG |
| | | TCTGCCTGCTCAACAACTTCTACCCGAGGGAAGCCAAAGTGCAGTGGAA |
| | | AGTGGACAACGCGCTCCAGTCGGGAAACTCCCAGGAGTCCGTGACCGAA |
| | | CAGGACTCCAAGGACAGCACTTATTCCCTGTCCTCCACTCTGACGCTGTC |
| | | AAAGGCCGACTACGAGAAGCACAAGGTCTACGCCTGCGAAGTGACCCAT |
| | | CAGGGGCTTTCCTCGCCCGTGACTAAGAGCTTCAATCGGGGCGAATGC |
| 159 | heavy chain with | XVQLQESGPGLVKPSETLSLTCTVSGGSINNYYWSWIRQPPGKGLEWIGYI |
| | pyroglutamate | FYSGTTNYNPSLKSRVTISVDTSKNQFSLKLSSVTAADTAVYYCARDVGIA |
| | post-translational | ARNSYYYVMDVWGQGTTVTVSSASTKGPSVFPLAPSSKSTSGGTAALGCL |
| | modification (aa) | VKDYFPEPVTVSWNSGALTSGVHTFPAVLQSSGLYSLSSVVTVPSSSLGTQT |
| | (P20-V1) | YICNVNHKPSNTKVDKRVEPKSCDKTHTCPPCPAPEAEGAPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG, wherein X |
| | | is pyroglutamate |
| 160 | VH region with | XVQLQESGPGLVKPSETLSLTCTVSGGSINNYYWSWIRQPPGKGLEWIGYI |
| | pyroglutamate | FYSGTTNYNPSLKSRVTISVDTSKNQFSLKLSSVTAADTAVYYCARDVGIA |
| | post-translational | ARNSYYYVMDVWGQGTTVTVSS, wherein X is pyroglutamate |
| | modification (aa) | |
| | (P20-V1) | |
| 161 | Burosumab | DIVDAF |
| | heavy chain | |
| | CDR3 (aa) | |
| 162 | heavy chain with | XVQLVQSGAEVKKPGASVKVSCKASGYTFTSYYMHWVRQAPGQGLEWM |
| | pyroglutamate | GIINPRGGYTTYAQKFQGRVTMTRDTSTSTVYMELSSLRSEDTAVYYCAKE |
| | post-translational | GRVSPTDYYGMDVWGQGTTVTVSSASTKGPSVFPLAPSSKSTSGGTAALG |
| | modification (aa) | CLVKDYFPEPVTVSWNSGALTSGVHTFPAVLQSSGLYSLSSVVTVPSSSLGT |
| | (P8-V1) | QTYICNVNHKPSNTKVDKRVEPKSCDKTHTCPPCPAPEAEGAPSVFLFPPKP |
| | | KDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPG, |
| | | wherein X is pyroglutamate |
| 163 | VH region with | XVQLVQSGAEVKKPGASVKVSCKASGYTFTSYYMHWVRQAPGQGLEWM |
| | pyroglutamate | GIINPRGGYTTYAQKFQGRVTMTRDTSTSTVYMELSSLRSEDTAVYYCAKE |
| | post-translational | GRVSPTDYYGMDVWGQGTTVTVSS, wherein X is pyroglutamate |
| | modification (aa) | |
| | (P8-V1) | |

1-3. (canceled)

4. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 34; and
the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 35.

5. The anti-IL-18Rβ antibody or antigen-binding fragment thereof of claim 4, wherein:
the V_{H} region comprises a heavy chain complementarity determining region 1 (CDR-H1) comprising the amino acid sequence of SEQ ID NO: 8, a heavy chain complementarity determining region 2 (CDR-H2) comprising the amino acid sequence of SEQ ID NO: 9, and a heavy chain complementarity determining region 3 (CDR-H3) comprising the amino acid sequence of SEQ ID NO: 10; and
the V_{L} region comprises a light chain complementarity determining region 1 (CDR-L1) comprising the amino acid sequence of SEQ ID NO: 22, a light chain complementarity determining region 2 (CDR-L2) comprising the amino acid sequence of SEQ ID NO: 36, and a light chain complementarity determining region 3 (CDR-L3) comprising the amino acid sequence of SEQ ID NO: 24.

6-14. (canceled)

15. The anti-IL-18Rβ antibody or antigen-binding fragment thereof of claim 5, wherein the V_{H} region comprises an amino acid sequence having at least, or at least about 85% sequence identity to the amino acid sequence of SEQ ID NO: 34; and the V_{L} region comprises an amino acid sequence having at least about 85% sequence identity to the amino acid sequence of SEQ ID NO: 35.

16. The anti-IL-18Rβ antibody or antigen-binding fragment thereof of claim 5, wherein the V_{H} region comprises an amino acid sequence having at least 95% sequence identity to the amino acid sequence of SEQ ID NO: 34; and the V_{L} region comprises an amino acid sequence having at least 95% sequence identity to the amino acid sequence of SEQ ID NO: 35.

17. (canceled)

18. The anti-IL-18Rβ antibody or antigen-binding fragment thereof of claim 5, wherein the V_{H} region comprises the amino acid sequence of SEQ ID NO: 34 and the V_{L} region comprises the amino acid sequence of SEQ ID NO: 35.

19. The anti-IL-18Rβ antibody or antigen-binding fragment thereof of claim 5, wherein the antibody or antigen-binding fragment thereof is an antibody comprising a constant domain, wherein the constant domain is an IgG1 constant domain.

20. (canceled)

21. The anti-IL-18Rβ antibody or antigen-binding fragment thereof of claim 5, wherein the antibody comprises:
a heavy chain constant domain comprising an amino acid sequence having at least about 95% sequence identity to the amino acid sequence of SEQ ID NO: 30; and
a light chain constant domain comprising an amino acid sequence having at least about 95% sequence identity to the amino acid sequence of SEQ ID NO: 31.

22. The anti-IL-18Rβ antibody or antigen-binding fragment thereof of claim 5, wherein the antibody comprises:
a heavy chain constant domain comprising the amino acid sequence of SEQ ID NO: 30; and
a light chain constant domain comprising the amino acid sequence of SEQ ID NO: 31.

23. The anti-IL-18Rβ antibody or antigen-binding fragment thereof of claim 5, wherein the antibody comprises:
a heavy chain comprising an amino acid sequence having at least about 90% sequence identity to the amino acid sequence of SEQ ID NO: 32; and
a light chain comprising an amino acid sequence having at least about 90% sequence identity to the amino acid sequence of SEQ ID NO: 33.

24. The anti-IL-18Rβ antibody or antigen-binding fragment thereof of claim 5, wherein the antibody comprises:
a heavy chain comprising an amino acid sequence having at least 95% sequence identity to the amino acid sequence of SEQ ID NO: 32; and
a light chain comprising an amino acid sequence having at least 95% sequence identity to the amino acid sequence of SEQ ID NO: 33.

25. The anti-IL-18Rβ antibody or antigen-binding fragment thereof of claim 5, wherein the antibody comprises a heavy chain comprising the amino acid sequence of SEQ ID NO: 32, and a light chain comprising the amino acid sequence of SEQ ID NO: 33.

26. (canceled)

27. An anti-interleukin 18 receptor beta (IL-18Rβ) antibody or antigen-binding fragment thereof, the antibody or antigen-binding fragment thereof comprising a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2), and a heavy chain complementarity determining region 3 (CDR-H3) contained within the amino acid sequence of SEQ ID NO: 75; and
the V_{L} region comprises a light chain complementarity determining region 1 (CDR-L1), a light chain complementarity determining region 2 (CDR-L2) and a light chain complementarity determining region 3 (CDR-L3) contained within the amino acid sequence of SEQ ID NO: 76.

28. The anti-IL-18Rβ antibody or antigen-binding fragment thereof of claim 27, wherein:
the V_{H} region comprises a heavy chain complementarity determining region 1 (CDR-H1) comprising the amino acid sequence of SEQ ID NO: 52, a heavy chain complementarity determining region 2 (CDR-H2) comprising the amino acid sequence of SEQ ID NO: 53, and a heavy chain complementarity determining region 3 (CDR-H3) comprising the amino acid sequence of SEQ ID NO: 54; and
the V_{L} region comprises a light chain complementarity determining region 1 (CDR-L1) comprising the amino acid sequence of SEQ ID NO: 66, a light chain complementarity determining region 2 (CDR-L2) comprising an amino acid sequence selected from the group consisting of SEQ ID NOs: 67, and a light chain complementarity determining region 3 (CDR-L3) comprising the amino acid sequence of SEQ ID NO: 68.

29-54. (canceled)

55. The anti-IL-18Rβ antibody or antigen-binding fragment thereof of claim 5, wherein the antibody or antigen-binding fragment thereof binds to cynomolgus IL-18Rβ at pH 7.4 with an equilibrium dissociation constant (K_{D}) that is less than about 2 nM and binds to human IL-18Rβ at pH 7.4 with an equilibrium dissociation constant (K_{D}) that is less than about 2 nM.

56. (canceled)

57. The anti-IL-18Rβ antibody or antigen-binding fragment thereof of claim 5, wherein the antibody or antigen-binding fragment thereof has a K_{D} ratio of K_{D} at pH 5 to K_{D} at pH 7 of about 0.8 to about 1.30.

58-63. (canceled)

64. The anti-IL-18Rβ antibody or antigen-binding fragment thereof of claim 25, comprising at least one post-translational modification of the amino acid sequence, wherein the at least one post-translational modification comprises a post-translational modification of a heavy chain N-terminal glutamine (Q) to a pyroglutamate.

65. A multi-specific antibody, comprising a first antigen-binding domain that binds to IL-18Rβ and a second antigen-binding domain that binds to a second antigen, wherein the first antigen-binding domain comprises the anti-IL-18Rβ antibody or antigen-binding fragment thereof of claim 5.

66. (canceled)

67. A conjugate, comprising the anti-IL-18Rβ antibody or antigen-binding fragment thereof of claim 5, and a heterologous molecule or moiety.

68. (canceled)

69. A nucleic acid(s) encoding the anti-IL-18Rβ antibody or antigen-binding fragment thereof of claim 5, or a heavy chain or a light chain thereof.

70-77. (canceled)

78. A vector, comprising the nucleic acid(s) of claim 69.

79. (canceled)

80. (canceled)

81. A host cell comprising the nucleic acid(s) of claim 69.

82-84. (canceled)

85. A composition comprising the anti-IL-18Rβ antibody or antigen-binding fragment thereof of claim 5.

86. (canceled)

87. A method of treatment, comprising administering the anti-IL-18Rβ antibody or antigen-binding fragment thereof of claim 5, to a subject having a disease or disorder.

88. The method of claim 87, further comprising administering one or more additional therapeutic agents.

89. The method of claim 88, wherein the one or more additional therapeutic agents comprises an aminosalicylate, a corticosteroid, a thiopurine, a methotrexate, a tumor necrosis factor (TNF) inhibitor, an α4β7 integrin inhibitor, a TNF-α inhibitor, an IL-23 inhibitor, a dual IL-12 and IL-23 inhibitor, a tumor necrosis factor-like cytokine 1A (TL1A) inhibitor, an IL-2-CD25 fusion protein, a bispecific anti-TL1A/anti-TNF-α binding protein, an E-type prostanoid receptor 4 (EP4) agonist, a TYK2 inhibitor, a sphingosine-1-phosphate receptor (S1PR) agonist, a Janus kinase (JAK) inhibitor, an interkeukin-1 receptor associated kinase 4 (IRAK-4) inhibitor, a toll-like receptor 7 and 8 (TLR7/8) inhibitor, or an IL-22 inhibitor.

90-94. (canceled)

95. The method of claim 87, wherein the disease or disorder is an immune-mediated disease.

96. The method of claim 87, wherein the disease or disorder is an inflammatory disease.

97. The method of claim 87, wherein the disease or disorder is an inflammatory bowel disease.

98. The method of claim 97, wherein the inflammatory bowel disease is Crohn's disease.

99. The method of claim 97, wherein the inflammatory bowel disease is ulcerative colitis.

100. The method of claim 87, wherein the disease or disorder is chronic obstructive pulmonary disease (COPD).

101-103. (canceled)

104. The method of claim 87, wherein the disease or disorder is inflammatory bowel disease, chronic obstructive pulmonary disease (COPD), type 1 diabetes, type 2 diabetes, liver disease, organ transplant rejection, multiple sclerosis, arthritis, psoriasis, heart disease, macrophage activation syndrome (MAS), adult-onset Still's disease (AOSD), hemophagocytic lymphohistiocytosis (HLH), dry eye disease (DED), cytokine release syndrome (CRS), systemic inflammatory response syndrome SIRS), autoinflammation with infantile enterocolitis (AIFEC), XIAP deficiency, NLRC4 gain-of-function mutation, sarcoidosis, atopic dermatitis, Behçet's disease, systemic lupus erythematosus (SLE), acute coronary syndrome, allergies, amyotrophic lateral sclerosis (ALS), asthma, Celiac disease, or age-related macular degeneration (AMD), osteoporosis, Parkinson's disease, Grave's disease, Hashimoto's thyroiditis, Addison's disease, dermatomyositis, myasthenia gravis, pernicious anemia, Sjogren syndrome, vasculitis, uveitis, sepsis, atherosclerosis, ankylosing spondylitis, acute respiratory syndrome coronavirus (SARS-CoV), or acute respiratory syndrome coronavirus 2 (SARS-CoV-2).

105-127. (canceled)
